(19) United States
(12) Patent Application Publication
(10) Pub. No.: US 2018/0352814 A1
(43) Pub. Date: Dec. 13, 2018

(54) OXADIAZOLE COMPOUNDS AND USE FOR SAME

(71) Applicant: SUMITOMO CHEMICAL COMPANY, LIMITED, Tokyo (JP)

(72) Inventors: Hiroto TAMASHIMA, Takarazuka-shi (JP); Yuya YOSHIMOTO, Takarazuka-shi (JP); Yuki SUGITA, Takarazuka-shi (JP); Masaoki YAMAMOTO, Takarazuka-shi (JP)

(73) Assignee: SUMITOMO CHEMICAL COMPANY, LIMITED, Tokyo (JP)

(21) Appl. No.: 16/062,731

(22) PCT Filed: Dec. 21, 2016
(86) PCT No.: PCT/JP2016/088079

§ 371 Date: Jun. 15, 2018

(30) Foreign Application Priority Data
Dec. 25, 2015 (JP) 2015-253231
May 26, 2016 (JP) 2016-104930

(51) Int. Cl. A01N 47/22; C07D 271/06

(52) U.S. Cl. CPC A01N 47/22; C07D 271/06

(57) ABSTRACT

Disclosed are an oxadiazole compound represented by formula (I):

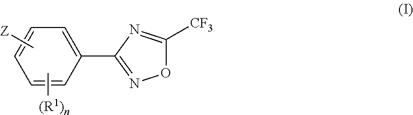

Wherein
Z represents OC(═X)NR²R³ or OC(═X)YR⁴, etc.
R¹ represents a halogen atom or a C1-4 alkoxy group, etc.
n represents an integer of any one of 0 to 4, etc.
R² represents a C1-10 alkyl group having one or more halogen atoms, or a C3-10 cycloalkyl group, etc.
R³ represents a hydrogen atom or a C1-10 alkyl group, etc.
R⁴ represents a C1-10 alkyl group having one or more halogen atoms, or a C3-10 cycloalkyl group, etc., and
X and Y each independently represents an oxygen atom or a sulfur atom, and use thereof for controlling plant diseases.

### TECHNICAL FIELD

The present invention relates to an oxadiazole compound and use for same.

### BACKGROUND ART

Various compounds have hitherto been developed so as control plant diseases (see, for example, Patent Documents 1 and 2).

### PRIOR ART DOCUMENT

### Patent Document

Patent Document 1: JP 63-162680 A

Patent Document 2: WO2015/185485 A

### DISCLOSURE OF THE INVENTION

### Problems to be Solved by the Invention

An object of the present invention is to provide compounds having excellent control activity against plant diseases.

### Means for Solving the Problems

The present inventors have intensively studied so as to find a compound having excellent control activity against plant diseases and found that a compound represented by formula (I) mentioned below has excellent control activity against plant diseases.

The present invention includes the followings.

[1] An oxadiazole compound represented by formula (I):

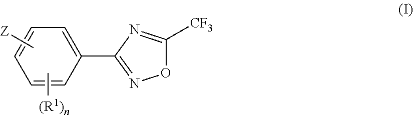

wherein

Z represents OC(═X)NR²R³, OC(═X)YR⁴, OC(═X)R⁵, NHC(═X)NR⁶R⁷, NHC(═X)YR⁸, NHC(═X)R⁹, SC(═X)NR¹⁰R¹¹, SC(═X)YR¹², or SC(═X)R¹³,

R¹ represents a halogen atom, a C1-C4 alkyl group optionally having one or more halogen atoms, a C1-C4 alkoxy group optionally having one or more halogen atoms, a cyano group, or a nitro group,

n represents an integer of any one of 0 to 4, wherein when n is 2 or more, plural R¹(s) may be the same or different to each other,

R² represents a C1-C10 alkyl group having one or more substituents selected from Group P¹, a C3-C4 alkenyl group having one or more substituents selected from Group P¹, a C5-C10 alkenyl group optionally having one or more substituents selected from Group P¹, a C3-C10 alkynyl group optionally having one or more substituents selected from Group P¹, a C3-C10 cycloalkyl group optionally having one or more substituents selected from Group P³, a C1-C4 alkoxy group having one or more substituents selected from Group P¹, a C5-C10 alkoxy group optionally having one or more substituents selected from Group P¹, a C3-C10 alkenyloxy group optionally having one or more substituents selected from Group P¹, or a C3-C10 alkynyloxy group optionally having one or more substituents selected from Group P¹,

R³, R⁷, and R¹¹ each independently represents a hydrogen atom, or a C1-C10 chain hydrocarbon group optionally having one or more substituents selected from Group P¹,

R⁴ and R⁵ each independently represents a C1-C10 alkyl group having one or more substituents selected from Group P¹, a C3-C4 alkenyl group having one or more substituents selected from Group P¹, a C5-C10 alkenyl group optionally having one or more substituents selected from Group P¹, a C3-C10 alkynyl group optionally having one or more substituents selected from Group P¹, a C3-C10 cycloalkyl group optionally having one or more substituents selected from Group P³, or a phenyl group having one or more substituents selected from Group P³,

R⁶ represents a C1-C4 alkyl group having one or more substituents selected from Group P², a C5-C10 alkyl group optionally having one or more substituents selected from Group P¹, a C3-C10 alkenyl group optionally having one or more substituents selected from Group P¹, a C3-C10 alkynyl group optionally having one or more substituents selected from Group P¹, a C3-C10 cycloalkyl group optionally having one or more substituents selected from Group P³, a C1-C4 alkoxy group having one or more substituents selected from Group P¹, a C5-C10 alkoxy group optionally having one or more substituents selected from Group P¹, or a phenyl group having one or more substituents selected from Group P³,

R⁸ and R⁹ each independently represents a C1-C10 alkyl group having one or more substituents selected from Group P², a C5-C10 haloalkyl group, a C3-C4 alkenyl group having one or more substituents selected from Group P¹, a C5-C10 alkenyl group optionally having one or more substituents selected from Group P¹, a C3-C10 alkynyl group optionally having one or more substituents selected from Group P¹, or a C3-C10 cycloalkyl group optionally having one or more substituents selected from Group P³,

R¹⁰, R¹², and R¹³ each independently represents a C1-C10 chain hydrocarbon group optionally having one or more substituents selected from Group P¹, a C3-C10 cycloalkyl group optionally having one or more substituents selected from Group P³, a C1-C10 alkoxy group optionally having one or more substituents selected from Group P¹, a C3-C10 alkenyloxy group optionally having one or more substituents selected from Group P¹, a C3-C10 alkynyloxy group optionally having one or more substituents selected from Group P¹, or a phenyl group having one or more substituents selected from Group P³, and

X and Y each independently represents an oxygen atom or a sulfur atom:

Group P¹: Group consisting of a halogen atom, a C1-C4 alkoxy group optionally having one or more halogen atoms, a cyano group, and a nitro group;

Group P²: Group consisting of a C1-C4 alkoxy group optionally having one or more halogen atoms, a cyano group, and a nitro group; and

Group P³: Group consisting of a halogen atom, a C1-C4 alkyl group optionally having one or more halogen atoms, a C1-C4 alkoxy group optionally having one or more halogen atoms, a cyano group, and a nitro group (hereinafter referred to as the present compound).

[2] The compound according to [1], wherein Z is OC(═X)NR²R³.
[3] The compound according to [2], wherein X is an oxygen atom and Z is bonded at the para-position with respect to the oxadiazole ring.
[4] The compound according to any one of [1] to [3], wherein n is 0.
[5] The compound according to any one of [1] to [4], wherein R³ is a methyl group.
[6] The compound according to any one of [1] to [5], wherein R² is a C1-C10 alkyl group which has a C1-C4 alkoxy group optionally having one or more halogen atoms.
[7] The compound according to any one of [1] to [5], wherein R² is a C1-C10 alkyl group having a cyano group.
[8] The compound according to any one of [1] to [5], wherein R² is a C3-C10 cycloalkyl group optionally having one or more substituents selected from Group P³.
[9] The compound according to [1], wherein Z is OC(═X)NR²R³, OC(═X)R⁵, NHC(═X)NR⁶R⁷, or NHC(═X)R⁹, R¹ is a halogen atom or a C1-C4 alkyl group, n is 0 or 1, X is an oxygen atom, and Z is bonded at the meta- or para-position with respect to the oxadiazole ring.
[10] The compound according to [9], wherein R¹ is a fluorine atom or a methyl group,
R² is a C1-C10 alkyl group having one or more substituents selected from Group P¹, a C3-C10 alkynyl group optionally having one or more substituents selected from Group P¹, or a C3-C10 cycloalkyl group optionally having one or more substituents selected from Group P³,
R³ is a hydrogen atom or a methyl group,
R⁵ is a C3-C10 cycloalkyl group optionally having one or more substituents selected from Group P³ or a phenyl group optionally having one or more substituents selected from Group P³,
R⁶ is a C1-C4 alkyl group having one or more substituents selected from Group P² or a C3-C10 cycloalkyl group optionally having one or more substituents selected from Group P³,
R⁷ is a methyl group,
R⁹ is a C1-C10 alkyl group having one or more substituents selected from Group P² or a C3-C10 cycloalkyl group optionally having one or more substituents selected from Group P³.
[11] A plant disease control agent comprising the compound according to any one of [1] to [10] (hereinafter also referred to as the present control agent).
[12] A method for controlling plant diseases, which comprises treating plants or soil with an effective amount of the compound according to any one of [1] to [10].
[13] Use of the compound according to any one of [1] to [10] for controlling plant diseases.
[14] A composition comprising the oxadiazole compound according to any one of [1] to [10], and one or more selected from the group consisting of an insecticide, an acaricide, a nematicidal agent, a plant growth regulator, a synergist, and another plant disease control agent.

### Effects of the Invention

According to the present invention, plant diseases can be controlled.

### MODE FOR CARRYING OUT THE INVENTION

Substituents as used herein will be described below.

As used herein, “optionally having one or more halogen atoms” means that, when having two or more halogen atoms, those halogen atoms may be the same or different to each other.

The term “CX-CY” as used herein means that the number of carbon atoms is in a range of X to Y. For example, the term “C1-C6” means that the number of carbon atoms is in a range of 1 to 6.

“Halogen atom” represents a fluorine atom, a chlorine atom, a bromine atom, or an iodine atom.

“Chain hydrocarbon group” represents an alkyl group, an alkenyl group, and an alkynyl group.

Examples of “alkyl group” include a methyl group, an ethyl group, a propyl group, an isopropyl group, a 1,1-dimethylpropyl group, a 1,2-dimethylpropyl group, a 1-ethylpropyl group, a butyl group, a tert-butyl group, a pentyl group, and a hexyl group.

Examples of “alkenyl group” include a vinyl group, a 1-propenyl group, a 2-propenyl group, a 1-methyl-1-propenyl group, a 1-methyl-2-propenyl group, a 1,2-dimethyl-1-propenyl group, a 1,1-dimethyl-2-propenyl group, a 1-ethyl-1-propenyl group, a 1-ethyl-2-propenyl group, a 3-butenyl group, a 4-pentenyl group, and a 5-hexenyl group.

Examples of “alkynyl group” include an ethynyl group, a 1-propynyl group, a 2-propynyl group, a 1-methyl-2-propynyl group, a 1,1-dimethyl-2-propynyl group, a 1-ethyl-2-propynyl group, a 2-butynyl group, a 4-pentynyl group, and a 5-hexynyl group.

“C5-C10 haloalkyl group” represents a group in which a hydrogen atom of a C5-C10 alkyl group is substituted with a halogen atom, and examples thereof include a 5-chloropentyl group, a 6-chloro-6,6-difluorohexyl group, a perfluorohexyl group, and a perfluorodecyl group.

Examples of “cycloalkyl group” include a cyclopropyl group, a cyclobutyl group, a cyclopentyl group, and a cyclohexyl group.

“C1-C4 alkoxy-C1-C10 alkyl group optionally having one or more halogen atoms” represents a C1-C10 alkyl group to which a C1-C4 alkoxy optionally having one or more halogen atoms is attached, and examples thereof include a methoxymethyl group, a methoxyethyl group, a methoxydecanyl group, a trifluoromethoxymethyl group, a trifluoromethoxydecanyl group, a 4-chlorobutoxymethyl group, and a 4-chlorobutoxydecanyl group.

Examples of the aspect of the present compound include the following compounds.

An oxadiazole compound in which Z is bonded at the para-position with respect to the oxadiazole ring in the present compound.
An oxadiazole compound in which Z is bonded at the meta-position with respect to the oxadiazole ring in the present compound.
An oxadiazole compound in which n is 0 and Z is bonded at the para-position with respect to the oxadiazole ring in the present compound.
An oxadiazole compound in which n is 0 and Z is bonded at the meta-position with respect to the oxadiazole ring in the present compound.
An oxadiazole compound in which Z is OC(═X)NR²R³ in the present compound.
An oxadiazole compound in which Z is OC(═O)NR²R³ in the present compound.
An oxadiazole compound in which Z is OC(═S)NR²R³ in the present compound.
An oxadiazole compound in which Z is OC(═X)YR⁴ in the present compound.
An oxadiazole compound in which Z is OC(═O)OR⁴ in the present compound.
An oxadiazole compound in which Z is OC(═O)SR⁴ in the present compound.
An oxadiazole compound in which Z is OC(═S)OR⁴ in the present compound.
An oxadiazole compound in which Z is OC(═X)R⁵ in the present compound.
An oxadiazole compound in which Z is OC(═O)R⁵ in the present compound.
An oxadiazole compound in which Z is OC(═S)R⁵ in the present compound.
An oxadiazole compound in which Z is NHC(═X)NR⁶R⁷ in the present compound.
An oxadiazole compound in which Z is NHC(═O)NR⁶R⁷ in the present compound.
An oxadiazole compound in which Z is NHC(═S)NR⁶R⁷ in the present compound.
An oxadiazole compound in which Z is NHC(═X)YR⁸ in the present compound.
An oxadiazole compound in which Z is NHC(═O)OR⁸ in the present compound.
An oxadiazole compound in which Z is NHC(═O)SR⁸ in the present compound.
An oxadiazole compound in which Z is NHC(═S)OR⁸ in the present compound.
An oxadiazole compound in which Z is NHC(═S)SR⁸ in the present compound.
An oxadiazole compound in which Z is NHC(═X)R⁹ in the present compound.
An oxadiazole compound in which Z is NHC(═O)R⁹ in the present compound.
An oxadiazole compound in which Z is NHC(═S)R⁹ in the present compound.
An oxadiazole compound in which Z is SC(═X)NR²R³ in the present compound.
An oxadiazole compound in which Z is SC(═O)NR²R³ in the present compound.
An oxadiazole compound in which Z is SC(═S)NR²R³ in the present compound.
An oxadiazole compound in which Z is SC(═X)YR⁴ in the present compound.
An oxadiazole compound in which Z is SC(═O)OR⁴ in the present compound.
An oxadiazole compound in which Z is SC(═O)SR⁴ in the present compound.
An oxadiazole compound in which Z is SC(═S)OR⁴ in the present compound.
An oxadiazole compound in which Z is SC(═X)R⁵ in the present compound.
An oxadiazole compound in which Z is SC(═O)R⁵ in the present compound.
An oxadiazole compound in which Z is SC(═S)R⁵ in the present compound.
An oxadiazole compound in which Z is OC(═X)NR²R³ and Z is bonded at the para-position with respect to the oxadiazole ring in the present compound.
An oxadiazole compound in which Z is OC(═O)NR²R³ and Z is bonded at the para-position with respect to the oxadiazole ring in the present compound.
An oxadiazole compound in which Z is OC(═S)NR²R³ and Z is bonded at the para-position with respect to the oxadiazole ring in the present compound.
An oxadiazole compound in which Z is OC(═X)YR⁴ and Z is bonded at the para-position with respect to the oxadiazole ring in the present compound.
An oxadiazole compound in which Z is OC(═O)OR⁴ and Z is bonded at the para-position with respect to the oxadiazole ring in the present compound.
An oxadiazole compound in which Z is OC(═O)SR⁴ and Z is bonded at the para-position with respect to the oxadiazole ring in the present compound.
An oxadiazole compound in which Z is OC(═S)OR⁴ and Z is bonded at the para-position with respect to the oxadiazole ring in the present compound.
An oxadiazole compound in which Z is OC(═X)R⁵ and Z is bonded at the para-position with respect to the oxadiazole ring in the present compound.
An oxadiazole compound in which Z is OC(═O)R⁵ and Z is bonded at the para-position with respect to the oxadiazole ring in the present compound.
An oxadiazole compound in which Z is OC(═S)R⁵ and Z is bonded at the para-position with respect to the oxadiazole ring in the present compound.
An oxadiazole compound in which Z is NHC(═X)NR⁶R⁷ and Z is bonded at the para-position with respect to the oxadiazole ring in the present compound.
An oxadiazole compound in which Z is NHC(═O)NR⁶R⁷ and Z is bonded at the para-position with respect to the oxadiazole ring in the present compound.
An oxadiazole compound in which Z is NHC(═S)NR⁶R⁷ and Z is bonded at the para-position with respect to the oxadiazole ring in the present compound.
An oxadiazole compound in which Z is NHC(═X)YR⁸ and Z is bonded at the para-position with respect to the oxadiazole ring in the present compound.
An oxadiazole compound in which Z is NHC(═O)OR⁸ and Z is bonded at the para-position with respect to the oxadiazole ring in the present compound.
An oxadiazole compound in which Z is NHC(═O)SR⁸ and Z is bonded at the para-position with respect to the oxadiazole ring in the present compound.
An oxadiazole compound in which Z is NHC(═S)OR⁸ and Z is bonded at the para-position with respect to the oxadiazole ring in the present compound.
An oxadiazole compound in which Z is NHC(═S)SR⁸ and Z is bonded at the para-position with respect to the oxadiazole ring in the present compound.
An oxadiazole compound in which Z is NHC(═X)R⁹ and Z is bonded at the para-position with respect to the oxadiazole ring in the present compound.
An oxadiazole compound in which Z is NHC(═O)R⁹ and Z is bonded at the para-position with respect to the oxadiazole ring in the present compound.
An oxadiazole compound in which Z is NHC(═S)R⁹ and Z is bonded at the para-position with respect to the oxadiazole ring in the present compound.
An oxadiazole compound in which Z is SC(═X)NR²R³ and Z is bonded at the para-position with respect to the oxadiazole ring in the present compound.
An oxadiazole compound in which Z is SC(═O)NR²R³ and Z is bonded at the para-position with respect to the oxadiazole ring in the present compound.
An oxadiazole compound in which Z is SC(═S)NR²R³ and Z is bonded at the para-position with respect to the oxadiazole ring in the present compound.
An oxadiazole compound in which Z is SC(═X)YR⁴ and Z is bonded at the para-position with respect to the oxadiazole ring in the present compound.
An oxadiazole compound in which Z is SC(═O)OR⁴ and Z is bonded at the para-position with respect to the oxadiazole ring in the present compound.
An oxadiazole compound in which Z is SC(═O)SR⁴ and Z is bonded at the para-position with respect to the oxadiazole ring in the present compound.
An oxadiazole compound in which Z is SC(═S)OR⁴ and Z is bonded at the para-position with respect to the oxadiazole ring in the present compound.
An oxadiazole compound in which Z is SC(═X)R⁵ and Z is bonded at the para-position with respect to the oxadiazole ring in the present compound.
An oxadiazole compound in which Z is SC(═O)R⁵ and Z is bonded at the para-position with respect to the oxadiazole ring in the present compound.
An oxadiazole compound in which Z is SC(═S)R⁵ and Z is bonded at the para-position with respect to the oxadiazole ring in the present compound.

### [Aspect 1]

A compound in which R², R⁴, R⁵, R⁶, R⁸, R⁹, R¹⁰, R¹², and R¹³ each independently is a C1-C4 alkoxy-C1-C10 alkyl group optionally having one or more halogen atoms, a cyano-C1-C10 alkyl group, or a C3-C10 cycloalkyl group optionally having one or more substituents selected from Group P³, and Z is bonded at the para-position or the meta-position with respect to the oxadiazole ring in the present compound.

### [Aspect 2]

A compound in which R², R⁴, R⁵, R⁶, R⁸, R⁹, R¹⁰, R¹², and R¹³ each independently is a C1-C4 alkoxy-C1-C10 alkyl group optionally having one or more halogen atoms, and Z is bonded at the para-position or the meta-position with respect to the oxadiazole ring in the present compound.

### [Aspect 3]

A compound in which R², R⁴, R⁵, R⁶, R⁸, R⁹, R¹⁰, R¹², and R¹³ each independently is a cyano-C1-C10 alkyl group, and Z is bonded at the para-position or the meta-position with respect to the oxadiazole ring in the present compound.

### [Aspect 4]

A compound in which R², R⁴, R⁵, R⁶, R⁸, R⁹, R¹⁰, R¹², and R¹³ each independently is a C3-C10 cycloalkyl group optionally having one or more substituents selected from Group P³, and Z is bonded at the para-position or the meta-position with respect to the oxadiazole ring in the present compound.

### [Aspect 5]

A compound in which R², R⁴, R⁵, R⁶, R⁸, R⁹, R¹⁰, R¹², and R¹³ each independently is a C1-C4 alkoxy-C1-C6 alkyl group, a cyano-C1-C6 alkyl group, or a C3-C6 cycloalkyl group, and Z is bonded at the para-position or the meta-position with respect to the oxadiazole ring in the present compound.

### [Aspect 6]

A compound in which R², R⁴, R⁵, R⁶, R⁸, R⁹, R¹⁰, R¹², and R¹³ each independently is a C1-C4 alkoxy-C1-C6 alkyl group, and Z is bonded at the para-position or the meta-position with respect to the oxadiazole ring in the present compound.

### [Aspect 7]

A compound in which R², R⁴, R⁵, R⁶, R⁸, R⁹, R¹⁰, R¹², and R¹³ each independently is a cyano-C1-C6 alkyl group, and Z is bonded at the para-position or the meta-position with respect to the oxadiazole ring in the present compound.

### [Aspect 8]

A compound in which R², R⁴, R⁵, R⁶, R⁸, R⁹, R¹⁰, R¹², and R¹³ each independently is a C3-C6 cycloalkyl group, and Z is bonded at the para-position or the meta-position with respect to the oxadiazole ring in the present compound.

### [Aspect 9]

A compound in which R², R⁴, R⁵, R⁶, R⁸, R⁹, R¹⁰, R¹², and R¹³ each independently is a methoxyethyl group, a cyano-C2-C3 alkyl group, a cyclohexyl group, or a cyclopropyl group, and Z is bonded at the para-position or the meta-position with respect to the oxadiazole ring in the present compound.

### [Aspect 10]

A compound in which R², R⁴, R⁵, R⁶, R⁸, R⁹, R¹⁰, R¹², and R¹³ are methoxyethyl groups, and Z is bonded at the para-position or the meta-position with respect to the oxadiazole ring in the present compound.

### [Aspect 11]

A compound in which R², R⁴, R⁵, R⁶, R⁸, R⁹, R¹⁰, R¹², and R¹³ each independently a cyano-C2-C3 alkyl group, and Z is bonded at the para-position or the meta-position with respect to the oxadiazole ring in the present compound.

### [Aspect 12]

A compound in which R², R⁴, R⁵, R⁶, R⁸, R⁹, R¹⁰, R¹², and R¹³ each independently is a cyclohexyl group or a cyclopropyl group, and Z is bonded at the para-position or the meta-position with respect to the oxadiazole ring in the present compound.

### [Aspect 13]

A compound in which Z is OC(═X)NR²R³, OC(═X)YR⁴, OC(═X)R⁵, NHC(═X)NR⁶R⁷, NHC(═X)YR⁸, or NHC(═X)R⁹, R², R⁴, R⁵, R⁶, R⁸, and R⁹ each independently is a C1-C4 alkoxy-C1-C10 alkyl group optionally having one or more halogen atoms, a cyano-C1-C10 alkyl group, or a C3-C10 cycloalkyl group optionally having one or more substituents selected from Group P³, and Z is bonded at the para-position or the meta-position with respect to the oxadiazole ring in the present compound.

### [Aspect 14]

A compound in which Z is OC(═X)NR²R³, OC(═X)YR⁴, OC(═X)R⁵, NHC(═X)NR⁶R⁷, NHC(═X)YR⁸, or NHC(═X)R⁹, R², R⁴, R⁵, R⁶, R⁸, and R⁹ each independently is a C1-C4 alkoxy-C1-C10 alkyl group optionally having one or more halogen atoms, and Z is bonded at the para-position or the meta-position with respect to the oxadiazole ring in the present compound.

### [Aspect 15]

A compound in which Z is OC(═X)NR²R³, OC(═X)YR⁴, OC(═X)R⁵, NHC(═X)NR⁶R⁷, NHC(═X)YR⁸, or NHC(═X)R⁹, R², R⁴, R⁵, R⁶, R⁸, and R⁹ each independently is a cyano-C1-C10 alkyl group, and Z is bonded at the para-position or the meta-position with respect to the oxadiazole ring in the present compound.

### [Aspect 16]

A compound in which Z is OC(═X)NR²R³, OC(═X)YR⁴, OC(═X)R⁵, NHC(═X)NR⁶R⁷, NHC(═X)YR⁸, or NHC(═X)R⁹, R², R⁴, R⁵, R⁶, R⁸, and R⁹ each independently is a C3-C10 cycloalkyl group optionally having one or more substituents selected from Group P³, and Z is bonded at the para-position or the meta-position with respect to the oxadiazole ring in the present compound.

### [Aspect 17]

A compound in which Z is OC(═X)NR²R³, OC(═X)YR⁴, OC(═X)R⁵, NHC(═X)NR⁶R⁷, NHC(═X)YR⁸, or NHC(═X)R⁹, R², R⁴, R⁵, R⁶, R⁸, and R⁹ each independently is a C1-C4 alkoxy-C1-C6 alkyl group, a cyano-C1-C6 alkyl group, or a C3-C6 cycloalkyl group, and Z is bonded at the para-position or the meta-position with respect to the oxadiazole ring in the present compound.

### [Aspect 18]

A compound in which Z is OC(═X)NR²R³, OC(═X)YR⁴, OC(═X)R⁵, NHC(═X)NR⁶R⁷, NHC(═X)YR⁸, or NHC(═X)R⁹, R², R⁴, R⁵, R⁶, R⁸, and R⁹ each independently is a C1-C4 alkoxy-C1-C6 alkyl group, and Z is bonded at the para-position or the meta-position with respect to the oxadiazole ring in the present compound.

### [Aspect 19]

A compound in which Z is OC(═X)NR²R³, OC(═X)YR⁴, OC(═X)R⁵, NHC(═X)NR⁶R⁷, NHC(═X)YR⁸, or NHC(═X)R⁹, R², R⁴, R⁵, R⁶, R⁸, and R⁹ each independently is a cyano-C1-C6 alkyl group, and Z is bonded at the para-position or the meta-position with respect to the oxadiazole ring in the present compound.

### [Aspect 20]

A compound in which Z is OC(═X)NR²R³, OC(═X)YR⁴, OC(═X)R⁵, NHC(═X)NR⁶R⁷, NHC(═X)YR⁸, or NHC(═X)R⁹, R², R⁴, R⁵, R⁶, R⁸, and R⁹ each independently is a C3-C6 cycloalkyl group, and Z is bonded at the para-position or the meta-position with respect to the oxadiazole ring in the present compound.

### [Aspect 21]

A compound in which Z is OC(═X)NR²R³, OC(═X)YR⁴, OC(═X)R⁵, NHC(═X)NR⁶R⁷, NHC(═X)YR⁸, or NHC(═X)R⁹, R², R⁴, R⁵, R⁶, R⁸, and R⁹ each independently is a methoxyethyl group, a cyano-C2-C3 alkyl group, a cyclohexyl group, or a cyclopropyl group, and Z is bonded at the para-position or the meta-position with respect to the oxadiazole ring in the present compound.

### [Aspect 22]

A compound in which Z is OC(═X)NR²R³, OC(═X)YR⁴, OC(═X)R⁵, NHC(═X)NR⁶R⁷, NHC(═X)YR⁸, or NHC(═X)R⁹, R², R⁴, R⁵, R⁶, R⁸, and R⁹ is a methoxyethyl group, and Z is bonded at the para-position or the meta-position with respect to the oxadiazole ring in the present compound.

### [Aspect 23]

A compound in which Z is OC(═X)NR²R³, OC(═X)YR⁴, OC(═X)R⁵, NHC(═X)NR⁶R⁷, NHC(═X)YR⁸, or NHC(═X)R⁹, R², R⁴, R⁵, R⁶, R⁸, and R⁹ each independently is a cyano-C2-C3 alkyl group, and Z is bonded at the para-position or the meta-position with respect to the oxadiazole ring in the present compound.

### [Aspect 24]

A compound in which Z is OC(═X)NR²R³, OC(═X)YR⁴, OC(═X)R⁵, NHC(═X)NR⁶R⁷, NHC(═X)YR⁸, or NHC(═X)R⁹, R², R⁴, R⁵, R⁶, R⁸, and R⁹ each independently is a cyclohexyl group or a cyclopropyl group, and Z is bonded at the para-position or the meta-position with respect to the oxadiazole ring in the present compound.

### [Aspect 25]

A compound in which Z is OC(═X)NR²R³ or NHC(═X)NR⁶R⁷, R² and R⁶ each independently is a C1-C4 alkoxy-C1-C10 alkyl group optionally having one or more halogen atoms, a cyano-C1-C10 alkyl group, or a C3-C10 cycloalkyl group optionally having one or more substituents selected from Group P³, and Z is bonded at the para-position or the meta-position with respect to the oxadiazole ring in the present compound.

### [Aspect 26]

A compound in which Z is OC(═X)NR²R³ or NHC(═X)NR⁶R⁷, R² and R⁶ each independently is a C1-C4 alkoxy-C1-C10 alkyl group optionally having one or more halogen atoms, and Z is bonded at the para-position or the meta-position with respect to the oxadiazole ring in the present compound.

### [Aspect 27]

A compound in which Z is OC(═X)NR²R³ or NHC(═X)NR⁶R⁷, R² and R⁶ each independently a cyano-C1-C10 alkyl group, and Z is bonded at the para-position or the meta-position with respect to the oxadiazole ring in the present compound.

### [Aspect 28]

A compound in which Z is OC(═X)NR²R³ or NHC(═X)NR⁶R⁷, R² and R⁶ each independently is a C3-C10 cycloalkyl group optionally having one or more substituents selected from Group P³, and Z is bonded at the para-position or the meta-position with respect to the oxadiazole ring in the present compound.

### [Aspect 29]

A compound in which Z is OC(═X)NR²R³ or NHC(═X)NR⁶R⁷, R² and R⁶ each independently a C1-C4 alkoxy-C1-C6 alkyl group, a cyano-C1-C6 alkyl group, or a C3-C6 cycloalkyl group, and Z is bonded at the para-position or the meta-position with respect to the oxadiazole ring in the present compound.

### [Aspect 30]

A compound in which Z is OC(═X)NR²R³ or NHC(═X)NR⁶R⁷, R² and R⁶ each independently is a C1-C4 alkoxy-C1-C6 alkyl group, and Z is bonded at the para-position or the meta-position with respect to the oxadiazole ring in the present compound.

### [Aspect 31]

A compound in which Z is OC(═X)NR²R³ or NHC(═X)NR⁶R⁷, R² and R⁶ each independently is a cyano-C1-C6 alkyl group, and Z is bonded at the para-position or the meta-position with respect to the oxadiazole ring in the present compound.

### [Aspect 32]

A compound in which Z is OC(═X)NR²R³ or NHC(═X)NR⁶R⁷, R² and R⁶ each independently is a C3-C6 cycloalkyl group, and Z is bonded at the para-position or the meta-position with respect to the oxadiazole ring in the present compound.

### [Aspect 33]

A compound in which Z is OC(═X)NR²R³ or NHC(═X)NR⁶R⁷, R² and R⁶ each independently is a methoxyethyl group, a cyano-C2-C3 alkyl group, a cyclohexyl group, or a cyclopropyl group, and Z is bonded at the para-position or the meta-position with respect to the oxadiazole ring in the present compound.

### [Aspect 34]

A compound in which Z is OC(═X)NR²R³ or NHC(═X)NR⁶R⁷, R² and R⁶ are methoxyethyl groups, and Z is bonded at the para-position or the meta-position with respect to the oxadiazole ring in the present compound.

### [Aspect 35]

A compound in which Z is OC(═X)NR²R³ or NHC(═X)NR⁶R⁷, R² and R⁶ each independently is a cyano-C2-C3 alkyl group, and Z is bonded at the para-position or the meta-position with respect to the oxadiazole ring in the present compound.

### [Aspect 36]

A compound in which Z is OC(═X)NR²R³ or NHC(═X)NR⁶R⁷, R² and R⁶ each independently is a cyclohexyl group or a cyclopropyl group, and Z is bonded at the para-position or the meta-position with respect to the oxadiazole ring in the present compound.

### [Aspect 37]

A compound in which Z is OC(═X)NR²R³, R² is a C1-C4 alkoxy-C1-C10 alkyl group optionally having one or more halogen atoms, a cyano-C1-C10 alkyl group, or a C3-C10 cycloalkyl group optionally having one or more substituents selected from Group P³, and Z is bonded at the para-position or the meta-position with respect to the oxadiazole ring in the present compound.

### [Aspect 38]

A compound in which Z is OC(═X)NR²R³, R² is a C1-C4 alkoxy-C1-C10 alkyl group optionally having one or more halogen atoms, and Z is bonded at the para-position or the meta-position with respect to the oxadiazole ring in the present compound.

### [Aspect 39]

A compound in which Z is OC(═X)NR²R³, R² is a cyano-C1-C10 alkyl group, and Z is bonded at the para-position or the meta-position with respect to the oxadiazole ring in the present compound.

### [Aspect 40]

A compound in which Z is OC(═X)NR²R³, R² is a C3-C10 cycloalkyl group optionally having one or more substituents selected from Group P³, and Z is bonded at the para-position or the meta-position with respect to the oxadiazole ring in the present compound.

### [Aspect 41]

A compound in which Z is OC(═X)NR²R³, R² is a C1-C4 alkoxy-C1-C6 alkyl group, a cyano-C1-C6 alkyl group, or a C3-C6 cycloalkyl group, and Z is bonded at the para-position or the meta-position with respect to the oxadiazole ring in the present compound.

### [Aspect 42]

A compound in which Z is OC(═X)NR²R³, R² is a C1-C4 alkoxy-C1-C6 alkyl group, and Z is bonded at the para-position or the meta-position with respect to the oxadiazole ring in the present compound.

### [Aspect 43]

A compound in which Z is OC(═X)NR²R³, R² is a cyano-C1-C6 alkyl group, and Z is bonded at the para-position or the meta-position with respect to the oxadiazole ring in the present compound.

### [Aspect 44]

A compound in which Z is OC(═X)NR²R³, R² is a C3-C6 cycloalkyl group, and Z is bonded at the para-position or the meta-position with respect to the oxadiazole ring in the present compound.

### [Aspect 45]

A compound in which Z is OC(═X)NR²R³, R² is a methoxyethyl group, a cyano-C2-C3 alkyl group, a cyclohexyl group, or a cyclopropyl group, and Z is bonded at the para-position or the meta-position with respect to the oxadiazole ring in the present compound.

### [Aspect 46]

A compound in which Z is OC(═X)NR²R³, R² is a methoxyethyl group, and Z is bonded at the para-position or the meta-position with respect to the oxadiazole ring in the present compound.

### [Aspect 47]

A compound in which Z is OC(═X)NR²R³, R² is a cyano-C2-C3 alkyl group, and Z is bonded at the para-position or the meta-position with respect to the oxadiazole ring in the present compound.

### [Aspect 48]

A compound in which Z is OC(═X)NR²R³, R² is a cyclohexyl group or a cyclopropyl group, and Z is bonded at the para-position or the meta-position with respect to the oxadiazole ring in the present compound.

### [Aspect 49]

A compound in which Z is NHC(═X)NR⁶R⁷, R⁶ is a C1-C4 alkoxy-C1-C10 alkyl group optionally having one or more halogen atoms, a cyano-C1-C10 alkyl group, or a C3-C10 cycloalkyl group optionally having one or more substituents selected from Group P³, and Z is bonded at the para-position or the meta-position with respect to the oxadiazole ring in the present compound.

### [Aspect 50]

A compound in which Z is NHC(═X)NR⁶R⁷, R⁶ is a C1-C4 alkoxy-C1-C10 alkyl group optionally having one or more halogen atoms, and Z is bonded at the para-position or the meta-position with respect to the oxadiazole ring in the present compound.

### [Aspect 51]

A compound in which Z is NHC(═X)NR⁶R⁷, R⁶ is a cyano-C1-C10 alkyl group, and Z is bonded at the para-position or the meta-position with respect to the oxadiazole ring in the present compound.

### [Aspect 52]

A compound in which Z is NHC(═X)NR⁶R⁷, R⁶ is a C3-C10 cycloalkyl group optionally having one or more substituents selected from Group P³, and Z is bonded at the para-position or the meta-position with respect to the oxadiazole ring in the present compound.

### [Aspect 53]

A compound in which Z is NHC(═X)NR⁶R⁷, R⁶ is a C1-C4 alkoxy-C1-C6 alkyl group, a cyano-C1-C6 alkyl group, or a C3-C6 cycloalkyl group, and Z is bonded at the para-position or the meta-position with respect to the oxadiazole ring in the present compound.

### [Aspect 54]

A compound in which Z is NHC(═X)NR⁶R⁷, R⁶ is a C1-C4 alkoxy-C1-C6 alkyl group, and Z is bonded at the para-position or the meta-position with respect to the oxadiazole ring in the present compound.

### [Aspect 55]

A compound in which Z is NHC(═X)NR⁶R⁷, R⁶ is a cyano-C1-C6 alkyl group, and Z is bonded at the para-position or the meta-position with respect to the oxadiazole ring in the present compound.

### [Aspect 56]

A compound in which Z is NHC(═X)NR⁶R⁷, R⁶ is a C3-C6 cycloalkyl group, and Z is bonded at the para-position or the meta-position with respect to the oxadiazole ring in the present compound.

### [Aspect 57]

A compound in which Z is NHC(═X)NR⁶R⁷, R⁶ is a methoxyethyl group, a cyano-C2-C3 alkyl group, a cyclohexyl group, or a cyclopropyl group, and Z is bonded at the para-position or the meta-position with respect to the oxadiazole ring in the present compound.

### [Aspect 58]

A compound in which Z is NHC(═X)NR⁶R⁷, R⁶ is a methoxyethyl group, and Z is bonded at the para-position or the meta-position with respect to the oxadiazole ring in the present compound.

### [Aspect 59]

A compound in which Z is NHC(═X)NR⁶R⁷, R⁶ is a cyano-C2-C3 alkyl group, and Z is bonded at the para-position or the meta-position with respect to the oxadiazole ring in the present compound.

### [Aspect 60]

A compound in which Z is NHC(═X)NR⁶R⁷, R⁶ is a cyclohexyl group or a cyclopropyl group, and Z is bonded at the para-position or the meta-position with respect to the oxadiazole ring in the present compound.

### [Aspect 61]

A compound in which R³, R⁷, and R¹¹ each independently is a methyl group or a hydrogen atom in any one of Aspects 1 to 12.

### [Aspect 62]

A compound in which R³, R⁷, and R¹¹ are methyl groups in any one of Aspects 1 to 12.

### [Aspect 63]

A compound in which R³ and R⁷ each independently is a methyl group or a hydrogen atom in any one of Aspects 13 to 36.

### [Aspect 64]

A compound in which R³ and R⁷ are methyl groups in any one of Aspects 13 to 36.

### [Aspect 65]

A compound in which R³ is a methyl group or a hydrogen atom in any one of Aspects 37 to 48.

### [Aspect 66]

A compound in which R³ is a methyl group in any one of Aspects 37 to 48.

### [Aspect 67]

A compound in which R⁷ is a methyl group or a hydrogen atom in any one of Aspects 49 to 60.

### [Aspect 68]

A compound in which R⁷ is a methyl group in any one of Aspects 49 to 60.

### [Aspect 69]

A compound in which X is an oxygen atom in any one of Aspects 1 to 60.

### [Aspect 70]

A compound in which n is 0 or 1 in any one of Aspects 1 to 60.

### [Aspect 71]

A compound in which n is 0 in any one of Aspects 1 to 60.

### [Aspect 72]

A compound in which Z is bonded at the para-position with respect to the oxadiazole ring in any one of Aspects 1 to 60.

### [Aspect 73]

A compound in which X is an oxygen atom and n is 0 or 1 in any one of Aspects 1 to 60.

### [Aspect 74]

A compound in which X is an oxygen atom and n is 0 in any one of Aspects 1 to 60.

### [Aspect 75]

A compound in which X is an oxygen atom, and Z is bonded at the para-position with respect to the oxadiazole ring in any one of Aspects 1 to 60.

### [Aspect 76]

A compound in which n is 0 or 1, and Z is bonded at the para-position with respect to the oxadiazole ring in any one of Aspects 1 to 60.

### [Aspect 77]

A compound in which n is 0, and Z is bonded at the para-position with respect to the oxadiazole ring in any one of Aspects 1 to 60.

### [Aspect 78]

A compound in which X is an oxygen atom, n is 0 or 1, and Z is bonded at the para-position with respect to the oxadiazole ring in any one of Aspects 1 to 60.

### [Aspect 79]

A compound in which X is an oxygen atom, n is 0, and Z is bonded at the para-position with respect to the oxadiazole ring in any one of Aspects 1 to 60.

### [Aspect 80]

A compound in which R³, R⁷, and R¹¹ each independently is a methyl group or a hydrogen atom, and X is an oxygen atom in any one of Aspects 1 to 12.

### [Aspect 81]

A compound in which R³, R⁷, and R¹¹ each independently is a methyl group or a hydrogen atom, and n is 0 or 1 in any one of Aspects 1 to 12.

### [Aspect 82]

A compound in which R³, R⁷, and R¹¹ each independently is a methyl group or a hydrogen atom, and n is 0 in any one of Aspects 1 to 12.

### [Aspect 83]

A compound in which R³, R⁷, and R¹¹ each independently is a methyl group or a hydrogen atom, and Z is bonded at the para-position with respect to the oxadiazole ring in any one of Aspects 1 to 12.

### [Aspect 84]

A compound in which R³, R⁷, and R¹¹ are methyl groups, and X is an oxygen atom in any one of Aspects 1 to 12.

### [Aspect 85]

A compound in which R³, R⁷, and R¹¹ are methyl groups, and n is 0 or 1 in any one of Aspects 1 to 12.

### [Aspect 86]

A compound in which R³, R⁷, and R¹¹ are methyl groups, and n is 0 in any one of Aspects 1 to 12.

### [Aspect 87]

A compound in which R³, R⁷, and R¹¹ are methyl groups, and Z is bonded at the para-position with respect to the oxadiazole ring in any one of Aspects 1 to 12.

### [Aspect 88]

A compound in which R³ and R⁷ each independently is a methyl group or a hydrogen atom, and X is an oxygen atom in any one of Aspects 13 to 36.

### [Aspect 89]

A compound in which R³ and R⁷ each independently is a methyl group or a hydrogen atom, and n is 0 or 1 in any one of Aspects 13 to 36.

### [Aspect 90]

A compound in which R³ and R⁷ each independently is a methyl group or a hydrogen atom, and n is 0 in any one of Aspects 13 to 36.

### [Aspect 91]

A compound in which R³ and R⁷ each independently is a methyl group or a hydrogen atom, and Z is bonded at the para-position with respect to the oxadiazole ring in any one of Aspects 13 to 36.

### [Aspect 92]

A compound in which R³ and R⁷ are methyl groups, and X is an oxygen atom in any one of Aspects 13 to 36.

### [Aspect 93]

A compound in which R³ and R⁷ are methyl groups, and n is 0 or 1 in any one of Aspects 13 to 36.

### [Aspect 94]

A compound in which R³ and R⁷ are methyl groups, and n is 0 in any one of Aspects 13 to 36.

### [Aspect 95]

A compound in which R³ and R⁷ are methyl groups, and Z is bonded at the para-position with respect to the oxadiazole ring in any one of Aspects 13 to 36.

### [Aspect 96]

A compound in which R³ is a methyl group or a hydrogen atom, and X is an oxygen atom in any one of Aspects 37 to 48.

### [Aspect 97]

A compound in which R³ is a methyl group or a hydrogen atom, and n is 0 or 1 in any one of Aspects 37 to 48.

### [Aspect 98]

A compound in which R³ is a methyl group or a hydrogen atom, and n is 0 in any one of Aspects 37 to 48.

### [Aspect 99]

A compound in which R³ is a methyl group or a hydrogen atom, and Z is bonded at the para-position with respect to the oxadiazole ring in any one of Aspects 37 to 48.

### [Aspect 100]

A compound in which R³ is a methyl group, and X is an oxygen atom in any one of Aspects 37 to 48.

### [Aspect 101]

A compound in which R³ is a methyl group, and n is 0 or 1 in any one of Aspects 37 to 48.

### [Aspect 102]

A compound in which R³ is a methyl group, and n is 0 in any one of Aspects 37 to 48.

### [Aspect 103]

A compound in which R³ is methyl group, and Z is bonded at the para-position with respect to the oxadiazole ring any one of Aspects 37 to 48.

### [Aspect 104]

A compound in which R⁷ is a methyl group or a hydrogen atom, and X is an oxygen atom in any one of Aspects 49 to 60.

### [Aspect 105]

A compound in which R⁷ is a methyl group or a hydrogen atom, and n is 0 or 1 in any one of Aspects 49 to 60.

### [Aspect 106]

A compound in which R⁷ is a methyl group or a hydrogen atom, and n is 0 in any one of Aspects 49 to 60.

### [Aspect 107]

A compound in which R⁷ is a methyl group or a hydrogen atom, and Z is bonded at the para-position with respect to the oxadiazole ring in any one of Aspects 49 to 60.

### [Aspect 108]

A compound in which R⁷ is a methyl group, and X is an oxygen atom in any one of Aspects 49 to 60.

### [Aspect 109]

A compound in which R⁷ is a methyl group, and n is 0 or 1 in any one of Aspects 49 to 60.

### [Aspect 110]

A compound in which R⁷ is a methyl group, and n is 0 in any one of Aspects 49 to 60.

### [Aspect 111]

A compound in which R⁷ is a methyl group, and Z is bonded at the para-position with respect to the oxadiazole ring in any one of Aspects 49 to 60.

### [Aspect 112]

A compound in which R³, R⁷, and R¹¹ each independently is a methyl group or a hydrogen atom, X is an oxygen atom, and n is 0 or 1 in any one of Aspects 1 to 12.

### [Aspect 113]

A compound in which R³, R⁷, and R¹¹ each independently is a methyl group or a hydrogen atom, X is an oxygen atom, and n is 0 in any one of Aspects 1 to 12.

### [Aspect 114]

A compound in which R³, R⁷, and R¹¹ each independently is a methyl group or a hydrogen atom, X is an oxygen atom, n is 0 or 1, and Z is bonded at the para-position with respect to the oxadiazole ring in any one of Aspects 1 to 12.

### [Aspect 115]

A compound in which R³, R⁷, and R¹¹ each independently is a methyl group or a hydrogen atom, X is an oxygen atom, n is 0, and Z is bonded at the para-position with respect to the oxadiazole ring in any one of Aspects 1 to 12.

### [Aspect 116]

A compound in which R³, R⁷, and R¹¹ are methyl groups, X is an oxygen atom, and n is 0 or 1 in any one of Aspects 1 to 12.

### [Aspect 117]

A compound in which R³, R⁷, and R¹¹ are methyl groups, X is an oxygen atom, and n is 0 in any one of Aspects 1 to 12.

### [Aspect 118]

A compound in which R³, R⁷, and R¹¹ are methyl groups, X is an oxygen atom, n is 0 or 1, and Z is bonded at the para-position with respect to the oxadiazole ring in any one of Aspects 1 to 12.

### [Aspect 119]

A compound in which R³, R⁷, and R¹¹ are methyl groups, X is an oxygen atom, n is 0, and Z is bonded at the para-position with respect to the oxadiazole ring in any one of Aspects 1 to 12.

### [Aspect 120]

A compound in which R³ and R⁷ each independently is a methyl group or a hydrogen atom, X is an oxygen atom, and n is 0 or 1 in any one of Aspects 13 to 36.

### [Aspect 121]

A compound in which R³ and R⁷ each independently is a methyl group or a hydrogen atom, X is an oxygen atom, and n is 0 in any one of Aspects 13 to 36.

### [Aspect 122]

A compound in which R³ and R⁷ each independently is a methyl group or a hydrogen atom, X is an oxygen atom, n is 0 or 1, and Z is bonded at the para-position with respect to the oxadiazole ring in any one of Aspects 13 to 36.

### [Aspect 123]

A compound in which R³ and R⁷ each independently is a methyl group or a hydrogen atom, X is an oxygen atom, n is 0, and Z is bonded at the para-position with respect to the oxadiazole ring in any one of Aspects 13 to 36.

### [Aspect 124]

A compound in which R³ and R⁷ are methyl groups, X is an oxygen atom, and n is 0 or 1 in any one of Aspects 13 to 36.

### [Aspect 125]

A compound in which R³ and R⁷ are methyl groups, X is an oxygen atom, and n is 0 in any one of Aspects 13 to 36.

### [Aspect 126]

A compound in which R³ and R⁷ are methyl groups, X is an oxygen atom, n is 0 or 1, and Z is bonded at the para-position with respect to the oxadiazole ring in any one of Aspects 13 to 36.

### [Aspect 127]

A compound in which R³ and R⁷ are methyl groups, X is an oxygen atom, n is 0, and Z is bonded at the para-position with respect to the oxadiazole ring in any one of Aspects 13 to 36.

### [Aspect 128]

A compound in which R³ is a methyl group or a hydrogen atom, X is an oxygen atom, and n is 0 or 1 in any one of Aspects 37 to 48.

### [Aspect 129]

A compound in which R³ is a methyl group or a hydrogen atom, X is an oxygen atom, and n is 0 in any one of Aspects 37 to 48.

### [Aspect 130]

A compound in which R³ is a methyl group or a hydrogen atom, X is an oxygen atom, n is 0 or 1, and Z is bonded at the para-position with respect to the oxadiazole ring in any one of Aspects 37 to 48.

### [Aspect 131]

A compound in which R³ is a methyl group or a hydrogen atom, X is an oxygen atom, n is 0, and Z is bonded at the para-position with respect to the oxadiazole ring in any one of Aspects 37 to 48.

### [Aspect 132]

A compound in which R³ is a methyl group, X is an oxygen atom, and n is 0 or 1 in any one of Aspects 37 to 48.

### [Aspect 133]

A compound in which R³ is a methyl group, X is an oxygen atom, and n is 0 in any one of Aspects 37 to 48.

### [Aspect 134]

A compound in which R³ is a methyl group, X is an oxygen atom, n is 0 or 1, and Z is bonded at the para-position with respect to the oxadiazole ring in any one of Aspects 37 to 48.

### [Aspect 135]

A compound in which R³ is a methyl group, X is an oxygen atom, n is 0, and Z is bonded at the para-position with respect to the oxadiazole ring in any one of Aspects 37 to 48.

### [Aspect 136]

A compound in which R⁷ is a methyl group or a hydrogen atom, X is an oxygen atom, and n is 0 or 1 in any one of Aspects 49 to 60.

### [Aspect 137]

A compound in which R⁷ is a methyl group or a hydrogen atom, X is an oxygen atom, and n is 0 in any one of Aspects 49 to 60.

### [Aspect 138]

A compound in which R⁷ is a methyl group or a hydrogen atom, X is an oxygen atom, n is 0 or 1, and Z is bonded at the para-position with respect to the oxadiazole ring in any one of Aspects 49 to 60.

### [Aspect 139]

A compound in which R⁷ is a methyl group or a hydrogen atom, X is an oxygen atom, n is 0, and Z is bonded at the para-position with respect to the oxadiazole ring in any one of Aspects 49 to 60.

### [Aspect 140]

A compound in which R⁷ is a methyl group, X is an oxygen atom, and n is 0 or 1 in any one of Aspects 49 to 60.

### [Aspect 141]

A compound in which R⁷ is a methyl group, X is an oxygen atom, and n is 0 in any one of Aspects 49 to 60.

### [Aspect 142]

A compound in which R⁷ is a methyl group, X is an oxygen atom, n is 0 or 1, and Z is bonded at the para-position with respect to the oxadiazole ring in any one of Aspects 49 to 60.

### [Aspect 143]

A compound in which R⁷ is a methyl group, X is an oxygen atom, n is 0, and Z is bonded at the para-position with respect to the oxadiazole ring in any one of Aspects 49 to 60.

Next, a process for producing the present compound will be described.

The present compound can be produced, for example, by the following Production Processes.

### Production Process A

Among the present compounds, a compound in which Z is OC(═X)NHR² (hereinafter referred to as the compound (I-1)) can be produced by reacting compound represented by formula (Q1) (hereinafter referred to as the compound (Q1)) with a compound represented by formula (Q2) (hereinafter referred to as the compound (Q2)):

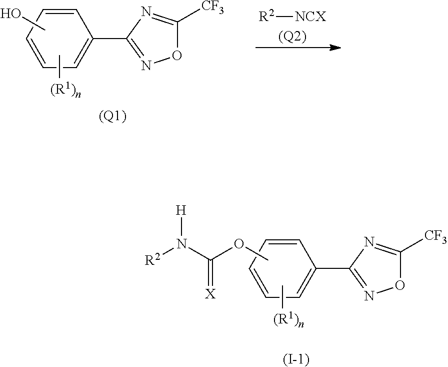

wherein the symbols are the same as defined above.

The reaction is usually performed in a solvent.

Examples of the solvent to be used in the reaction include hydrocarbons such as hexane, cyclohexane, toluene, and xylene (hereinafter referred to as hydrocarbons); ethers such as diethyl ether, tetrahydrofuran, 1,4-dioxane, ethylene glycol dimethyl ether, methyl tert-butyl ether, and diisopropyl ether ethers (hereinafter referred to as ethers); halogenated hydrocarbons such as chloroform, dichloromethane, and chlorobenzene (hereinafter referred to as halogenated hydrocarbons); amides such as N,N′-dimethylformamide, 1,3-dimethyl-2-imidazolidinone, and N-methylpyrrolidone (hereinafter referred to as amides); esters such as ethyl acetate and methyl acetate (hereinafter referred to as esters); sulfoxides such as dimethyl sulfoxide (hereinafter referred to as sulfoxides); nitriles such as acetonitrile and propionitrile (hereinafter referred to as nitriles); and mixtures thereof.

A base may be added in the reaction. Examples of the base to be used in the reaction include organic bases such as triethylamine, pyridine, 2,2′-bipyridine, and diazabicycloundecene (hereinafter referred to as organic bases); carbonates such as sodium carbonate and potassium carbonate; and hydrogen carbonates such as sodium hydrogen carbonate.

In the reaction, the compound (Q2) is usually used in the proportion within a range of 1 to 10 mols, and the base is usually used in the proportion within a range of 0.01 to 10 mols, based on 1 mol of the compound (Q1).

The reaction temperature of the reaction is usually within a range of −20 to 150° C. The reaction time is usually within a range of 0.1 to 120 hours.

After completion of the reaction, the compound (I-1) can be isolated by performing post-treatment operations such as mixing a reaction mixture with water, extraction of the reaction mixture with an organic solvent, and drying and concentration of the organic layer.

The compound (Q1) and the compound (Q2) are known compounds or can be produced in accordance with a known method.

### Production Process B

Among the present compounds, a compound in which Z is OC(═X)NR²R³ (hereinafter referred to as the compound (I-2)) can be produced by reacting the compound (Q1) with a compound represented by formula (Q3) (hereinafter referred to as the compound (Q3)) in the presence of a base:

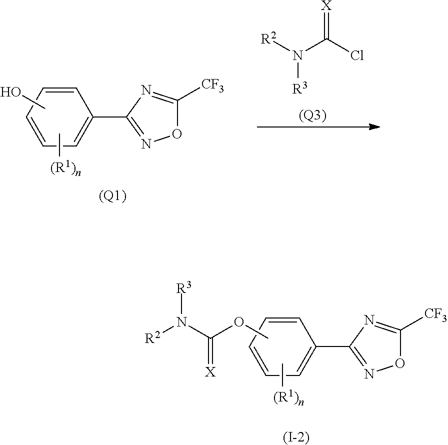

wherein the symbols are the same as defined above.

The reaction is usually performed in a solvent.

Examples of the solvent to be used in the reaction include hydrocarbons, ethers, halogenated hydrocarbons, amides, esters, sulfoxides, nitriles, waters, and mixtures thereof.

Examples of the base to be used in the reaction include organic bases; alkyl metal hydroxides such as sodium hydroxide, potassium hydroxide, and lithium hydroxide; carbonates such as sodium carbonate and potassium carbonate; and hydrogen carbonates such as sodium hydrogen carbonate.

In the reaction, the compound (Q3) is usually used in the proportion within a range of 1 to 10 mols, and the base is usually used in the proportion within a range of 0.5 to 10 mols, based on 1 mol of the compound (Q1).

The reaction temperature of the reaction is usually within a range of −20 to 150° C. The reaction time is usually within a range of 0.1 to 120 hours.

After completion of the reaction, the compound (I-2) can be isolated by performing post-treatment operations such as mixing a reaction mixture with water, extraction of the reaction mixture with an organic solvent, and drying and concentration of the organic layer.

The compound (Q1) and the compound (Q3) are known compounds or can be produced in accordance with a known method.

### Production Process C

Among the present compounds, a compound in which Z is OC(═X)YR⁴ can be produced by reacting the compound (Q1) with a compound represented by formula (Q4) (hereinafter referred to as the compound (Q4)). Among the present compounds, a compound in which Z is OC(═X)R⁵ can be produced by reacting the compound (Q1) with a compound represented by formula (Q5) (hereinafter referred to as the compound (Q5)):

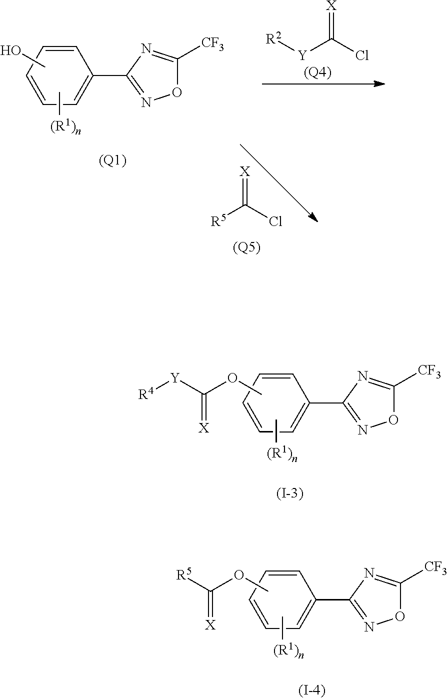

wherein the symbols are the same as defined above.

The reaction can be performed in accordance with the method mentioned in Production Process B.

The compound (Q4) and the compound (Q5) are known compounds or can be produced in accordance with a known method.

### Production Process D

Among the present compounds, a compound in which Z is NHC(═X)NR⁶R⁷ can be produced by reacting a compound represented by formula (Q6) (hereinafter referred to as the compound (Q6)) with a compound represented by formula (Q7) (hereinafter referred to as the compound (Q7)). Among the present compounds, a compound in which Z is NHC(═X)YR⁸ can be produced by reacting the compound (Q6) with a compound represented by formula (Q8) (hereinafter referred to as the compound (Q8)). Among the present compounds, a compound in which Z is NHC(═X)R⁹ can be produced by reacting the compound (Q6) with a compound represented by formula (Q9) (hereinafter referred to as the compound (Q9)):

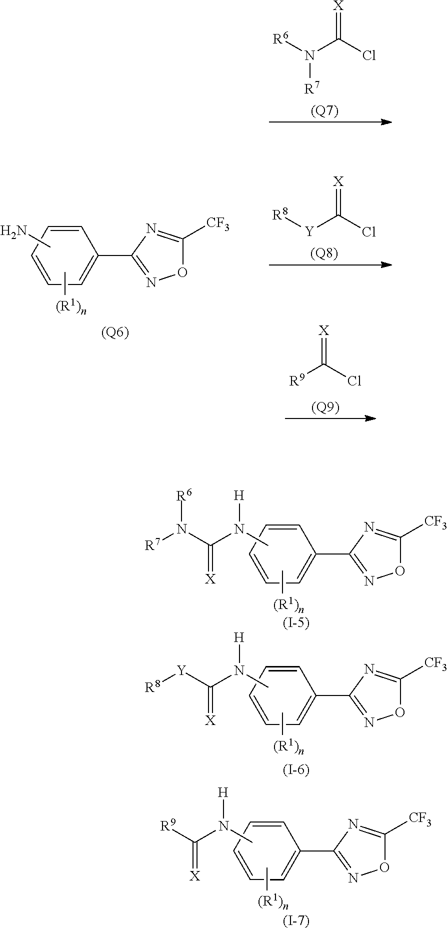

wherein the symbols are the same as defined above.

The reaction can be performed in accordance with the method mentioned in Production Process B.

The compound (Q6), the compound (Q7), the compound (Q8), and the compound (Q9) are known compounds, or can be produced in accordance with a known method.

### Production Process E

Among the present compounds, a compound in which Z is SC(═X)NR¹⁰R¹¹ can be produced by reacting a compound represented by formula (Q10) (hereinafter referred to as the compound (Q10)) with a compound represented by formula (Q1) (hereinafter referred to as the compound (Q11)). Among the present compounds, a compound in which Z is SC(═X)YR¹² can be produced by reacting the compound (Q10) with a compound represented by formula (Q12) (hereinafter referred to as the compound (Q12)). Among the present compounds, a compound in which Z is SC(═X)R¹³ can be produced by reacting the compound (Q10) with a compound represented by formula (Q13) (hereinafter referred to as the compound (Q13)):

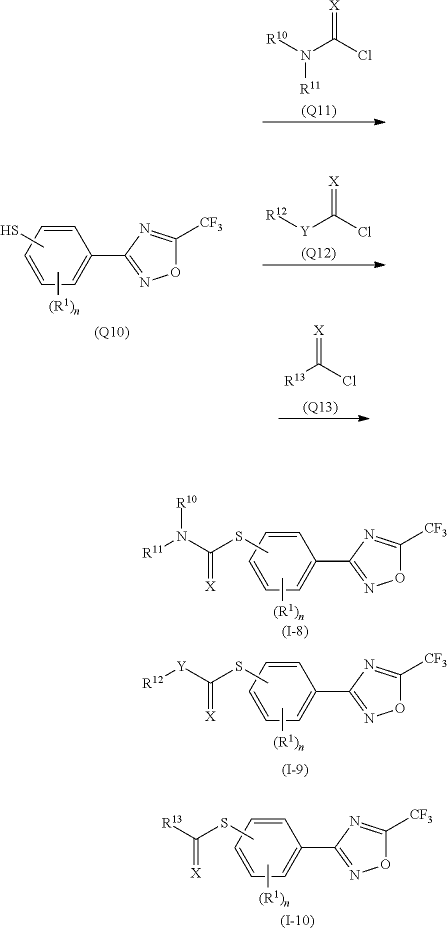

wherein the symbols are the same as defined above.

The reaction can be performed in accordance with the method mentioned in Production Process B.

The compound (Q10), the compound (Q11), the compound (Q12), and the compound (Q13) are known compounds, or can be produced in accordance with a known method.

### Production Process F

The compound (I-2) can also be produced by reacting a compound represented by formula (Q14) (hereinafter referred to as the compound (Q14)) with a compound represented by formula (Q15) (hereinafter referred to as the compound (Q15)) in the presence of a base:

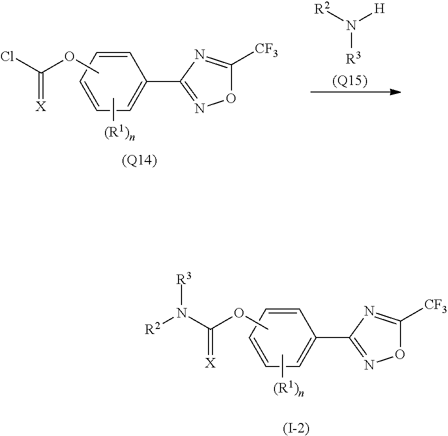

wherein the symbols are the same as defined above.

The reaction is usually performed in a solvent. Examples of the solvent to be used in the reaction include hydrocarbons, ethers, halogenated hydrocarbons, amides, esters, sulfoxides, nitriles, waters, and mixtures thereof.

The compound (Q15) may be in the form of an acid addition salt. Examples of the acid addition salt of the compound (Q15) include hydrochlorides or sulfates.

Examples of the base to be used in the reaction include organic bases such as diisopropylethylamine and pyridine; alkali metal hydroxides such as sodium hydroxide and potassium hydroxide; alkali metal carbonates such as sodium carbonate and potassium carbonate; and alkali metal hydrogen carbonates such as sodium hydrogen carbonate.

In the reaction, the compound (Q15) is usually used in the proportion within a range of 1 to 10 mols, and the base is usually used in the proportion within a range of 1 to 10 mols, based on 1 mol of the compound (Q14).

The reaction temperature of the reaction is usually within a range of −20 to 150° C. The reaction time is usually within a range of 0.1 to 120 hours.

After completion of the reaction, the compound (I-2) can be isolated by performing post-treatment operations such as mixing a reaction mixture with water, extraction of the reaction mixture with an organic solvent, and drying and concentration of the organic layer.

### Production Process G

The compound (Q14) can be produced by reacting the compound (Q1) with phosgene, diphosgene, triphosgene, or thiophosgene in the presence of a base:

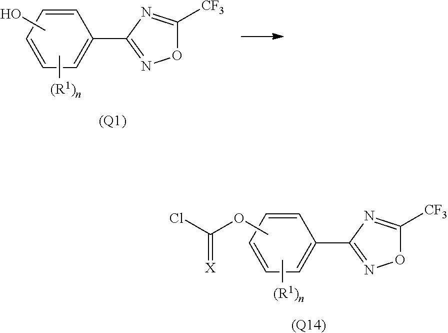

wherein the symbols are the same as defined above.

The reaction is usually performed in a solvent. Examples of the solvent to be used in the reaction include hydrocarbons, ethers, halogenated hydrocarbons, amides, esters, sulfoxides, nitriles, waters, and mixtures thereof.

Examples of the base to be used in the reaction include organic bases such as diisopropylethylamine and pyridine; alkali metal hydroxides such as sodium hydroxide and potassium hydroxide; alkali metal carbonates such as sodium carbonate and potassium carbonate; and alkali metal hydrogen carbonates such as sodium hydrogen carbonate.

In the reaction, phosgene is usually used in the proportion within a range of 1 to 10 mols, and the base is usually used in the proportion within a range of 1 to 10 mols, based on 1 mol of the compound (Q1).

In the reaction, diphosgene is usually used in the proportion within a range of 0.5 to 5 mols, and the base is usually used in the proportion within a range of 1 to 10 mols, based on 1 mol of the compound (Q1).

In the reaction, triphosgene is usually used in the proportion within a range of 0.3 to 3 mols, and the base is usually used in the proportion within a range of 1 to 10 mols, based on 1 mol of the compound (Q1).

In the reaction, thiophosgene is usually used in the proportion within a range of 1 to 10 mols, and the base is usually used in the proportion within a range of 1 to 10 mols, based on 1 mol of the compound (Q1).

The reaction temperature of the reaction is usually within a range of −20 to 150° C. The reaction time is usually within a range of 0.1 to 120 hours.

After completion of the reaction, the compound (Q14) can be obtained by concentrating the reaction mixture. The reaction mixture may be subjected to the method mentioned in Production Process F without being concentrated.

### Production Process H

The compound (I-7) can be produced by reacting the compound (Q6) with a compound represented by the formula (Q16) in the presence of a condensing agent:

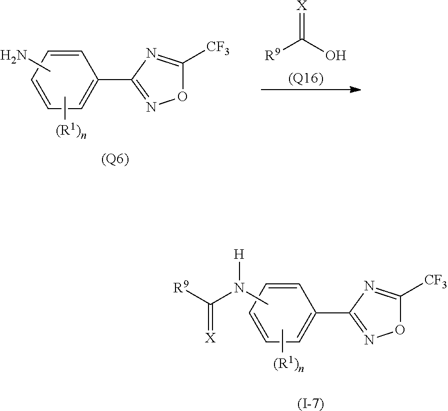

wherein the symbols are the same as defined above.

The reaction can be performed in accordance with the method mentioned in Organic Letters, 2002, 4, 4041.

### Production Process I

The compound (I-11) can be produced by reacting the compound (Q1) with a compound represented by the formula (Q17) (hereinafter referred to as the compound (Q17)):

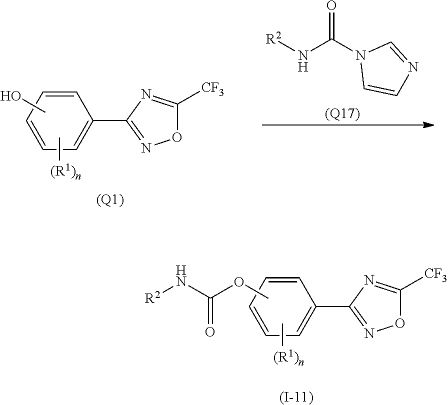

wherein the symbols are the same as defined above.

The reaction can be performed in accordance with the method mentioned in Organic Letters, 2012, 14, 2814.

### Production Process J

The compound (Q17) can be produced by reacting a compound represented by the formula (Q18) with 1,1′-carbonyldiimidazole:

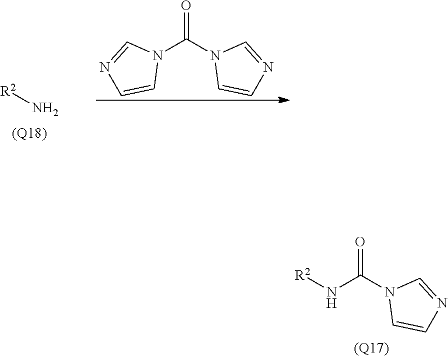

wherein the symbols are the same as defined above.

The reaction can be performed in accordance with the method mentioned in Organic Letters, 2012, 14, 2814.

The respective compounds produced by the methods mentioned in Production Process A, Production Process B, Production Process C, Production Process D, Production Process E, Production Process F, Production Process G, Production Process H, Production Process I, and Production Process J can be isolated and purified by other known means, for example, concentration, concentration under reduced pressure, extraction, partition, crystallization, recrystallization, and chromatography.

Although a form used for the present compound may be the present compound as itself, the present compound is usually used after mixing with solid carriers, liquid carriers, and/or surfactants, and optionally adding auxiliary agents for formulation, such as stickers, dispersers, and stabilizers to thereby formulate into wettable powders, water dispersible granules, flowables, granules, dry flowables, emulsifiable concentrates, aqueous solutions, oil solutions, smoking agents, aerosols, microcapsules, and the like. In these formulations, the present compound is usually contained within a range of 0.1 to 99%, and preferably 0.2 to 90% by weight.

Examples of the solid carriers include clays (for example, kaolin, diatomaceous earth, Fubasami clay, bentonite, and acid clay), synthetic hydrated silicon oxide, talcs or other inorganic minerals (for example, sericite, quartz powder, sulfur powder, activated charcoal, and calcium carbonate) in the form of fine powders or particulates.

Examples of the liquid carries include water, alcohols, ketones (for example, acetone, methyl ethyl ketone, and cyclohexanone), aromatic hydrocarbons (for example, benzene, toluene, xylene, ethylbenzene, and methyl naphthalene), aliphatic hydrocarbons (for example, hexane and kerosene), esters, nitriles, ethers, amides, and halogenated hydrocarbons.

Examples of the surfactants include alkyl sulfates, alkyl sulfonates, alkyl aryl sulfonates, alkyl aryl ethers, and polyoxyethylenated compounds thereof, polyethylene glycol ethers, polyhydric alcohol esters, and sugar alcohol derivatives.

Examples of other auxiliary agents for formulation include stickers, dispersers, and stabilizers, specifically casein, gelatin, polysaccharides (for example, starch, gum arabic, cellulose derivatives, and alginic acid), lignin derivatives, bentonite, saccharides, water-soluble synthetic polymers (for example, polyvinyl alcohol, polyvinylpyrrolidone, and polyacrylic acids), PAP (acidic isopropyl phosphate), BHT (2,6-di-tert-butyl-4-methylphenol), BHA (a mixture of 2-tert-butyl-4-methoxyphenol and 3-tert-butyl-4-methoxyphenol), vegetable oils, mineral oils, fatty acids or esters thereof, and the like.

The present compound may be used as a mixture with various oils such as mineral oils or vegetable oils, or surfactants. Specific examples of oils or surfactants, which can be used as a mixture with various oils or surfactants, include Nimbus (registered trademark), Assist (registered trademark), Aureo (registered trademark), Iharol (registered trademark), Silwet L-77 (registered trademark), BreakThru (registered trademark), Sundancell (registered trademark), Induce (registered trademark), Penetrator (registered trademark), AgriDex (registered trademark), Lutensol A8 (registered trademark), NP-7 (registered trademark), Triton (registered trademark), Nufilm (registered trademark), Emulgator NP7 (registered trademark), Emulad (registered trademark), TRITON X 45 (registered trademark), AGRAL 90 (registered trademark), AGROTIN (registered trademark), ARPON (registered trademark), EnSpray N (registered trademark), BANOLE (registered trademark), and the like.

The present compound is applied as the present control agent. The method for applying the present control agent is not particularly limited, as long as the applying form is a form by which the present control agent may be applied substantially, and includes, for example, an application to plants such as a foliage application; an application to area for cultivating plants such as a submerged treatment; an application to soil such as seed disinfection; and an application to harmful arthropods.

The application amount of the present compound in the control method of the present invention varies depending on kinds of plants to be treated, kinds and occurrence frequency of plant diseases to be controlled, formulation forms, application timing, application method, application place, weather conditions, and the like. When applied to stems and leaves of plants, or soil where plants are to be cultivated, the amount of the present compound is usually within a range of 1 to 500 g per 1,000 m².

The emulsifiable concentrates, wettable powders, and flowables are usually applied after dilution with water. In that case, the concentration of the present compound is usually within a range of 0.0005 to 2% by weight. The dusts, granules, and the like are usually applied as they are without being diluted.

The present compound can be used as an agent for controlling plant diseases in agricultural lands such as fields, paddy fields, lawns, and orchards. The present compound can control diseases occurred in the agricultural lands for cultivating the following “plants”. The present compound can control harmful arthropods in the agricultural lands.

Crops: corn, rice, wheat, barley, rye, oat, sorghum, cotton, soybean, peanut, buckwheat, beet, rapeseed, sunflower, sugar cane, tobacco, and the like; Vegetables: solanaceous vegetables (for example, eggplant, tomato, pimento, pepper, and potato), cucurbitaceous vegetables (for example, cucumber, pumpkin, zucchini, water melon, and melon), cruciferous vegetables (for example, Japanese radish, white turnip, horseradish, kohlrabi, Chinese cabbage, cabbage, leaf mustard, broccoli, and cauliflower), asteraceous vegetables (for example, burdock, crown daisy, artichoke, and lettuce), liliaceous vegetables (for example, green onion, onion, garlic, and asparagus), umbelliferous vegetables (for example, carrot, parsley, celery, and parsnip), chenopodiaceous vegetables (for example, spinach and Swiss chard), lamiaceous vegetables (for example, Perilla frutescens, mint, and basil), strawberry, sweet potato, Dioscorea japonica, colocasia, and the like; Flowers; Ornamental foliage plants; Fruits: pomaceous fruits (for example, apple, pear, Japanese pear, Chinese quince, and quince), stone fruits (for example, peach, plum, nectarine, Prunus mume, cherry fruit, apricot, and prune), citrus fruits (for example, Citrus unshiu, orange, lemon, lime, and grapefruit), nuts (for example, chestnut, walnuts, hazelnuts, almond, pistachio, cashew nuts, and macadamia nuts), berry fruits (for example, blueberry, cranberry, blackberry, and raspberry), grape, persimmon, olive, loquat, banana, coffee, date palm, coconuts, and the like; Trees other than fruit trees: tea, mulberry, flowering plant, roadside trees (for example, ash, birch, dogwood, Eucalyptus, Ginkgo biloba, lilac, maple, Quercus, poplar, Judas tree, Liquidambar formosana, plane tree, zelkova, Japanese arborvitae, fir wood, hemlock, juniper, Pinus, Picea, and Taxus cuspidate); and the like.

The above-mentioned “plants” include genetically modified crops.

Examples of plant diseases which can be controlled by the present compound include plant pathogens such as filamentous fungus and bacteria, and specifically include the following examples, but are not limited thereto.

Rice diseases: blast (Magnaporthe grisea), brown spot (Cochliobolus miyabeanus), sheath blight (Rhizoctonia solani), bakanae disease (Gibberella fujikuroi), and downy mildew (Sclerophthora macrospora); Wheat diseases: powdery mildew (Blumeria graminis), fusarium blight (Fusarium gaminearum, Fusarium avenaceum, Fusarium culmorum, Microdochium nivale), stripe rust (Puccinia striiformis), stem rust (Puccinia graminis), leaf rust (Puccinia recondita), pink snow rot (Microdochium nivale, Microdochium majus), typhula snow blight (Typhula incarnate, Typhula ishikariensis), loose smut (Ustilago tritici), stinking smut (Tilletia caries, Tilletia controversa), eyespot (Pseudocercosporella herpotrichoides), leaf blotch (Septoria tritici), glume blotch (Stagonospora nodorum), tan spot (Pyrenophora tritici-repentis), seeding blight caused by bacteria of the genus of (Rhizoctonia solani), and take-all disease (Gaeumannomyces graminis); Barley diseases: powdery mildew (Blumeria graminis), fusarium blight (Fusarium graminearum, Fusarium avenaceum, Fusarium culmorum, Microdochium nivale), stripe rust (Puccinia striiformis), stem rust (Puccinia graminis), leaf rust (Puccinia hordei), Dwarf leaf rust (Puccinia hordei), loose smut (Ustilago nuda), scald (Rhynchosporium secalis), net blotch (Pyrenophora teres), spot blotch (Cochliobolus sativus), leaf stripe (Pyrenophora graminea), Ramularia leaf spot (Ramularia collo-cygni), and seeding blight caused by bacteria of the genus (Rhizoctonia solani); Corn diseases: rust (Puccinia sorghi), southern rust (Puccinia polysora), northern leaf blight (Setosphaeria turcica), tropical rust (Physopella zeae), southern leaf blight (Cochliobolus heterostrophus), anthracnose (Colletotrichum graminicola), gray leaf spot (Cercospora zeae-maydis), eyespot (Kabatiella zeae), phaeosphaeria leaf spot (Phaeosphaeria maydis), Stenocarpella maydis, Stenocarpella macrospora, stalk rot disease (Fusarium graminearum, Fusarium verticillioides, Colletotrichum graminicola), and smut (Ustilago maydis); Cotton diseases: anthracnose (Colletotrichum gossypii), grey mildew (Ramularia areola), Alternaria leaf spot (Alternaria macrospora, Alternaria gossypii), and Black root rot caused by bacteria from the genus of Thielaviopsis (Thielaviopsis basicola); Coffee diseases: rust (Hemileia vastatrix), leaf spot (Cercospora coffeicola); Rape seed diseases: sclerotinia rot (Sclerotinia sclerotiorum), black spot (Alternaria brassicae), and black leg (Phoma lingam); Sugar cane diseases: rust (Puccinia melanocephala, Puccinia kuehnii) and smut (Ustilago scitaminea); Sunflower diseases: rust (Puccinia helianthi) and downy mildew (Plasmopara halstedii); Citrus diseases: melanose (Diaporthe citri), scab (Elsinoe fawcetti), fruit rot (Penicillium digitatum, Penicillium italicum), and brown rot (Phytophthora parasitica, Phytophthora citrophthora); Apple diseases: blossom blight (Monilinia mali), canker (Valsa ceratosperma), powdery mildew (Podosphaera leucotricha), alternaria leaf spot (Alternaria alternata apple pathotype), scab (Venturia inaequalis), bitter rot (Glomerella cingulata), brown spot (Diplocarpon mali), ring spot (Botryosphaeria berengeriana), and crown rot (Phytophthora cactorum); Pear diseases: scab (Venturia nashicola, Venturia pirina), black spot (Alternaria alternata Japanese pear pathotype), and rust (Gymnosporangium haraeanum); Peach diseases: brown rot (Monilinia fructicola), scab (Cladosporium carpophilum), and Phomopsis rot (Phomopsis sp.); Grapes diseases: anthracnose (Elsinoe ampelina), ripe rot (Glomerella cingulata), powdery mildew (Uncinula necator), rust (Phakopsora ampelopsidis), black rot (Guignardia bidwellii), and downy mildew (Plasmopara viticola); Japanese persimmon diseases: anthracnose (Gloeosporium kaki) and leaf spot (Cercospora kaki, Mycosphaerella nawae); Diseases of gourd family: anthracnose (Colletotrichum lagenarium), powdery mildew (Sphaerotheca fuliginea), gummy stem blight (Didymella bryoniae), target spot (Corynespora cassiicola), fusarium wilt (Fusarium oxysporum), downy mildew (Pseudoperonospora cubensis), phytophthora rot (Phytophthora sp.), and damping-off (Pythium sp.); Tomato diseases: early blight (Alternaria solani), leaf mold (Cladosporium fulvum), leaf mold (Pseudocercospora fuligena), and late blight (Phytophthora infestans), powdery mildew (Leveillula taurica); Eggplant diseases: brown spot (Phomopsis vexans) and powdery mildew (Erysiphe cichoracearum); Cruciferous vegetables diseases: alternaria leaf spot (Alternaria japonica), white spot (Cercosporella brassicae), clubroot (Plasmodiophora brassicae), and downy mildew (Peronospora parasitica); Welsh onion diseases: rust (Puccinia allii); Soybean diseases: purple stain (Cercospora kikuchii), sphaceloma scad (Elsinoe glycines), pod and stem blight (Diaporthe phaseolorum var. sojae), rust (Phakopsora pachyrhizi), target spot (Corynespora cassiicola), anthracnose (Colletotrichum glycines, Colletotrichum truncatum), Rhizoctonia aerial blight (Rhizoctonia solani), septoria brown spot (Septoria glycines), frog eye leaf spot (Cercospora sojina), stem rot (Sclerotinia sclerotiorum), powdery mildew (Microsphaera diffusa), phytophthora blight (Phytophthora sojae), downy mildew (Peronospora manshurica), and sudden death syndrome (Fusarium virguliforme); Kidney bean diseases: stem rot (Sclerotinia sclerotiorum), rust (Uromyces appendiculatus), angular leaf spot (Phaeoisariopsis griseola), and anthracnose (Colletotrichum lindemuthianum); Peanut diseases: early leaf spot (Cercospora personata), late leaf spot (Cercospora arachidicola), and southern blight (Sclerotium rolfsii); Garden pea diseases: powdery mildew (Erysiphe pisi); Potato diseases: early blight (Alternaria solani), late blight (Phytophthora infestans), pink rot (Phytophthora erythroseptica), powdery scab (Spongospora subterranea f. sp. subterranea), and verticillium wilt (Verticillium albo-atrum, Verticillium dahliae, Verticillium nigrescens); Strawberry diseases: powdery mildew (Sphaerotheca humuli); Tea diseases: net blister blight (Exobasidium reticulatum), white scab (Elsinoe leucospila), gray blight (Pestalotiopsis sp.), and anthracnose (Colletotrichum theae-sinensis); Tobacco diseases: brown spot (Alternaria longipes), anthracnose (Colletotrichum tabacum), downy mildew (Peronospora tabacina), and black shank (Phytophthora nicotianae); Sugar beet diseases: cercospora leaf spot (Cercospora beticola), leaf blight (Thanatephorus cucumeris), root rot (Thanatephorus cucumeris), and aphanomyces root rot (Aphanomyces cochlioides), rust (Uromyces betae); Rose diseases: black spot (Diplocarpon rosae) and powdery mildew (Sphaerotheca pannosa); Chrysanthemum diseases: leaf blight (Septoria chrysanthemi-indici) and white rust (Puccinia horiana); Onion diseases: botrytis leaf blight (Botrytis cinerea, Botrytis byssoidea, Botrytis squamosa), gray-mold neck rot (Botrytis allii), and small sclerotial rot (Botrytis squamosa); various crops diseases: gray mold (Botrytis cinerea) and sclerotinia rot (Sclerotinia sclerotiorum); Japanese radish diseases: alternaria leaf spot (Alternaria brassicicola); Turfgrass diseases: dollar spot (Sclerotinia homoeocarpa) and brown patch and large patch (Rhizoctonia solani); and Banana diseases: Sigatoka disease (Mycosphaerella fijiensis, Mycosphaerella musicola).

Seed diseases or diseases in the early growth phase in various crops caused by bacteria from genera of Aspergillus, Penicillium, Fusarium, Gibberella, Tricoderma, Thielaviopsis, Rhizopus, Mucor, Corticium, Phoma, Rhizoctonia, Diplodia, and the like. Viral diseases intermediated by genera of Polymyxa, Olpidium, or the like in various crops.

Rice damping-off (Burkholderia plantarii); cucumber bacterial blight (Pseudomonas syringae pv. Lachrymans); eggplant bacterial wilt disease (Ralstonia solanacearum), citrus canker (Xanthomonas citri); Chinese cabbage soft rod (Erwinia carotovora) and the like.

Examples of harmful arthropods, which can be controlled by the present compound, include the followings.

Hemiptera: planthoppers (Delphacidae) such as small brown planthopper (Laodelphax striatellus), brown rice planthopper (Nilaparvata lugens), white-backed rice planthopper (Sogatella furcifera), corn planthopper (Peregrinus maidis), wheat planthopper (Javesella pellucida), sugarcane leafhopper (Perkinsiella saccharicida), and Tagosodes orizicolus; leafhoppers (Cicadellidae) such as green rice leafhopper (Nephotettix cincticeps), Taiwan green rice leafhopper (Nephotettix virescens), rice green leafhopper (Nephotettix nigropictus), zig-zag rice leafhopper (Recilia dorsalis), tea green leafhopper (Empoasca onukii), potato leafhopper (Empoasca fabae), corn leafhopper (Dalbulus maidis), and white giant leafhopper (Cofana spectra); froghopper (Cercopidae) such as Mahanarva posticata and Mahanarva fimbriolata; aphids (Aphididae) such as bean aphid (Aphis fabae), soybean aphid (Aphis glycines), cotton aphid (Aphis gossypii), green apple aphid (Aphis pomi), spirea aphid (Aphis spiraecola), green peach aphid (Myzus persicae), peach leaf-curl aphid (Brachycaudus helichrysi), cabbage aphid (Brevicoryne brassicae), Rosy apple aphid (Dysaphis plantaginea), false cabbage aphid (Lipaphis erysimi), tulip aphid (Macrosiphum euphorbiae), potato aphid (Aulacorthum solani), lettuce aphid (Nasonovia ribisnigri), oat bird-cherry aphid (Rhopalosiphum padi), corn aphid (Rhopalosiphum maidis), tropical citrus aphid (Toxoptera citricida), mealy plum aphid (Hyalopterus pruni), cane aphid (Melanaphis sacchari), rice root aphid (Tetraneura nigriabdominalis), Sugarcane woolly aphid (Ceratovacuna lanigera), and Woolly apple aphid (Eriosoma lanigerum); spruce aphids (Phylloxeridae) such as grape phylloxera (Daktulosphaira vitifoliae), Pecan phylloxera (Phylloxera devastatrix), Pecan leaf phylloxera (Phylloxera notabilis), and Southern pecan leaf phylloxera (Phylloxera russellae); adelgid (Adelgidae) such as hemlock woolly adelgid (Adelges tsugae), Adelges piceae, and woolly larch aphid (Aphrastasia pectinatae); stink bugs (Pentatomidae) such as Japanese black rice bug (Scotinophara lurida), Malayan rice black bug (Scotinophara coarctata), green stink bug (Nezara antennata), white spotted spined bug (Eysarcoris aeneus), lewis spined bug (Eysarcoris lewisi), white spotted bug (Eysarcoris ventralis), white spotted stink bug (Eysarcoris annamita), brown winged green bug (Halyomorpha halys), southern green stink bug (Nezara viridula), Brown stink bug (Euschistus heros), Red banded stink bug (Piezodorus guildinii), Oebalus pugnax, and Dichelops melacanthus; burrower bugs (Cydnidae) such as Burrower brown bug (Scaptocoris castanea); broad-headed bugs (Alydidae) such as bean bug (Riptortus pedestris), rice bug (Leptocorisa chinensis), and rice seed bug (Leptocorisa acuta); coreid bugs (Coreidae) such as rice stink bug (Cletus punctiger), and Australian leaf-footed bug (Leptoglossus australis); lygaeid bugs (Lygaeidae) such as Oriental chinch bug (Cavelerius saccharivorus), seed bug (Togo hemipterus), and chinch bug (Blissus leucopterus); plant bugs (Miridae) such as rice leaf bug (Trigonotylus caelestialium), sorghum plant bug (Stenotus rubrovittatus), wheat leaf bug (Stenodema calcarata), and tarnished plant bug (Lygus lineolaris); whiteflies (Aleyrodidae) such as greenhouse whitefly (Trialeurodes vaporariorum), tobacco whitefly (Bemisia tabaci), citrus whitefly (Dialeurodes citri), orange spiny whitefly (Aleurocanthus spiniferus), camellia spiny whitefly (Aleurocanthus camelliae), and Pealius euryae; armored scales (Diaspididae) such as armored scale (Abgrallaspis cyanophylli), California red scale (Aonidiella aurantii), san jose scale (Diaspidiotus perniciosus), mulberry scale (Pseudaulacaspis pentagona), arrowhead snow scale (Unaspis yanonensis), and citrus snow scale (Unaspis citri); scales (Coccidae) such as red wax scale (Ceroplastes rubens); Margarodidae such as cottony cushion scale (Icerya purchasi), and iceplant scale (Icerya seychellarum); mealybugs (Pseudococcidae) such as solanum mealybug (Phenacoccus solani), cotton mealybug (Phenacoccus solenopsis), Japanese mealybug (Planococcus kraunhiae), Comstock mealybug (Pseudococcus comstocki), citrus mealybug (Planococcus citri), citrophilus mealybug (Pseudococcus calceolariae), longtail mealybug (Pseudococcus longispinus), and tuttle mealybug (Brevennia rehi); psyllids (Psyllidae) such as Asian citrus psyllid (Diaphorina citri), African citrus psyllid (Trioza erytreae), pear psyllid (Cacopsylla pyrisuga), Chinese pear psyllid (Cacopsylla chinensis), potato psyllid (Bactericera cockerelli), and pear psylla (Cacopsylla pyricola); lace bugs (Tingidae) such as sycamore lace bag (Corythucha ciliata), chrysanthemum lace bug (Corythucha marmorata), pear lace bug (Stephanitis nashi), and azalea lace bug (Stephanitis pyrioides); bed bugs (Cimicidae) such as bed bug (Cimex lectularius); and cicada (Cicadidae) such as giant cicada (Quesada gigas).

Lepidoptera: pyralid moths (Crambidae) such as rice stem borer (Chilo suppressalis), darkheaded stem borer (Chilo polychrysus), white stem borer (Scirpophaga innotata), yellow stem borer (Scirpophaga incertulas), Rupela albina, rice leafroller (Cnaphalocrocis medinalis), Marasmia patnalis, rice leaf roller (Marasmia exigua), cotton leaf roller (Notarcha derogata), oriental corn borer (Ostrinia furnacalis), European corn borer (Ostrinia nubilalis), cabbage webworm (Hellula undalis), Herpetogramma luctuosale, bluegrass webworm (Pediasia teterrellus), rice caseworm (Nymphula depunctalis), and sugarcane borer (Diatraea saccharalis); snout moths (Pyralidae) such as lesser corn stalk borer (Elasmopalpus lignosellus) and Indian mealmoth (Plodia interpunctella); owlet moths (Noctuidae) such as common cutworm (Spodoptera litura), beet armyworm (Spodoptera exigua), armyworm (Mythimna separata), cabbage armyworm (Mamestra brassicae), pink borer (Sesamia inferens), lawn armyworm (Spodoptera mauritia), green rice caterpillar (Naranga aenescens), fall armyworm (Spodoptera frugiperda), African armyworm (Spodoptera exempta), black cutworm (Agrotis ipsilon), beet semi-looper (Autographa nigrisigna), rice looper (Plusia festucae), soybean looper (Chrysodeixis includens), Heliothis spp. such as Trichoplusia spp. and oriental tobacco budworm (Heliothis virescens), Helicoverpa spp. such as corn earworm (Helicoverpa armigera) and American cotton bollworm (Helicoverpa zea), velvetbean caterpillar (Anticarsia gemmatalis), cotton leafworm (Alabama argillacea), and hop vine borer (Hydraecia immanis); white butterflies (Pieridae) such as common white (Pieris rapae); tortricid moths (Tortricidae) such as oriental fruit moth (Grapholita molesta), Grapholita dimorpha, soybean pod borer (Leguminivora glycinivorella), azuki bean podworm (Matsumuraeses azukivora), summer fruit tortrix (Adoxophyes orana fasciata), smaller tea tortrix (Adoxophyes honmai), oriental tea tortrix (Homona magnanima), apple tortrix (Archips fuscocupreanus), codling moth (Cydia pomonella), Sugarcane shoot borer (Tetramoera schistaceana), bean shoot borer (Epinotia aporema), and citrus fruit borer (Ecdytolopha aurantiana); leafblotch miners (Gracillariidae) such as tea leafroller (Caloptilia theivora) and apple leafminer (Phyllonorycter ringoneella); fruitworm moths (Carposinidae) such as peach fruit moth (Carposina sasakii); lyonetiid moths (Lyonetiidae) such as coffee leaf miner (Leucoptera coffeella), peach leaf miner (Lyonetia clerkella), and apple leaf miner (Lyonetia prunifoliella); tussock moths (Lymantriidae) such as Lymantria spp., e.g. gypsy moth (Lymantria dispar) and Euproctis spp., e.g. tea tussock moth (Euproctis pseudoconspersa); diamondback moths (Plutellidae) such as diamondback (Plutella xylostella); gelechiid moths (Gelechiidae) such as peach twig borer (Anarsia lineatella), Helcystogramma triannulella, pink bollworm (Pectinophora gossypiella), potato tuberworm (Phthorimaea operculella), and Tuta absoluta; tiger moths (Arctiidae) such as fall webworm (Hyphantria cunea); sun moths (Castniidae) such as giant sugarcane borer (Telchin licus); carpenter moths (Cossidae) such as carpenter moth (Cossus insularis); geometrid moths (Geometridae) such as mugwort looper (Ascotis selenaria); slug moths (Limacodidae) such as bluestriped nettle grab (Parasa lepida); Stathmopodidae such as persimmon fruit moth (Stathmopoda masinissa); Sphingidae such as death's-head-moth (Acherontia lachesis); clear-winged moths (Sesiidae) such as Nokona feralis; skippers (Hesperiidae) such as rice skipper (Parnara guttata).

Thysanoptera: thrips (Thripidae) such as yellow citrus thrips (Frankliniella occidentalis), melon thrips (Thrips palmi), yellow tea thrips (Scirtothrips dorsalis), onion thrips (Thrips tabaci), flower thrips (Frankliniella intonsa), rice thrips (Stenchaetothrips biformis), and poinsettia thrips (Echinothrips americanus); tube thrips (Phlaeothripidae) such as rice aculeated thrips (Haplothrips aculeatus).

Diptera: anthomyiid flies (Anthomyiidae) such as seedcorn maggot (Delia platura) and onion maggot (Delia antiqua); picture-winged flies (Ulidiidae) such as sugar beet root maggot (Tetanops myopaeformis); leafminers (Agromyzidae) such as rice leafminer (Agromyza oryzae), tomato leafminer (Liriomyza sativae), bean leafminer (Liriomyza trifolii), and garden pea leafminer (Chromatomyia horticola); grass flies (Chloropidae) such as rice stem maggot (Chlorops oryzae); fruit flies (Tephritidae) such as melon fly (Bactrocera cucurbitae), oriental fruit fly (Bactrocera dorsalis), solanum fruit fly (Bactrocera latifrons), olive fly (Bactrocera oleae), Queensland fruit fly (Bactrocera tryoni), and Mediterranean fruit fly (Ceratitis capitata); shore flies (Ephydridae) such as rice leafminer (Hydrellia griseola), oriental rice whorl maggot (Hydrellia philippina), rice whorl maggot (Hydrellia sasakii); wine flies (Drosophilidae) such as spotted wing drosophila (Drosophila suzukii); phorid flies (Phoridae) such as humpbacked fly (Megaselia spiracularis); moth flies (Psychodidae) such as bath room fly (Clogmia albipunctata); moth flies (Sciaridae) such as Bradysia agrestis Sasakawa (Bradysia difformis); gall midges (Cecidomyiidae) such as Hessian fly (Mayetiola destructor) and rice gall midge (Orseolia oryzae); stalk-eyed flies (Diopsidae) such as Diopsis macrophthalma; crane flies (Tipulidae) such as house fly (Tipula aino), common cranefly (Tipula oleracea), and European cranefly (Tipula paludosa).

Coleoptera: leaf beetles (Chrysomelidae) such as western corn rootworm (Diabrotica virgifera virgifera), southern corn rootworm (Diabrotica undecimpunctata howardi), northern corn rootworm (Diabrotica barberi), Mexican corn rootworm (Diabrotica virgifera zeae), banded cucumber beetle (Diabrotica balteata), cucurbit beetle (Diabrotica speciosa), bean leaf beetle (Cerotoma trifurcata), cereal leaf beetle (Oulema melanopus), cucurbit leaf beetle (Aulacophora femoralis), yellow striped flea beetle (Phyllotreta striolata), cabbage flea beetle (Phyllotreta cruciferae), western black flea beetle (Phyllotreta pusilla), cabbage stem flea beetle (Psylliodes chrysocephala), colorado potato beetle (Leptinotarsa decemlineata), rice leaf beetle (Oulema oryzae), grape colaspis (Colaspis brunnea), corn flea beetle (Chaetocnema pulicaria), Chaetocnema confinis, potato flea beetle (Epitrix cucumeris), rice hispa (Dicladispa armigera), southern corn leaf beetle (Myochrous denticollis), Laccoptera quadrimaculata, and tobacco flea beetle (Epitrix hirtipennis); ground beetles (Carabidae) such as seedcorn beetle (Stenolophus lecontei) and slender seedcorn beetle (Clivina impressifrons); chafers (Scarabaeidae) such as cupreous chafer (Anomala cuprea), soybean beetle (Anomala rufocuprea), sugarcane white grub (Anomala albopilosa), Japanese beetle (Popillia japonica), soybean beetle (Heptophylla picea), European chafer (Rhizotrogus majalis), western carrot beetle (Tomarus gibbosus), Holotrichia spp., Phyllophaga spp. such as June beetle (Phyllophaga crinita), and Diloboderus spp. Such as Diloboderus abderus; weevils (Curculionidae) such as coffee bean weevil (Araecerus coffeae), sweet potato weevil (Cylas formicarius), west indian sweet potato weevil (Euscepes postfasciatus), alfalfa weevil (Hypera postica), maize weevil (Sitophilus zeamais), rice plant weevil (Echinocnemus squameus), rice water weevil (Lissorhoptrus oryzophilus), Asiatic palm weevil (Rhabdoscelus lineaticollis), boll weevil (Anthonomus grandis), hunting billbug (Sphenophorus venatus), southern corn billbug (Sphenophorus callosus), soybean stalk weevil (Sternechus subsignatus), sugarcane weevil (Sphenophorus levis), Scepticus griseus, Scepticus uniformis, Mexican bean weevil (Zabrotes subfasciatus), pine shoot beetle (Tomicus piniperda), coffee berry borer (Hypothenemus hampei), Aracanthus spp. such as Aracanthus mourei, and cotton root borer (Eutinobothrus brasiliensis); darkling beetles (Tenebrionidae) such as red flour beetle (Tribolium castaneum) and confused flour beetle (Tribolium confusum); ladybirds (Coccinellidae) such as twenty-eight-spotted ladybird (Epilachna vigintioctopunctata); bark beetles (Bostrychidae) such as powder post beetle (Lyctus brunneus); spider beetles (Ptinidae); longicorn beetles (Cerambycidae) such as white-spotted longicorn beetle (Anoplophora malasiaca) and Migdolus fryanus; click beetles (Elateridae) such as sugarcane wireworm (Melanotus okinawensis), barley wireworm (Agriotes fuscicollis), click beetle (Melanotus legatus), Anchastus spp., Conoderus spp., Ctenicera spp., Limonius spp., and Aeolus spp.; and staphylinids (Staphylinidae) such as rove beetle (Paederus fuscipes).

Orthoptera: grasshoppers (Acrididae) such as Asiatic locusts (Locusta migratoria), Moroccan locust (Dociostaurus maroccanus), Australian plague locust (Chortoicetes terminifera), red locust (Nomadacris septemfasciata), brown locust (Locustana pardalina), tree locust (Anacridium melanorhodon), Italian locust (Calliptamus italicus), differential grasshopper (Melanoplus differentialis), two striped grasshopper (Melanoplus bivittatus), migratory grasshopper (Melanoplus sanguinipes), red-legged grasshopper (Melanoplus femurrubrum), clearwinged grasshopper (Camnula pellucida), desert locust (Schistocerca gregaria), yellow-winged locust (Gastrimargus musicus), spur-throated locust (Austracris guttulosa), rice grasshopper (Oxya yezoensis), Japanese grasshopper (Oxya japonica), and Bombay locust (Patanga succincta); mole crickets (Gryllotalpidae) such as mole cricket (Gryllotalpa orientalis); house crickets (Gryllidae) such as European house cricket (Acheta domesticus) and Emma field cricket (Teleogryllus emma); long-horned grasshoppers (Tettigoniidae) such as Mormon cricket (Anabrus simplex).

Hymenoptera: sawflies (Tenthredinidae) such as Cabbage sawfly (Athalia rosae) and Cabbage sawfly (Athalia japonica); Fire ant (Solenopsis spp.); and ants (Formicidae) such as Brown leaf-cutting ant (Atta capiguara).

Blattodea: German cockroaches (Blattellidae) such as German cockroach (Blattella germanica); cockroaches (Blattidae) such as smoky-brown cockroach (Periplaneta fuliginosa), American cockroach (Periplaneta americana), brown cockroach (Periplaneta brunnea), and oriental cockroach (Blatta orientalis); termites (Termitidae) such as Japanese subterranean termite (Reticulitermes speratus), formosan subterranean termite (Coptotermes formosanus), western drywood termite (Incisitermes minor), drywood termite (Cryptotermes domesticus), Taiwan termite (Odontotermes formosanus), Kosyun termite (Neotermes koshunensis), Satsuma termite (Glyptotermes satsumensis), Nakajima termite (Glyptotermes nakajimai), Katan termite (Glyptotermes fuscus), Japanese damp-wood termite (Hodotermopsis sjostedti), Koshu formosan termite (Coptotermes guangzhoensis), Amami termite (Reticulitermes amamianus), Miyatake termite (Reticulitermes miyatakei), Kanmon termite (Reticulitermes kanmonensis), Takasago termite (Nasutitermes takasagoensis), Nitobe termite (Pericapritermes nitobei), Musya termite (Sinocapritermes mushae), Cornitermes cumulans, and the like.

### EXAMPLES

The present invention will be described in more detail below by way of Production Examples, Formulation Examples, and Test Examples, but the present invention is not limited to these Examples.

### Production Example 1

A mixture of 0.30 g of 3-(4-hydroxyphenyl)-5-(trifluoromethyl)-1,2,4-oxadiazole, 0.29 g of cyclopentyl isocyanate, 0.26 g of triethylamine, and 10 mL of tetrahydrofuran was stirred at room temperature for 3 hours. The mixture thus obtained was concentrated under reduced pressure and then the residue thus obtained was subjected to silica gel column chromatography (hexane:ethyl acetate=2:1) to obtain 0.28 g of the present compound 1 shown below.

Present Compound 1:

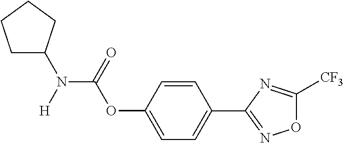 [SMILES: O=C(NC1CCCC1)Oc1ccc(-c2noc(C(F)(F)F)n2)cc1]

¹H-NMR (CDCl₃, 25° C.) δ: 8.11 (2H, d), 7.31 (2H, d), 5.02 (1H, d), 4.12-4.03 (1H, m), 2.09-2.01 (2H, m), 1.76-1.62 (4H, m), 1.55-1.48 (2H, m).

Compounds produced in accordance with the method mentioned in Production Example 1 and physical properties thereof are shown below.

Compounds in which R¹⁷ is shown below in the compound represented by formula (a):

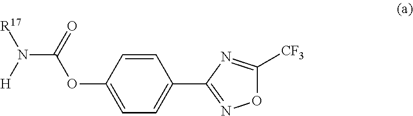

Present Compound 2 (R¹⁷: CH₂CH₂Cl):

¹H-NMR (CDCl₃, 25° C.) δ: 8.14 (2H, d), 7.32 (2H, d), 5.53 (1H, br), 3.73-3.70 (2H, m), 3.68-3.64 (2H, m).

Present Compound 3 (R¹⁷: CH₂CH₂CH₂Cl):

¹H-NMR (CDCl₃, 25° C.) δ: 8.14 (2H, d), 7.31 (2H, d), 5.26 (1H, br), 3.66 (2H, t), 3.48 (2H, dt), 2.13-2.06 (2H, m).

### Production Example 2

To a mixture of 0.13 g of triphosgene and 10 mL of chloroform, a mixture of 0.30 g of 3-(4-hydroxyphenyl)-5-(trifluoromethyl)-1,2,4-oxadiazole, 0.22 mL of diisopropylethylamine, and 10 mL of tetrahydrofuran was added dropwise, followed by stirring at room temperature for 1 hour. To the mixture thus obtained, a mixture of 0.21 mL of N-(2-methoxyethyl)methylamine, 0.33 mL of diisopropylethylamine, and 10 mL of chloroform was added dropwise, followed by stirring at room temperature overnight. To the reaction solution, an aqueous saturated sodium hydrogen carbonate solution was added, and the mixture was extracted with chloroform. The organic layer was washed with a saturated saline solution, dried over anhydrous magnesium sulfate, and then concentrated under reduced pressure. The residue thus obtained was subjected to silica gel column chromatography (hexane:ethyl acetate=4:1) to obtain 0.23 g of the present compound 4 shown below.

Present Compound 4

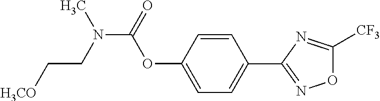 [SMILES: COCCN(C)C(=O)Oc1ccc(-c2noc(C(F)(F)F)n2)cc1]

¹H-NMR (CDCl₃, 25° C.) δ: 8.13 (2H, d), 7.32 (1H, d), 7.29 (1H, d), 3.67-3.53 (4H, m), 3.41 (1.5H, s), 3.40 (1.5H, s), 3.19 (1.5H, s), 3.09 (1.5H, s).

Compounds produced in accordance with the method mentioned in Production Example 2 and physical properties thereof are shown below.

Compounds in which R¹⁸ and R¹⁹ are shown below in the compound represented by formula (b):

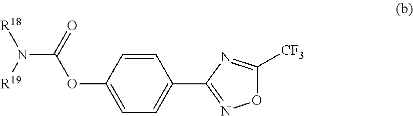

Present Compound 5 (R¹⁸: CH₃, R¹⁹: CH₂CN)

¹H-NMR (CDCl₃, 25° C.) δ: 8.17 (2H, d), 7.32 (2H, d), 4.41 (0.8H, s), 4.38 (1.2H, s), 3.28 (1.8H, s), 3.17 (1.2H, s).

Present Compound 6 (R¹⁸: CH₃, R¹⁹: CH₂CH₂CN)

¹H-NMR (CDCl₃, 25° C.) δ: 8.15 (2H, d), 7.32 (2H, d), 3.80 (0.8H, t), 3.67 (1.2H, t), 3.28 (1.8H, s), 3.14 (1.2H, s), 2.77-2.70 (2H, m).

Present Compound 7 (R¹⁸: CH₃, R¹⁹: cyclohexyl group)

¹H-NMR (CDCl₃, 25° C.) δ: 8.12 (2H, d), 7.30 (1.2H, d), 7.29 (0.8H, d), 4.08-3.97 (1H, m), 2.97 (1.8H, s), 2.91 (1.2H, s), 1.92-1.75 (4H, m), 1.74-1.64 (1H, m), 1.58-1.32 (4H, m), 1.19-1.04 (1H, m).

Present Compound 8 (R¹⁸: CH₃, R¹⁹: cyclopropyl group)

¹H-NMR (CDCl₃, 25° C.) δ:8.13 (2H, d), 7.30 (2H, d), 3.15-2.94 (3H, br m), 2.81-2.70 (1H, br m), 0.91-0.74 (4H, br m).

Present Compound 9 (R¹⁸: H, R¹⁹: CH₂CH₂OCH₂CH₃)

¹H-NMR (CDCl₃, 25° C.) δ: 8.12 (2H, d), 7.32 (2H, d), 5.52-5.44 (1H, br m), 3.61-3.45 (6H, m), 1.24 (3H, t).

Present Compound 10 (R¹⁸: CH₃, R¹⁹: propargyl group)

¹H-NMR (CDCl₃, 25° C.) δ: 8.14 (2H, d), 7.32 (2H, d), 4.29-4.19 (2H, m), 3.20 (1.5H, s), 3.10 (1.5H, s), 2.38-2.30 (1H, m).

Compounds in which R²⁴ and R²⁵ are shown below in the compound represented by formula (c):

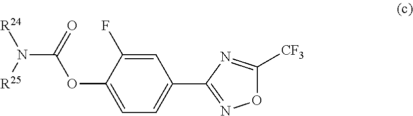

Present Compound 11 (R²⁴: CH₃, R²⁵: CH₂CH₂OCH₃)

¹H-NMR (CDCl₃, 25° C.) δ: 7.94-7.89 (2H, m), 7.43-7.34 (1H, m), 3.67-3.53 (4H, m), 3.42-3.38 (3H, m), 3.20 (1.5H, s), 3.10 (1.5H, s).

Present Compound 12 (R²⁴: CH₃, R²⁵: CH₂CN)

¹H-NMR (CDCl₃, 25° C.) δ: 7.98-7.93 (2H, m), 7.45-7.37 (1H, m), 4.43 (0.8H, s), 4.37 (1.2H, s), 3.29 (1.8H, s), 3.18 (1.2H, s).

Present Compound 13 (R²⁴: CH₃, R²⁵: CH₂CH₂CN)

¹H-NMR (CDCl₃, 25° C.) δ: 7.97-7.90 (2H, m), 7.46-7.36 (1H, m), 3.80 (0.8H, t), 3.67 (1.2H, t), 3.29 (1.8H, s), 3.16 (1.2H, s), 2.76 (0.8H, t), 2.72 (1.2H, t).

Present Compound 14 (R²⁴: CH₃, R²⁵: cyclohexyl group)

¹H-NMR (CDCl₃, 25° C.) δ: 7.94-7.88 (2H, m), 7.41-7.33 (1H, m), 4.08-3.97 (1H, m), 2.99 (1.7H, s), 2.92 (1.3H, s), 1.92-1.63 (5H, m), 1.54-1.06 (5H, m).

Present Compound 15 (R²⁴: CH₃, R²⁵: cyclopropyl group)

¹H-NMR (CDCl₃, 25° C.) δ: 7.95-7.89 (2H, m), 7.42-7.35 (1H, m), 3.18-2.96 (3H, br m), 2.77 (1H, br s), 0.92-0.72 (4H, m).

Compounds in which R²⁶ and R²⁷ are shown below in the compound represented by formula (d):

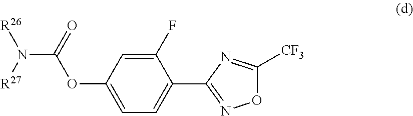

Present Compound 16 (R²⁶: CH₃, R²⁷: CH₂CH₂OCH₃)

¹H-NMR (CDCl₃, 25° C.) δ: 8.12-8.05 (1H, m), 7.21-7.10 (2H, m), 3.65-3.51 (4H, m), 3.39 (3H, s), 3.17 (1.5H, s), 3.08 (1.5H, s).

Present Compound 17 (R²⁶: CH₃, R²⁷: cyclohexyl group)

¹H-NMR (CDCl₃, 25° C.) δ: 8.13-8.05 (1H, m), 7.20-7.10 (2H, m), 4.07-3.93 (1H, m), 2.96 (1.7H, s), 2.91 (1.3H, s), 1.92-1.63 (5H, m), 1.55-1.04 (5H, m).

Present Compound 18 (R²⁶: CH₃, R²⁷: cyclopropyl group)

¹H-NMR (CDCl₃, 25° C.) δ: 8.12-8.05 (1H, m), 7.19-7.12 (2H, m), 3.15-2.94 (3H, br m), 2.76 (1H, br s), 0.92-0.72 (4H, m).

Present Compound 19

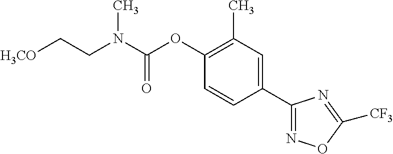 [SMILES: COCCN(C)C(=O)Oc1ccc(-c2noc(C(F)(F)F)n2)cc1C]

¹H-NMR (CDCl₃, 25° C.) δ: 8.01-7.93 (2H, m), 7.30-7.19 (1H, m), 3.69-3.52 (4H, m), 3.42-3.38 (3H, m), 3.20 (1.5H, s), 3.08 (1.5H, s), 2.30 (3H, s).

Compounds in which R²⁸ and R²⁹ are shown below in the compound represented by formula (e):

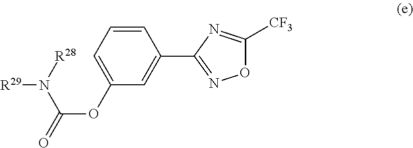

Present Compound 20 (R²⁸: CH₃, R²⁹: CH₂CH₂OCH₃)

¹H-NMR (CDCl₃, 25° C.) δ: 7.98-7.94 (1H, m), 7.91-7.86 (1H, m), 7.55-7.49 (1H, m), 7.40-7.32 (1H, m), 3.65-3.52 (4H, m), 3.43-3.37 (3H, m), 3.18 (1.5H, s), 3.08 (1.5H, s).

Present Compound 21 (R²⁸: CH₃, R²⁹: cyclopropyl group)

¹H-NMR (CDCl₃, 25° C.) δ: 7.98-7.94 (1H, m), 7.90-7.87 (1H, m), 7.55-7.49 (1H, m), 7.38-7.34 (1H, m), 3.13-2.98 (3H, br m), 2.76 (1H, br s), 0.91-0.76 (4H, m).

### Reference Production Example 1-1

To a mixture of 5.34 g of N′,4-dihydroxy-3-fluorobenzenecarboximidamide, 7.44 g of pyridine, and 48 mL of dimethylformamide, 7.25 g of trifluoroacetic anhydride was added dropwise, followed by stirring at 80° C. for 5 hours. The reaction solution was diluted with ethyl acetate and then washed sequentially with 2N hydrochloric acid, water, an aqueous saturated sodium hydrogen carbonate solution, and a saturated saline solution. The organic layer was dried over anhydrous sodium sulfate and then concentrated under reduced pressure to obtain 7.22 g of an intermediate 1 shown below.

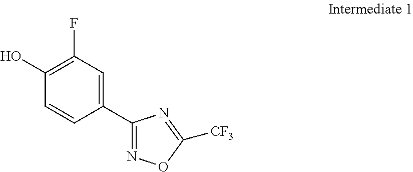 [SMILES: Oc1ccc(-c2noc(C(F)(F)F)n2)cc1F]

¹H-NMR (CDCl₃, 25° C.) δ: 7.87-7.84 (2H, m), 7.16-7.12 (1H, m), 5.75 (1H, br s).

### Reference Production Example 1-2

Compounds produced in accordance with the method mentioned in Production Example 1-1 and physical properties thereof are shown below.

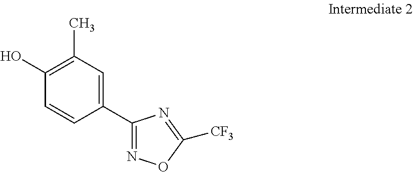 [SMILES: Cc1cc(-c2noc(C(F)(F)F)n2)ccc1O]

¹H-NMR (CDCl₃, 25° C.) δ: 7.91-7.90 (1H, m), 7.87-7.84 (1H, m), 6.88 (1H, d, J=8.5 Hz), 5.19 (1H, s), 2.32 (3H, s).

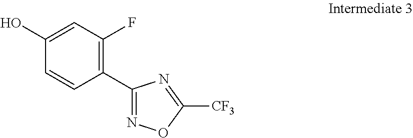 [SMILES: Oc1ccc(-c2noc(C(F)(F)F)n2)c(F)c1]

¹H-NMR (CDCl₃, 25° C.) δ: 7.99-7.95 (1H, m), 6.81-6.75 (2H, m), 6.32 (1H, br s).

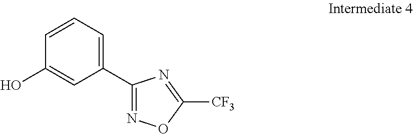 [SMILES: Oc1cccc(-c2noc(C(F)(F)F)n2)c1]

¹H-NMR (CDCl₃, 25° C.) δ: 7.70 (1H, d), 7.58 (1H, s), 7.40 (1H, t), 7.08-7.03 (1H, m), 5.38 (1H, s).

### Production Example 3

To a mixture of 0.22 g of 2-methoxyethylamine and 5 mL of acetonitrile, 0.57 g of 1,1′-carbonyldiimidazole was added at 0° C., followed by stirring at room temperature for 1 hour. To this reaction solution, 0.81 g of 3-(4-hydroxyphenyl)-5-(trifluoromethyl)-1,2,4-oxadiazole was added at room temperature, followed by stirring at 80° C. for 4 hours. To this reaction solution, an aqueous saturated sodium hydrogen carbonate solution was added, and the mixture was extracted with ethyl acetate. The organic layer was washed with water and a saturated saline solution, dried over anhydrous magnesium sulfate, and then concentrated under reduced pressure. The residue thus obtained was subjected to silica gel column chromatography (hexane:ethyl acetate=4:1) to obtain 0.13 g of the present compound 22 shown below.

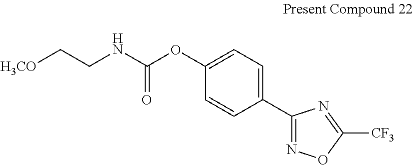 [SMILES: COCCNC(=O)Oc1ccc(-c2noc(C(F)(F)F)n2)cc1]

¹H-NMR (CDCl₃, 25° C.) δ: 8.12 (2H, d), 7.32 (2H, d), 5.51-5.41 (1H, br m), 3.57-3.45 (4H, m), 3.41 (3H, s).

Compounds produced in accordance with the method mentioned in Production Example 3 and physical properties thereof are shown below.

Compounds in which R¹⁸ and R¹⁹ are shown below in the compound represented by formula (b):

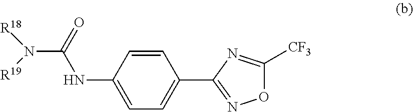

Present Compound 23 (R¹⁸: H, R¹⁹: CH₂CH₂OCH₂CH₂CH₃)

¹H-NMR (CDCl₃, 25° C.) δ: 8.12 (2H, d), 7.31 (2H, d), 5.52-5.44 (1H, br m), 3.61-3.41 (6H, m), 1.68-1.57 (2H, m), 0.95 (3H, t).

Present Compound 24 (R¹⁸: H, R¹⁹: CH₂CN)

¹H-NMR (CDCl₃, 25° C.) δ: 8.16 (2H, d), 7.33 (2H, d), 5.51-5.44 (1H, br m), 4.26 (2H, d).

Present Compound 25 (R¹⁸: H, R¹⁹: CH₂CH₂CN)

¹H-NMR (CDCl₃, 25° C.) δ: 8.15 (2H, d), 7.32 (2H, d), 5.57-5.48 (1H, br m), 3.59 (2H, q), 2.72 (2H, t).

Present Compound 26 (R¹⁸: H, R¹⁹: CH₂CH₂CH₂CN)

¹H-NMR (CDCl₃, 25° C.) δ: 8.13 (2H, d), 7.31 (2H, d), 5.31-5.22 (1H, br m), 3.45 (2H, q), 2.49 (2H, t), 2.05-1.96 (2H, m).

Present Compound 27 (R¹⁸: H, R¹⁹: cyclopentyl group)

¹H-NMR (CDCl₃, 25° C.) δ: 8.12 (2H, d), 7.31 (2H, d), 5.07-4.96 (1H, br m), 4.13-4.03 (1H, m), 2.11-1.99 (2H, m), 1.81-1.59 (4H, m), 1.57-1.45 (2H, m).

Present Compound 28 (R¹⁸: H, R¹⁹: cyclopropyl group)

¹H-NMR (CDCl₃, 25° C.) δ: 8.12 (2H, d), 7.31 (2H, d), 5.27 (1H, br s), 2.79-2.66 (1H, m), 0.86-0.79 (2H, m), 0.69-0.63 (2H, m).

### Production Example 4

To a mixture of 0.34 g of triphosgene and 10 mL of chloroform, a mixture of 0.60 g of 3-(4-aminophenyl)-5-(trifluoromethyl)-1,2,4-oxadiazole hydrochloride, 0.63 mL of triethylamine, and 15 mL of chloroform was added dropwise, followed by stirring at room temperature for 1 hour. This reaction solution was concentrated under reduced pressure and a mixture of 0.12 mL of N-(2-methoxyethyl)methylamine, 0.32 mL of triethylamine, and 10 mL of chloroform was added dropwise to a mixture of the residue thus obtained and 10 mL of chloroform, followed by stirring at room temperature for 1 hour. This reaction solution was filtered and then concentrated under reduced pressure. The residue thus obtained was subjected to silica gel column chromatography (hexane:ethyl acetate=1:1) to obtain 0.27 g of the present compound 29 shown below.

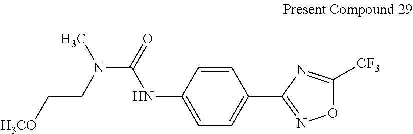 [SMILES: COCCN(C)C(=O)Nc1ccc(-c2noc(C(F)(F)F)n2)cc1]

¹H-NMR (CDCl₃, 25° C.) δ: 8.39-8.26 (1H), 8.06-7.91 (2H), 7.53-7.34 (2H), 3.62-3.64 (2H, m), 3.50-3.52 (5H, m), 3.01 (3H, s)

Compounds produced in accordance with the method mentioned in Production Example 4 and physical properties thereof are shown below.

Compounds in which R³⁰ and R³¹ are shown below in the compound represented by formula (f):

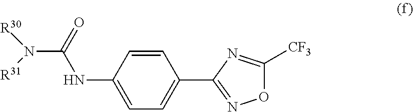

Present Compound 30 (R³⁰: CH₃, R³¹: CH₂CN)

¹H-NMR (CDCl₃, 25° C.) δ: 8.23 (2H, d), 8.05 (2H, d), 7.58 (2H, d), 7.56 (2H, d), 4.40 (2H, s), 4.19 (2H, s), 3.20 (3H, s), 3.04 (3H, s)

Present Compound 31 (R³⁰: CH₃, R³¹: CH₂CH₂CN)

¹H-NMR (CDCl₃, 25° C.) δ: 8.05 (2H, d), 7.56 (2H, d), 6.56 (1H, s), 3.68-3.71 (2H, m), 3.24 (3H, s), 2.69-2.73 (2H, m)

Present Compound 32 (R³⁰: CH₃, R³¹: cyclohexyl group)

¹H-NMR (CDCl₃, 25° C.) δ: 8.02 (2H, d), 7.57 (2H, d), 6.47 (1H, s), 4.05-4.14 (1H, m), 2.90 (3H, s), 1.81-1.85 (2H, m), 1.73-1.76 (2H, m), 1.66-1.71 (1H, m), 1.34-1.47 (4H, m), 1.04-1.15 (1H, m)

### Production Example 5

To a mixture of 0.30 g of 3-(4-aminophenyl)-5-(trifluoromethyl)-1,2,4-oxadiazole hydrochloride, 0.41 mL of triethylamine, and 10 mL of tetrahydrofuran, 0.11 mL of methoxyacetyl chloride was added dropwise, followed by stirring at room temperature for 1 hour. This reaction solution was filtered and then the filtrate was concentrated under reduced pressure. To the residue thus obtained, water was added, and the solution was extracted with ethyl acetate. The organic layer thus obtained was washed with a saturated saline solution, dried over anhydrous magnesium sulfate, and then concentrated under reduced pressure. The residue thus obtained was subjected to silica gel column chromatography (hexane:ethyl acetate=1:1) to obtain 0.35 g of the present compound 33 shown below.

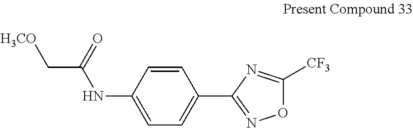 [SMILES: COCC(=O)Nc1ccc(-c2noc(C(F)(F)F)n2)cc1]

¹H-NMR (CDCl₃, 25° C.) δ: 8.43 (1H, s), 8.09 (2H, d), 7.75 (2H, d), 4.04 (2H, s), 3.52 (3H, s)

Compounds produced in accordance with the method mentioned in Production Example 5 and physical properties thereof are shown below.

Compounds in which R³² is shown below in the compound represented by formula (g):

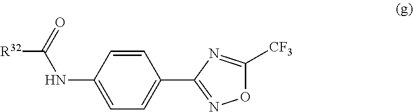

Present Compound 34 (R³²: cyclohexyl group)

¹H-NMR (CDCl₃, 25° C.) δ: 8.06 (2H, d), 7.70 (2H, d), 7.33 (1H, s), 2.22-2.37 (2H, m), 1.19-1.98 (9H, m)

Present Compound 35 (R³²: cyclopropyl group)

¹H-NMR (CDCl₃, 25° C.) δ: 8.06 (2H, d), 7.68 (2H, d), 7.50 (1H, s), 1.49-1.55 (1H, m), 1.10-1.14 (2H, m), 0.87-0.92 (2H, m)

### Production Example 6

To a mixture of 0.21 g of 1-cyano-cyclopropanecarboxylic acid and 10 mL of tetrahydrofuran, 0.22 mL of oxalyl chloride and 20 mL of dimethylformamide were sequentially added dropwise and, after stirring at room temperature for 1 hour, the reaction solution was concentrated under reduced pressure. To a mixture of the residue thus obtained, 0.40 g of 3-(4-aminophenyl)-5-(trifluoromethyl)-1,2,4-oxadiazole hydrochloride, and 10 mL of tetrahydrofuran, 0.52 mL of triethylamine was added dropwise, followed by stirring at room temperature for 1 hour. This reaction solution was filtered and then the filtrate was concentrated under reduced pressure. To the residue thus obtained, water was added, and the solution was extracted with ethyl acetate. The organic layer thus obtained was washed with a saturated saline solution, dried over anhydrous magnesium sulfate, and then concentrated under reduced pressure to obtain 0.23 g of the present compound 36 shown below.

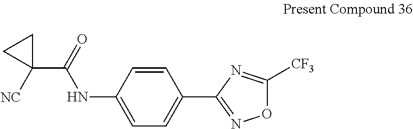 [SMILES: N#CC1(C(=O)Nc2ccc(-c3noc(C(F)(F)F)n3)cc2)CC1]

¹H-NMR (CDCl₃, 25° C.) δ: 8.13 (2H, d), 7.97 (1H, s), 7.71 (2H, d), 3.73-3.86 (2H, m), 2.56-2.65 (1H, m), 2.42-2.50 (1H, m)

Compounds produced in accordance with the method mentioned in Production Example 6 and physical properties thereof are shown below.

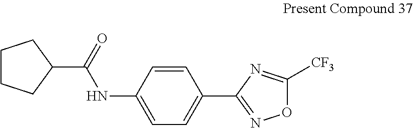 [SMILES: O=C(Nc1ccc(-c2noc(C(F)(F)F)n2)cc1)C1CCCC1]

¹H-NMR (CDCl₃, 25° C.) δ: 8.07 (2H, d), 7.70 (2H, d), 2.66-2.75 (1H, m), 1.59-2.02 (8H, m)

### Production Example 7

To a mixture of 0.60 g of 3-(4-aminophenyl)-5-(trifluoromethyl)-1,2,4-oxadiazole hydrochloride and 20 mL of pyridine, 0.22 g of cyanoacetic acid was added and then 0.50 g of 1-ethyl-3-(3-dimethylaminopropyl)carbodiimide hydrochloride was added, followed by stirring at room temperature for 1 hour. To this reaction solution, ethyl acetate was added and, after filtering with Celite, the filtrate was concentrated under reduced pressure. The residue thus obtained was subjected to silica gel column chromatography (ethyl acetate:hexane=1:1) to obtain 0.65 g of the present compound 38 shown below.

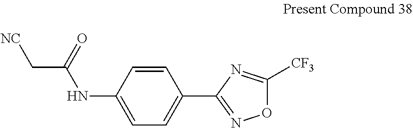 [SMILES: N#CCC(=O)Nc1ccc(-c2noc(C(F)(F)F)n2)cc1]

¹H-NMR (CDCl₃, 25° C.) δ: 8.18 (2H, d), 7.85 (1H, s), 7.75 (2H, d), 3.65 (2H, s)

Compounds produced in accordance with the method mentioned in Production Example 7 and physical properties thereof are shown below.

Compounds in which R³² is shown below in the compound represented by formula (g):

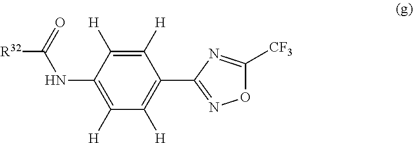

Present Compound 39 (R³²: 1-methylcyclohexyl group)

¹H-NMR (CDCl₃, 25° C.) δ: 8.09 (2H, d), 7.74 (2H, d), 7.50 (1H, s), 2.01-2.06 (3H, m), 1.22-1.67 (10H, m)

Present Compound 40 (R³²: 1-methylcyclobutyl group)

¹H-NMR (CDCl₃, 25° C.) δ: 8.10 (2H, d), 7.74 (2H, d), 7.20 (1H, s), 2.52-2.60 (2H, m), 1.84-2.14 (4H, m), 1.56 (3H, s)

Present Compound 41 (R³²: 1-methylcyclopropyl group)

¹H-NMR (CDCl₃, 25° C.) δ: 8.08 (2H, d), 7.71 (2H, d), 7.61 (1H, s), 1.34-1.37 (2H, m), 1.30 (3H, s), 0.73-0.76 (2H, m)

### Production Example 8

A mixture of 0.50 g of 3-(4-hydroxyphenyl)-5-(trifluoromethyl)-1,2,4-oxadiazole, 0.41 g of 4-methoxybenzoyl chloride, 0.39 g of potassium carbonate, and 2 mL of dimethylformamide was stirred at room temperature for 16 hours. The reaction solution was diluted with ethyl acetate, and then washed with water and a saturated saline solution. The organic layer was dried over anhydrous sodium sulfate and then concentrated under reduced pressure. The residue thus obtained was subjected to silica gel column chromatography (ethyl acetate:hexane=1:4) to obtain 0.45 g of the present compound 42 shown below.

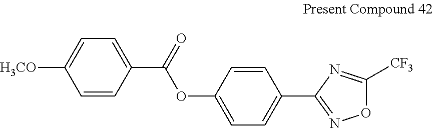 [SMILES: COc1ccc(C(=O)Oc2ccc(-c3noc(C(F)(F)F)n3)cc2)cc1]

¹H-NMR (CDCl₃, 25° C.) δ: 8.21-8.15 (4H, m), 7.39 (2H, d), 7.00 (2H, d), 3.91 (3H, s).

Compounds produced in accordance with the method mentioned in Production Example 8 and physical properties thereof are shown below.

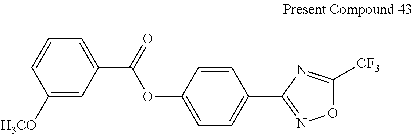 [SMILES: COc1cccc(C(=O)Oc2ccc(-c3noc(C(F)(F)F)n3)cc2)c1]

¹H-NMR (CDCl₃, 25° C.) δ: 8.21-8.18 (2H, m), 7.81 (1H, dt), 7.70 (1H, dd), 7.45-7.38 (3H, m), 7.21-7.18 (1H, m), 3.88 (3H, s).

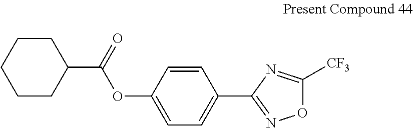 [SMILES: O=C(Oc1ccc(-c2noc(C(F)(F)F)n2)cc1)C1CCCCC1]

¹H-NMR (CDCl₃, 25° C.) δ: 8.14 (2H, d), 7.25 (5H, d), 2.63-2.55 (1H, m), 2.12-2.04 (2H, m), 1.88-1.79 (2H, m), 1.75-1.56 (3H, m), 1.44-1.24 (3H, m).

As used herein, Me represents a methyl group, Et represents an ethyl group, Pr represents a propyl group, i-Pr represents an isopropyl group, c-Pr represents a cyclopropyl group, c-Bu represents a cyclobutyl group, c-Pen represents a cyclopentyl group, c-Hex represents a cyclohexyl group, c-Hep represents a cycloheptyl group, OMe represents a methoxy group, and OEt represents an ethoxy group.

As used herein, substituent numbers A1 to substituent number A5087 represent combinations of R²⁰, R²¹, R²², and R²³ in the below-mentioned compound represented by formula (2), compound represented by formula (3), compound represented by formula (4), compound represented by formula (5), compound represented by formula (6), compound represented by formula (7), compound represented by formula (8), or compound represented by formula (9), and are hereinafter referred to as [substituent numbers; R²⁰, R²¹, R²², R²³].

For example, a substituent number A2 represents a combination in which R²⁰, R²¹, and R²³ are hydrogen atoms, and R²² is OC(═O)NHCHF₂.

[A1;H,H,OC(═O)NHCH₂F,H], [A2;H,H,OC(═O)NHCHF₂,H], [A3;H,H,OC(═O)NHCF₃,H], [A4;H,H,OC(═O)NH(CH₂)₂F,H], [A5;H,H,OC(═O)NHCH₂CHF₂,H], [A6;H,H,OC(═O)NHCH₂CF₃,H], [A7;H,H,OC(═O)NH(CH₂)₃F,H], [A8;H,H,OC(═O)NH(CH₂)₂CHF₂,H], [A9;H,H,OC(═O)NH(CH₂)₂CF₃,H], [A10;H,H,OC(═O)NHCH₂CH(CF₃)₂,H], [A11;H,H,OC(═O)NHCH₂Cl,H], [A12;H,H,OC(═O)NH(CH₂)₂Cl,H], [A13;H,H,OC(═O)NH(CH₂)₃Cl,H], [A14;H,H,OC(═O)NH(CH₂)₄Cl,H], [A15;H,H,OC(═O)NH(CH₂)₅Cl,H], [A16;H,H,OC(═O)NHCH₂Br,H], [A17;H,H,OC(═O)NH(CH₂)₂Br,H], [A18;H,H,OC(═O)NH(CH₂)₃Br,H], [A19;H,H,OC(═O)NH(CH₂)₄Br,H], [A20;H,H,OC(═O)NH(CH₂)₅Br,H], [A21;H,H,OC(═O)NHCH₂OMe,H], [A22;H,H,OC(═O)NH(CH₂)₂OMe,H], [A23;H,H,OC(═O)NH(CH₂)₃₀Me,H], [A24;H,H,OC(═O)NH(CH₂)₄₀Me,H], [A25;H,H,OC(═O)NHCH₂OEt,H], [A26;H,H,OC(═O)NH(CH₂)₂OEt,H], [A27;H,H,OC(═O)NH(CH₂)₃OEt,H], [A28;H,H,OC(═O)NHCH₂CN,H], [A29;H,H,OC(═O)NH(CH₂)₂CN,H], [A30;H,H,OC(═O)NH(CH₂)₃CN,H], [A31;H,H,OC(═O)NH(CH₂)₄CN,H], [A32;H,H,OC(═O)NH(CH₂)₅CN,H], [A33;H,H,OC(═O)NHCH₂C═CHF,H], [A34;H,H,OC(═O)NHCH₂C═CF₂,H], [A35;H,H,OC(═O)NH(CH₂)₂C═CHF,H], [A36;H,H,OC(═O)NHCHCH₂C═CF₂,H], [A37;H,H,OC(═O)NH(CH₂)₃C═CHF,H], [A38;H,H,OC(═O)NHCH₂CHCH₂C═CF₂,H], [A39;H,H,OC(═O)NHCH₂C═CHCl,H], [A40;H,H,OC(═O)NHCH₂C═CCl₂,H], [A41;H,H,OC(═O)NH(CH₂)₂C═CHCl,H], [A42;H,H,OC(═O)NHCHCH₂C═CCl₂,H], [A43;H,H,OC(═O)NH(CH₂)₃C═CHCl,H], [A44;H,H,OC(═O)NHCH₂CHCH₂C═CCl₂,H], [A45;H,H,OC(═O)NHCH₂C═CH,H], [A46;H,H,OC(═O)NHCH(Me)C═CH,H], [A47;H,H,OC(═O)NHCH₂C═CMe,H], [A48;H,H,OC(═O)NHCH₂C=CEt,H], [A49;H,H,OC(═O)NHCH₂C=CEt,H], [A50;H,H,OC(═O)NH(CH₂)₂C═CH,H], [A51;H,H,OC(═O)NH(CH₂)₂C═CMe,H], [A52;H,H,OC(═O)NH(CH₂)₃C═CH,H], [A53;H,H,OC(═O)NH(CH₂)₄C═CH,H], [A54;H,H,OC(═O)NHc-Pr,H], [A55;H,H,OC(═O)NHc-Bu,H], [A56;H,H,OC(═O)NHc-Pen,H], [A57;H,H,OC(═O)NHc-Hex,H], [A58;H,H,OC(═O)NHc-Hep,H], [A59;H,H,OC(═O)NHOCH₂F,H], [A60;H,H,OC(═O)NHOCHF₂,H], [A61;H,H,OC(═O)NHOCF₃,H], [A62;H,H,OC(═O)NHO(CH₂)₂F,H], [A63;H,H,OC(═O)NHOCH₂CHF₂,H], [A64;H,H,OC(═O)NHOCH₂CF₃,H], [A65;H,H,OC(═O)NHO(CH₂)₃F,H], [A66;H,H,OC(═O)NHO(CH₂)₂CHF₂,H], [A67;H,H,OC(═O)NHO(CH₂)₂CF₃,H], [A68;H,H,OC(═O)NHOCH₂CH(CF₃)₂,H], [A69;H,H,OC(═O)NHOCH₂CH═CH₂,H], [A70;H,H,OC(═O)NHOCH₂CH═CHF,H], [A71;H,H,OC(═O)NHOCH₂CH═CF₂,H], [A72;H,H,OC(═O)NHOCH₂CH═CHCl,H], [A73;H,H,OC(═O)NHOCH₂CH═CCl₂,H], [A74;H,H,OC(═O)NHOCH₂C═CH,H], [A75;H,H,OC(═O)NHOCH(Me)C═CH,H], [A76;H,H,OC(═O)NHOCH₂C═CMe,H], [A77;H,H,OC(═O)NMeCH₂F,H], [A78;H,H,OC(═O)NMeCHF₂,H], [A79;H,H,OC(═O)NMeCF₃,H], [A80;H,H,OC(═O)NMe(CH₂)₂F,H], [A81;H,H,OC(═O)NMeCH₂CHF₂,H], [A82;H,H,OC(═O)NMeCH₂CF₃,H], [A83;H,H,OC(═O)NMe(CH₂)₃F,H], [A84;H,H,OC(═O)NMe(CH₂)₂CHF₂,H], [A85;H,H,OC(═O)NMe(CH₂)₂CF₃,H], [A86;H,H,OC(═O)NMeCH₂CH(CF₃)₂,H], [A87;H,H,OC(═O)NMeCH₂Cl,H], [A88;H,H,OC(═O)NMe(CH₂)₂Cl,H], [A89;H,H,OC(═O)NMe(CH₂)₃Cl,H], [A90;H,H,OC(═O)NMe(CH₂)₄Cl,H], [A91;H,H,OC(═O)NMe(CH₂)₅Cl,H], [A92;H,H,OC(═O)NMeCH₂Br,H], [A93;H,H,OC(═O)NMe(CH₂)₂Br,H], [A94;H,H,OC(═O)NMe(CH₂)₃Br,H], [A95;H,H,OC(═O)NMe(CH₂)₄Br,H], [A96;H,H,OC(═O)NMe(CH₂)₅Br,H], [A97;H,H,OC(═O)NMeCH₂OMe,H], [A98;H,H,OC(═O)NMe(CH₂)₂OMe,H], [A99;H,H,OC(═O)NMe(CH₂)₃OMe,H][A100;H,H,OC(═O)NMe(CH₂)₄OMe,H]

[A101;H,H,OC(═O)NMeCH₂OEt,H], [A102;H,H,OC(═O)NMe(CH₂)₂OEt,H], [A103;H,H,OC(═O)NMe(CH₂)₃OEt,H], [A104;H,H,OC(═O)NMeCH₂CN,H], [A105;H,H,OC(═O)NMe(CH₂)₂CN,H][A106;H,H,OC(═O)NMe(CH₂)₃CN,H][A107;H,H,OC(═O)NMe(CH₂)₄CN,H][A108;H,H,OC(═O)NMe(CH₂)₅CN,H][A109;H,H,OC(═O)NMeCH₂C═CHF,H], [A110;H,H,OC(═O)NMeCH₂C═CF₂,H][A111;H,H,OC(═O)NMe(CH₂)₂C═CHF,H], [A112;H,H,OC(═O)NMeCHCH₂C═CF₂,H], [A113;H,H,OC(═O)NMe(CH)₃C═CHF,H], [A114;H,H,OC(═O)NMeCH₂CHCH₂C═CF₂,H], [A115;H,H,OC(═O)NMeCH₂C═CHCl,H], [A116;H,H,OC(═O)NMeCH₂C═CCl₂,H][A117;H,H,OC(═O)NMe(CH₂)₂C═CHCl,H], [A118;H,H,OC(═O)NMeCHCH₂C═CCl₂,H][A119;H,H,OC(═O)NMe(CH₂)₃C═CHCl,H], [A120;H,H,OC(═O)NMeCH₂CHCH₂C═CCl₂,H][A121;H,H,OC(═O)NMeCH₂C═CH,H], [A122;H,H,OC(═O)NMeCH(Me)C═CH,H][A123;H,H,OC(═O)NMeCH₂C═CMe,H][A124;H,H,OC(═O)NMeCH₂C=CEt,H], [A125;H,H,OC(═O)NMeCH₂C=CEt,H][A126;H,H,OC(═O)NMe(CH₂₂C═CH,H], [A127;H,H,OC(═O)NMe(CH₂₂C═CMe,H], [A128;H,H,OC(═O)NMe(CH₂)₃C═CH,H], [A129;H,H,OC(═O)NMe(CH)₄C═CH,H], [A130;H,H,OC(═O)NMec-Pr,H], [A131;H,H,OC(═O)NMec-Bu,H], [A132;H,H,OC(═O)NMec-Pen,H], [A133;H,H,OC(═O)NMec-Hex,H], [A134;H,H,OC(═O)NMec-Hep,H], [A135;H,H,OC(═O)NMeOCH₂F,H], [A136;H,H,OC(═O)NMeOCHF₂,H], [A137;H,H,OC(═O)NMeOCF₃,H], [A138;H,H,OC(═O)NMeO(CH₂)₂F,H], [A139;H,H,OC(═O)NMeOCH₂CHF₂,H], [A140;H,H,OC(═O)NMeOCH₂CF₃,H], [A141;H,H,OC(═O)NMeO(CH₂))F,H][A142;H,H,OC(═O)NMeO(CH₂)₂CHF₂,H], [A143;H,H,OC(═O)NMeO(CH₂)₂CF₃,H][A144;H,H,OC(═O)NMeOCH₂CH(CF₃)₂,H], [A145;H,H,OC(═O)NMeOCH₂CH═CH₂,H], [A146;H,H,OC(═O)NMeOCH₂CH═CHF,H][A147;H,H,OC(═O)NMeOCH₂CH═CF₂,H], [A148;H,H,OC(═O)NMeOCH₂CH═CHCl,H], [A149;H,H,OC(═O)NMeOCH₂CH═CCl₂,H], [A150;H,H,OC(═O)NMeOCH₂C═CH,H], [A151;H,H,OC(═O)NMeOCH(Me)C═CH,H], [A152;H,H,OC(═O)NMeCH₂C═CMe,H], [A153;H,H,OC(═O)NEtCH₂F,H], [A154;H,H,OC(═O)NEtCHF₂,H], [A155;H,H,OC(═O)NEtCF₃,H], [A156;H,H,OC(═O)NEt(CH₂)₂F,H], [A157;H,H,OC(═O)NEtCH₂CHF₂,H], [A158;H,H,OC(═O)NEtCH₂CF₃,H], [A159;H,H,OC(═O)NEt(CH₂)₃F,H], [A160;H,H,OC(═O)NEt(CH₂)₂CHF₂,H], [A161;H,H,OC(═O)NEt(CH₂)₂CF₃,H], [A162;H,H,OC(═O)NEtCH₂CH(CF₃)₂,H][A163;H,H,OC(═O)NEtCH₂Cl,H], [A164;H,H,OC(═O)NEt(CH₂)₂Cl,H], [A165;H,H,OC(═O)NEt(CH₂)₃Cl,H], [A166;H,H,OC(═O)NEt(CH₂)₄Cl,H], [A167;H,H,OC(═O)NEt(CH₂)₅Cl,H], [A168;H,H,OC(═O)NEtCH₂Br,H], [A169;H,H,OC(═O)NEt(CH₂)₂Br,H], [A170;H,H,OC(═O)NEt(CH₂)₃Br,H], [A171;H,H,OC(═O)NEt(CH₂)₄Br,H], [A172;H,H,OC(═O)NEt(CH₂)₅Br,H], [A173;H,H,OC(═O)NEtCH₂OMe,H], [A174;H,H,OC(═O)NEt(CH₂OMe,H], [A175;H,H,OC(═O)NEt(CH₂)₃OMe,H], [A176;H,H,OC(═O)NEt(CH₂)₄OMe,H], [A177;H,H,OC(═O)NEtCH₂OEt,H], [A178;H,H,OC(═O)NEt(CH₂)₂Et,H], [A179;H,H,OC(═O)NEt(CH₂)₃OEt,H], [A180;H,H,OC(═O)NEtCH₂CN,H], [A181;H,H,OC(═O)NEt(CH₂)₂CN,H], [A182;H,H,OC(═O)NEt(CH₂)₃CN,H], [A183;H,H,OC(═O)NEt(CH₂)₄CN,H], [A184; H,H,OC(═O)NEt(CH₂)₅CN,H], [A185;H,H,OC(═O)NEtCH₂C═CHF,H][A186;H,H,OC(═O)NEtCH₂C═CF₂,H][A187;H,H,OC(═O)NEt(CH₂)₂C═CHF,H], [A188;H,H,OC(═O)NEtCHCH₂C═CF₂,H], [A189;H,H,OC(═O)NEt(CH₂) C═CHF,H], [A190;H,H,OC(═O)NEtCH₂CHCH₂C═CF₂,H][A191;H,H,OC(═O)NEtCH₂C═CHCl,H][A192;H,H,OC(═O)NEtCH₂C═CCl₂,H], [A193;H,H,OC(═O)NEt(CH₂)₂C═CHCl,H], [A194;H,H,OC(═O)NEtCHCH₂C═CCl₂,H], [A195;H,H,OC(═O)NEt(CH₂)₃C═CHCl,H], [A196;H,H,OC(═O)NEtCH₂CHCH₂C═CCl₂,H], [A197;H,H,OC(═O)NEtCH₂C═CH,H][A198;H,H,OC(═O)NEtCH(Me)C═CH,H][A199;H,H,OC(═O)NEtCH₂C═CMe,H], [A200;H,H,OC(═O)NEtCH₂C=CEt,H]

[A201;H,H,OC(═O)NEtCH₂C=CEt,H][A202;H,H,OC(═O)NEt(CH₂)₂C═CH,H][A203;H,H,OC(═O)NEt(CH₂)₂C═CMe,H], [A204;H,H,OC(═O)NEt(CH₂)₃C═CH,H], [A205;H,H,OC(═O)NEt(CH₂)₄C═CH,H], [A206;H,H,OC(═O)NEtc-Pr,H], [A207;H,H,OC(═O)NEtc-Bu,H], [A208;H,H,OC(═O)NEtc-Pen,H], [A209;H,H,OC(═O)NEtc-Hex,H], [A210;H,H,OC(═O)NEtc-Hep,H], [A211;H,H,OC(═O)NEtOCH₂F,H], [A212;H,H,OC(═O)NEtOCHF₂,H], [A213;H,H,OC(═O)NEtOCF₃,H], [A214;H,H,OC(═O)NEtO(CH₂)₂F,H], [A215;H,H,OC(═O)NEtOCH₂CHF₂,H], [A216;H,H,OC(═O)NEtOCH₂CF₃,H], [A217;H,H,OC(═O)NEtO(CH₂)₃F,H][A218;H,H,OC(═O)NEtO(CH₂)CHF₂,H][A219;H,H,OC(═O)NEtO(CH₂)₂CF₃,H], [A220;H,H,OC(═O)NEtOCH₂CH(CF₃)₂,H][A221;H,H,OC(═O)NEtOCH₂CH═CH₂,H], [A222;H,H,OC(═O)NEtOCH₂CH═CHF,H], [A223;H,H,OC(═O)NEtOCH₂CH═CF₂,H], [A224;H,H,OC(═O)NEtOCH₂CH═CHCl,H], [A225;H,H,OC(═O)NEtOCH₂CH═CCl₂,H], [A226;H,H,OC(═O)NEtOCH₂C═CH,H], [A227;H,H,OC(═O)NEtOCH(Me)C═CH,H], [A228;H,H,OC(═O)NEtOCH₂C═CMe,H], [A229;H,H,OC(═O)NPrCH₂F,H], [A230;H,H,OC(═O)NPrCHF₂,H], [A231;H,H,OC(═O)NPrCF₃,H], [A232;H,H,OC(═O)NPr(CH₂)₂F,H], [A233;H,H,OC(═O)NPrCH₂CHF₂,H], [A234;H,H,OC(═O)NPrCH₂CF₃,H], [A235;H,H,OC(═O)NPr(CH₂)₃F,H], [A236;H,H,OC(═O)NPr(CH₂)CHF₂,H][A237;H,H,OC(═O)NPr(CH₂) CF₃,H][A238;H,H,OC(═O)NPrCH₂CH(CF₃)₂,H], [A239;H,H,OC(═O)NPrCH₂Cl,H], [A240;H,H,OC(═O)NPr(CH₂)₂Cl,H], [A241;H,H,OC(═O)NPr(CH₂) a C1,H][A242;H,H,OC(═O)NPr(CH₂)₄Cl,H][A243;H,H,OC(═O)NPr(CH₂)₅Cl,H], [A244;H,H,OC(═O)NPrCH₂Br,H], [A245;H,H,OC(═O)NPr(CH₂)₂Br,H], [A246;H,H,OC(═O)NPr(CH₂) a Br,H][A247;H,H,OC(═O)NPr(CH₂)₄Br,H][A248;H,H,OC(═O)NPr(CH₂)₅Br,H], [A249;H,H,OC(═O)NPrCH₂OMe,H], [A250;H,H,OC(═O)NPr(CH₂)₂OMe,H][A251;H,H,OC(═O)NPr(CH₂)₃OMe,H], [A252;H,H,OC(═O)NPr(CH₂)₄OMe,H], [A253;H,H,OC(═O)NPrCH₂OEt,H], [A254;H,H,OC(═O)NPr(CH₂)₂OEt,H][A255;H,H,OC(═O)NPr(CH₂)₃OEt,H], [A256;H,H,OC(═O)NPrCH₂CN,H], [A257;H,H,OC(═O)NPr(CH₂)₂CN,H][A258;H,H,OC(═O)NPr(CH₂)₃CN,H], [A259;H,H,OC(═O)NPr(CH₂)₄CN,H][A260;H,H,OC(═O)NPr(CH₂)₅CN,H][A261;H,H,OC(═O)NPrCH₂C═CHF,H], [A262;H,H,OC(═O)NPrCH₂C═CF₂,H], [A263;H,H,OC(═O)NPr(CH₂)₂C═CHF,H], [A264;H,H,OC(═O)NPrCHCH₂C═CF₂,H], [A265;H,H,OC(═O)NPr(CH₂)₃C═CHF,H], [A266;H,H,OC(═O)NPrCH₂CHCH₂C═CF₂,H][A267;H,H,OC(═O)NPrCH₂C═CHCl,H][A268;H,H,OC(═O)NPrCH₂C═CCl₂,H], [A269;H,H,OC(═O)NPr(CH₂)₂C═CHCl,H], [A270;H,H,OC(═O)NPrCHCH₂C═CCl₂,H], [A271;H,H,OC(═O)NPr(CH₂)₃C═CHCl,H], [A272;H,H,OC(═O)NPrCH₂CHCH₂C═CCl₂,H][A273;H,H,OC(═O)NPrCH₂C═CH,H], [A274;H,H,OC(═O)NPrCH(Me)C═CH,H], [A275;H,H,OC(═O)NPrCH₂C═CMe,H], [A276;H,H,OC(═O)NPrCH₂C=CEt,H], [A277;H,H,OC(═O)NPrCH₂C=CEt,H], [A278;H,H,OC(═O)NPr(CH₂)₂C═CH,H], [A279;H,H,OC(═O)NPr(CH₂)₂C═CMe,H], [A280;H,H,OC(═O)NPr(CH₂)₃C═CH,H], [A281;H,H,OC(═O)NPr(CH₂)₄C═CH,H], [A282;H,H,OC(═O)NPrc-Pr,H], [A283;H,H,OC(═O)NPrc-Bu,H], [A284;H,H,OC(═O)NPrc-Pen,H], [A285;H,H,OC(═O)NPrc-Hex,H], [A286;H,H,OC(═O)NPrc-Hep,H], [A287;H,H,OC(═O)NPrOCH₂F,H], [A288;H,H,OC(═O)NPrOCHF₂,H], [A289;H,H,OC(═O)NPrOCF₃,H], [A290;H,H,OC(═O)NPrO(CH₂)₂F,H], [A291;H,H,OC(═O)NPrOCH₂CHF₂,H], [A292;H,H,OC(═O)NPrOCH₂CF₃,H], [A293;H,H,OC(═O)NPrO(CH₂)₃F,H][A294;H,H,OC(═O)NPrO(CH₂)₂CHF₂,H], [A295;H,H,OC(═O)NPrO(CH₂)₂CF₃,H][A296;H,H,OC(═O)NPrOCH₂CH(CF₃)₂,H], [A297;H,H,OC(═O)NPrOCH₂CH═CH₂,H], [A298;H,H,OC(═O)NPrOCH₂CH═CHF,H][A299;H,H,OC(═O)NPrOCH₂CH═CF₂,H], [A300;H,H,OC(═O)NPrOCH₂CH═CHCl,H]

[A301;H,H,OC(═O)NPrOCH₂CH═CCl₂,H][A302;H,H,OC(═O)NPrOCH₂C═CH,H], [A303;H,H,OC(═O)NPrOCH(Me)C═CH,H][A304;H,H,OC(═O)NPrOCH₂C═CMe,H], [A305;H,H,OC(═O)N(i-Pr)CH₂F,H], [A306;H,H,OC(═O)N(i-Pr)CHF₂,H], [A307;H,H,OC(═O)N(i-Pr)CF₃,H], [A308;H,H,OC(═O)N(i-Pr)(CH₂)₂F,H], [A309;H,H,OC(═O)N(i-Pr) CH₂CHF₂,H], [A310;H,H,OC(═O)N(i-Pr)CH₂CF₃,H], [A311;H,H,OC(═O)N(i-Pr)(CH₂)₃F,H], [A312;H,H,OC(═O)N(i-Pr)(CH₂)₂CHF₂,H], [A313;H,H,OC(═O)N(i-Pr)(CH₂)₂CF₃,H], [A314;H,H,OC(═O)N(i-Pr)CH₂CH(CF₃)₂,H], [A315;H,H,OC(═O)N(i-Pr) CH₂Cl,H], [A316;H,H,OC(═O)N(i-Pr)(CH₂)₂Cl,H], [A317;H,H,OC(═O)N(i-Pr)(CH₂)₃Cl,H], [A318;H,H,OC(═O)N(i-Pr)(CH₂)₄Cl,H], [A319;H,H,OC(═O)N(i-Pr)(CH₂)₅Cl,H], [A320;H,H,OC(═O)N(i-Pr)CH₂Br,H], [A321;H,H,OC(═O)N(i-Pr)(CH₂)₂Br,H], [A322;H,H,OC(═O)N(i-Pr)(CH₂)₃Br,H], [A323;H,H,OC(═O)N(i-Pr)(CH₂)₄Br,H], [A324;H,H,OC(═O)N(i-Pr)(CH₂)₅Br,H], [A325;H,H,OC(═O)N(i-Pr) CH₂OMe,H], [A326;H,H,OC(═O)N(i-Pr)(CH₂)₂OMe,H], [A327;H,H,OC(═O)N(i-Pr)(CH₂)₃OMe,H], [A328;H,H,OC(═O)N(i-Pr)(CH₂)₄OMe,H], [A329;H,H,OC(═O)N(i-Pr)CH₂OEt,H], [A330;H,H,OC(═O)N(i-Pr)(CH₂)₂OEt,H], [A331;H,H,OC(═O)N(i-Pr)(CH₂)₃OEt,H], [A332;H,H,OC(═O)N(i-Pr)CH₂CN,H], [A333;H,H,OC(═O)N(i-Pr)(CH₂)₂CN,H], [A334;H,H,OC(═O)N(i-Pr)(CH₂)₃CN,H], [A335;H,H,OC(═O)N(i-Pr)(CH₂)₄CN,H], [A336;H,H,OC(═O)N(i-Pr)(CH₂)₅CN,H], [A337;H,H,OC(═O)N(i-Pr) CH₂C═CHF,H], [A338;H,H,OC(═O)N(i-Pr) CH₂C═CF₂,H], [A339;H,H,OC(═O)N(i-Pr)(CH₂)₂C═CHF,H], [A340;H,H,OC(═O)N(i-Pr)CHCH₂C═CF₂,H], [A341;H,H,OC(═O)N(i-Pr)(CH₂)₃C═CHF,H], [A342;H,H,OC(═O)N(i-Pr) CH₂CHCH₂C═CF₂,H], [A343;H,H,OC(═O)N(i-Pr)CH₂C═CHCl,H], [A344;H,H,OC(═O)N(i-Pr) CH₂C═CCl₂,H], [A345;H,H,OC(═O)N(i-Pr)(CH₂)₂C═CHCl,H], [A346;H,H,OC(═O)N(i-Pr)CHCH₂C═CCl₂,H], [A347;H,H,OC(═O)N(i-Pr)(CH₂)₃C═CHCl,H], [A348;H,H,OC(═O)N(i-Pr) CH₂CHCH₂C═CCl₂,H], [A349;H,H,OC(═O)N(i-Pr)CH₂C═CH,H], [A350;H,H,OC(═O)N(i-Pr)CH(Me)C═CH,H], [A351;H,H,OC(═O)N(i-Pr)CH₂C═CMe,H], [A352;H,H,OC(═O)N(i-Pr)CH₂C=CEt,H], [A353;H,H,OC(═O)N(i-Pr) CH₂C=CEt,H], [A354;H,H,OC(═O)N(i-Pr)(CH₂₂C═CH,H], [A355;H,H,OC(═O)N(i-Pr)(CH₂)₂C═CMe,H], [A356;H,H,OC(═O)N(i-Pr)(CH₂)₃C═CH,H], [A357;H,H,OC(═O)N(i-Pr)(CH₂)₄C═CH,H], [A358;H,H,OC(═O)N(i-Pr)c-Pr,H], [A359;H,H,OC(═O)N(i-Pr) c-Bu,H], [A360;H,H,OC(═O)N(i-Pr)c-Pen,H], [A361;H,H,OC(═O)N(i-Pr) c-Hex,H], [A362;H,H,OC(═O)N(i-Pr) c-Hep,H], [A363;H,H,OC(═O)N(i-Pr)OCH₂F,H], [A364;H,H,OC(═O)N(i-Pr)OCHF₂,H], [A365;H,H,OC(═O)N(i-Pr)OCF₃,H], [A366;H,H,OC(═O)N(i-Pr)O(CH₂)₂F,H], [A367;H,H,OC(═O)N(i-Pr)OCH₂CHF₂,H], [A368;H,H,OC(═O)N(i-Pr)OCH₂CF₃,H], [A369;H,H,OC(═O)N(i-Pr)O(CH₂)₃F,H], [A370;H,H,OC(═O)N(i-Pr)O(CH₂)₂CHF₂,H], [A371;H,H,OC(═O)N(i-Pr)O(CH₂)₂CF₃,H], [A372;H,H,OC(═O)N(i-Pr)OCH₂CH(CF₃)₂,H], [A373;H,H,OC(═O)N(i-Pr)OCH₂CH═CH₂,H], [A374;H,H,OC(═O)N(i-Pr)OCH₂CH═CHF,H], [A375;H,H,OC(═O)N(i-Pr)OCH₂CH═CF₂,H], [A376;H,H,OC(═O)N(i-Pr)OCH₂CH═CHCl,H], [A377;H,H,OC(═O)N(i-Pr)OCH₂CH═CCl₂,H], [A378;H,H,OC(═O)N(i-Pr)OCH₂C═CH,H], [A379;H,H,OC(═O)N(i-Pr)OCH(Me)C═CH,H], [A380;H,H,OC(═O)N(i-Pr)OCH₂C═CMe,H], [A381;H,H,OC(═S)NHCH₂F,H], [A382;H,H,OC(═S)NHCHF₂,H], [A383;H,H,OC(═S)NHCF₃,H], [A384;H,H,OC(═S)NH(CH₂)₂F,H], [A385;H,H,OC(═S)NHCH₂CHF₂,H], [A386;H,H,OC(═S)NHCH₂CF₃,H], [A387;H,H,OC(═S)NH(CH₂)₃F,H], [A388;H,H,OC(═S)NH(CH₂)₂CHF₂,H], [A389;H,H,OC(═S)NH(CH₂)₂CF₃,H][A390;H,H,OC(═S)NHCH₂CH(CF₃)₂,H], [A391;H,H,OC(═S)NHCH₂Cl,H], [A392;H,H,OC(═S)NH(CH₂)₂Cl,H], [A393;H,H,OC(═S)NH(CH₂)₃Cl,H], [A394;H,H,OC(═S)NH(CH₂)₄Cl,H], [A395;H,H,OC(═S)NH(CH₂)₅Cl,H], [A396;H,H,OC(═S)NHCH₂Br,H], [A397;H,H,OC(═S)NH(CH₂)₂Br,H], [A398;H,H,OC(═S)NH(CH₂)₃Br,H], [A399;H,H,OC(═S)NH(CH₂)₄Br,H], [A400;H,H,OC(═S)NH(CH₂)₅Br,H]

[A401;H,H,OC(═S)NHCH₂OMe,H], [A402;H,H,OC(═S)NH(CH₂)₂OMe,H], [A403;H,H,OC(═S)NH(CH₂)₃OMe,H], [A404;H,H,OC(═S)NH(CH₂)₄OMe,H], [A405;H,H,OC(═S)NHCH₂OEt,H], [A406;H,H,OC(═S)NH(CH₂)₂OEt,H], [A407;H,H,OC(═S)NH(CH₂)₃OEt,H], [A408;H,H,OC(═S)NHCH₂CN,H], [A409;H,H,OC(═S)NH(CH₂)₂CN,H], [A410;H,H,OC(═S)NH(CH₂)₃CN,H], [A411;H,H,OC(═S)NH(CH₂)₄CN,H], [A412;H,H,OC(═S)NH(CH₂)₅CN,H], [A413;H,H,OC(═S)NHCH₂C═CHF,H], [A414;H,H,OC(═S)NHCH₂C═CF₂,H], [A415;H,H,OC(═S)NH(CH₂)₂C═CHF,H][A416;H,H,OC(═S)NHCHCH₂C═CF₂,H][A417;H,H,OC(═S)NH(CH₂)₃C═CHF,H], [A418;H,H,OC(═S)NHCH₂CHCH₂C═CF₂,H][A419;H,H,OC(═S)NHCH₂C═CHCl,H], [A420;H,H,OC(═S)NHCH₂C═CCl₂,H][A421;H,H,OC(═S)NH(CH₂)₂C═CHCl,H], [A422;H,H,OC(═S)NHCHCH₂C═CCl₂,H], [A423;H,H,OC(═S)NH(CH₂)₃C═CHCl,H], [A424;H,H,OC(═S)NHCH₂CHCH₂C═CCl₂,H], [A425;H,H,OC(═S)NHCH₂C═CH,H], [A426;H,H,OC(═S)NHCH(Me)C═CH,H], [A427;H,H,OC(═S)NHCH₂C═CMe,H], [A428;H,H,OC(═S)NHCH₂C=CEt,H], [A429;H,H,OC(═S)NHCH₂C=CEt,H], [A430;H,H,OC(═S)NH(CH₂)₂C═CH,H], [A431;H,H,OC(═S)NH(CH₂)₂C═CMe,H], [A432;H,H,OC(═S)NH(CH₂)₃C═CH,H][A433;H,H,OC(═S)NH(CH₂)₄C═CH,H], [A434;H,H,OC(═S)NHc-Pr,H], [A435;H,H,OC(═S)NHc-Bu,H], [A436;H,H,OC(═S)NHc-Pen,H], [A437;H,H,OC(═S)NHc-Hex,H], [A438;H,H,OC(═S)NHc-Hep,H], [A439;H,H,OC(═S)NHOCH₂F,H], [A440;H,H,OC(═S)NHOCHF₂,H], [A441;H,H,OC(═S)NHOCF₃,H], [A442;H,H,OC(═S)NHO(CH₂)₂F,H], [A443;H,H,OC(═S)NHOCH₂CHF₂,H], [A444;H,H,OC(═S)NHOCH₂CF₃,H], [A445;H,H,OC(═S)NHO(CH₂)₃F,H], [A446;H,H,OC(═S)NHO(CH₂)₂CHF₂,H], [A447;H,H,OC(═S)NHO(CH₂)₂CF₃,H][A448;H,H,OC(═S)NHOCH₂CH(CF₃)₂,H], [A449;H,H,OC(═S)NHOCH₂CH═CH₂,H], [A450;H,H,OC(═S)NHOCH₂CH═CHF,H], [A451;H,H,OC(═S)NHOCH₂CH═CF₂,H][A452;H,H,OC(═S)NHOCH₂CH═CHCl,H], [A453;H,H,OC(═S)NHOCH₂CH═CCl₂,H][A454;H,H,OC(═S)NHOCH₂C═CH,H], [A455;H,H,OC(═S)NHOCH(Me)C═CH,H], [A456;H,H,OC(═S)NHOCH₂C═CMe,H], [A457;H,H,OC(═S)NMeCH₂F,H], [A458;H,H,OC(═S)NMeCHF₂,H], [A459;H,H,OC(═S)NMeCF₃,H], [A460;H,H,OC(═S)NMe(CH₂)₂F,H], [A461;H,H,OC(═S)NMeCH₂CHF₂,H], [A462;H,H,OC(═S)NMeCH₂CF₃,H], [A463;H,H,OC(═S)NMe(CH₂)F,H], [A464;H,H,OC(═S)NMe(CH₂)CHF₂,H], [A465;H,H,OC(═S)NMe(CH₂)₂CF₃,H], [A466;H,H,OC(═S)NMeCH₂CH(CF₃)₂,H], [A467;H,H,OC(═S)NMeCH₂Cl,H], [A468;H,H,OC(═S)NMe(CH₂)₂Cl,H], [A469;H,H,OC(═S)NMe(CH₂)₃Cl,H], [A470;H,H,OC(═S)NMe(CH₂)₄Cl,H], [A471;H,H,OC(═S)NMe(CH₂)₅Cl,H], [A472;H,H,OC(═S)NMeCH₂Br,H], [A473;H,H,OC(═S)NMe(CH₂)₂Br,H], [A474;H,H,OC(═S)NMe(CH₂)₃Br,H], [475;H,H,OC(═S)NMe(CH₂)₄Br,H], [A476;H,H,OC(═S)NMe(CH₂)₅Br,H], [A477;H,H,OC(═S)NMeCH₂OMe,H], [A478;H,H,OC(═S)NMe(CH₂)₂OMe,H], [A479;H,H,OC(═S)NMe(CH)₃OMe,H], [A480;H,H,OC(═S)NMe(CH₂)₄OMe,H], [A481;H,H,OC(═S)NMeCH₂OEt,H], [A482;H,H,OC(═S)NMe(CH₂)₂OEt,H][A483;H,H,OC(═S)NMe(CH₂)₃OEt,H], [A484;H,H,OC(═S)NMeCH₂CN,H], [A485;H,H,OC(═S)NMe(CH₂)₂CN,H], [A486;H,H,OC(═S)NMe(CH₂)₃CN,H], [A487;H,H,OC(═S)NMe(CH₂)₄CN,H], [A488;H,H,OC(═S)NMe(CH₂)₅CN,H], [A489;H,H,OC(═S)NMeCH₂C═CHF,H], [A490;H,H,OC(═S)NMeCH₂C═CF₂,H], [A491;H,H,OC(═S)NMe(CH₂)₂C═CHF,H], [A492;H,H,OC(═S)NMeCHCH₂C═CF₂,H][A493;H,H,OC(═S)NMe(CH₂)₃C═CHF,H], [A494;H,H,OC(═S)NMeCH₂CHCH₂C═CF₂,H], [A495;H,H,OC(═S)NMeCH₂C═CHCl,H], [A496;H,H,OC(═S)NMeCH₂C═CCl₂,H], [A497;H,H,OC(═S)NMe(CH₂)₂C═CHCl,H], [A498;H,H,OC(═S)NMeCHCH₂C═CCl₂,H][A499;H,H,OC(═S)NMe(CH)₃C═CHCl,H], [A500;H,H,OC(═S)NMeCH₂CHCH₂C═CCl₂,H],

[A501;H,H,OC(═S)NMeCH₂C═CH,H], [A502;H,H,OC(═S)NMeCH(Me)C═CH,H][A503;H,H,OC(═S)NMeCH₂C═CMe,H], [A504;H,H,OC(═S)NMeCH₂C=CEt,H], [A505;H,H,OC(═S)NMeCH₂C=CEt,H], [A506;H,H,OC(═S)NMe(CH₂)₂C═CH,H], [A507;H,H,OC(═S)NMe(CH)₂C═CMe,H], [A508;H,H,OC(═S)NMe(CH₂)₃C═CH,H], [A509;H,H,OC(═S)NMe(CH₂)₄C═CH,H], [A510;H,H,OC(═S)NMec-Pr,H], [A511;H,H,OC(═S)NMec-Bu,H], [A512;H,H,OC(═S)NMec-Pen,H], [A513;H,H,OC(═S)NMec-Hex,H], [A514;H,H,OC(═S)NMec-Hep,H], [A515;H,H,OC(═S)NMeOCH₂F,H], [A516;H,H,OC(═S)NMeOCHF₂,H], [A517;H,H,OC(═S)NMeOCF₃,H], [A518;H,H,OC(═S)NMeO(CH₂)₂F,H], [A519;H,H,OC(═S)NMeOCH₂CHF₂,H], [A520;H,H,OC(═S)NMeOCH₂CF₃,H], [A521;H,H,OC(═S)NMeO(CH₂)₃F,H][A522;H,H,OC(═S)NMeO(CH₂)₂CHF₂,H][A523;H,H,OC(═S)NMeO(CH₂)₂CF₃,H][A524;H,H,OC(═S)NMeOCH₂CH(CF₃)₂,H], [A525;H,H,OC(═S)NMeOCH₂CH═CH₂,H], [A526;H,H,OC(═S)NMeOCH₂CH═CHF,H][A527;H,H,OC(═S)NMeOCH₂CH═CF₂,H][A528;H,H,OC(═S)NMeOCH₂CH═CHCl,H], [A529;H,H,OC(═S)NMeOCH₂CH═CCl₂,H], [A530;H,H,OC(═S)NMeOCH₂C═CH,H], [A531;H,H,OC(═S)NMeOCH(Me)C═CH,H][A532;H,H,OC(═S)NMeOCH₂C═CMe,H], [A533;H,H,OC(═S)NEtCH₂F,H], [A534;H,H,OC(═S)NEtCHF₂,H], [A535;H,H,OC(═S)NEtCF₃,H], [A536;H,H,OC(═S)NEt(CH₂)₂F,H], [A537;H,H,OC(═S)NEtCH₂CHF₂,H], [A538;H,H,OC(═S)NEtCH₂CF₃,H], [A539;H,H,OC(═S)NEt(CH₂)₃F,H], [A540;H,H,OC(═S)NEt(CH₂)₂CHF₂,H], [A541;H,H,OC(═S)NEt(CH₂)₂CF₃,H], [A542;H,H,OC(═S)NEtCH₂CH(CF₃)₂,H], [A543;H,H,OC(═S)NEtCH₂Cl,H], [A544;H,H,OC(═S)NEt(CH₂)₂Cl,H], [A545;H,H,OC(═S)NEt(CH₂)₃Cl,H][A546;H,H,OC(═S)NEt(CH₂)₄Cl,H][A547;H,H,OC(═S)NEt(CH₂)₅Cl,H], [A548;H,H,OC(═S)NEtCH₂Br,H], [A549;H,H,OC(═S)NEt(CH₂)₂Br,H][A550;H,H,OC(═S)NEt(CH₂)₃Br,H], [A551;H,H,OC(═S)NEt(CH₂)₄Br,H], [A552;H,H,OC(═S)NEt(CH₂)₅Br,H], [A553;H,H,OC(═S)NEtCH₂OMe,H], [A554;H,H,OC(═S)NEt(CH₂)₂OMe,H][A555;H,H,OC(═S)NEt(CH₂)₃OMe,H][A556;H,H,OC(═S)NEt(CH₂)₄OMe,H], [A557;H,H,OC(═S)NEtCH₂OEt,H], [A558;H,H,OC(═S)NEt(CH₂)₂OEt,H][A559;H,H,OC(═S)NEt(CH₂)₃OEt,H], [A560;H,H,OC(═S)NEtCH₂CN,H], [A561;H,H,OC(═S)NEt(CH₂)₂CN,H][A562;H,H,OC(═S)NEt(CH₂)₃CN,H][A563;H,H,OC(═S)NEt(CH₂)₄CN,H], [A564;H,H,OC(═S)NEt(CH₂)₅CN,H][A565;H,H,OC(═S)NEtCH₂C═CHF,H][A566;H,H,OC(═S)NEtCH₂C═CF₂,H], [A567;H,H,OC(═S)NEt(CH₂)₂C═CHF,H][A568;H,H,OC(═S)NEtCHCH₂C═CF₂,H], [A569;H,H,OC(═S)NEt(CH₂) a C═CHF,H], [A570;H,H,OC(═S)NEtCH₂CHCH₂C═CF₂,H], [A571;H,H,OC(═S)NEtCH₂C═CHCl,H], [A572;H,H,OC(═S)NEtCH₂C═CCl₂,H], [A573;H,H,OC(═S)NEt(CH₂)₂C═CHCl,H], [A574;H,H,OC(═S)NEtCHCH₂C═CCl₂,H], [A575;H,H,OC(═S)NEt(CH₂)₃C═CHCl,H], [A576;H,H,OC(═S)NEtCH₂CHCH₂C═CCl₂,H][A577;H,H,OC(═S)NEtCH₂C═CH,H], [A578;H,H,OC(═S)NEtCH(Me)C═CH,H], [A579;H,H,OC(═S)NEtCH₂C═CMe,H], [A580;H,H,OC(═S)NEtCH₂C=CEt,H], [A581;H,H,OC(═S)NEtCH₂C=CEt,H], [A582;H,H,OC(═S)NEt(CH₂)₂C═CH,H][A583;H,H,OC(═S)NEt(CH₂)₂C═CMe,H], [A584;H,H,OC(═S)NEt(CH₂)₃C═CH,H], [A585;H,H,OC(═S)NEt(CH₂)₄C═CH,H], [A586;H,H,OC(═S)NEtc-Pr,H], [A587;H,H,OC(═S)NEtc-Bu,H], [A588;H,H,OC(═S)NEtc-Pen,H], [A589;H,H,OC(═S)NEtc-Hex,H], [A590;H,H,OC(═S)NEtc-Hep,H], [A591;H,H,OC(═S)NEtOCH₂F,H], [A592;H,H,OC(═S)NEtOCHF₂,H], [A593;H,H,OC(═S)NEtOCF₃,H], [A594;H,H,OC(═S)NEtO(CH₂)₂F,H], [A595;H,H,OC(═S)NEtOCH₂CHF₂,H], [A596;H,H,OC(═S)NEtOCH₂CF₃,H], [A597;H,H,OC(═S)NEtO(CH₂)₃F,H][A598;H,H,OC(═S)NEtO(CH₂)₂CHF₂,H][A599;H,H,OC(═S)NEtO(CH₂)₂CF₃,H], [A600;H,H,OC(═S)NEtOCH₂CH(CF₃)₂,H],

[A601;H,H,OC(═S)NEtOCH₂CH═CH₂,H], [A602;H,H,OC(═S)NEtOCH₂CH═CHF,H], [A603;H,H,OC(═S)NEtOCH₂CH═CF₂,H], [A604;H,H,OC(═S)NEtOCH₂CH═CHCl,H], [A605;H,H,OC(═S)NEtOCH₂CH═CCl₂,H], [A606;H,H,OC(═S)NEtOCH₂C═CH,H], [A607;H,H,OC(═S)NEtOCH(Me)C═CH,H][A608;H,H,OC(═S)NEtOCH₂C═CMe,H], [A609;H,H,OC(═S)NPrCH₂F,H], [A610;H,H,OC(═S)NPrCHF₂,H], [A611;H,H,OC(═S)NPrCF₃,H], [A612;H,H,OC(═S)NPr(CH₂)₂F,H], [A613;H,H,OC(═S)NPrCH₂CHF₂,H], [A614;H,H,OC(═S)NPrCH₂CF₃,H], [A615;H,H,OC(═S)NPr(CH₂)₃F,H], [A616;H,H,OC(═S)NPr(CH₂)₂CHF₂,H][A617;H,H,OC(═S)NPr(CH₂₂CF₃,H], [A618;H,H,OC(═S)NPrCH₂CH(CF₃)₂,H], [A619;H,H,OC(═S)NPrCH₂Cl,H], [A620;H,H,OC(═S)NPr(CH₂₂Cl,H], [A621;H,H,OC(═S)NPr(CH₂)₃Cl,H], [A622;H,H,OC(═S)NPr(CH₂)₄Cl,H][A623;H,H,OC(═S)NPr(CH₂)₅Cl,H], [624;H,H,OC(═S)NPrCH₂Br,H], [A625;H,H,OC(═S)NPr(CH₂)₂Cl,H], [A626;H,H,OC(═S)NPr(CH₂)₃Br,H][A627;H,H,OC(═S)NPr(CH₂)₄Br,H][A628;H,H,OC(═S)NPr(CH₂)₅Br,H], [A629;H,H,OC(═S)NPrCH₂OMe,H], [A630;H,H,OC(═S)NPr(CH₂)₂OMe,H], [A631;H,H,OC(═S)NPr(CH₂)₃OMe,H][A632;H,H,OC(═S)NPr(CH₂)₄OMe,H], [A633;H,H,OC(═S)NPrCH₂OEt,H], [A634;H,H,OC(═S)NPr(CH₂)₂OEt,H][A635;H,H,OC(═S)NPr(CH₂)₃OEt,H], [A636;H,H,OC(═S)NPrCH₂CN,H], [A637;H,H,OC(═S)NPr(CH₂)₂CN,H], [A638;H,H,OC(═S)NPr(CH₂)₃CN,H][A639;H,H,OC(═S)NPr(CH₂)₄CN,H][A640;H,H,OC(═S)NPr(CH₂)₅CN,H][A641;H,H,OC(═S)NPrCH₂C═CHF,H], [A642;H,H,OC(═S)NPrCH₂C═CF₂,H], [A643;H,H,OC(═S)NPr(CH₂)₂C═CHF,H], [A644;H,H,OC(═S)NPrCHCH₂C═CF₂,H][A645;H,H,OC(═S)NPr(CH₂) a C═CHF,H], [A646;H,H,OC(═S)NPrCH₂CHCH₂C═CF₂,H][A647;H,H,OC(═S)NPrCH₂C═CHCl,H], [A648;H,H,OC(═S)NPrCH₂C═CCl₂,H][A649;H,H,OC(═S)NPr(CH₂)₂C═CHCl,H], [A650;H,H,OC(═S)NPrCHCH₂C═CCl₂,H], [A651;H,H,OC(═S)NPr(CH₂)₃C═CHCl,H], [A652;H,H,OC(═S)NPrCH₂CHCH₂C═CCl₂,H], [A653;H,H,OC(═S)NPrCH₂C═CH,H], [A654;H,H,OC(═S)NPrCH(Me)C═CH,H][A655;H,H,OC(═S)NPrCH₂C═CMe,H], [A656;H,H,OC(═S)NPrCH₂C=CEt,H], [A657;H,H,OC(═S)NPrCH₂C=CEt,H], [A658;H,H,OC(═S)NPr(CH₂)₂C═CH,H], [A659;H,H,OC(═S)NPr(CH₂)₂C═CMe,H], [A660;H,H,OC(═S)NPr(CH₂)₃C═CH,H], [A661;H,H,OC(═S)NPr(CH₂)₄C═CH,H], [A662;H,H,OC(═S)NPrc-Pr,H], [A663;H,H,OC(═S)NPrc-Bu,H], [A664;H,H,OC(═S)NPrc-Pen,H], [A665;H,H,OC(═S)NPrc-Hex,H], [A666;H,H,OC(═S)NPrc-Hep,H], [A667;H,H,OC(═S)NPrOCH₂F,H], [A668;H,H,OC(═S)NPrOCHF₂,H], [A669;H,H,OC(═S)NPrOCF₃,H], [A670;H,H,OC(═S)NPrO(CH₂)₂F,H], [A671;H,H,OC(═S)NPrOCH₂CHF₂,H], [A672;H,H,OC(═S)NPrOCH₂CF₃,H], [A673;H,H,OC(═S)NPrO(CH₂)₃F,H], [A674;H,H,OC(═S)NPrO(CH₂)₂CHF₂,H][A675;H,H,OC(═S)NPrO(CH₂)₂CF₃,H][A676;H,H,OC(═S)NPrOCH₂CH(CF₃)₂,H], [A677;H,H,OC(═S)NPrOCH₂CH═CH₂,H][A678;H,H,OC(═S)NPrOCH₂CH═CHF,H], [A679;H,H,OC(═S)NPrOCH₂CH═CF₂,H][A680;H,H,OC(═S)NPrOCH₂CH═CHCl,H], [A681;H,H,OC(═S)NPrOCH₂CH═CCl₂,H][A682;H,H,OC(═S)NPrOCH₂C═CH,H], [A683;H,H,OC(═S)NPrOCH(Me)C═CH,H], [A684;H,H,OC(═S)NPrOCH₂C═CMe,H], [A685;H,H,OC(═S)N(i-Pr)CH₂F,H], [A686;H,H,OC(═S)N(i-Pr)CHF₂,H], [A687;H,H,OC(═S)N(i-Pr)CF₃,H], [A688;H,H,OC(═S)N(i-Pr)(CH₂)₂F,H], [A689;H,H,OC(═S)N(i-Pr)CH₂CHF₂,H], [A690;H,H,OC(═S)N(i-Pr)CH₂CF₃,H], [A691;H,H,OC(═S)N(i-Pr)(CH₂)₃F,H], [A692;H,H,OC(═S)N(i-Pr)(CH₂)₂CHF₂,H], [A693;H,H,OC(═S)N(i-Pr)(CH₂)₂CF₃,H], [A694;H,H,OC(═S)N(i-Pr)CH₂CH(CF₃)₂,H], [A695;H,H,OC(═S)N(i-Pr) CH₂Cl,H], [A696;H,H,OC(═S)N(i-Pr)(CH₂)₂Cl,H], [A697;H,H,OC(═S)N(i-Pr)(CH₂)₃Cl,H], [A698;H,H,OC(═S)N(i-Pr)(CH₂)₄Cl,H], [A699;H,H,OC(═S)N(i-Pr)(CH₂)₅Cl,H], [A700;H,H,OC(═S)N(i-Pr) CH₂Br,H],

[A701;H,H,OC(═S)N(i-Pr)(CH₂)₂Br,H], [A702;H,H,OC(═S)N(i-Pr)(CH₂)₃Br,H], [A703;H,H,OC(═S)N(i-Pr)(CH₂)₄Br,H], [A704;H,H,OC(═S)N(i-Pr)(CH₂)₅Br,H], [A705;H,H,OC(═S)N(i-Pr)CH₂OMe,H], [A706;H,H,OC(═S)N(i-Pr)(CH₂)₂OMe,H], [A707;H,H,OC(═S)N(i-Pr)(CH₂)₃OMe,H], [A708;H,H,OC(═S)N(i-Pr)(CH₂)₄OMe,H], [A709;H,H,OC(═S)N(i-Pr)CH₂OEt,H], [A710;H,H,OC(═S)N(i-Pr)(CH₂)₂OEt,H], [A711;H,H,OC(═S)N(i-Pr)(CH₂)₃OEt,H], [A712;H,H,OC(═S)N(i-Pr)CH₂CN,H], [A713;H,H,OC(═S)N(i-Pr)(CH₂)₂CN,H], [A714;H,H,OC(═S)N(i-Pr)(CH₂)₃CN,H], [A715;H,H,OC(═S)N(i-Pr)(CH₂)₄CN,H], [A716;H,H,OC(═S)N(i-Pr)(CH₂)₅CN,H], [A717;H,H,OC(═S)N(i-Pr) CH₂C═CHF,H], [A718;H,H,OC(═S)N(i-Pr) CH₂C═CF₂,H], [A719;H,H,OC(═S)N(i-Pr)(CH₂)₂C═CHF,H], [A720;H,H,OC(═S)N(i-Pr) CHCH₂C═CF₂,H], [A721;H,H,OC(═S)N(i-Pr)(CH₂)₃C═CHF,H], [A722;H,H,OC(═S)N(i-Pr) CH₂CHCH₂C═CF₂,H], [A723;H,H,OC(═S)N(i-Pr)CH₂C═CHCl,H], [A724;H,H,OC(═S)N(i-Pr) CH₂C═CCl₂,H], [A725;H,H,OC(═S)N(i-Pr)(CH₂)₂C═CHCl,H], [A726;H,H,OC(═S)N(i-Pr)CHCH₂C═CCl₂,H], [A727;H,H,OC(═S)N(i-Pr)(CH₂)₃C═CHCl,H], [A728;H,H,OC(═S)N(i-Pr) CH₂CHCH₂C═CCl₂,H], [A729;H,H,OC(═S)N(i-Pr)CH₂C═CH,H], [A730;H,H,OC(═S)N(i-Pr)CH(Me)C═CH,H], [A731;H,H,OC(═S)N(i-Pr)CH₂C═CMe,H], [A732;H,H,OC(═S)N(i-Pr)CH₂C=CEt,H], [A733;H,H,OC(═S)N(i-Pr) CH₂C=CEt,H], [A734;H,H,OC(═S)N(i-Pr)(CH₂)₂C═CH,H], [A735;H,H,OC(═S)N(i-Pr)(CH₂)₂C═CMe,H], [A736;H,H,OC(═S)N(i-Pr)(CH₂)₃C═CH,H], [A737;H,H,OC(═S)N(i-Pr)(CH₂)₄C═CH,H], [A738;H,H,OC(═S)N(i-Pr)c-Pr,H], [A739;H,H,OC(═S)N(i-Pr)c-Bu,H], [A740;H,H,OC(═S)N(i-Pr)c-Pen,H], [A741;H,H,OC(═S)N(i-Pr) c-Hex,H], [A742;H,H,OC(═S)N(i-Pr) c-Hep,H], [A743;H,H,OC(═S)N(i-Pr)OCH₂F,H], [A744;H,H,OC(═S)N(i-Pr)OCHF₂,H], [A745;H,H,OC(═S)N(i-Pr)OCF₃,H], [A746;H,H,OC(═S)N(i-Pr)O(CH₂)₂F,H], [A747;H,H,OC(═S)N(i-Pr)OCH₂CHF₂,H], [A748;H,H,OC(═S)N(i-Pr)OCH₂CF₃,H], [A749;H,H,OC(═S)N(i-Pr)O(CH₂)₃F,H], [A750;H,H,OC(═S)N(i-Pr)O(CH₂)₂CHF₂,H], [A751;H,H,OC(═S)N(i-Pr)O(CH₂)₂CF₃,H], [A752;H,H,OC(═S)N(i-Pr)OCH₂CH(CF₃)₂,H], [A753;H,H,OC(═S)N(i-Pr)OCH₂CH═CH₂,H], [A754;H,H,OC(═S)N(i-Pr)OCH₂CH═CHF,H], [A755;H,H,OC(═S)N(i-Pr)OCH₂CH═CF₂,H], [A756;H,H,OC(═S)N(i-Pr)OCH₂CH═CHCl,H], [A757;H,H,OC(═S)N(i-Pr)OCH₂CH═CCl₂,H], [A758;H,H,OC(═S)N(i-Pr)OCH₂C═CH,H], [A759;H,H,OC(═S)N(i-Pr)OCH(Me)C═CH,H], [A760;H,H,OC(═S)N(i-Pr)OCH₂C═CMe,H], [A761;H,H,OC(═O)O CH₂F,H], [A762;H,H,OC(═O)O CHF₂,H], [A763;H,H,OC(═O)O CF₃,H], [A764;H,H,OC(═O)O(CH₂)₂F,H], [A765;H,H,OC(═O)O CH₂CHF₂,H], [A766;H,H,OC(═O)O CH₂CF₃,H], [A767;H,H,OC(═O)O (CH₂)₃F,H], [A768;H,H,OC(═O)O (CH₂)₂CHF₂,H], [A769;H,H,OC(═O)O (CH₂)₂CF₃,H], [A770;H,H,OC(═O)O CH₂CH(CF₃)₂,H], [A771;H,H,OC(═O)O CH₂Cl,H], [A772;H,H,OC(═O)O (CH₂₂Cl,H], [A773;H,H,OC(═O)O (CH₂)₃Cl,H], [A774;H,H,OC(═O)O (CH₂)₄Cl,H], [A775;H,H,OC(═O)O (CH₂)₅Cl,H], [A776;H,H,OC(═O)O CH₂Br,H], [A777;H,H,OC(═O)O (CH₂)₂Br,H], [A778;H,H,OC(═O)O (CH₂)₃Br,H], [A779;H,H,OC(═O)O (CH₂)₄Br,H], [A780;H,H,OC(═O)O (CH₂)₅Br,H], [A781;H,H,OC(═O)O CH₂OMe,H], [A782;H,H,OC(═O)O (CH₂)₂OMe,H], [A783;H,H,OC(═O)O (CH₂)₃OMe,H], [A784;H,H,OC(═O)O (CH₂)₄OMe,H], [A785;H,H,OC(═O)O CH₂OEt,H], [A786;H,H,OC(═O)O (CH₂)₂OEt,H], [A787;H,H,OC(═O)O (CH₂)₃OEt,H], [A788;H,H,OC(═O)O CH₂CN,H], [A789;H,H,OC(═O)O (CH₂)₂CN,H], [A790;H,H,OC(═O)O (CH₂)₃CN,H], [A791;H,H,OC(═O)O (CH₂)₄CN,H], [A792;H,H,OC(═O)O (CH₂)₅CN,H], [A793;H,H,OC(═O)O CH₂C═CHF,H], [A794;H,H,OC(═O)O CH₂C═CF₂,H], [A795;H,H,OC(═O)O (CH₂)₂C═CHF,H], [A796;H,H,OC(═O)O CHCH₂C═CF₂,H], [A797;H,H,OC(═O)O (CH₂)₃C═CHF,H], [A798;H,H,OC(═O)O CH₂CHCH₂C═CF₂,H], [A799;H,H,OC(═O)O CH₂C═CHCl,H], [A800;H,H,OC(═O)O CH₂C═CCl₂,H],

[A801;H,H,OC(═O)O (CH₂)₂C═CHCl,H], [A802;H,H,OC(═O)O CHCH₂C═CCl₂,H], [A803;H,H,OC(═O)O (CH₂)₃C═CHCl,H], [A804;H,H,OC(═O)O CH₂CHCH₂C═CCl₂,H], [A805;H,H,OC(═O)O CH₂C═CH,H], [A806;H,H,OC(═O)O CH(Me)C═CH,H], [A807;H,H,OC(═O)O CH₂C═CMe,H], [A808;H,H,OC(═O)O CH₂C=CEt,H], [A809;H,H,OC(═O)O CH₂C=CEt,H], [A810;H,H,OC(═O)O (CH₂)₂C═CH,H], [A811;H,H,OC(═O)O (CH₂)₂C═CMe,H], [A812;H,H,OC(═O)O (CH₂)₃C═CH,H], [A813;H,H,OC(═O)O (CH₂)₄C═CH,H], [A814;H,H,OC(═O)O c-Pr,H], [A815;H,H,OC(═O)O c-Bu,H], [A816;H,H,OC(═O)O c-Pen,H], [A817;H,H,OC(═O)Oc-Hex,H], [A818;H,H,OC(═O)O c-Hep,H], [A819;H,H,OC(═O)O OCH₂F,H], [A820;H,H,OC(═O)O OCHF₂,H], [A821;H,H,OC(═O)O OCF₃,H], [A822;H,H,OC(═O)O O(CH₂)₂F,H], [A823;H,H,OC(═O)O OCH₂CHF₂,H], [A824;H,H,OC(═O)O OCH₂CF₃,H], [A825;H,H,OC(═O)O O(CH₂)₃F,H], [A826;H,H,OC(═O)O O(CH₂)₂CHF₂,H], [A827;H,H,OC(═O)O O(CH₂)₂CF₃,H], [A828;H,H,OC(═O)O OCH₂CH(CF₃)₂,H], [A829;H,H,OC(═O)O OCH₂CH═CH₂,H], [A830;H,H,OC(═O)O OCH₂CH═CHF,H], [A831;H,H,OC(═O)O OCH₂CH═CF₂,H], [A832;H,H,OC(═O)O OCH₂CH═CHCl,H], [A833;H,H,OC(═O)O OCH₂CH═CCl₂,H], [A834;H,H,OC(═O)O OCH₂C═CH,H], [A835;H,H,OC(═O)O OCH(Me)C═CH,H], [A836;H,H,OC(═O)O OCH₂C═CMe,H], [A837;H,H,OC(═S)O CH₂F,H], [A838;H,H,OC(═S)O CHF₂,H], [A839;H,H,OC(═S)O CF₃,H], [A840;H,H,OC(═S) O(CH₂)₂F,H], [A841;H,H,OC(═S)O CH₂CHF₂,H], [A842;H,H,OC(═S)O CH₂CF₃,H], [A843;H,H,OC(═S) O(CH₂)₃F,H], [A844;H,H,OC(═S) O(CH₂)₂CHF₂,H], [A845;H,H,OC(═S)O (CH₂)CF₃,H], [A846;H,H,OC(═S)O CH₂CH(CF₃)₂,H], [A847;H,H,OC(═S)O CH₂Cl,H], [A848;H,H,OC(═S) O(CH₂)₂Cl,H], [A849;H,H,OC(═S) O(CH₂)₃Cl,H], [A850;H,H,OC(═S) O(CH₂)₄Cl,H], [A851;H,H,OC(═S) O(CH₂)₅Cl,H], [A852;H,H,OC(═S)O CH₂Br,H], [A853;H,H,OC(═S) O(CH₂)₂Br,H], [A854;H,H,OC(═S) O(CH₂)₃Br,H], [A855;H,H,OC(═S) O(CH₂)₄Br,H], [A856;H,H,OC(═S) O(CH₂)₅Br,H], [A857;H,H,OC(═S)O CH₂OMe,H], [A858;H,H,OC(═S) O(CH₂)₂OMe,H], [A859;H,H,OC(═S) O(CH₂)₃OMe,H], [A860;H,H,OC(═S)O (CH₂)₄OMe,H], [A861;H,H,OC(═S)O CH₂OEt,H], [A862;H,H,OC(═S)O (CH₂)₂OEt,H], [A863;H,H,OC(═S) O(CH₂)₃OEt,H], [A864;H,H,OC(═S)O CH₂CN,H], [A865;H,H,OC(═S) O(CH₂)₂CN,H], [A866;H,H,OC(═S) O(CH₂)₃CN,H], [A867;H,H,OC(═S) O(CH₂)₄CN,H], [A868;H,H,OC(═S) O(CH₂)₅CN,H], [A869;H,H,OC(═S)O CH₂C═CHF,H], [A870;H,H,OC(═S)O CH₂C═CF₂,H], [A871;H,H,OC(═S)O (CH₂)₂C═CHF,H], [A872;H,H,OC(═S)O CHCH₂C═CF₂,H], [A873;H,H,OC(═S) O(CH₂)₃C═CHF,H], [A874;H,H,OC(═S)O CH₂CHCH₂C═CF₂,H], [A875;H,H,OC(═S)O CH₂C═CHCl,H], [A876;H,H,OC(═S)O CH₂C═CCl₂,H], [A877;H,H,OC(═S)O (CH₂)₂C═CHCl,H], [A878;H,H,OC(═S)O CHCH₂C═CCl₂,H], [A879;H,H,OC(═S) O(CH₂)₃C═CHCl,H], [A880;H,H,OC(═S)O CH₂CHCH₂C═CCl₂,H], [A881;H,H,OC(═S)O CH₂C═CH,H], [A882;H,H,OC(═S)O CH(Me)C═CH,H], [A883;H,H,OC(═S)O CH₂C═CMe,H], [A884;H,H,OC(═S)O CH₂C=CEt,H], [A885;H,H,OC(═S)O CH₂C=CEt,H], [A886;H,H,OC(═S) O(CH₂)₂C═CH,H], [A887;H,H,OC(═S) O(CH₂)₂C═CMe,H], [A888;H,H,OC(═S)O (CH₂)₃C═CH,H], [A889;H,H,OC(═S) O(CH₂)₄C═CH,H], [A890;H,H,OC(═S)O c-Pr,H], [A891;H,H,OC(═S)O c-Bu,H], [A892;H,H,OC(═S)O c-Pen,H], [A893;H,H,OC(═S)O c-Hex,H], [A894;H,H,OC(═S)O c-Hep,H], [A895;H,H,OC(═S)O OCH₂F,H], [A896;H,H,OC(═S)O OCHF₂,H], [A897;H,H,OC(═S)O OCF₃,H], [A898;H,H,OC(═S)O O(CH₂)₂F,H], [A899;H,H,OC(═S)O OCH₂CHF₂,H], [A900;H,H,OC(═S)O OCH₂CF₃,H],

[A901;H,H,OC(═S)O O(CH₂)₃F,H], [A902;H,H,OC(═S)O O(CH₂)₂CHF₂,H], [A903;H,H,OC(═S)O O(CH₂)₂CF₃,H], [A904;H,H,OC(═S)O OCH₂CH(CF₃)₂,H], [A905;H,H,OC(═S)O OCH₂CH═CH₂,H], [A906;H,H,OC(═S)O OCH₂CH═CHF,H], [A907;H,H,OC(═S)O OCH₂CH═CF₂,H], [A908;H,H,OC(═S)O OCH₂CH═CHCl,H], [A909;H,H,OC(═S)O OCH₂CH═CCl₂,H], [A910;H,H,OC(═S)O OCH₂C═CH,H], [A911;H,H,OC(═S)O OCH(Me)C═CH,H], [A912;H,H,OC(═S)O OCH₂C═CMe, H], [A913;H,H,OC(═O)S CH₂F,H], [A914;H,H,OC(═O)S CHF₂,H], [A915;H,H,OC(═O)S CF₃,H], [A916;H,H,OC(═O)S (CH₂)₂F,H], [A917;H,H,OC(═O)S CH₂CHF₂,H], [A918;H,H,OC(═O)S CH₂CF₃,H], [A919;H,H,OC(═O)S (CH₂)₃F,H], [A920;H,H,OC(═O)S (CH₂)₂CHF₂,H], [A921;H,H,OC(═O)S (CH₂)₂CF₃,H], [A922;H,H,OC(═O)S CH₂CH(CF₃)₂,H], [A923;H,H,OC(═O)S CH₂Cl,H], [A924;H,H,OC(═O)S (CH₂₂Cl,H], [A925;H,H,OC(═O)S (CH₂)₃Cl,H], [A926;H,H,OC(═O)S (CH₂)₄Cl,H], [A927;H,H,OC(═O)S (CH₂)₅Cl,H], [A928;H,H,OC(═O)S CH₂Br,H], [A929;H,H,OC(═O)S (CH₂)₂Br,H], [A930;H,H,OC(═O)S (CH₂)₃Br,H], [A931;H,H,OC(═O)S (CH₂)₄Br,H], [A932;H,H,OC(═O)S (CH₂)₅Br,H], [A933;H,H,OC(═O)S CH₂OMe,H], [A934;H,H,OC(═O)S (CH₂)₂OMe,H], [A935;H,H,OC(═O)S (CH₂)₃OMe,H], [A936;H,H,OC(═O)S (CH₂)₄OMe,H], [A937;H,H,OC(═O)S CH₂OEt,H], [A938;H,H,OC(═O)S (CH₂)₂OEt,H], [A939;H,H,OC(═O)S (CH₂)₃OEt,H], [A940;H,H,OC(═O)S CH₂CN,H], [A941;H,H,OC(═O)S (CH₂)₂CN,H], [A942;H,H,OC(═O)S (CH₂)₃CN,H], [A943;H,H,OC(═O)S (CH₂)₄CN,H], [A944;H,H,OC(═O)S (CH₂)₅CN,H], [A945;H,H,OC(═O)S CH₂C═CHF,H], [A946;H,H,OC(═O)S CH₂C═CF₂,H], [A947;H,H,OC(═O)S (CH₂)₂C═CHF,H], [A948;H,H,OC(═O)S CHCH₂C═CF₂,H], [A949;H,H,OC(═O)S (CH₂)₃C═CHF,H], [A950;H,H,OC(═O)S CH₂CHCH₂C═CF₂,H], [A951;H,H,OC(═O)S CH₂C═CHCl,H], [A952;H,H,OC(═O)S CH₂C═CCl₂,H], [A953;H,H,OC(═O)S (CH₂)₂C═CHCl,H], [A954;H,H,OC(═O)S CHCH₂C═CCl₂,H], [A955;H,H,OC(═O)S (CH₂)₃C═CHCl,H], [A956;H,H,OC(═O)S CH₂CHCH₂C═CCl₂,H], [A957;H,H,OC(═O)S CH₂C═CH,H], [A958;H,H,OC(═O)S CH(Me)C═CH,H], [A959;H,H,OC(═O)S CH₂C═CMe,H], [A960;H,H,OC(═O)S CH₂C=CEt,H], [A961;H,H,OC(═O)S CH₂C=CEt,H], [A962;H,H,OC(═O)S (CH₂)₂C═CH,H], [A963;H,H,OC(═O)S (CH₂)₂C═CMe,H], [A964;H,H,OC(═O)S (CH₂)₃C═CH,H], [A965;H,H,OC(═O)S (CH₂)₄C═CH,H], [A966;H,H,OC(═O)S c-Pr,H], [A967;H,H,OC(═O)S c-Bu,H], [A968;H,H,OC(═O)S c-Pen,H], [A969;H,H,OC(═O)S c-Hex,H], [A970;H,H,OC(═O)S c-Hep,H], [A971;H,H,OC(═O)S OCH₂F,H], [A972;H,H,OC(═O)S OCHF₂,H], [A973;H,H,OC(═O)S OCF₃,H], [A974;H,H,OC(═O)S O(CH₂)₂F,H], [A975;H,H,OC(═O)S OCH₂CHF₂,H], [A976;H,H,OC(═O)S OCH₂CF₃,H], [A977;H,H,OC(═O)S O(CH₂)₃F,H], [A978;H,H,OC(═O)S O(CH₂)₂CHF₂,H], [A979;H,H,OC(═O)S O(CH₂)₂CF₃,H], [A980;H,H,OC(═O)S OCH₂CH(CF₃)₂,H], [A981;H,H,OC(═O)S OCH₂CH═CH₂,H], [A982;H,H,OC(═O)S OCH₂CH═CHF,H], [A983;H,H,OC(═O)S OCH₂CH═CF₂,H], [A984;H,H,OC(═O)S OCH₂CH═CHCl,H], [A985;H,H,OC(═O)S OCH₂CH═CCl₂,H], [A986;H,H,OC(═O)S OCH₂C═CH,H], [A987;H,H,OC(═O)S OCH(Me)C═CH,H], [A988;H,H,OC(═O)S OCH₂C═CMe,H], [A989;H,H,OC(═S)S CH₂F,H], [A990;H,H,OC(═S)S CHF₂,H], [A991;H,H,OC(═S)S CF₃,H], [A992;H,H,OC(═S)S (CH₂)₂F,H], [A993;H,H,OC(═S)S CH₂CHF₂,H], [A994;H,H,OC(═S)S CH₂CF₃,H], [A995;H,H,OC(═S)S (CH₂)₃F,H], [A996;H,H,OC(═S)S (CH₂)₂CHF₂,H], [A997;H,H,OC(═S)₅S (CH₂)₂CF₃,H], [A998;H,H,OC(═S)S CH₂CH(CF₃)₂,H], [A999;H,H,OC(═S)S CH₂Cl,H], [A1000;H,H,OC(═S)S (CH₂)₂Cl,H],

[A1001;H,H,OC(═S)S (CH₂)₃Cl,H], [A1002;H,H,OC(═S)S (CH₂)₄Cl,H], [A1003;H,H,OC(═S)S (CH₂)₅Cl,H], [A1004;H,H,OC(═S)S CH₂Br,H], [A1005;H,H,OC(═S)S (CH₂)₂Br,H], [A1006;H,H,OC(═S)S (CH₂)₃Br,H], [A1007;H,H,OC(═S)S (CH₂)₄Br,H], [A1008;H,H,OC(═S)S (CH₂)₅Br,H], [A1009;H,H,OC(═S)S CH₂OMe,H], [A1010;H,H,OC(═S)S (CH₂)₂OMe,H], [A1011;H,H,OC(═S)S (CH₂)₃OMe,H], [A1012;H,H,OC(═S)S (CH₂)₄OMe,H], [A1013;H,H,OC(═S)S CH₂OEt,H], [A1014;H,H,OC(═S)S (CH₂)₂OEt,H], [A1015;H,H,OC(═S)S (CH₂)₃OEt,H], [A1016;H,H,OC(═S)S CH₂CN,H], [A1017;H,H,OC(═S)S (CH₂₂CN,H], [A1018;H,H,OC(═S)S (CH₂)₃CN,H], [A1019;H,H,OC(═S)S (CH₂)₄CN,H], [A1020;H,H,OC(═S)S (CH₂)₅CN,H], [A1021;H,H,OC(═S)S CH₂C═CHF,H], [A1022;H,H,OC(═S)S CH₂C═CF₂,H], [A1023;H,H,OC(═S)S (CH₂)₂C═CHF,H], [A1024;H,H,OC(═S)S CHCH₂C═CF₂,H], [A1025;H,H,OC(═S)S (CH₂)₃C═CHF,H], [A1026;H,H,OC(═S)S CH₂CHCH₂C═CF₂,H], [A1027;H,H,OC(═S)S CH₂C═CHCl,H], [A1028;H,H,OC(═S)S CH₂C═CCl₂,H], [A1029;H,H,OC(═S)S (CH₂)₂C═CHCl,H], [A1030;H,H,OC(═S)S CHCH₂C═CCl₂,H], [A1031;H,H,OC(═S)S (CH₂)₃C═CHCl,H], [A1032;H,H,OC(═S)S CH₂CHCH₂C═CCl₂,H], [A1033;H,H,OC(═S)S CH₂C═CH,H], [A1034;H,H,OC(═S)S CH(Me)C═CH,H], [A1035;H,H,OC(═S)S CH₂C═CMe,H], [A1036;H,H,OC(═S)S CH₂C=CEt,H], [A1037;H,H,OC(═S)S CH₂C=CEt,H], [A1038;H,H,OC(═S)S (CH₂)₂C═CH,H], [A1039;H,H,OC(═S)S (CH₂)₂C═CMe,H], [A1040;H,H,OC(═S)S (CH₂)₃C═CH,H], [A1041;H,H,OC(═S)S (CH₂)₄C═CH,H], [A1042;H,H,OC(═S)S c-Pr,H], [A1043;H,H,OC(═S)S c-Bu,H], [A1044;H,H,OC(═S)S c-Pen,H], [A1045;H,H,OC(═S)S c-Hex,H], [A1046;H,H,OC(═S)S c-Hep,H], [A1047;H,H,OC(═S)S OCH₂F,H], [A1048;H,H,OC(═S)S OCHF₂,H], [A1049;H,H,OC(═S)S OCF₃,H], [A1050;H,H,OC(═S)S O(CH₂)₂F,H], [A1051;H,H,OC(═S)S OCH₂CHF₂,H], [A1052;H,H,OC(═S)S OCH₂CF₃,H], [A1053;H,H,OC(═S)S O(CH₂)₃F,H], [A1054;H,H,OC(═S)S O(CH₂)₂CHF₂,H], [A1055;H,H,OC(═S)S O(CH₂)₂CF₃,H], [A1056;H,H,OC(═S)S OCH₂CH(CF₃)₂,H], [A1057;H,H,OC(═S)S OCH₂CH═CH₂,H], [A1058;H,H,OC(═S)S OCH₂CH═CHF,H], [A1059;H,H,OC(═S)S OCH₂CH═CF₂,H], [A1060;H,H,OC(═S)S OCH₂CH═CHCl,H], [A1061;H,H,OC(═S)S OCH₂CH═CCl₂,H], [A1062;H,H,OC(═S)S OCH₂C═CH,H], [A1063;H,H,OC(═S)S OCH(Me)C═CH,H], [A1064;H,H,OC(═S)S OCH₂C═CMe,H], [A1065;H,H,OC(═O) CH₂F,H], [A1066;H,H,OC(═O)CHF₂,H], [A1067;H,H,OC(═O) CF₃,H], [A1068;H,H,OC(═O) (CH₂)₂F,H], [A1069;H,H,OC(═O) CH₂CHF₂,H], [A1070;H,H,OC(═O) CH₂CF₃,H], [A1071;H,H,OC(═O) (CH₂)₃F,H], [A1072;H,H,OC(═O) (CH₂)₂CHF₂,H], [A1073;H,H,OC(═O) (CH₂)₂CF₃,H], [A1074;H,H,OC(═O) CH₂CH(CF₃)₂,H], [A1075;H,H,OC(═O) CH₂Cl,H], [A1076;H,H,OC(═O) (CH₂)₂Cl,H], [A1077;H,H,OC(═O) (CH₂)₃Cl,H], [A1078;H,H,OC(═O) (CH₂)₄Cl,H], [A1079;H,H,OC(═O) (CH₂)₅Cl,H], [A1080;H,H,OC(═O) CH₂Br,H], [A1081;H,H,OC(═O) (CH₂)₂Br,H], [A1082;H,H,OC(═O) (CH₂)₃Br,H], [A1083;H,H,OC(═O) (CH₂)₄Br,H], [A1084;H,H,OC(═O) (CH₂)₅Br,H], [A1085;H,H,OC(═O) CH₂OMe,H], [A1086;H,H,OC(═O) (CH₂)₂OMe,H], [A1087;H,H,OC(═O) (CH₂)₃OMe,H], [A1088;H,H,OC(═O) (CH₂)₄OMe,H], [A1089;H,H,OC(═O) CH₂OEt,H], [A1090;H,H,OC(═O) (CH₂)₂OEt,H], [A1091;H,H,OC(═O) (CH₂)₃OEt,H], [A1092;H,H,OC(═O) CH₂CN,H], [A1093;H,H,OC(═O) (CH₂)₂CN,H], [A1094;H,H,OC(═O) (CH₂)₃CN,H], [A1095;H,H,OC(═O) (CH₂)₄CN,H], [A1096;H,H,OC(═O) (CH₂)₅CN,H], [A1097;H,H,OC(═O) CH₂C═CHF,H], [A1098;H,H,OC(═O) CH₂C═CF₂,H], [A1099;H,H,OC(═O) (CH₂)₂C═CHF,H], [A1100;H,H,OC(═O) CHCH₂C═CF₂,H],

[A1101;H,H,OC(═O) (CH₂)₃C═CHF,H], [A1102;H,H,OC(═O) CH₂CHCH₂C═CF₂,H], [A1103;H,H,OC(═O) CH₂C═CHCl,H], [A1104;H,H,OC(═O) CH₂C═CCl₂,H], [A1105;H,H,OC(═O) (CH₂)₂C═CHCl,H], [A1106;H,H,OC(═O) CHCH₂C═CCl₂,H], [A1107;H,H,OC(═O) (CH₂)₃C═CHCl,H], [A1108;H,H,OC(═O) CH₂CHCH₂C═CCl₂,H], [A1109;H,H,OC(═O) CH₂C═CH,H], [A1107;H,H,OC(═O) (CH₂)₃C=CHCl,H], [A1108;H,H,OC(═O) CH₂C═CMe,H], [A1112;H,H,OC(═O) CH₂C=CEt,H], [A1113;H,H,OC(═O) CH₂C=CEt,H], [A1114;H,H,OC(═O) (CH₂)₂C═CH,H], [A1115;H,H,OC(═O) (CH₂)₂C═CMe,H], [A1116;H,H,OC(═O) (CH₂)₃C═CH,H], [A1117;H,H,OC(═O) (CH₂)₄C═CH,H], [A1118;H,H,OC(═O) c-Pr,H], [A1119;H,H,OC(═O) c-Bu,H], [A1120;H,H,OC(═O) c-Pen,H], [A1121;H,H,OC(═O) c-Hex,H], [A1122;H,H,OC(═O) c-Hep,H], [A1123;H,H,OC(═O) OCH₂F,H], [A1124;H,H,OC(═O) OCHF₂,H], [A1125;H,H,OC(═O)OCF₃,H], [A1126;H,H,OC(═O) O(CH₂)₂F,H], [A1127;H,H,OC(═O) OCH₂CHF₂,H], [A1128;H,H,OC(═O) OCH₂CF₃,H], [A1129;H,H,OC(═O) O(CH₂)₃F,H], [A1130;H,H,OC(═O) O(CH₂)₂CHF₂,H], [A1131;H,H,OC(═O) O(CH₂)₂CF₃,H], [A1132;H,H,OC(═O) OCH₂CH(CF₃)₂,H], [A1133;H,H,OC(═O) OCH₂CH═CH₂,H], [A1134;H,H,OC(═O) OCH₂CH═CHF,H], [A1135;H,H,OC(═O) OCH₂CH═CF₂,H], [A1136;H,H,OC(═O) OCH₂CH═CHCl,H], [A1137;H,H,OC(═O) OCH₂CH═CCl₂,H], [A1138;H,H,OC(═O) OCH₂C═CH,H], [A1139;H,H,OC(═O) OCH(Me)C═CH,H], [A1140;H,H,OC(═O) OCH₂C═CMe,H], [A1141;H,H,OC(═S) CH₂F,H], [A1142;H,H,OC(═S) CHF₂,H], [A1143;H,H,OC(═S) CF₃,H], [A1144;H,H,OC(═S) (CH₂)₂F,H], [A1145;H,H,OC(═S) CH₂CHF₂,H], [A1146;H,H,OC(═S) CH₂CF₃,H], [A1147;H,H,OC(═S) (CH₂)₃F,H], [A1148;H,H,OC(═S) (CH₂)₂CHF₂,H], [A1149;H,H,OC(═S) (CH₂)₂CF₃,H], [A1150;H,H,OC(═S) CH₂CH(CF₃)₂,H], [A1151;H,H,OC(═S) CH₂Cl,H], [A1152;H,H,OC(═S) (CH₂)₂Cl,H], [A1153;H,H,OC(═S) (CH₂)₃Cl,H], [A1154;H,H,OC(═S) (CH₂)₄Cl,H], [A1155;H,H,OC(═S) (CH₂)₅Cl,H], [A1156;H,H,OC(═S) CH₂Br,H], [A1157;H,H,OC(═S) (CH₂)₂Br,H], [A1158;H,H,OC(═S) (CH₂)₃Br,H], [A1159;H,H,OC(═S) (CH₂)₄Br,H], [A1160;H,H,OC(═S) (CH₂)₅Br,H], [A1161;H,H,OC(═S) CH₂OMe,H], [A1162;H,H,OC(═S) (CH₂)₂OMe,H], [A1163;H,H,OC(═S) (CH₂)₃OMe,H], [A1164;H,H,OC(═S) (CH₂)₄OMe,H], [A1165;H,H,OC(═S) CH₂OEt,H], [A1166;H,H,OC(═S) (CH₂)₂OEt,H], [A1167;H,H,OC(═S) (CH₂)₃OEt,H], [A1168;H,H,OC(═S) CH₂CN,H], [A1169;H,H,OC(═S) (CH₂)₂CN,H], [A1170;H,H,OC(═S) (CH₂)₃CN,H], [A1171;H,H,OC(═S) (CH₂)₄CN,H], [A1172;H,H,OC(═S) (CH₂)₅CN,H], [A1173;H,H,OC(═S) CH₂C═CHF,H], [A1174;H,H,OC(═S) CH₂C═CF₂,H], [A1175;H,H,OC(═S) (CH₂)₂C═CHF,H], [A1176;H,H,OC(═S) CHCH₂C═CF₂,H], [A1177;H,H,OC(═S) (CH₂)₃C═CHF,H], [A1178;H,H,OC(═S) CH₂CHCH₂C═CF₂,H], [A1179;H,H,OC(═S) CH₂C═CHCl,H], [A1180;H,H,OC(═S) CH₂C═CCl₂,H], [A1181;H,H,OC(═S) (CH₂)₂C═CHCl,H], [A1182;H,H,OC(═S) CHCH₂C═CCl₂,H], [A1183;H,H,OC(═S) (CH₂)₃C═CHCl,H], [A1184;H,H,OC(═S) CH₂CHCH₂C═CCl₂,H], [A1185;H,H,OC(═S) CH₂C═CH,H], [A1186;H,H,OC(═S) CH(Me)C═CH,H], [A1187;H,H,OC(═S) CH₂C═CMe,H], [A1188;H,H,OC(═S) CH₂C=CEt,H], [A1189;H,H,OC(═S) CH₂C=CEt,H], [A1190;H,H,OC(═S) (CH₂)₂C═CH,H], [A1191;H,H,OC(═S) (CH₂)₂C═CMe,H], [A1192;H,H,OC(═S) (CH₂)₃C═CH,H], [A1193;H,H,OC(═S) (CH₂)₄C═CH,H], [A1194;H,H,OC(═S) c-Pr,H], [A1195;H,H,OC(═S) c-Bu,H], [A1196;H,H,OC(═S) c-Pen,H], [A1197;H,H,OC(═S) c-Hex,H], [A1198;H,H,OC(═S) c-Hep,H], [A1199;H,H,OC(═S) OCH₂F,H], [A1200;H,H,OC(═S) OCHF₂,H],

[A1201;H,H,OC(═S)OCF₃,H], [A1202;H,H,OC(═S)O (CH₂)₂F,H], [A1203;H,H,OC(═S) OCH₂CHF₂,H], [A1204;H,H,OC(═S) OCH₂CF₃,H], [A1205;H,H,OC(═S)O (CH₂)₃F,H], [A1206;H,H,OC(═S) O(CH₂)₂CHF₂,H], [A1207;H,H,OC(═S)O (CH₂)₂CF₃,H], [A1208;H,H,OC(═S) OCH₂CH(CF₃)₂,H], [A1209;H,H,OC(═S) OCH₂CH═CH₂,H], [A1210;H,H,OC(═S) OCH₂CH═CHF,H], [A12111;H,H,OC(═S) OCH₂CH═CF₂,H], [A1212;H,H,OC(═S) OCH₂CH═CHCl,H], [A1213;H,H,OC(═S) OCH₂CH═CCl₂,H], [A1214;H,H,OC(═S) OCH₂C═CH,H], [A1215;H,H,OC(═S) OCH(Me)C═CH,H], [A1216;H,H,OC(═S) OCH₂C═CMe,H], [A1217;H,H,NHC(═O)NHCH₂F,H], [A1218;H,H,NHC(═O)NHCHF₂,H], [A1219;H,H,NHC(═O)NHCF₃,H], [A1220;H,H,NHC(═O)NH(CH₂)₂F,H], [A1221;H,H,NHC(═O)NHCH₂CHF₂,H], [A1222;H,H,NHC(═O)NHCH₂CF₃,H], [A1223;H,H,NHC(═O)NH(CH₂)₃F,H], [A1224;H,H,NHC(═O)NH(CH₂)₂CHF₂,H], [A1225;H,H,NHC(═O)NH(CH₂)₂CF₃,H], [A1226;H,H,NHC(═O)NHCH₂CH(CF₃)₂,H], [A1227;H,H,NHC(═O)NHCH₂Cl,H], [A1228;H,H,NHC(═O)NH(CH₂)₆Cl,H], [A1229;H,H,NHC(═O)NH(CH₂)₃Cl,H], [A1230;H,H,NHC(═O)NH(CH₂) Cl,H], [A1231;H,H,NHC(═O)NH(CH₂)₅Cl,H], [A1232;H,H,NHC(═O)NHCH₂Br,H], [A1233;H,H,NHC(═O)NH(CH₂)₂Br,H], [A1234;H,H,NHC(═O)NH(CH₂)₃Br,H], [A1235;H,H,NHC(═O)NH(CH₂)₄Br,H], [A1236;H,H,NHC(═O)NH(CH₂)₅Br,H], [A1237;H,H,NHC(═O)NHCH₂OMe,H], [A1238;H,H,NHC(═O)NH(CH₂)₂₀Me,H], [A1239;H,H,NHC(═O)NH(CH₂)₃OMe,H], [A1240;H,H,NHC(═O)NH(CH₂)₄OMe,H], [A1241;H,H,NHC(═O)NHCH₂OEt,H], [A1242;H,H,NHC(═O)NH(CH₂)₂OEt,H], [A1243;H,H,NHC(═O)NH(CH₂)₃OEt,H], [A1244;H,H,NHC(═O)NHCH₂CN,H], [A1245;H,H,NHC(═O)NH(CH₂)₂CN,H], [A1246;H,H,NHC(═O)NH(CH₂)₃CN,H], [A1247;H,H,NHC(═O)NH(CH₂)₄CN,H], [A1248;H,H,NHC(═O)NH(CH₂)₅CN,H], [A1249;H,H,NHC(═O)NHCH₂C═CHF,H], [A1250;H,H,NHC(═O)NHCH₂C═CF₂,H], [A1251;H,H,NHC(═O)NH(CH₂)₂C═CHF,H], [A1252;H,H,NHC(═O)NHCHCH₂C═CF₂,H], [A1253;H,H,NHC(═O)NH(CH₂)₃C═CHF,H], [A1254;H,H,NHC(═O)NHCH₂CHCH₂C═CF₂,H], [A1255;H,H,NHC(═O)NHCH₂C═CHCl,H], [A1256;H,H,NHC(═O)NHCH₂C═CCl₂,H], [A1257;H,H,NHC(═O)NH(CH₂)₂C═CHCl,H], [A1258;H,H,NHC(═O)NHCHCH₂C═CCl₂,H], [A1259;H,H,NHC(═O)NH(CH₂)₃C═CHCl,H], [A1260;H,H,NHC(═O)NHCH₂CHCH₂C═CCl₂,H], [A1261;H,H,NHC(═O)NHCH₂C═CH,H], [A1262;H,H,NHC(═O)NHCH(Me)C═CH,H], [A1263;H,H,NHC(═O)NHCH₂C═CMe,H], [A1264;H,H,NHC(═O)NHCH₂C=CEt,H], [A1265;H,H,NHC(═O)NHCH₂C=CEt,H][A1266;H,H,NHC(═O)NH(CH₂)₂C═CH,H], [A1267;H,H,NHC(═O)NH(CH₂)₂C═CMe,H], [A1268;H,H,NHC(═O)NH(CH₂)₃C═CH,H], [A1269;H,H,NHC(═O)NH(CH₂)₄C═CH,H], [A1270;H,H,NHC(═O)NHc-Pr,H], [A1271;H,H,NHC(═O)NHc-Bu,H], [A1272;H,H,NHC(═O)NHc-Pen,H], [A1273;H,H,NHC(═O)NHc-Hex,H], [A1274;H,H,NHC(═O)NHc-Hep,H], [A1275;H,H,NHC(═O)NHOCH₂F,H], [A1276;H,H,NHC(═O)NHOCHF₂,H], [A1277;H,H,NHC(═O)NHOCF₃,H], [A1278;H,H,NHC(═O)NHO(CH₂)₂F,H], [A1279;H,H,NHC(═O)NHOCH₂CHF₂,H], [A1280;H,H,NHC(═O)NHOCH₂CF₃,H], [A1281;H,H,NHC(═O)NHO(CH₂)₃F,H][A1282;H,H,NHC(═O)NHO(CH₂)₂CHF₂,H], [A1283;H,H,NHC(═O)NHO(CH₂)₂CF₃,H], [A1284;H,H,NHC(═O)NHOCH₂CH (C CF₃)₂,H], [A1285;H,H,NHC(═O)NHOCH₂CH═CH₂,H][A1286;H,H,NHC(═O)NHOCH₂CH═CHF,H][A1287;H,H,NHC(═O)NHOCH₂CH═CF₂,H], [A1288;H,H,NHC(═O)NHOCH₂CH═CHCl,H], [A1289;H,H,NHC(═O)NHOCH₂CH═CCl₂,H], [A1290;H,H,NHC(═O)NHOCH₂C═CH,H][A1291;H,H,NHC(═O)NHOCH(Me)C═CH,H][A1292;H,H,NHC(═O)NHOCH₂C═CMe,H][A1293;H,H,NHC(═O)NMeCH₂F,H], [A1294;H,H,NHC(═O)NMeCHF₂,H], [A1295;H,H,NHC(═O)NMeCF₃,H], [A1296;H,H,NHC(═O)NMe(CH₂)₂F,H], [A1297;H,H,NHC(═O)NMeCH₂CHF₂,H], [A1298;H,H,NHC(═O)NMeCH₂CF₃,H], [A1299;H,H,NHC(═O)NMe(CH₂)₃F,H][A1300;H,H,NHC(═O)NMe(CH₂)₂CHF₂,H],

[A1301;H,H,NHC(═O)NMe(CH₂)₂CF₃,H], [A1302;H,H,NHC(═O)NMeCH₂CH(CF₃)₂,H][A1303;H,H,NHC(═O)NMeCH₂Cl,H][A1304;H,H,NHC(═O)NMe(CH₂)₂Cl,H], [A1305;H,H,NHC(═O)NMe(CH₂)₃Cl,H][A1306;H,H,NHC(═O)NMe(CH₂)₄Cl,H][A1307;H,H,NHC(═O)NMe(CH₂)₅Cl,H][A1308;H,H,NHC(═O)NMeCH₂Br,H][A1309;H,H,NHC(═O)NMe(CH₂)₂Br,H], [A1310;H,H,NHC(═O)NMe(CH₂)₃Br,H], [A1311;H,H,NHC(═O)NMe(CH₂)₄Br,H][A1312;H,H,NHC(═O)NMe(CH₂)₅Br,H][A1313;H,H,NHC(═O)NMeCH₂OMe,H][A1314;H,H,NHC(═O)NMe(CH₂)₂OMe,H], [A1315;H,H,NHC(═O)NMe(CH₂)₃OMe,H][A1316;H,H,NHC(═O)NMe(CH₂)₄OMe,H][A1317;H,H,NHC(═O)NMeCH₂OEt,H][A1318;H,H,NHC(═O)NMe(CH₂)₂OEt,H][A1319;H,H,NHC(═O)NMe(CH)OEt,H], [A1320;H,H,NHC(═O)NMeCH₂CN,H][A1321;H,H,NHC(═O)NMe(CH₂)₂CN,H][A1322;H,H,NHC(═O)NMe(CH₂) CN,H], [A1323;H,H,NHC(═O)NMe(CH₂)₄CN,H][A1324;H,H,NHC(═O)NMe(CH₂)₅CN,H], [A1325;H,H,NHC(═O)NMeCH₂C═CHF,H][A1326;H,H,NHC(═O)NMeCH₂C═CF₂,H][A1327;H,H,NHC(═O)NMe(CH₂)₂C═CHF,H], [A1328;H,H,NHC(═O)NMeCHCH₂C═CF₂,H][A1329;H,H,NHC(═O)NMe(CH₂)₃C═CHF,H], [A1330;H,H,NHC(═O)NMeCH₂CHCH₂C═CF₂,H], [A1331;H,H,NHC(═O)NMeCH₂C═CHCl,H][A1332;H,H,NHC(═O)NMeCH₂C═CCl₂,H], [A1333;H,H,NHC(═O)NMe(CH₂)₂C═CHCl,H], [A1334;H,H,NHC(═O)NMeCHCH₂C═CCl₂,H], [A1335;H,H,NHC(═O)NMe(CH₂)₃C═CHCl,H], [A1336;H,H,NHC(═O)NMeCH₂CHCH₂C═CCl₂,H][A1337;H,H,NHC(═O)NMeCH₂C═CH,H][A1338;H,H,NHC(═O)NMeCH(Me)C═CH,H][A1339;H,H,NHC(═O)NMeCH₂C═CMe,H], [A1340;H,H,NHC(═O)NMeCH₂C=CEt,H][A1341;H,H,NHC(═O)NMeCH₂C=CEt,H][A1342;H,H,NHC(═O)NMe(CH₂)₂C═CH,H][A1343;H,H,NHC(═O)NMe(CH₂)₂C═CMe,H][A1344;H,H,NHC(═O)NMe(CH)₃C═CH,H], [A1345;H,H,NHC(═O)NMe(CH₂)₄C═CH,H], [A1346;H,H,NHC(═O)NMec-Pr,H], [A1347;H,H,NHC(═O)NMec-Bu,H], [A1348;H,H,NHC(═O)NMec-Pen,H], [A1349;H,H,NHC(═O)NMec-Hex,H], [A1350;H,H,NHC(═O)NMec-Hep,H], [A1351;H,H,NHC(═O)NMeOCH₂F,H], [A1352;H,H,NHC(═O)NMeOCHF₂,H], [A1353;H,H,NHC(═O)NMeOCF₃,H], [A1354;H,H,NHC(═O)NMeO(CH₂)₂F,H][A1355;H,H,NHC(═O)NMeOCH₂CHF₂,H], [A1356;H,H,NHC(═O)NMeOCH₂CF₃,H], [A1357;H,H,NHC(═O)NMeO(CH₂)₃F,H][A1358;H,H,NHC(═O)NMeO(CH₂)₂CHF₂,H], [A1359;H,H,NHC(═O)NMeO(CH₂)₂CF₃,H][A1360;H,H,NHC(═O)NMeOCH₂CH(CF₃)₂,H][A1361;H,H,NHC(═O)NMeOCH₂CH═CH₂,H][A1362;H,H,NHC(═O)NMeOCH₂CH═CHF,H][A1363;H,H,NHC(═O)NMeOCH₂CH═CF₂,H], [A1364;H,H,NHC(═O)NMeOCH₂CH═CHCl,H], [A1365;H,H,NHC(═O)NMeOCH₂CH═CCl₂,H], [A1366;H,H,NHC(═O)NMeOCH₂C═CH,H], [A1367;H,H,NHC(═O)NMeOCH(Me)C═CH,H], [A1368;H,H,NHC(═O)NMeOCH₂C═CMe,H], [A1369;H,H,NHC(═O)NEtCH₂F,H], [A1370;H,H,NHC(═O)NEtCHF₂,H], [A1371;H,H,NHC(═O)NEtCF₃,H], [A1372;H,H,NHC(═O)NEt(CH₂)₂F,H], [A1373;H,H,NHC(═O)NEtCH₂CHF₂,H], [A1374;H,H,NHC(═O)NEtCH₂CF₃,H], [A1375;H,H,NHC(═O)NEt(CH₂)₃F,H], [A1376;H,H,NHC(═O)NEt(CH₂)₂CHF₂,H], [A1377;H,H,NHC(═O)NEt(CH₂)₂CF₃,H], [A1378;H,H,NHC(═O)NEtCH₂CH(CF₃)₂,H], [A1379;H,H,NHC(═O)NEtCH₂Cl,H], [A1380;H,H,NHC(═O)NEt(CH₂)₂Cl,H], [A1381;H,H,NHC(═O)NEt(CH₂)₃Cl,H], [A1382;H,H,NHC(═O)NEt(CH₂)₄Cl,H], [A1383;H,H,NHC(═O)NEt(CH₂)₅C,H], [A1384;H,H,NHC(═O)NEtCH₂Br,H], [A1385;H,H,NHC(═O)NEt(CH₂)₂Br,H], [A1386;H,H,NHC(═O)NEt(CH₂)₃Br,H], [A1387;H,H,NHC(═O)NEt(CH₂)₄Br,H], [A1388;H,H,NHC(═O)NEt(CH₂)₅Br,H], [A1389;H,H,NHC(═O)NEtCH₂OMe,H], [A1390;H,H,NHC(═O)NEt(CH₂)₂₀Me,H], [A1391;H,H,NHC(═O)NEt(CH₂)₃OMe,H], [A1392;H,H,NHC(═O)NEt(CH₂)₄OMe,H], [A1393;H,H,NHC(═O)NEtCH₂OEt,H], [A1394;H,H,NHC(═O)NEt(CH₂)₂OEt,H], [A1395;H,H,NHC(═O)NEt(CH₂)₃OEt,H], [A1396;H,H,NHC(═O)NEtCH₂CN,H], [A1397;H,H,NHC(═O)NEt(CH₂)₂CN,H], [A1398;H,H,NHC(═O)NEt(CH₂)₃CN,H], [A1399;H,H,NHC(═O)NEt(CH₂)₄CN,H], [A1400;H,H,NHC(═O)NEt(CH₂)₅CN,H],

[A1401;H,H,NHC(═O)NEtCH₂C═CHF,H], [A1402;H,H,NHC(═O)NEtCH₂C═CF₂,H], [A1403;H,H,NHC(═O)NEt(CH₂)₂C═CHF,H], [A1404;H,H,NHC(═O)NEtCHCH₂C═CF₂,H], [A1405;H,H,NHC(═O)NEt(CH₂)₃C═CHF,H], [A1406;H,H,NHC(═O)NEtCH₂CHCH₂C═CF₂,H], [A1407;H,H,NHC(═O)NEtCH₂C═CHCl,H], [A1408;H,H,NHC(═O)NEtCH₂C═CCl₂,H], [A1409;H,H,NHC(═O)NEt(CH₂)₂C═CHCl,H], [A1410;H,H,NHC(═O)NEtCHCH₂C═CCl₂,H], [A1411;H,H,NHC(═O)NEt(CH₂)₃C═CHCl,H], [A1412;H,H,NHC(═O)NEtCH₂CHCH₂CHC═CCl₂,H], [A1413;H,H,NHC(═O)NEtCH₂C═CH,H], [A1414;H,H,NHC(═O)NEtCH(Me)C═CH,H], [A1415;H,H,NHC(═O)NEtCH₂C═CMe,H], [A1416;H,H,NHC(═O)NEtCH₂C=CEt,H], [A1417;H,H,NHC(═O)NEtCH₂C=CEt,H], [A1418;H,H,NHC(═O)NEt(CH₂)₂C C═CH,H], [A1419;H,H,NHC(═O)NEt(CH₂)₂C═CMe,H], [A1420;H,H,NHC(═O)NEt(CH₂)₃C═CH,H], [A1421;H,H,NHC(═O)NEt(CH₂)₄C═CH,H], [A1422;H,H,NHC(═O)NEtc-Pr,H], [A1423;H,H,NHC(═O)NEtc-Bu,H], [A1424;H,H,NHC(═O)NEtc-Pen,H], [A1425;H,H,NHC(═O)NEtc-Hex,H], [A1426;H,H,NHC(═O)NEtc-Hep,H], [A1427;H,H,NHC(═O)NEtOCH₂F,H], [A1428;H,H,NHC(═O)NEtOCHF₂,H], [A1429;H,H,NHC(═O)NEtOCF₃,H], [A1430;H,H,NHC(═O)NEtO(CH₂)₂F,H], [A1431;H,H,NHC(═O)NEtOCH₂CHF₂,H], [A1432;H,H,NHC(═O)NEtOCH₂CF₃,H], [A1433;H,H,NHC(═O)NEtO(CH)₃F,H], [A1434;H,H,NHC(═O)NEtO(CH₂)₂CHF₂,H], [A1435;H,H,NHC(═O)NEtO(CH₂)₂CF₃,H], [A1436;H,H,NHC(═O)NEtOCH₂CH(CF₃)₂,H], [A1437;H,H,NHC(═O)NEtOCH₂CH═CH₂,H], [A1438;H,H,NHC(═O)NEtOCH₂CH═CHF,H], [A1439;H,H,NHC(═O)NEtOCH₂CH═CF₂,H], [A1440;H,H,NHC(═O)NEtOCH₂CH═CHCl,H], [A1441;H,H,NHC(═O)NEtOCH₂CH═CCl₂,H], [A1442;H,H,NHC(═O)NEtOCH₂C═CH,H], [A1443;H,H,NHC(═O)NEtOCH(Me)C═CH,H], [A1444;H,H,NHC(═O)NEtOCH₂C═CMe,H], [A1445;H,H,NHC(═O)NPrCH₂F,H], [A1446;H,H,NHC(═O)NPrCHF₂,H], [A1447;H,H,NHC(═O)NPrCF₃,H], [A1448;H,H,NHC(═O)NPr(CH₂)₂F,H], [A1449;H,H,NHC(═O)NPrCH₂CHF₂,H], [A1450;H,H,NHC(═O)NPrCH₂CF₃,H], [A1451;H,H,NHC(═O)NPr(CH₂)₃F,H], [A1452;H,H,NHC(═O)NPr(CH₂)₂CHF₂,H], [A1453;H,H,NHC(═O)NPr(CH₂)₂CF₃,H], [A1454;H,H,NHC(═O)NPrCH₂CH(CF₃)₂,H], [A1455;H,H,NHC(═O)NPrCH₂Cl,H], [A1456;H,H,NHC(═O)NPr(CH₂)₂Cl,H], [A1457;H,H,NHC(═O)NPr(CH₂)₃Cl,H], [A1458;H,H,NHC(═O)NPr(CH₂)₄Cl,H], [A1459;H,H,NHC(═O)NPr(CH₂)₅C,H], [A1460;H,H,NHC(═O)NPrCH₂Br,H], [A461;H,H,NHC(═O)NPr(CH₂)₂Br,H], [A1462;H,H,NHC(═O)NPr(CH₂)₃Br,H], [A1463;H,H,NHC(═O)NPr(CH₂)₄Br,H], [A1464;H,H,NHC(═O)NPr(CH₂)₅Br,H], [A1465;H,H,NHC(═O)NPrCH₂OMe,H], [A1466;H,H,NHC(═O)NPr(CH₂)₂OMe,H], [A1467;H,H,NHC(═O)NPr(CH₂)₃OMe,H], [A1468;H,H,NHC(═O)NPr(CH₂)₄OMe,H], [A1469;H,H,NHC(═O)NPrCH₂OEt,H], [A1470;H,H,NHC(═O)NPr(CH₂)₂OEt,H], [A1471;H,H,NHC(═O)NPr(CH₂)₃OEt,H][A1472;H,H,NHC(═O)NPrCH₂CN,H], [A1473;H,H,NHC(═O)NPr(CH₂)₂CN,H], [A1474;H,H,NHC(═O)NPr(CH₂)₃CN,H], [A1475;H,H,NHC(═O)NPr(CH₂)₄CN,H], [A1476;H,H,NHC(═O)NPr(CH₂)₅CN,H], [A1477;H,H,NHC(═O)NPrCH₂C═CHF,H], [A1478;H,H,NHC(═O)NPrCH₂C═CF₂,H], [A1479;H,H,NHC(═O)NPr(CH₂)₂C═CHF,H], [A1480;H,H,NHC(═O)NPrCHCH₂C═CF₂,H], [A1481;H,H,NHC(═O)NPr(CH₂)₃C═CHF,H], [A1482;H,H,NHC(═O)NPrCH₂CHCH₂C═CF₂,H], [A1483;H,H,NHC(═O)NPrCH₂C═CHCl,H], [A1484;H,H,NHC(═O)NPrCH₂C═CCl₂,H], [A1485;H,H,NHC(═O)NPr(CH₂)₂C═CHCl,H], [A1486;H,H,NHC(═O)NPrCHCH₂C═CCl₂,H], [A1487;H,H,NHC(═O)NPr(CH₂)₃C═CHCl,H], [A1488;H,H,NHC(═O)NPrCH₂CHCH₂C═CCl₂,H], [A1489;H,H,NHC(═O)NPrCH₂C═CH,H], [A1490;H,H,NHC(═O)NPrCH(Me)C═CH,H], [A1491;H,H,NHC(═O)NPrCH₂C═CMe,H][A1492;H,H,NHC(═O)NPrCH₂C=CEt,H][A1493;H,H,NHC(═O)NPrCH₂C=CEt,H][A1494;H,H,NHC(═O)NPr(CH₂)₂C═CH,H], [A1495;H,H,NHC(═O)NPr(CH₂)₂C═CMe,H], [A1496;H,H,NHC(═O)NPr(CH₂)₃C═CH,H], [A1497;H,H,NHC(═O)NPr(CH₂)₄C═CH,H], [A1498;H,H,NHC(═O)NPrc-Pr,H], [A1499;H,H,NHC(═O)NPrc-Bu,H], [A1500;H,H,NHC(═O)NPrc-Pen,H],

[A1501;H,H,NHC(═O)NPrc-Hex,H], [A1502;H,H,NHC(═O)NPrc-Hep,H], [A1503;H,H,NHC(═O)NPrOCH₂F,H], [A1504;H,H,NHC(═O)NPrOCHF₂,H], [A1505;H,H,NHC(═O)NPrOCF₃,H], [A1506;H,H,NHC(═O)NPrO(CH₂)₂F,H][A1507;H,H,NHC(═O)NPrOCH₂CHF₂,H], [A1508;H,H,NHC(═O)NPrOCH₂CF₃,H], [A1509;H,H,NHC(═O)NPrO(CH₂)₃F,H][A1510;H,H,NHC(═O)NPrO(CH₂)₂CHF₂,H], [A1511;H,H,NHC(═O)NPrO(CH₂)₂CF₃,H], [A1512;H,H,NHC(═O)NPrOCH₂CH(CF₃)₂,H], [A1513;H,H,NHC(═O)NPrOCH₂CH═CH₂,H], [A1514;H,H,NHC(═O)NPrOCH₂CH═CHF,H][A1515;H,H,NHC(═O)NPrOCH₂CH═CF₂,H][A1516;H,H,NHC(═O)NPrOCH₂CH═CHCl,H][A1517;H,H,NHC(═O)NPrOCH₂CH═CCl₂,H], [A1518;H,H,NHC(═O)NPrOCH₂C═CH,H][A1519;H,H,NHC(═O)NPrOCH(Me)C═CH,H][A1520;H,H,NHC(═O)NPrOCH₂C═CMe,H], [A1521;H,H,NHC(═O)N(i-Pr)CH₂F,H], [A1522;H,H,NHC(═O)N(i-Pr)CHF₂,H], [A1523;H,H,NHC(═O)N(i-Pr)CF₃,H], [A1524;H,H,NHC(═O)N(i-Pr)(CH₂)₂F,H], [A1525;H,H,NHC(═O)N(i-Pr)CH₂CHF₂,H], [A1526;H,H,NHC(═O)N(i-Pr)CH₂CF₃,H], [A1527;H,H,NHC(═O)N(i-Pr)(CH₂)₃F,H], [A1528;H,H,NHC(═O)N(i-Pr)(CH₂)₂CHF₂,H], [A1529;H,H,NHC(═O)N(i-Pr)(CH₂)₂CF₃,H], [A1530;H,H,NHC(═O)N(i-Pr)CH₂CH(CF₃)₂,H], [A1531;H,H,NHC(═O)N(i-Pr)CH₂Cl,H], [A1532;H,H,NHC(═O)N(i-Pr)(CH₂)₂Cl,H], [A1533;H,H,NHC(═O)N(i-Pr)(CH₂)₃Cl,H], [A1534;H,H,NHC(═O)N(i-Pr)(CH₂)₄Cl,H], [A1535;H,H,NHC(═O)N(i-Pr)(CH₂)₅Cl,H], [A1536;H,H,NHC(═O)N(i-Pr)CH₂Br,H], [A1537;H,H,NHC(═O)N(i-Pr)(CH₂)₂Br,H], [A1538;H,H,NHC(═O)N(i-Pr)(CH₂)₃Br,H], [A1539;H,H,NHC(═O)N(i-Pr)(CH₂)₄Br,H], [A1540;H,H,NHC(═O)N(i-Pr)(CH₂)₅Br,H], [A1541;H,H,NHC(═O)N(i-Pr)CH₂OMe,H], [A1542;H,H,NHC(═O)N(i-Pr)(CH₂)₂OMe,H], [A1543;H,H,NHC(═O)N(i-Pr)(CH₂)₃OMe,H], [A1544;H,H,NHC(═O)N(i-Pr)(CH₂)₄OMe,H], [A1545;H,H,NHC(═O)N(i-Pr)CH₂OEt,H], [A1546;H,H,NHC(═O)N(i-Pr)(CH₂)₂OEt,H], [A1547;H,H,NHC(═O)N(i-Pr)(CH₂)₃OEt,H], [A1548;H,H,NHC(═O)N(i-Pr)CH₂CN,H], [A1549;H,H,NHC(═O)N(i-Pr)(CH₂)₂CN,H], [A1550;H,H,NHC(═O)N(i-Pr)(CH₂)₃CN,H], [A1551;H,H,NHC(═O)N(i-Pr)(CH₂)₄CN,H], [A1552;H,H,NHC(═O)N(i-Pr)(CH₂)₅CN,H], [A1553;H,H,NHC(═O)N(i-Pr)CH₂C═CHF,H], [A1554;H,H,NHC(═O)N(i-Pr) CH₂C═CF₂,H], [A1555;H,H,NHC(═O)N(i-Pr)(CH₂)₂C═CHF,H], [A1556;H,H,NHC(═O)N(i-Pr) CHCH₂C═CF₂,H], [A1557;H,H,NHC(═O)N(i-Pr)(CH₂)₃C═CHF,H], [A1558;H,H,NHC(═O)N(i-Pr) CH₂CHCH₂C═CF₂,H], [A1559;H,H,NHC(═O)N(i-Pr)CH₂C═CHCl,H], [A1560;H,H,NHC(═O)N(i-Pr) CH₂C═CCl₂,H], [A1561;H,H,NHC(═O)N(i-Pr)(CH₂)₂C═CHCl,H], [A1562;H,H,NHC(═O)N(i-Pr) CHCH₂C═CCl₂,H], [A1563;H,H,NHC(═O)N(i-Pr)(CH₂)₃C═CHCl,H], [A1564;H,H,NHC(═O)N(i-Pr) CH₂CHCH₂C═CCl₂,H], [A1565;H,H,NHC(═O)N(i-Pr)CH₂C═CH,H], [A1566;H,H,NHC(═O)N(i-Pr)CH(Me)C═CH,H], [A1567;H,H,NHC(═O)N(i-Pr)CH₂C═CMe,H], [A1568;H,H,NHC(═O)N(i-Pr)CH₂C=CEt,H], [A1569;H,H,NHC(═O)N(i-Pr)CH₂C=CEt,H], [A1570;H,H,NHC(═O)N(i-Pr)(CH₂)₂C═CH,H], [A1571;H,H,NHC(═O)N(i-Pr)(CH₂)₂C═CMe,H], [A1572;H,H,NHC(═O)N(i-Pr)(CH₂)₃C═CH,H], [A1573;H,H,NHC(═O)N(i-Pr)(CH₂)₄C═CH,H], [A1574;H,H,NHC(═O)N(i-Pr)c-Pr,H], [A1575;H,H,NHC(═O)N(i-Pr)c-Bu,H], [A1576;H,H,NHC(═O)N(i-Pr)c-Pen,H], [A1577;H,H,NHC(═O)N(i-Pr)c-Hex,H], [A1578;H,H,NHC(═O)N(i-Pr)c-Hep,H], [A1579;H,H,NHC(═O)N(i-Pr) OCH₂F,H], [A1580;H,H,NHC(═O)N(i-Pr)OCHF₂,H], [A1581;H,H,NHC(═O)N(i-Pr)OCF₃,H], [A1582;H,H,NHC(═O)N(i-Pr)O(CH₂)₂F,H], [A1583;H,H,NHC(═O)N(i-Pr) OCH₂CHF₂,H], [A1584;H,H,NHC(═O)N(i-Pr) OCH₂CF₃,H], [A1585;H,H,NHC(═O)N(i-Pr)O(CH₂)₃F,H], [A1586;H,H,NHC(═O)N(i-Pr)O(CH₂)₂CHF₂,H], [A1587;H,H,NHC(═O)N(i-Pr)O(CH₂)₂CF₃,H], [A1588;H,H,NHC(═O)N(i-Pr) OCH₂CH(CF₃)₂,H], [A1589;H,H,NHC(═O)N(i-Pr) OCH₂CH═CH₂,H], [A1590;H,H,NHC(═O)N(i-Pr) OCH₂CH═CHF,H], [A1591;H,H,NHC(═O)N(i-Pr) OCH₂CH═CF₂,H], [A1592;H,H,NHC(═O)N(i-Pr) OCH₂CH═CHCl,H], [A1593;H,H,NHC(═O)N(i-Pr) OCH₂CH═CCl₂,H], [A1594;H,H,NHC(═O)N(i-Pr) OCH₂C═CH,H], [A1595;H,H,NHC(═O)N(i-Pr) OCH(Me)C═CH,H], [A1596;H,H,NHC(═O)N(i-Pr) OCH₂C═CMe,H], [A1597;H,H,NHC(═S)NHCH₂F,H], [A1598;H,H,NHC(═S)NHCHF₂,H], [A1599;H,H,NHC(═S)NHCF₃,H], [A1600;H,H,NHC(═S)NH(CH₂)₂F,H],

[A1601;H,H,NHC(═S)NHCH₂CHF₂,H], [A1602;H,H,NHC(═S)NHCH₂CF₃,H], [A1603;H,H,NHC(═S)NH(CH₂)₃F,H], [A1604;H,H,NHC(═S)NH(CH₂)₂CHF₂,H], [A1605;H,H,NHC(═S)NH(CH₂)₂CF₃,H], [A1606;H,H,NHC(═S)NHCH₂CH(CF₃)₂,H], [A1607;H,H,NHC(═S)NHCH₂Cl,H], [A1608;H,H,NHC(═S)NH(CH₂)₂Cl,H], [A1609;H,H,NHC(═S)NH(CH₂)₃₁,H], [A1610;H,H,NHC(═S)NH(CH₂)₄Cl,H], [A1611;H,H,NHC(═S)NH(CH₂)₅Cl,H], [A1612;H,H,NHC(═S)NHCH₂Br,H], [A1613;H,H,NHC(═S)NH(CH₂)₂Br,H], [A1614;H,H,NHC(═S)NH(CH₂)₃Br,H], [A1615;H,H,NHC(═S)NH(CH₂)₄Br,H], [A1616;H,H,NHC(═S)NH(CH₂)₅Br,H], [A1617;H,H,NHC(═S)NHCH₂OMe,H], [A1618;H,H,NHC(═S)NH(CH₂)₂OMe,H], [A1619;H,H,NHC(═S)NH(CH₂)₃OMe,H], [A1620;H,H,NHC(═S)NH(CH₂)₄OMe,H], [A1621;H,H,NHC(═S)NHCH₂OEt,H], [A1622;H,H,NHC(═S)NH(CH₂)₂OEt,H], [A1623;H,H,NHC(═S)NH(CH₂)₃OEt,H], [A1624;H,H,NHC(═S)NHCH₂CN,H], [A1625;H,H,NHC(═S)NH(CH₂)₂CN,H], [A1626;H,H,NHC(═S)NH(CH₂)₃CN,H], [A1627;H,H,NHC(═S)NH(CH₂)₄CN,H], [A628;H,H,NHC(═S)NH(CH₂)CN,H], [A629;H,H,NHC(═S)NHCH₂C═CHF,H], [A1630;H,H,NHC(═S)NHCH₂C═CF₂,H], [A1631;H,H,NHC(═S)NH(CH₂)₂C═CHF,H], [A1632;H,H,NHC(═S)NHCHCH₂C═CF₂,H], [A1633;H,H,NHC(═S)NH(CH₂)₃C═CHF,H], [A1634;H,H,NHC(═S)NHCH₂CHCH₂C═CF₂,H], [A1635;H,H,NHC(═S)NHCH₂C═CHCl,H], [A1636;H,H,NHC(═S)NHCH₂C═CCl₂,H], [A1637;H,H,NHC(═S)NH(CH₂)₂C═CHCl,H], [A1638;H,H,NHC(═S)NHCHCH₂C═CCl₂,H], [A1639;H,H,NHC(═S)NH(CH₂)₃C═CHCl,H], [A1640;H,H,NHC(═S)NHCH₂CHCH₂C═CCl₂,H], [A1641;H,H,NHC(═S)NHCH₂C═CH,H], [A1642;H,H,NHC(═S)NHCH(Me)C═CH,H], [A1643;H,H,NHC(═S)NHCH₂C═CMe,H], [A1644;H,H,NHC(═S)NHCH₂C=CEt,H], [A1645;H,H,NHC(═S)NHCH₂C=CEt,H], [A1646;H,H,NHC(═S)NH(CH₂)₂C═CH,H], [A1647;H,H,NHC(═S)NH(CH₂)₂C═CMe,H], [A1648;H,H,NHC(═S)NH(CH₂)₃C═CH,H], [A1649;H,H,NHC(═S)NH(CH₂)₄C═CH,H], [A1650;H,H,NHC(═S)NHc-Pr,H], [A1651;H,H,NHC(═S)NHc-Bu,H], [A1652;H,H,NHC(═S)NHc-Pen,H], [A1653;H,H,NHC(═S)NHc-Hex,H], [A1654;H,H,NHC(═S)NHc-Hep,H], [A1655;H,H,NHC(═S)NHOCH₂F,H], [A1656;H,H,NHC(═S)NHOCHF₂,H], [A1657;H,H,NHC(═S)NHOCF₃,H], [A1658;H,H,NHC(═S)NHO(CH₂)₂F,H], [A1659;H,H,NHC(═S)NHOCH₂CHF₂,H], [A1660;H,H,NHC(═S)NHOCH₂CF₃,H], [A1661;H,H,NHC(═S)NHO(CH₂)₃F,H], [A1662;H,H,NHC(═S)NHO(CH₂)₂CHF₂,H], [A1663;H,H,NHC(═S)NHO(CH₂)₂CF₃,H], [A1664;H,H,NHC(═S)NHOCH₂CH(CF₃)₂,H], [A1665;H,H,NHC(═S)NHOCH₂CH═CH₂,H], [A1666;H,H,NHC(═S)NHC(═S)NHOCH₂CH═CHF,H], [A1667;H,H,NHC(═S)NHOCH₂CH═CF₂,H], [A1668;H,H,NHC(═S)NHOCH₂CH═CHCl,H], [A1669;H,H,NHC(═S)NHOCH₂₂CH═CCl₂,H], [A1670;H,H,NHC(═S)NHOCH₂C═CH,H], [A1671;H,H,NHC(═S)NHOCH(Me)C═CH,H], [A1672;H,H,NHC(═S)NHOCH₂C═CMe,H], [A1673;H,H,NHC(═S)NMeCH₂F,H], [A1674;H,H,NHC(═S)NMeCHF₂,H], [A1675;H,H,NHC(═S)NMeCF₃,H], [A1676;H,H,NHC(═S)NMe(CH₂)₂F,H], [A1677;H,H,NHC(═S)NMeCH₂CHF₂,H], [A1678;H,H,NHC(═S)NMeCH₂CF₃,H], [A1679;H,H,NHC(═S)NMe(CH₂)₃F,H], [A1680;H,H,NHC(═S)NMe(CH₂)₂CHF₂,H], [A1681;H,H,NHC(═S)NMe(CH₂)₂CF₃,H], [A1682;H,H,NHC(═S)NMeCH₂CH(CF₃)₂,H], [A1683;H,H,NHC(═S)NMeCH₂Cl,H], [A1684;H,H,NHC(═S)NMe(CH₂)₂Cl,H], [A1685;H,H,NHC(═S)NMe(CH₂)₃Cl,H], [A1686;H,H,NHC(═S)NMe(CH₂)₄Cl,H], [A1687;H,H,NHC(═S)NMe(CH₂)₅Cl,H], [A1688;H,H,NHC(═S)NMeCH₂Br,H], [A1689;H,H,NHC(═S)NMe(CH₂)₂Br,H], [A1690;H,H,NHC(═S)NMe(CH₂)₃Br,H], [A1691;H,H,NHC(═S)NMe(CH₂)₄Br,H], [A1692;H,H,NHC(═S)NMe(CH₂)₅Br,H], [A1693;H,H,NHC(═S)NMeCH₂OMe,H], [A1694;H,H,NHC(═S)NMe(CH₂)₂OMe,H], [A1695;H,H,NHC(═S)NMe(CH₂)₃OMe,H], [A1696;H,H,NHC(═S)NMe(CH₂)₄OMe,H], [A1697;H,H,NHC(═S)NMeCH₂OEt,H], [A1698;H,H,NHC(═S)NMe(CH₂)₂OEt,H], [A1699;H,H,NHC(═S)NMe(CH₂)₃OEt,H], [A1700;H,H,NHC(═S)NMeCH₂CN,H],

[A1701;H,H,NHC(═S)NMe(CH₂)₂CN,H], [A1702;H,H,NHC(═S)NMe(CH₂)₃CN,H], [A1703;H,H,NHC(═S)NMe(CH₂)₄CN,H][A1704;H,H,NHC(═S)NMe(CH₂)₅CN,H][A1705;H,H,NHC(═S)NMeCH₂C═CHF,H][A1706;H,H,NHC(═S)NMeCH₂C═CF₂,H][A1707;H,H,NHC(═S)NMe(CH₂)₂C═CHF,H], [A1708;H,H,NHC(═S)NMeCHCH₂C═CF₂,H][A1709;H,H,NHC(═S)NMe(CH₂)₃C═CHF,H], [A1710;H,H,NHC(═S)NMeCH₂CHCH₂C═CF₂,H][A1711;H,H,NH(═S)NMeCH₂C═CHCl,H], [A1712;H,H,NHC(═S)NMeCH₂C═CHCl₂,H], [A1713;H,H,NHC(═S)NMe(CH₂)C═CHCl,H], [A1714;H,H,NHC(═S)NMeCHCH₂C═CCl₂,H][A1715;H,H,NHC(═S)NMe(CH₂)₃C═CHCl,H], [A1716;H,H,NHC(═S)NMeCH₂CHCH₂C═CCl₂,H][A1717;H,H,NHC(═S)NMeCH₂C═CH,H], [A1718;H,H,NHC(═S)NMeCH(Me)C═CH,H][A1719;H,H,NHC(═S)NMeCH₂C═CMe,H][A1720;H,H,NHC(═S)NMeCH₂C=CEt,H][A1721;H,H,NHC(═S)NMeCH₂C=CEt,H][A1722;H,H,NHC(═S)NMe(CH₂)₂C═CH,H], [A1723;H,H,NHC(═S)NMe(CH₂₂C═CMe,H][A1724;H,H,NHC(═S)NMe(CH₂)₃C═CH,H], [A1725;H,H,NHC(═S)NMe(CH₂)₄C═CH,H], [A1726;H,H,NHC(═S)NMec-Pr,H], [A1727;H,H,NHC(═S)NMec-Bu,H], [A1728;H,H,NHC(═S)NMec-Pen,H], [A1729;H,H,NHC(═S)NMec-Hex,H], [A1730;H,H,NHC(═S)NMec-Hep,H], [A1731;H,H,NHC(═S)NMeOCH₂F,H], [A1732;H,H,NHC(═S)NMeOCHF₂,H], [A1733;H,H,NHC(═S)NMeOCF₃,H], [A1734;H,H,NHC(═S)NMeO(CH₂)₂F,H][A1735;H,H,NHC(═S)NMeOCH₂CHF₂,H][A1736;H,H,NHC(═S)NMeOCH₂CF₃,H], [A1737;H,H,NHC(═S)NMeO(CH₂)₃F,H], [A1738;H,H,NHC(═S)NMeO(CH₂)₂CHF₂,H], [A1739;H,H,NHC(═S)NMeO(CH₂)₂CF₃,H], [A1740;H,H,NHC(═S)NMeOCH₂CH(CF₃)₂,H], [A1741;H,H,NHC(═S)NMeOCH₂CH═CH₂,H], [A1742;H,H,NHC(═S)NMeOCH₂CH═CHF,H], [A1743;H,H,NHC(═S)NMeOCH₂CH═CF₂,H], [A1744;H,H,NHC(═S)NMeOCH₂CH═CHCl,H], [A1745;H,H,NHC(═S)NMeOCH₂CH═CCl₂,H], [A1746;H,H,NHC(═S)NMeOCH₂C═CH,H], [A1747;H,H,NHC(═S)NMeOCH(Me)C═CH,H], [A1748;H,H,NHC(═S)NMeOCH₂C═CMe,H], [A1749;H,H,NHC(═S)NEtCH₂F,H], [A1750;H,H,NHC(═S)NEtCHF₂,H], [A1751;H,H,NHC(═S)NEtCF₃,H], [A1752;H,H,NHC(═S)NEt(CH₂)₂F,H], [A1753;H,H,NHC(═S)NEtCH₂CHF₂,H], [A1754;H,H,NHC(═S)NEtCH₂CF₃,H], [A1755;H,H,NHC(═S)NEt(CH₂)₃F,H], [A1756;H,H,NHC(═S)NEt(CH₂)₂CHF₂,H], [A1757;H,H,NHC(═S)NEt(CH₂)₂CF₃,H], [A1758;H,H,NHC(═S)NEtCH₂CH(CF₃)₂,H], [A1759;H,H,NHC(═S)NEtCH₂Cl,H], [A1760;H,H,NHC(═S)NEt(CH₂)₂Cl,H], [A1761;H,H,NHC(═S)NEt(CH₂)₃Cl,H], [A1762;H,H,NHC(═S)NEt(CH₂)₄₁C,H], [A1763;H,H,NHC(═S)NEt(CH₂)₅Cl,H], [A1764;H,H,NHC(═S)NEtCH₂Br,H], [A1765;H,H,NHC(═S)NEt(CH₂)₂Br,H], [A1766;H,H,NHC(═S)NEt(CH₂)₃Br,H], [A1767;H,H,NHC(═S)NEt(CH₂)₄Br,H], [A1768;H,H,NHC(═S)NEt(CH₂)₅Br,H], [A1769;H,H,NHC(═S)NEtCH₂OMe,H], [A1770;H,H,NHC(═S)NEt(CH₂)₂OMe,H], [A1771;H,H,NHC(═S)NEt(CH₂)₃OMe,H], [A1772;H,H,NHC(═S)NEt(CH₂)₄OMe,H], [A1773;H,H,NHC(═S)NEtCH₂OEt,H], [A1774;H,H,NHC(═S)NEt(CH₂)₂OEt,H], [A1775;H,H,NHC(═S)NEt(CH₂)₃OEt,H], [A1776;H,H,NHC(═S)NEtCH₂CN,H], [A1777;H,H,NHC(═S)NEt(CH₂)₂CN,H], [A1778;H,H,NHC(═S)NEt(CH₂)₃CN,H], [A1779;H,H,NHC(═S)NEt(CH₂)₄CN,H], [A1780;H,H,NHC(═S)NEt(CH₂₎CN,H], [A1781;H,H,NHC(═S)NEtCH₂C═CHF,H], [A1782;H,H,NHC(═S)NEtCH₂C═CF₂,H], [A1783;H,H,NHC(═S)NEt(CH₂)₂C═CHF,H], [A1784;H,H,NHC(═S)NEtCHCH₂C═CF₂,H], [A1785;H,H,NHC(═S)NEt(CH₂)₃C═CHF,H], [A1786;H,H,NHC(═S)NEtCH₂CH₂C═CF₂,H], [A1787;H,H,NHC(═S)NEtCH₂C═CHCl,H], [A1788;H,H,NHC(═S)NEtCH₂C═CCl₂,H], [A1789;H,H,NHC(═S)NEt(CH₂)₂C═CHCl,H], [A1790;H,H,NHC(═S)NEtCHCH₂C═CCl₂,H], [A1791;H,H,NHC(═S)NEt(CH₂)₃C═CHCl,H], [A1792;H,H,NHC(═S)NEtCH₂CH₂CHC═CCl₂,H], [A1793;H,H,NHC(═S)NEtCH₂C═CH,H], [A1794;H,H,NHC(═S)NEtCH(Me)C═CH,H], [A1795;H,H,NHC(═S)NEtCH₂C═CMe,H], [A1796;H,H,NHC(═S)NEtCH₂C=CEt,H], [A1797;H,H,NHC(═S)NEtCH₂C=CEt,H], [A1798;H,H,NHC(═S)NEt(CH₂)₂C═CH,H], [A1799;H,H,NHC(═S)NEt(CH₂)₂C═CMe,H], [A1800;H,H,NHC(═S)NEt(CH₂)₃C═CH,H],

[A1801;H,H,NHC(═S)NEt(CH₂)₄C═CH,H], [A1802;H,H,NHC(═S)NEtc-Pr,H], [A1803;H,H,NHC(═S)NEtc-Bu,H], [A1804;H,H,NHC(═S)NEtc-Pen,H], [A1805;H,H,NHC(═S)NEtc-Hex,H], [A1806;H,H,NHC(═S)NEtc-Hep,H], [A1807;H,H,NHC(═S)NEtOCH₂F,H], [A1808;H,H,NHC(═S)NEtOCHF₂,H], [A1809;H,H,NHC(═S)NEtOCF₃,H], [A1810;H,H,NHC(═S)NEtO(CH₂)₂F,H], [A1811;H,H,NHC(═S)NEtOCH₂CHF₂,H], [A1812;H,H,NHC(═S)NEtOCH₂CF₃,H], [A1813;H,H,NHC(═S)NEtO(CH₂)₃F,H], [A1814;H,H,NHC(═S)NEtO(CH₂)₂CHF₂,H], [A1815;H,H,NHC(═S)NEtO(CH₂)₂CF₃,H], [A1816;H,H,NHC(═S)NEtOCH₂CH(CF₃)₂,H], [A1817;H,H,NHC(═S)NEtOCH₂CH═CH₂,H], [A1818;H,H,NHC(═S)NEtOCH₂CH═CHF,H], [A1819;H,H,NHC(═S)NEtOCH₂CH═CF₂,H], [A1820;H,H,NHC(═S)NEtOCH₂CH═CHCl,H], [A1821;H,H,NHC(═S)NEtOCH₂CH═CCl₂,H], [A1822;H,H,NHC(═S)NEtOCH₂C═CH,H], [A1823;H,H,NHC(═S)NEtOCH(Me)C═CH,H], [A1824;H,H,NHC(═S)NEtOCH₂C═CMe,H], [A1825;H,H,NHC(═S)NPrCH₂F,H], [A1826;H,H,NHC(═S)NPrCHF₂,H], [A1827;H,H,NHC(═S)NPrCF₃,H], [A1828;H,H,NHC(═S)NPr(CH₂)₂F,H], [A1829;H,H,NHC(═S)NPrCH₂CHF₂,H], [A1830;H,H,NHC(═S)NPrCH₂CF₃,H], [A1831;H,H,NHC(═S)NPr(CH₂)₃F,H], [A1832;H,H,NHC(═S)NPr(CH₂)₂CHF₂,H], [A1833;H,H,NHC(═S)NPr(CH₂)₂CF₃,H], [A1834;H,H,NHC(═S)NPrCH₂CH(CF₃)₂,H], [A1835;H,H,NHC(═S)NPrCH₂Cl,H], [A1836;H,H,NHC(═S)NPr(CH₂)₂Cl,H][A1837;H,H,NHC(═S)NPr(CH₂)₃Cl,H], [A1838;H,H,NHC(═S)NPr(CH₂)₄Cl,H], [A1839;H,H,NHC(═S)NPr(CH₂)₅Cl,H], [A1840;H,H,NHC(═S)NPrCH₂Br,H][A1841;H,H,NHC(═S)NPr(CH₂)Br,H][A1842;H,H,NHC(═S)NPr(CH₃)Br,H], [A1843;H,H,NHC(═S)NPr(CH₂)₄Br,H], [A1844;H,H,NHC(═S)NPr(CH₂)₅Br,H], [A1845;H,H,NHC(═S)NPr(CH₂OMe,H], [A1846;H,H,NHC(═S)NPr(CH₂)₂OBr,H], [A1847;H,H,NHC(═S)NPr(CH₂)₃OMe,H], [A1848;H,H,NHC(═S)NPr(CH₂)₄OMe,H], [A1849;H,H,NHC(═S)NPrCH₂OEt,H][A1850;H,H,NHC(═S)NPr(CH₂)₂OEt,H], [A1851;H,H,NHC(═S)NPr(CH₂)₃OEt,H], [A1852;H,H,NHC(═S)NPrCH₂CN,H], [A1853;H,H,NHC(═S)NPr(CH₂)₂CN,H], [A1854;H,H,NHC(═S)NPr(CH₂)₃CN,H], [A1855;H,H,NHC(═S)NPr(CH₂)₄CN,H], [A18526;H,H,NHC(═S)NPr(CH₂)₅CN,H][A1857;H,H,NHC(═S)NPrCH₂CHF,H], [A1858;H,H,NHC(═S)NPrCH₂C═CF₂,H], [A1859;H,H,NHC(═S)NPr(CH₂)₂C═CHF,H], [A1860;H,H,NHC(═S)NPrCHCH₂C═CF₂,H][A1857;H,H,NHC(═S)NPr(CH₂)₃C═CHF,H], [A1862;H,H,NHC(═S)NPrCH₂CHCH₂C═CF₂,H][A1863;H,H,NHC(═S)NPrCH₂C═CHCl,H], [A1864;H,H,NHC(═S)NPrCH₂C═CCl₂,H][A1865;H,H,NHC(═S)NPr(CH₂)₂C═CHCl,H], [A1866;H,H,NHC(═S)NPrCHCH₂C═CCl₂,H][A1867;H,H,NHC(═S)NPr(CH₂)₃C═CHCl,H], [A1868;H,H,NHC(═S)NPrCH₂CHCH₂C═CCl₂,H], [A1869;H,H,NHC(═S)NPrCH₂C═CH,H], [A1870;H,H,NHC(═S)NPrCH(Me)C═CH,H][A1871;H,H,NHC(═S)NPrCH₂C═CMe,H], [A1872;H,H,NHC(═S)NPrCH₂C=CEt,H], [A1873;H,H,NHC(═S)NPrCH₂C=CEt,H], [A1874;H,H,NHC(═S)NPr(CH₂)₂C═CH,H], [A1875;H,H,NHC(═S)NPr(CH₂)₂C═CMe,H], [A1876;H,H,NHC(═S)NPr(CH₂)₃C═CH,H], [A1877;H,H,NHC(═S)NPr(CH₂)₄C═CH,H], [A1878;H,H,NHC(═S)NPrc-Pr,H], [A1879;H,H,NHC(═S)NPrc-Bu,H], [A1880;H,H,NHC(═S)NPrc-Pen,H], [A1881;H,H,NHC(═S)NPrc-Hex,H], [A1882;H,H,NHC(═S)NPrc-Hep,H], [A1883;H,H,NHC(═S)NPrOCH₂F,H], [A1884;H,H,NHC(═S)NPrOCHF₂,H], [A1885;H,H,NHC(═S)NPrOCF₃,H], [A1886;H,H,NHC(═S)NPrO(CH₂)₂F,H], [A1887;H,H,NHC(═S)NPrOCH₂CHF₂,H], [A1888;H,H,NHC(═S)NPrOCH₂CF₃,H], [A1889;H,H,NHC(═S)NPrO(CH₂)₃F,H], [A1890;H,H,NHC(═S)NPrO(CH₂)₂CHF₂,H], [A1891;H,H,NHC(═S)NPrO(CH₂)₂CF₃,H], [A1892;H,H,NHC(═S)NPrOCH₂CH(CF₃)₂,H], [A1893;H,H,NHC(═S)NPrOCH₂CH═CH₂,H][A1894;H,H,NHC(═S)NPrOCH₂CH═CHF,H][A1895;H,H,NHC(═S)NPrOCH₂CH═CF₂,H][A1896;H,H,NHC(═S)NPrOCH₂CH═CCl₂,H], [A1897;H,H,NHC(═S)NPrOCH₂CH═CCl₂,H], [A1898;H,H,NHC(═S)NPrOCH₂C═CH,H], [A1899;H,H,NHC(═S)NPrOCH(Me)C═CH,H][A1900;H,H,NHC(═S)NPrOCH₂C≡CMe,H],

[A1901;H,H,NHC(═S)N(i-Pr)CH₂F,H], [A1902;H,H,NHC(═S)N(i-Pr)CHF₂,H], [A1903;H,H,NHC(═S)N(i-Pr)CF₃,H], [A1904;H,H,NHC(═S)N(i-Pr)(CH₂)₂F,H], [A1905;H,H,NHC(═S)N(i-Pr)CH₂CHF₂,H], [A1906;H,H,NHC(═S)N(i-Pr)CH₂CF₃,H], [A1907;H,H,NHC(═S)N(i-Pr)(CH₂)₃F,H], [A1908;H,H,NHC(═S)N(i-Pr)(CH₂)₂CHF₂,H], [A1909;H,H,NHC(═S)N(i-Pr)(CH₂)₂CF₃,H], [A1910;H,H,NHC(═S)N(i-Pr)CH₂CH(CF₃),H], [A1911;H,H,NHC(═S)N(i-Pr)CH₂Cl,H], [A1912;H,H,NHC(═S)N(i-Pr)(CH₂)₂Cl,H], [A1913;H,H,NHC(═S)N(i-Pr)(CH₂)₃Cl,H], [A1914;H,H,NHC(═S)N(i-Pr)(CH₂)₄Cl,H], [A1915;H,H,NHC(═S)N(i-Pr)(CH₂)₅Cl,H], [A1916;H,H,NHC(═S)N(i-Pr)CH₂Br,H], [A1917;H,H,NHC(═S)N(i-Pr)(CH₂)₂Br,H], [A1918;H,H,NHC(═S)N(i-Pr)(CH₂)₃Br,H], [A1919;H,H,NHC(═S)N(i-Pr)(CH₂)₄Br,H], [A1920;H,H,NHC(═S)N(i-Pr)(CH₂)₅Br,H], [A1921;H,H,NHC(═S)N(i-Pr)CH₂OMe,H], [A1922;H,H,NHC(═S)N(i-Pr)(CH₂)₂OMe,H], [A1923;H,H,NHC(═S)N(i-Pr)(CH₂)₃OMe,H], [A1924;H,H,NHC(═S)N(i-Pr)(CH₂)₄OMe,H], [A1925;H,H,NHC(═S)N(i-Pr)CH₂OEt,H], [A1926;H,H,NHC(═S)N(i-Pr)(CH₂)₂OEt,H], [A1927;H,H,NHC(═S)N(i-Pr)(CH₂)₃OEt,H], [A1928;H,H,NHC(═S)N(i-Pr)CH₂CN,H], [A1929;H,H,NHC(═S)N(i-Pr)(CH₂)₂CN,H], [A1930;H,H,NHC(═S)N(i-Pr)(CH₂)₃CN,H], [A1931;H,H,NHC(═S)N(i-Pr)(CH₂)₄CN,H], [A1932;H,H,NHC(═S)N(i-Pr)(CH₂)₅CN,H], [A1933;H,H,NHC(═S)N(i-Pr) CH₂C═CHF,H], [A1934;H,H,NHC(═S)N(i-Pr) CH₂C═CF₂,H], [A1935;H,H,NHC(═S)N(i-Pr)(CH₂)₂C═CHF,H], [A1936;H,H,NHC(═S)N(i-Pr) CHCH₂C═CF₂,H], [A1937;H,H,NHC(═S)N(i-Pr)(CH₂)₃C═CHF,H], [A1938;H,H,NHC(═S)N(i-Pr) CH₂CHCH₂C═CF₂,H], [A1939;H,H,NHC(═S)N(i-Pr)CH₂C═CHCl,H], [A1940;H,H,NHC(═S)N(i-Pr)CH₂C═CCl₂,H], [A1941;H,H,NHC(═S)N(i-Pr)(CH₂)₂C═CHCl,H], [A1942;H,H,NHC(═S)N(i-Pr) CHCH₂C═CCl₂,H], [A1943;H,H,NHC(═S)N(i-Pr)(CH₂)₃C═CHCl,H], [A1944;H,H,NHC(═S)N(i-Pr) CH₂CHCH₂C═CCl₂,H], [A1945;H,H,NHC(═S)N(i-Pr) CH₂C═CH,H], [A1946;H,H,NHC(═S)N(i-Pr)CH(Me)C═CH,H], [A1947;H,H,NHC(═S)N(i-Pr)CH₂C═CMe,H], [A1948;H,H,NHC(═S)N(i-Pr)CH₂C=CEt,H], [A1949;H,H,NHC(═S)N(i-Pr) CH₂C=CEt,H], [A1950;H,H,NHC(═S)N(i-Pr)(CH₂)₂C═CH,H], [A1951;H,H,NHC(═S)N(i-Pr)(CH₂)₂C═CMe,H], [A1952;H,H,NHC(═S)N(i-Pr)(CH₂)₃C═CH,H], [A1953;H,H,NHC(═S)N(i-Pr)(CH₂)₄C═CH,H], [A1954;H,H,NHC(═S)N(i-Pr)c-Pr,H], [A1955;H,H,NHC(═S)N(i-Pr)c-Bu,H], [A1956;H,H,NHC(═S)N(i-Pr)c-Pen,H], [A1957;H,H,NHC(═S)N(i-Pr)c-Hex,H], [A1958;H,H,NHC(═S)N(i-Pr) c-Hep,H], [A1959;H,H,NHC(═S)N(i-Pr) OCH₂F,H], [A1960;H,H,NHC(═S)N(i-Pr)OCHF₂,H], [A1961;H,H,NHC(═S)N(i-Pr)OCF₃,H], [A1962;H,H,NHC(═S)N(i-Pr)O(CH₂)₂F,H], [A1963;H,H,NHC(═S)N(i-Pr) OCH₂CHF₂,H], [A1964;H,H,NHC(═S)N(i-Pr) OCH₂CF₃,H], [A1965;H,H,NHC(═S)N(i-Pr)O(CH₂)₃F,H], [A1966;H,H,NHC(═S)N(i-Pr)O(CH₂)₂CHF₂,H], [A1967;H,H,NHC(═S)N(i-Pr)O(CH₂)CF₃,H], [A1968;H,H,NHC(═S)N(i-Pr)OCH₂CH(CF₃)₂,H], [A1969;H,H,NHC(═S)N(i-Pr)OCH₂CH═CH₂,H], [A1970;H,H,NHC(═S)N(i-Pr)OCH₂CH═CHF,H], [A1971;H,H,NHC(═S)N(i-Pr)OCH₂CH═CF₂,H], [A1972;H,H,NHC(═S)N(i-Pr)OCH₂CH═CHCl,H], [A1973;H,H,NHC(═S)N(i-Pr)OCH₂CH═CCl₂,H], [A1974;H,H,NHC(═S)N(i-Pr)OCH₂C═CH,H], [A1975;H,H,NHC(═S)N(i-Pr)OCH(Me)C═CH,H], [A1976;H,H,NHC(═S)N(i-Pr)OCH₂C═CMe,H], [A1977;H,H,NHC(═O)OCH₂F,H], [A1978;H,H,NHC(═O)OCHF₂,H], [A1979;H,H,NHC(═O)OCF₃,H], [A1980;H,H,NHC(═O)O(CH₂)₂F,H], [A1981;H,H,NHC(═O)OCH₂CHF₂,H], [A1982;H,H,NHC(═O)OCH₂CF₃,H], [A1983;H,H,NHC(═O)O(CH₂)₃F,H], [A1984;H,H,NHC(═O)O(CH₂)₂CHF₂,H][A1985;H,H,NHC(═O)O(CH₂)₂CF₃,H], [A1986;H,H,NHC(═O)OCH₂CH(CF₃)₂,H][A1987;H,H,NHC(═O)O(CH₂Cl,H], [A1988;H,H,NHC(═O)O(CH₂)₂Cl,H], [A1989;H,H,NHC(═O)O(CH₂)₃Cl,H], [A1990;H,H,NHC(═O)O(CH₂)₄Cl,H], [A1991;H,H,NHC(═O)O(CH₂)₅Cl,H], [A1992;H,H,NHC(═O)OCH₂Br,H], [A1993;H,H,NHC(═O)O(CH₂)₂Br,H], [A1994;H,H,NHC(═O)O(CH₂)₃Br,H], [A1995;H,H,NHC(═O)O(CH₂)₄Br,H], [A1996;H,H,NHC(═O)O(CH₂)₅Br,H], [A1997;H,H,NHC(═O)OCH₂OMe,H], [A1998;H,H,NHC(═O)O(CH₂)₂OMe,H], [A1999;H,H,NHC(═O)O(CH₂)₃₀Me,H], [A2000;H,H,NHC(═O)O(CH₂)₄OMe,H],

[A2001;H,H,NHC(═O)OCH₂OEt,H], [A2002;H,H,NHC(═O)O(CH₂)₂Et,H], [A2003;H,H,NHC(═O)O(CH₂)₃OEt,H], [A2004;H,H,NHC(═O)OCH₂CN,H], [A2005;H,H,NHC(═O)O(CH₂)₂CN,H], [A2006;H,H,NHC(═O)O(CH₂)CN,H], [A2007;H,H,NHC(═O)O(CH₂)₄CN,H], [A2008;H,H,NHC(═O)O(CH₂)₅CN,H], [A2009;H,H,NHC(═O)OCH₂C═CHF,H][A2010;H,H,NHC(═O)OCH₂C═CF₂,H], [A2011;H,H,NHC(═O)O(CH₂)₂C═CHF,H], [A2012;H,H,NHC(═O)OCHCH₂C═CF₂,H], [A2013;H,H,NHC(═O)O(CH₂)₃C═CHF,H], [A2014;H,H,NHC(═O)OCH₂CHCH₂C═CF₂,H][A2015;H,H,NHC(═O)OCH₂C═CHCl,H][A2016;H,H,NHC(═O)OCH₂C═CCl₂,H], [A2017;H,H,NHC(═O)O(CH₂)₂C═CHCl,H], [A2018;H,H,NHC(═O)OCHCH₂C═CCl₂,H][A2019;H,H,NHC(═O)O(CH₂)₃C═CHCl,H], [A2020;H,H,NHC(═O)OCH₂CHCH₂C═CCl₂,H], [A2021;H,H,NHC(═O)OCH₂C═CH,H][A2022;H,H,NHC(═O)OCH(Me)C═CH,H], [A2023;H,H,NHC(═O)OCH₂C═CMe,H][A2024;H,H,NHC(═O)OCH₂C=CEt,H][A2025;H,H,NHC(═O)OCH₂C=CEt,H][A2026;H,H,NHC(═O)O(CH₂)₂C═CH,H], [A2027;H,H,NHC(═O)O(CH₂)₂C═CMe,H], [A2028;H,H,NHC(═O)O(CH₂)₃C═CH,H], [A2029;H,H,NHC(═O)O(CH₂)₄C═CH,H], [A2030;H,H,NHC(═O)Oc-Pr,H], [A2031;H,H,NHC(═O)Oc-Bu,H], [A2032;H,H,NHC(═O)Oc-Pen,H], [A2033;H,H,NHC(═O)Oc-Hex,H], [A2034;H,H,NHC(═O)Oc-Hep,H], [A2035;H,H,NHC(═S)OCH₂F,H], [A2036;H,H,NHC(═S)OCHF₂,H], [A2037;H,H,NHC(═S)OCF₃,H], [A2038;H,H,NHC(═S)O(CH₂)₂F,H], [A2039;H,H,NHC(═S)OCH₂CHF₂,H], [A2040;H,H,NHC(═S)OCH₂CF₃,H], [A2041;H,H,NHC(═S)O(CH₂)₃F,H], [A2042;H,H,NHC(═S)O(CH₂)₂CHF₂,H], [A2043;H,H,NHC(═S)O(CH₂)₂CF₃,H], [A2044;H,H,NHC(═S)OCH₂CH(CF₃)₂,H], [A2045;H,H,NHC(═S)OCH₂Cl,H], [A2046;H,H,NHC(═S)O(CH₂)₂Cl,H], [A2047;H,H,NHC(═S)O(CH₂)₃Cl,H], [A2048;H,H,NHC(═S)O(CH₂)₄Cl,H], [A2049;H,H,NHC(═S)O(CH₂)₅Cl,H], [A2050;H,H,NHC(═S)OCH₂Br,H], [A2051;H,H,NHC(═S)O(CH₂)₂Br,H], [A2052;H,H,NHC(═S)O(CH₂)₃Br,H], [A2053;H,H,NHC(═S)O(CH₂)₄Br,H], [A2054;H,H,NHC(═S)O(CH₂)₅Br,H], [A2055;H,H,NHC(═S)OCH₂OMe,H], [A2056;H,H,NHC(═S)O(CH₂)₂OMe,H], [A2057;H,H,NHC(═S)O(CH₂)₃OMe,H], [A2058;H,H,NHC(═S)O(CH₂)₄OMe,H], [A2059;H,H,NHC(═S)OCH₂OEt,H], [A2060;H,H,NHC(═S)O(CH₂)₂OEt,H], [A2061;H,H,NHC(═S)O(CH₂)₃OEt,H], [A2062;H,H,NHC(═S)OCH₂CN,H], [A2063;H,H,NHC(═S)O(CH₂)₂CN,H], [A2064;H,H,NHC(═S)O(CH₂)₃CN,H], [A2065;H,H,NHC(═S)O(CH₂)₄CN,H], [A2066;H,H,NHC(═S)O(CH₂)₅CN,H], [A2067;H,H,NHC(═S)OCH₂C═CHF,H], [A2068;H,H,NHC(═S)OCH₂C═CF₂,H], [A2069;H,H,NHC(═S)O(CH₂)₂C═CHF,H], [A2070;H,H,NHC(═S)OCHCH₂C═CF₂,H], [A2071;H,H,NHC(═S)O(CH₂)₃C═CHF,H][A2072;H,H,NHC(═S)OCH₂CHCH₂C═CF₂,H], [A2073;H,H,NHC(═S)OCH₂C═CHCl,H], [A2074;H,H,NHC(═S)OCH₂C═CCl₂,H], [A2075;H,H,NHC(═S)O(CH₂)₂C═CHCl,H], [A2076;H,H,NHC(═S)OCHCH₂C═CCl₂,H][A2077;H,H,NHC(═S)O(CH₂)₃C═CHCl,H], [A2078;H,H,NHC(═S)OCH₂CHCH₂C═CCl₂,H], [A2079;H,H,NHC(═S)OCH₂C═CH,H], [A2080;H,H,NHC(═S)OCH(Me)C═CH,H], [A2081;H,H,NHC(═S)OCH₂C═CMe,H], [A2082;H,H,NHC(═S)OCH₂C=CEt,H], [A2083;H,H,NHC(═S)OCH₂C=CEt,H], [A2084;H,H,NHC(═S)O(CH₂)₂C═CH,H], [A2085;H,H,NHC(═S)O(CH₂)₂C═CMe,H], [A2086;H,H,NHC(═S)O(CH₂)₃C═CH,H], [A2087;H,H,NHC(═S)O(CH₂)₄C═CH,H], [A2088;H,H,NHC(═S)Oc-Pr,H], [A2089;H,H,NHC(═S)Oc-Bu,H], [A2090;H,H,NHC(═S)Oc-Pen,H], [A2091;H,H,NHC(═S)Oc-Hex,H], [A2092;H,H,NHC(═S)Oc-Hep,H], [A2093;H,H,NHC(═O)SCH₂F,H], [A2094;H,H,NHC(═O)SCHF₂,H], [A2095;H,H,NHC(═O)SCF₃,H], [A2096;H,H,NHC(═O)S(CH₂)₂F,H], [A2097;H,H,NHC(═O)SCH₂CHF₂,H], [A2098;H,H,NHC(═O)SCH₂CF₃,H], [A2099;H,H,NHC(═O)S(CH₂)₃F,H], [A2100;H,H,NHC(═O)S(CH₂)₂CHF₂,H],

[A2101;H,H,NHC(═O)S(CH₂)₂CF₃,H], [A2102;H,H,NHC(═O)SCH₂CH(CF₃)₂,H][A2103;H,H,NHC(═O)SCH₂Cl,H], [A2104;H,H,NHC(═O)S(CH₂)₂Cl,H], [A2105;H,H,NHC(═O)S(CH₂) C1,H], [A2106;H,H,NHC(═O)S(CH₂)₄Cl,H], [A2107;H,H,NHC(═O)S(CH₂)₅Cl,H], [A2108;H,H,NHC(═O)SCH₂Br,H], [A2109;H,H,NHC(═O)S(CH₂)₂Br,H], [A2110;H,H,NHC(═O)S(CH₂)₃Br,H], [A2111;H,H,NHC(═O)S(CH₂)₄Br,H], [A2112;H,H,NHC(═O)S(CH₂)₅Br,H], [A2113;H,H,NHC(═O)SCH₂OMe,H], [A2114;H,H,NHC(═O)S(CH₂)₂OMe,H], [A2115;H,H,NHC(═O)S(CH₂)₃OMe,H], [A2116;H,H,NHC(═O)S(CH₂)₄OMe,H], [A2117;H,H,NHC(═O)SCH₂OEt,H], [A2118;H,H,NHC(═O)S(CH₂)₂OEt,H], [A2119;H,H,NHC(═O)S(CH₂)₃OEt,H], [A2120;H,H,NHC(═O)SCH₂CN,H], [A2121;H,H,NHC(═O)S(CH₂)₂CN,H], [A2122;H,H,NHC(═O)S(CH₂)₃CN,H], [A2123;H,H,NHC(═O)S(CH₂)₄CN,H], [A2124;H,H,NHC(═O)S(CH₂)₅CN,H], [A2125;H,H,NHC(═O)SCH₂C═CHF,H], [A2126;H,H,NHC(═O)SCH₂C═CF₂,H], [A2127;H,H,NHC(═O)S(CH₂)₂C═CHF,H], [A2128;H,H,NHC(═O)SCHCH₂C═CF₂,H], [A2129;H,H,NHC(═O)S(CH₂)₃C═CHF,H], [A2130;H,H,NHC(═O)SCH₂CHCH₂C═CF₂,H], [A2131;H,H,NHC(═O)SCH₂C═CHCl,H], [A2132;H,H,NHC(═O)SCH₂C═CCl₂,H], [A2133;H,H,NHC(═O)S (CH₂)₂C═CHCl,H], [A2134;H,H,NHC(═O)SCHCH₂C═CCl₂,H], [A2135;H,H,NHC(═O)S(CH₂)₃C═CHCl,H], [A2136;H,H,NHC(═O)SCH₂CHCH₂C═CCl₂,H], [A2137;H,H,NHC(═O)SCH₂C═CH,H], [A2138;H,H,NHC(═O)SCH(Me)C═CH,H], [A2139;H,H,NHC(═O)SCH₂C═CMe,H], [A2140;H,H,NHC(═O)SCH₂C=CEt,H], [A2141;H,H,NHC(═O)SCH₂C=CEt,H], [A2142;H,H,NHC(═O)S(CH₂)₂C═CH,H], [A2143;H,H,NHC(═O)S(CH₂)₂C═CMe,H], [A2144;H,H,NHC(═O)S(CH₂)₃C═CH,H], [A2145;H,H,NHC(═O)S(CH₂)₄C═CH,H], [A2146;H,H,NHC(═O)Sc—Pr,H], [A2147;H,H,NHC(═O)Sc—Bu,H], [A2148;H,H,NHC(═O)Sc—Pen,H], [A2149;H,H,NHC(═O)Sc—Hex,H], [A2150;H,H,NHC(═O)Sc—Hep,H], [A2151;H,H,NHC(═O)SOCH₂F,H], [A2152;H,H,NHC(═O)SOCHF₂,H], [A2153;H,H,NHC(═O)SOCF₃,H], [A2154;H,H,NHC(═S)SCH₂F,H], [A2155;H,H,NHC(═S)SCHF₂,H], [A2156;H,H,NHC(═S)SCF₃,H], [A2157;H,H,NHC(═S)S(CH₂)₂F,H], [A2158;H,H,NHC(═S)SCH₂CHF₂,H], [A2159;H,H,NHC(═S)SCH₂CF₃,H], [A2160;H,H,NHC(═S)S(CH₂))₃F,H], [A2161;H,H,NHC(═S)S(CH₂)₂CHF₂,H], [A2162;H,H,NHC(═S)S(CH₂)₂CF₃,H][A2163;H,H,NHC(═S)SCH₂CH(CF₃)₂,H][A2164;H,H,NHC(═S)SCH₂Cl,H], [A2165;H,H,NHC(═S)S(CH₂)₂Cl,H], [A2166;H,H,NHC(═S)S(CH₂)₃Cl,H], [A2167;H,H,NHC(═S)S(CH₂)₄Cl,H], [A2168;H,H,NHC(═S)S(CH₂)₅Cl,H], [A2169;H,H,NHC(═S)SCH₂Br,H], [A2170;H,H,NHC(═S)S(CH₂)₂Br,H], [A2171;H,H,NHC(═S)S(CH₂)₃Br,H], [A2172;H,H,NHC(═S)S(CH₂)₄Br,H], [A2173;H,H,NHC(═S)S(CH₂)₅Br,H], [A2174;H,H,NHC(═S)SCH₂OMe,H], [A2175;H,H,NHC(═S)S(CH₂)₂OMe,H], [A2176;H,H,NHC(═S)S(CH₂)₃OMe,H], [A2177;H,H,NHC(═S)S(CH₂)₄OMe,H], [A2178;H,H,NHC(═S)SCH₂OEt,H], [A2179;H,H,NHC(═S)S(CH₂)₂OEt,H], [A2180;H,H,NHC(═S)S(CH₂)₃OEt,H], [A2181;H,H,NHC(═S)SCH₂CN,H], [A2182;H,H,NHC(═S)S(CH₂)₂CN,H], [A2183;H,H,NHC(═S)S(CH₂)₃CN,H], [A2184;H,H,NHC(═S)S(CH₂)₄CN,H], [A2185;H,H,NHC(═S)S(CH₂)₅CN,H], [A2186;H,H,NHC(═S)SCH₂C═CHF,H], [A2187;H,H,NHC(═S)SCH₂C═CF₂,H], [A2188;H,H,NHC(═S)S(CH₂)₂C═CHF,H], [A2189;H,H,NHC(═S)SCHCH₂C═CF₂,H], [A2190;H,H,NHC(═S)S(CH₂)₃C═CHF,H], [A2191;H,H,NHC(═S)SCH₂CHCH₂C═CF₂,H], [A2192;H,H,NHC(═S)SCH₂C═CHCl,H], [A2193;H,H,NHC(═S)SCH₂C═CCl₂,H], [A2194;H,H,NHC(═S)S(CH₂)₂C═CHCl,H], [A2195;H,H,NHC(═S)SCHCH₂C═CCl₂,H], [A2196;H,H,NHC(═S)S(CH₂)₃C═CHCl,H], [A2197;H,H,NHC(═S)SCH₂CHCH₂C═CCl₂,H], [A2198;H,H,NHC(═S)SCH₂C═CH,H], [A2199;H,H,NHC(═S)SCH(Me)C═CH,H], [A2200;H,H,NHC(═S)SCH₂C═CMe,H],

[A2201;H,H,NHC(═S)SCH₂C=CEt,H], [A2202;H,H,NHC(═S)SCH₂C=CEt,H], [A2203;H,H,NHC(═S)S(CH₂)₂C═CH,H], [A2204;H,H,NHC(═S)S(CH₂)₂C═CMe,H], [A2205;H,H,NHC(═S)S(CH₂)₃C═CH,H], [A2206;H,H,NHC(═S)S(CH₂)₄C═CH,H], [A2207;H,H,NHC(═S)Sc—Pr,H], [A2208;H,H,NHC(═S)Sc—Bu,H], [A2209;H,H,NHC(═S)Sc—Pen,H], [A2210;H,H,NHC(═S)Sc—Hex,H], [A2211;H,H,NHC(═S)Sc—Hep,H], [A2212;H,H,NHC(═O) CH₂OMe,H], [A2213;H,H,NHC(═O) (CH₂)₂OMe,H], [A2214;H,H,NHC(═O) (CH₂)₃OMe,H], [A2215;H,H,NHC(═O) (CH₂)₄OMe,H], [A2216;H,H,NHC(═O) CH₂OEt,H], [A2217;H,H,NHC(═O) (CH₂)₂OEt,H], [A2218;H,H,NHC(═O) (CH₂)₃OEt,H], [A2219;H,H,NHC(═O) CH₂CN,H], [A2220;H,H,NHC(═O) (CH₂)₂CN,H], [A2221;H,H,NHC(═O) (CH₂)₃CN,H], [A2222;H,H,NHC(═O) (CH₂)₄CN,H], [A2223;H,H,NHC(═O) (CH₂)₅CN,H], [A2224;H,H,NHC(═O) CH₂C═CHF,H], [A2225;H,H,NHC(═O)CH₂C═CF₂,H], [A2226;H,H,NHC(═O) (CH₂)₂C═CHF,H], [A2227;H,H,NHC(═O) CHCH₂C═CF₂,H], [A2228;H,H,NHC(═O) (CH₂)₃C═CHF,H], [A2229;H,H,NHC(═O) CH₂CHCH₂C═CF₂,H], [A2230;H,H,NHC(═O)CH₂C═CHCl,H], [A2231;H,H,NHC(═O) CH₂C═CCl₂,H], [A2232;H,H,NHC(═O) (CH₂)₂C═CHCl,H], [A2233;H,H,NHC(═O)CHCH₂C═CCl₂,H], [A2234;H,H,NHC(═O) (CH₂)₃C═CHCl,H], [A2235;H,H,NHC(═O)CH₂CHCH₂C═CCl₂,H], [A2236;H,H,NHC(═O) CH₂C═CH,H], [A2237;H,H,NHC(═O)CH(Me)C═CH,H], [A2238;H,H,NHC(═O)CH₂C═CMe,H], [A2239;H,H,NHC(═O)CH₂C=CEt,H], [A2240;H,H,NHC(═O) CH₂C=CEt,H], [A2241;H,H,NHC(═O) (CH₂)₂C═CH,H], [A2242;H,H,NHC(═O) (CH₂)₂C═CMe,H], [A2243;H,H,NHC(═O) (CH₂)₃C═CH,H], [A2244;H,H,NHC(═O) (CH₂)₄C═CH,H], [A2245;H,H,NHC(═O)c-Pr,H], [A2246;H,H,NHC(═O)c-Bu,H], [A2247;H,H,NHC(═O)c-Pen,H], [A2248;H,H,NHC(═O)c-Hex,H], [A2249;H,H,NHC(═O)c-Hep,H], [A2250;H,H,NHC(═S)CH₂OMe,H], [A2251;H,H,NHC(═S) (CH₂)₂OMe,H], [A2252;H,H,NHC(═S) (CH₂)₃OMe,H], [A2253;H,H,NHC(═S) (CH₂)₄OMe,H], [A2254;H,H,NHC(═S)CH₂OEt,H], [A2255;H,H,NHC(═S) (CH₂)₂OEt,H], [A2256;H,H,NHC(═S) (CH₂)₃OEt,H], [A2257;H,H,NHC(═S)CH₂CN,H], [A2258;H,H,NHC(═S) (CH₂)₂CN,H], [A2259;H,H,NHC(═S) (CH₂)₃CN,H], [A2260;H,H,NHC(═S) (CH₂)₄CN,H], [A2261;H,H,NHC(═S) (CH₂)₅CN,H], [A2262;H,H,NHC(═S) CH₂C═CHF,H], [A2263;H,H,NHC(═S)CH₂C═CF₂,H], [A2264;H,H,NHC(═S) (CH₂)₂C═CHF,H], [A2265;H,H,NHC(═S) CHCH₂C═CF₂,H], [A2266;H,H,NHC(═S) (CH₂)₃C═CHF,H], [A2267;H,H,NHC(═S) CH₂CHCH₂C═CF₂,H], [A2268;H,H,NHC(═S)CH₂C═CHCl,H], [A2269;H,H,NHC(═S) CH₂C═CCl₂,H], [A2270;H,H,NHC(═S) (CH₂)₂C═CHCl,H], [A2271;H,H,NHC(═S) CHCH₂C═CCl₂,H], [A2272;H,H,NHC(═S) (CH₂)₃C═CHCl,H], [A2273;H,H,NHC(═S) CH₂CHCH₂C═CCl₂,H], [A2274;H,H,NHC(═S) CH₂C═CH,H], [A2275;H,H,NHC(═S)CH(Me)C═CH,H], [A2276;H,H,NHC(═S)CH₂C═CMe,H], [A2277;H,H,NHC(═S)CH₂C=CEt,H], [A2278;H,H,NHC(═S) CH₂C=CEt,H], [A2279;H,H,NHC(═S) (CH₂)₂C═CH,H], [A2280;H,H,NHC(═S) (CH₂)₂C═CMe,H], [A2281;H,H,NHC(═S) (CH₂)₃C═CH,H], [A2282;H,H,NHC(═S) (CH₂)₄C═CH,H], [A2283;H,H,NHC(═S)c-Pr,H], [A2284;H,H,NHC(═S)c-Bu,H], [A2285;H,H,NHC(═S)c-Pen,H], [A2286;H,H,NHC(═S)c-Hex,H], [A2287;H,H,NHC(═S)c-Hep,H], [A2288;H,H,SC(═O)NHMe,H], [A2289;H,H,SC(═O)NHEt,H], [A2290;H,H,SC(═O)NH(CH₂)₂Me,H][A2291;H,H,SC(═O)NH(CH₂)₃Me,H], [A2292;H,H,SC(═O)NH(CH₂)₄Me,H], [A2293;H,H,SC(═O)NHCH(Me)₂,H], [A2294;H,H,SC(═O)NHCH₂CH(Me)₂,H][A2295;H,H,SC(═O)NH(CH₂)₂CH(Me)₂,H], [A2296;H,H,SC(═O)NHC(Me)₃,H], [A2297;H,H,SC(═O)NHCH₂C(Me)₃,H], [A2298;H,H,SC(═O)NH(CH₂)₂C(Me)₃,H], [A2299;H,H,SC(═O)NHCH(Me)Et,H][A2300;H,H,SC(═O)NHCH(Me) (CH₂)₂Me,H],

[A2301;H,H,SC(═O)NHCH(Me) (CH₂)₃Me,H], [A2302;H,H,SC(═O)NHCH (Et)₂,H], [A2303;H,H,SC(═O)NHCH₂CH═CH₂,H], [A2304;H,H,SC(═O)NH(CH₂)₂CH═CH₂,H][A2305;H,H,SC(═O)NH(CH₂)₃CH═CH₂,H], [A2306;H,H,SC(═O)NHCH₂CH═CHMe,H], [A2307;H,H,SC(═O)NHCH₂CH═CHEt,H], [A2308;H,H,SC(═O)NHCH₂CH═C(Me)₂,H], [A2309;H,H,SC(═O)NHCH₂CH═C(Me)Et,H], [A2310;H,H,SC(═O)NHOMe,H], [A2311;H,H,SC(═O)NHOEt,H], [A2312;H,H,SC(═O)NHO(CH₂)₂Me,H], [A2313;H,H,SC(═O)NHO(CH₂)₃Me,H], [A2314;H,H,SC(═O)NHO(CH₂)₄Me,H], [A2315;H,H,SC(═O)NHOCH(Me)₂,H], [A2316;H,H,SC(═O)NHOCH₂CH(Me)₂,H], [A2317;H,H,SC(═O)NHO(CH₂)₂CH(Me)₂,H], [A2318;H,H,SC(═O)NHOC(Me)₃,H], [A2319;H,H,SC(═O)NHOCH₂C(Me)₃,H], [A2320;H,H,SC(═O)NHO(CH₂)₂C(Me)₃,H], [A2321;H,H,SC(═O)NHOCH(Me)Et,H], [A2322;H,H,SC(═O)NHOCH(Me)(CH₂)₂Me,H], [A2323;H,H,SC(═O)NHOCH(Me)(CH₂)₃Me,H], [A2324;H,H,SC(═O)NHOCH(Et)₂,H], [A2325;H,H,SC(═O)NHOCH₂CH═CH₂,H], [A2326;H,H,SC(═O)NHO(CH₂)₂CH═CH₂,H], [A2327;H,H,SC(═O)NHO(CH₂)₃CH═CH₂,H], [A2328;H,H,SC(═O)NHOCH₂CH═CHMe,H], [A2329;H,H,SC(═O)NHOCH₂CH═CHEt,H], [A2330;H,H,SC(═O)NHOCH₂CH═C(Me)₂,H], [A2331;H,H,SC(═O)NHOCH₂CH═C(Me)Et,H], [A2332;H,H,SC(═O)NHCH₂F,H], [A2333;H,H,SC(═O)NHCHF₂,H], [A2334;H,H,SC(═O)NHCF₃,H], [A2335;H,H,SC(═O)NH(CH₂)₂F,H], [A2336;H,H,SC(═O)NHCH₂CHF₂,H], [A2337;H,H,SC(═O)NHCH₂CF₃,H], [A2338;H,H,SC(═O)NH(CH₂)₃F,H], [A2339;H,H,SC(═O)NH(CH₂)₂CHF₂,H], [A2340;H,H,SC(═O)NH(CH₂)₂CF₃,H], [A2341;H,H,SC(═O)NHCH₂CH(CF₃)₂,H], [A2342;H,H,SC(═O)NHCH₂Cl,H], [A2343;H,H,SC(═O)NH(CH₂)₂Cl,H], [A2344;H,H,SC(═O)NH(CH₂)₃Cl,H], [A2345;H,H,SC(═O)NH(CH₂)₄Cl,H], [A2346;H,H,SC(═O)NH(CH₂)₅Cl,H], [A2347;H,H,SC(═O)NHCH₂Br,H], [A2348;H,H,SC(═O)NH(CH₂)₂Br,H], [A2349;H,H,SC(═O)NH(CH₂)₃Br,H][A2350;H,H,SC(═O)NH(CH₂)₄Br,H][A2351;H,H,SC(═O)NH(CH₂)₅Br,H], [A2352;H,H,SC(═O)NHCH₂OMe,H], [A2353;H,H,SC(═O)NH(CH₂)₂OMe,H], [A2354;H,H,SC(═O)NH(CH₂)₃OMe,H], [A2355;H,H,SC(═O)NH(CH₂)₄OMe,H], [A2356;H,H,SC(═O)NHCH₂OEt,H], [A2357;H,H,SC(═O)NH(CH₂)₂OEt,H][A2358;H,H,SC(═O)NH(CH₂)₃OEt,H], [A2359;H,H,SC(═O)NHCH₂CN,H], [A2360;H,H,SC(═O)NH(CH₂)₂CN,H], [A2361;H,H,SC(═O)NH(CH₂)₃CN,H], [A2362;H,H,SC(═O)NH(CH₂)₄CN,H][A2363;H,H,SC(═O)NH(CH₂)₅CN,H][A2364;H,H,SC(═O)NHCH₂C═CHF,H], [A2365;H,H,SC(═O)NHCH₂C═CF₂,H], [A2366;H,H,SC(═O)NH(CH₂₂C═CHF,H], [A2367;H,H,SC(═O)NHCHCH₂C═CF₂,H], [A2368;H,H,SC(═O)NH(CH₂)₃C═CHF,H], [A2369;H,H,SC(═O)NHCH₂CHCH₂C═CF₂,H], [A2370;H,H,SC(═O)NHCH₂C═CHCl,H], [A2371;H,H,SC(═O)NHCH₂C═CCl₂,H], [A2372;H,H,SC(═O)NH(CH₂)₂C═CHCl,H], [A2373;H,H,SC(═O)NHCHCH₂C═CCl₂,H], [A2374;H,H,SC(═O)NH(CH₂)₃C═CHCl,H], [A2375;H,H,SC(═O)NHCH₂CHCH₂C═CCl₂,H], [A2376;H,H,SC(═O)NHCH₂C═CH,H], [A2377;H,H,SC(═O)NHCH(Me)C═CH,H], [A2378;H,H,SC(═O)NHCH₂C═CMe,H], [A2379;H,H,SC(═O)NHCH₂C=CEt,H], [A2380;H,H,SC(═O)NHCH₂C=CEt,H], [A2381;H,H,SC(═O)NH(CH₂)₂C═CH,H], [A2382;H,H,SC(═O)NH(CH₂)₂C═CMe,H], [A2383;H,H,SC(═O)NH(CH₂)₃C═CH,H], [A2384;H,H,SC(═O)NH(CH₂)₄C═CH,H], [A2385;H,H,SC(═O)NHc-Pr,H], [A2386;H,H,SC(═O)NHc-Bu,H], [A2387;H,H,SC(═O)NHc-Pen,H], [A2388;H,H,SC(═O)NHc-Hex,H], [A2389;H,H,SC(═O)NHc-Hep,H], [A2390;H,H,SC(═O)NHOCH₂F,H], [A2391;H,H,SC(═O)NHOCHF₂,H], [A2392;H,H,SC(═O)NHOCF₃,H], [A2393;H,H,SC(═O)NHO(CH₂)₂F,H], [A2394;H,H,SC(═O)NHOCH₂CHF₂,H], [A2395;H,H,SC(═O)NHOCH₂CF₃,H], [A2396;H,H,SC(═O)NHO(CH₂)₃F,H][A2397;H,H,SC(═O)NHO(CH₂)₂CHF₂,H], [A2398;H,H,SC(═O)NHO(CH₂)₂CF₃,H][A2399;H,H,SC(═O)NHOCH₂CH(CF₃)₂,H][A2400;H,H,SC(═O)NHOCH₂CH═CH₂,H],

[A2401;H,H,SC(═O)NHOCH₂CH═CHF,H], [A2402;H,H,SC(═O)NHOCH₂CH═CF₂,H], [A2403;H,H,SC(═O)NHOCH₂CH═CHCl,H], [A2404;H,H,SC(═O)NHOCH₂CH═CCl₂,H], [A2405;H,H,SC(═O)NHOCH₂C═CH,H], [A2406;H,H,SC(═O)NHOCH(Me)C═CH,H], [A2407;H,H,SC(═O)NHOCH₂C═CMe,H], [A2408;H,H,SC(═O)NMeMe,H], [A2409;H,H,SC(═O)NMeEt,H], [A2410;H,H,SC(═O)NMe(CH₂)₂Me,H], [A2411;H,H,SC(═O)NMe(CH₂)₃Me,H], [A2412;H,H,SC(═O)NMe(CH₂)₄Me,H], [A2413;H,H,SC(═O)NMeCH(Me)₂,H], [A2414;H,H,SC(═O)NMeCH₂CH(Me)₂,H], [A2415;H,H,SC(═O)Ne(CH₂)₂CH(Me)₂,H], [A2416;H,H,SC(═O)NMeC(Me)₃,H], [A2417;H,H,SC(═O)NMeCH₂C(Me)₃,H], [A2418;H,H,SC(═O)NMe(CH₂)₂C(Me)₃,H], [A2419;H,H,SC(═O)NMeCH(Me)Et,H], [A2420;H,H,SC(═)NMeCH(Me)(CH₂)₂Me,H], [A2421;H,H,SC(═)NMeCH(Me)(CH₂)₃Me,H], [A2422;H,H,SC(═O)NMeCH(Et)₂,H], [A2423;H,H,SC(═)NMeCH₂CH═CH₂,H], [A2424;H,H,SC(═O)NMe(CH₂)₂CH═CH₂,H], [A2425;H,H,SC(═O)NMe(CH₂)₃CH═CH₂,H], [A2426;H,H,SC(═O)NMeCH₂CH═CHMe,H], [A2427;H,H,SC(═O)NMeCH₂CH═CHEt,H], [A2428;H,H,SC(═O)NMeCH₂CH═C(Me)₂,H], [A2429;H,H,SC(═O)NMeCH₂CH═C(Me)Et,H], [A2430;H,H,SC(═)NMeOMe,H], [A2431;H,H,SC(═O)NMeOEt,H], [A2432;H,H,SC(═O)NMeO(CH₂)₂Me,H], [A2433;H,H,SC(═O)NMeO(CH₂)₃Me,H], [A2434;H,H,SC(═O)NMeO(CH)₄Me,H], [A2435;H,H,SC(═)NMeOCH(Me)₂,H], [A2436;H,H,SC(═O)NMeOCH₂CH(Me)₂,H], [A2437;H,H,SC(═O)NMeO(CH₂)₂CH(Me)₂,H][A2438;H,H,SC(═O)NMeOC(Me)₃,H], [A2439;H,H,SC(═O)NMeOCH₂C(Me)₃,H], [A2440;H,H,SC(═O)NMeO(CH₂)₂C(Me)₃,H][A2441;H,H,SC(═)NMeOCH(Me)Et,H], [A2442;H,H,SC(═O)NMeOCH(Me)(CH₂)₂Me,H], [A2443;H,H,SC(═O)NMeOCH(Me)(CH₂)₃Me,H], [A2444;H,H,SC(═O)NMeOCH(Et)₂,H], [A2445;H,H,SC(═O)NMeOCH₂CH═CH₂,H], [A2446;H,H,SC(═O)NMeO(CH₂)₂CH═CH₂,H], [A2447;H,H,SC(═O)NMeO(CH₂)₃CH═CH₂,H], [A2448;H,H,SC(═)NMeOCH₂CH═CHMe,H], [A2449;H,H,SC(═)NMeOCH₂CH═CHEt,H], [A2450;H,H,SC(═O)NMeCH₂CH═C(Me)₂,H], [A2451;H,H,SC(═O)NMeCH₂CH═C(Me)Et,H], [A2452;H,H,SC(═O)NMeCH₂F,H], [A2453;H,H,SC(═O)NMeCHF₂,H], [A2454;H,H,SC(═O)NMeCF₃,H], [A2455;H,H,SC(═O)NMe(CH₂)₂F,H], [A2456;H,H,SC(═O)NMeCH₂CHF₂,H], [A2457;H,H,SC(═O)NMeCH₂CF₃,H], [A2458;H,H,SC(═O)NMe(CH₂)₃F,H], [A2459;H,H,SC(═O)NMe(CH₂)₂CHF₂,H], [A2460;H,H,SC(═O)NMe(CH₂)₂CF₃,H], [A2461;H,H,SC(═O)NMeCH₂CH(CF₃)₂,H], [A2462;H,H,SC(═O)NMeCH₂Cl,H], [A2463;H,H,SC(═O)NMe(CH₂)₂Cl,H], [A2464;H,H,SC(═O)NMe(CH₂)₃Cl,H], [A2465;H,H,SC(═O)NMe(CH₂)₄Cl,H], [A2466;H,H,SC(═O)NMe(CH₂)₅Cl,H], [A2467;H,H,SC(═O)NMeCH₂Br,H], [A2468;H,H,SC(═O)NMe(CH₂)₂Br,H], [A2469;H,H,SC(═O)NMe(CH₂)₃Br,H], [A2470;H,H,SC(═O)NMe(CH₂)₄Br,H], [A2471;H,H,SC(═O)NMe(CH₂)₅Br,H], [A2472;H,H,SC(═O)NMeCH₂OMe,H], [A2473;H,H,SC(═O)NMe(CH₂)₂OMe,H], [A2474;H,H,SC(═O)NMe(CH₂)₃OMe,H], [A2475;H,H,SC(═O)NMe(CH₂)₄OMe,H], [A2476;H,H,SC(═O)NMeCH₂OEt,H], [A2477;H,H,SC(═O)NMe(CH₂)₂OEt,H], [A2478;H,H,SC(═O)NMe(CH₂)₃OEt,H], [A2479;H,H,SC(═O)NMeCH₂CN,H], [A2480;H,H,SC(═O)NMe(CH₂)₂CN,H], [A2481;H,H,SC(═O)NMe(CH₂)₃CN,H], [A2482;H,H,SC(═O)NMe(CH₂)₄CN,H], [A2483;H,H,SC(═O)NMe(CH₂)₅CN,H], [A2484;H,H,SC(═O)NMeCH₂C═CHF,H], [A2485;H,H,SC(═O)NMeCH₂C═CF₂,H], [A2486;H,H,SC(═O)NMe(CH₂)₂C═CHF,H], [A2487;H,H,SC(═O)NMeCHCH₂C═CF₂,H], [A2488;H,H,SC(═O)NMe(CH)₃C═CHF,H], [A2489;H,H,SC(═O)NMeCH₂CHCH₂C═CF₂,H], [A2490;H,H,SC(═O)NMeCH₂C═CHCl,H], [A2491;H,H,SC(═O)NMeCH₂C═CCl₂,H], [A2492;H,H,SC(═O)NMe(CH₂)₂C═CHCl,H], [A2493;H,H,SC(═O)NMeCHCH₂C═CCl₂,H], [A2494;H,H,SC(═O)NMe(CH₂)₃C═CHCl,H], [A2495;H,H,SC(═O)NMeCH₂CHCH₂C═CCl₂,H], [A2496;H,H,SC(═O)NMeCH₂C═CH,H], [A2497;H,H,SC(═O)NMeCH(Me)C═CH,H], [A2498;H,H,SC(═O)NMeCH₂C═CMe,H], [A2499;H,H,SC(═O)NMeCH₂C=CEt,H], [A2500;H,H,SC(═O)NMeCH₂C=CEt,H],

[A2501;H,H,SC(═O)NMe(CH₂₂C═CH,H][A2502;H,H,SC(═O)NMe(CH)₂C═CMe,H], [A2503;H,H,SC(═O)NMe(CH₂)₃C═CH,H], [A2504;H,H,SC(═O)NMe(CH₂)₄C═CH,H], [A2505;H,H,SC(═O)NMec-Pr,H], [A2506;H,H,SC(═O)NMec-Bu,H], [A2507;H,H,SC(═O)NMec-Pen,H], [A2508;H,H,SC(═O)NMec-Hex,H], [A2509;H,H,SC(═O)NMec-Hep,H], [A2510;H,H,SC(═O)NMeOCH₂F,H], [A2511;H,H,SC(═O)NMeOCHF₂,H], [A2512;H,H,SC(═O)NMeOCF₃,H], [A2513;H,H,SC(═O)NMeO(CH₂)₂F,H], [A2514;H,H,SC(═O)NMeOCH₂CHF₂,H], [A2515;H,H,SC(═O)NMeOCH₂CF₃,H], [A2516;H,H,SC(═O)NMeO(CH₂)₃F,H][A2517;H,H,SC(═O)NMeO(CH₂)₂CHF₂,H][A2518;H,H,SC(═O)NMeO(CH₂)₂CF₃,H], [A2519;H,H,SC(═O)NMeOCH₂CH(CF₃)₂,H], [A2520;H,H,SC(═O)NMeOCH₂CH═CH₂,H], [A2521;H,H,SC(═O)NMeOCH₂CH═CHF,H], [A2522;H,H,SC(═O)NMeOCH₂CH═CF₂,H], [A2523;H,H,SC(═O)NMeOCH₂CH═CHCl,H], [A2524;H,H,SC(═O)NMeOCH₂CH═CCl₂,H], [A2525;H,H,SC(═O)NMeOCH₂C═CH,H], [A2526;H,H,SC(═O)NMeOCH(Me)C═CH,H], [A2527;H,H,SC(═O)NMeOCH₂C═CMe,H], [A2528;H,H,SC(═O)NEtMe,H], [A2529;H,H,SC(═O)NEtEt,H], [A2530;H,H,SC(═O)NEt(CH₂)₂Me,H], [A2531;H,H,SC(═O)NEt(CH₂)₃Me,H], [A2532;H,H,SC(═O)NEt(CH₂)₄Me,H], [A2533;H,H,SC(═O)NEtCH(Me)₂,H], [A2534;H,H,SC(═O)NEtCH₂CH(Me)₂,H], [A2535;H,H,SC(═O)NEt(CH₂)₂CH(Me)₂,H], [A2536;H,H,SC(═O)NEtC(Me)₃,H], [A2537;H,H,SC(═O)NEtCH₂C(Me)₃,H], [A2538;H,H,SC(═O)NEt(CH₂)₂C(Me)₃,H], [A2539;H,H,SC(═O)NEtCH(Me)Et,H], [A2540;H,H,SC(═O)NEtCH(Me)(CH₂)₂Me,H], [A2541;H,H,SC(═O)NEtCH(Me)(CH₂)₃Me,H], [A2542;H,H,SC(═O)NEtCH(Et)₂,H], [A2543;H,H,SC(═O)NEtCH₂CH═CH₂,H], [A2544;H,H,SC(═O)NEt(CH₂)₂CH═CH₂,H], [A2545;H,H,SC(═O)NEt(CH₂)₃CH═CH₂═CH,H], [A2546;H,H,SC(═O)NEtCH₂CH═CHMe,H], [A2547;H,H,SC(═O)NEtCH₂CH═CHEt,H], [A2548;H,H,SC(═O)NEtCH₂CH═C(Me)₂,H], [A2549;H,H,SC(═O)NEtCH₂CH═C(Me)Et,H], [A2550;H,H,SC(═O)NEtOMe,H], [A2551;H,H,SC(═O)NEtOEt,H], [A2552;H,H,SC(═O)NEtO(CH₂)₂Me,H][A2553;H,H,SC(═O)NEtO(CH₂)₃Me,H][A2554;H,H,SC(═O)NEtO(CH₂)₄Me,H][A2555;H,H,SC(═O)NEtOCH(Me)₂,H][A2556;H,H,SC(═O)NEtOCH₂CH(Me)₂,H], [A2557;H,H,SC(═O)NEtO(CH₂)₂CH(Me)₂,H][A2558;H,H,SC(═O)NEtOC(Me)₃,H][A2559;H,H,SC(═O)NEtOCH₂C(Me)₃,H], [A2560;H,H,SC(═O)NEtO(CH₂)₂C(Me),H][A2561;H,H,SC(═O)NEtOCH(Me)Et,H], [A2562;H,H,SC(═O)NEtOCH(Me)(CH₂)₂Me,H], [A2563;H,H,SC(═O)NEtOCH(Me)(CH₂)₃Me,H], [A2564;H,H,SC(═O)NEtOCH(Et)₂,H][A2565;H,H,SC(═O)NEtOCH₂CH═CH₂,H], [A2566;H,H,SC(═O)NEtO(CH₂)₂CH═CH₂,H], [A2567;H,H,SC(═O)NEtO(CH₂)₃CH═CH₂,H], [A2568;H,H,SC(═O)NEtOCH₂CH═CHMe,H], [A2569;H,H,SC(═O)NEtOCH₂CH═CHEt,H], [A2570;H,H,SC(═O)NEtOCH₂CH═C(Me)₂,H], [A2571;H,H,SC(═O)NEtOCH₂CH═C(Me)Et,H], [A2572;H,H,SC(═O)NEtCH₂F,H], [A2573;H,H,SC(═O)NEtCHF₂,H], [A2574;H,H,SC(═O)NEtCF₃,H], [A2575;H,H,SC(═O)NEt(CH₂)₂F,H], [A2576;H,H,SC(═O)NEtCH₂CHF₂,H], [A2577;H,H,SC(═O)NEtCH₂CF₃,H], [A2578;H,H,SC(═O)NEt(CH₂)₃F,H], [A2579;H,H,SC(═O)NEt(CH₂)₂CHF₂,H], [A2580;H,H,SC(═O)NEt(CH₂)₂CF₃,H], [A2581;H,H,SC(═O)NEtCH₂CH(CF₃)₂,H][A2582;H,H,SC(═O)NEtCH₂Cl,H], [A2583;H,H,SC(═O)NEt(CH₂)₂Cl,H], [A2584;H,H,SC(═O)NEt(CH₂)₃Cl,H], [A2585;H,H,SC(═O)NEt(CH₂)₄Cl,H], [A2586;H,H,SC(═O)NEt(CH₂)₅Cl,H], [A2587;H,H,SC(═O)NEtCH₂Br,H], [A2588;H,H,SC(═O)NEt(CH₂)₂Br,H], [A2589;H,H,SC(═O)NEt(CH₂)₃Br,H], [A2590;H,H,SC(═O)NEt(CH₂)₄Br,H], [A2591;H,H,SC(═O)NEt(CH₂)₅Br,H], [A2592;H,H,SC(═O)NEtCH₂OMe,H], [A2593;H,H,SC(═O)NEt(CH₂)₂OMe,H], [A2594;H,H,SC(═O)NEt(CH₂)₃OMe,H], [A2595;H,H,SC(═O)NEt(CH₂)₄OMe,H], [A2596;H,H,SC(═O)NEtCH₂OEt,H], [A2597;H,H,SC(═O)NEt(CH₂)₂OEt,H], [A2598;H,H,SC(═O)NEt(CH₂)₃OEt,H], [A2599;H,H,SC(═O)NEtCH₂CN,H], [A2600;H,H,SC(═O)NEt(CH₂)₂CN,H],

[A2601;H,H,SC(═O)NEt(CH₂)₃CN,H], [A2602;H,H,SC(═O)NEt(CH₂)₄CN,H], [A2603;H,H,SC(═O)NEt(CH₂)₅CN,H], [A2604;H,H,SC(═O)NEtCH₂C═CHF,H], [A2605;H,H,SC(═O)NEtCH₂C═CF₂,H], [A2606;H,H,SC(═O)NEt(CH₂)₂C═CHF,H], [A2607;H,H,SC(═O)NEtCHCH₂C═CF₂,H], [A2608;H,H,SC(═O)NEt(CH₂)₃C═CHF,H], [A2609;H,H,SC(═O)NEtCH₂CHCH₂C═CF₂,H], [A2610;H,H,SC(═O)NEtCH₂C═CHCl,H], [A2611;H,H,SC(═O)NEtCH₂C═CCl₂,H], [A2612;H,H,SC(═O)NEt(CH₂)₂C═CHCl,H], [A2613;H,H,SC(═O)NEtCH₂CHCH₂C═CCl₂,H], [A2614;H,H,SC(═O)NEt(CH₂)₃C═CHCl,H], [A2615;H,H,SC(═O)NEtCH₂CHCH₂C═CCl₂,H], [A2616;H,H,SC(═O)NEtCH₂C═CH,H], [A2617;H,H,SC(═O)NEtCH(Me)C═CH,H], [A2618;H,H,SC(═O)NEtCH₂C═CMe,H], [A2619;H,H,SC(═O)NEtCH₂C=CEt,H], [A2620;H,H,SC(═O)NEtCH₂C=CEt,H], [A2621;H,H,SC(═O)NEt(CH₂)₂C═CH,H], [A2622;H,H,SC(═O)NEt(CH₂)₂C═CMe,H], [A2623;H,H,SC(═O)NEt(CH₂)₃C═CH,H], [A2624;H,H,SC(═O)NEt(CH₂)₄C═CH,H], [A2625;H,H,SC(═O)NEtc-Pr,H], [A2626;H,H,SC(═O)NEtc-Bu,H], [A2627;H,H,SC(═O)NEtc-Pen,H], [A2628;H,H,SC(═O)NEtc-Hex,H], [A2629;H,H,SC(═O)NEtc-Hep,H], [A2630;H,H,SC(═O)NEtOCH₂F,H], [A2631;H,H,SC(═O)NEtOCHF₂,H], [A2632;H,H,SC(═O)NEtOCF₃,H], [A2633;H,H,SC(═O)NEtO(CH₂)₂F,H], [A2634;H,H,SC(═O)NEtOCH₂CHF₂,H], [A2635;H,H,SC(═O)NEtOCH₂CF₃,H], [A2636;H,H,SC(═O)NEtO(CH₂)₃F,H], [A2637;H,H,SC(═O)NEtO(CH₂)₂CHF₂,H], [A2638;H,H,SC(═O)NEtO(CH₂)₂CF₃,H], [A2639;H,H,SC(═O)NEtOCH₂CH(CF₃)₂,H], [A2640;H,H,SC(═O)NEtOCH₂CH═CH₂,H], [A2641;H,H,SC(═O)NEtOCH₂CH═CHF,H], [A2642;H,H,SC(═O)NEtOCH₂CH═CF₂,H], [A2643;H,H,SC(═O)NEtOCH₂CH═CHCl,H], [A2644;H,H,SC(═O)NEtOCH₂CH═CCl₂,H], [A2645;H,H,SC(═O)NEtOCH₂C═CH,H], [A2646;H,H,SC(═O)NEtOCH(Me)C═CH,H], [A2647;H,H,SC(═O)NEtOCH₂C═CMe,H], [A2648;H,H,SC(═O)NPrMe,H], [A2649;H,H,SC(═O)NPrEt,H], [A2650;H,H,SC(═O)NPr(CH₂)₂Me,H], [A2651;H,H,SC(═O)NPr(CH₂)₃Me,H], [A2652;H,H,SC(═O)NPr(CH₂)₄Me,H], [A2653;H,H,SC(═O)NPrCH(Me)₂,H], [A2654;H,H,SC(═O)NPrCH₂CH(Me)₂,H], [A2655;H,H,SC(═O)NPr(CH₂)₂CH(Me)₂,H], [A2656;H,H,SC(═O)NPrC(Me)₃,H], [A2657;H,H,SC(═O)NPrCH₂C(Me)₃,H], [A2658;H,H,SC(═O)NPr(CH₂)₂C C(Me)₃,H][A2659;H,H,SC(═O)NPrCH(Me)Et,H], [A2660;H,H,SC(═O)NPrCH(Me)(CH₂)₂Me,H], [A2661;H,H,SC(═O)NPrCH(Me)(CH₂)₃Me,H], [A2662;H,H,SC(═O)NPrCH(Et)₂,H], [A2663;H,H,SC(═O)NPrCH₂CH₂═CH,H], [A2664;H,H,SC(═O)NPr(CH₂₎CH═CH₂,H], [A2665;H,H,SC(═O)NPr(CH₂)₃CH═CH₂,H], [A2666;H,H,SC(═O)NPrCH₂CH═CHMe,H], [A2667;H,H,SC(═O)NPrCH₂CH═CHEt,H], [A2668;H,H,SC(═O)NPrCH₂CH═C(Me)₂,H], [A2669;H,H,SC(═O)NPrCH₂CH═C(Me)Et,H], [A2670;H,H,SC(═O)NPrOMe,H], [A2671;H,H,SC(═O)NPrOEt,H], [A2672;H,H,SC(═O)NPrO(CH₂)₂Me,H], [A2673;H,H,SC(═O)NPrO(CH₂)₃Me,H], [A2674;H,H,SC(═O)NPrO(CH₂)₄Me,H], [A2675;H,H,SC(═O)NPrOCH(Me)₂,H], [A2676;H,H,SC(═O)NPrOCH₂CH(Me)₂,H], [A2677;H,H,SC(═O)NPrO(CH₂)₂CH(Me)₂,H], [A2678;H,H,SC(═O)NPrOC(Me)₃,H], [A2679;H,H,SC(═O)NPrOCH₂C(Me)₃,H], [A2680;H,H,SC(═O)NPrO(CH₂)₂C(Me)₃,H], [A2681;H,H,SC(═O)NPrOCH(Me)Et,H], [A2682;H,H,SC(═O)NPrOCH(Me)(CH₂)₂Me,H], [A2683;H,H,SC(═O)NPrOCH(Me)(CH₂)₃Me,H], [A2684;H,H,SC(═O)NPrOCH(Et)₂,H], [A2685;H,H,SC(═O)NPrOCH₂CH═CH₂,H], [A2686;H,H,SC(═O)NPrO(CH₂)₂CH═CH₂,H], [A2687;H,H,SC(═O)NPrO(CH₂)₃CH═CH₂,H], [A2688;H,H,SC(═O)NPrOCH₂CH═CHMe,H], [A2689;H,H,SC(═O)NPrOCH₂CH═CHEt,H], [A2690;H,H,SC(═O)NPrOCH₂CH═C(Me)₂,H], [A2691;H,H,SC(═O)NPrOCH₂CH═C(Me)Et,H], [A2692;H,H,SC(═O)NPrCH₂F,H], [A2693;H,H,SC(═O)NPrCHF₂,H], [A2694;H,H,SC(═O)NPrCF₃,H], [A2695;H,H,SC(═O)NPr(CH₂)₂F,H], [A2696;H,H,SC(═O)NPrCH₂CHF₂,H], [A2697;H,H,SC(═O)NPrCH₂CF₃,H], [A2698;H,H,SC(═O)NPr(CH₂)₃F,H], [A2699;H,H,SC(═O)NPr(CH₂)₂CHF₂,H], [A2700;H,H,SC(═O)NPr(CH₂)₂CF₃,H],

[A2701;H,H,SC(═O)NPrCH₂CH(CF₃)₂,H], [A2702;H,H,SC(═O)NPrCH₂Cl,H], [A2703;H,H,SC(═O)NPr(CH₂)₂Cl,H], [A2704;H,H,SC(═O)NPr(CH₂)₃Cl,H], [A2705;H,H,SC(═O)NPr(CH₂)₄Cl,H], [A2706;H,H,SC(═O)NPr(CH₂)₅Cl,H], [A2707;H,H,SC(═O)NPrCH₂Br,H], [A2708;H,H,SC(═O)NPr(CH₂)₂Br,H], [A2709;H,H,SC(═O)NPr(CH₂)₃Br,H], [A2710;H,H,SC(═O)NPr(CH₂)₄Br,H], [A2711;H,H,SC(═O)NPr(CH₂)₅Br,H], [A2712;H,H,SC(═O)NPrCH₂OMe,H], [A2713;H,H,SC(═O)NPr(CH₂)₂OMe,H], [A2714;H,H,SC(═O)NPr(CH₂)₃OMe,H], [A2715;H,H,SC(═O)NPr(CH₂)₄OMe,H], [A2716;H,H,SC(═O)NPrCH₂OEt,H], [A2717;H,H,SC(═O)NPr(CH₂)₂OEt,H], [A2718;H,H,SC(═O)NPr(CH₂)₃OEt,H][A2719;H,H,SC(═O)NPrCH₂CN,H], [A2720;H,H,SC(═O)NPr(CH₂)₂CN,H], [A2721;H,H,SC(═O)NPr(CH₂)₃CN,H][A2722;H,H,SC(═O)NPr(CH₂)₄CN,H][A2723;H,H,SC(═O)NPr(CH₂)₅CN,H], [A2724;H,H,SC(═O)NPrCH₂C═CHF,H], [A2725;H,H,SC(═O)NPrCH₂C═CF₂,H], [A2726;H,H,SC(═O)NPr(CH₂)₂C═CHF,H], [A2727;H,H,SC(═O)NPrCHCH₂C═CF₂,H], [A2728;H,H,SC(═O)NPr(CH₂)₃C═CHF,H], [A2729;H,H,SC(═O)NPrCH₂CHCH₂C═CF₂,H], [A2730;H,H,SC(═O)NPrCH₂C═CHCl,H], [A2731;H,H,SC(═O)NPrCH₂C═CCl₂,H], [A2732;H,H,SC(═O)NPr(CH₂)₂C═CHCl,H], [A2733;H,H,SC(═O)NPrCHCH₂C═CCl₂,H], [A2734;H,H,SC(═O)NPr(CH₂)₃C═CHCl,H], [A2735;H,H,SC(═O)NPrCH₂CHCH₂C═CCl₂,H], [A2736;H,H,SC(═O)NPrCH₂C═CH,H], [A2737;H,H,SC(═O)NPrCH(Me)C═CH,H], [A2738;H,H,SC(═O)NPrCH₂C═CMe,H], [A2739;H,H,SC(═O)NPrCH₂C=CEt,H], [A2740;H,H,SC(═O)NPrCH₂C=CEt,H], [A2741;H,H,SC(═O)NPr(CH₂)₂C═CH,H][A2742;H,H,SC(═O)NPr(CH₂)₂C═CMe,H], [A2743;H,H,SC(═O)NPr(CH₂)₃C═CH,H], [A2744;H,H,SC(═O)NPr(CH₂)₄C═CH,H], [A2745;H,H,SC(═O)NPrc-Pr,H], [A2746;H,H,SC(═O)NPrc-Bu,H], [A2747;H,H,SC(═O)NPrc-Pen,H], [A2748;H,H,SC(═O)NPrc-Hex,H], [A2749;H,H,SC(═O)NPrc-Hep,H], [A2750;H,H,SC(═O)NPrOCH₂F,H], [A2751;H,H,SC(═O)NPrOCHF₂,H], [A2752;H,H,SC(═O)NPrOCF₃,H], [A2753;H,H,SC(═O)NPrO(CH₂)₂F,H][A2754;H,H,SC(═O)NPrOCH₂CHF₂,H], [A2755;H,H,SC(═O)NPrOCH₂CF₃,H], [A2756;H,H,SC(═O)NPrO(CH₂)₃F,H][A2757;H,H,SC(═O)NPrO(CH₂)₂CHF₂,H], [A2758;H,H,SC(═O)NPrO(CH₂)₂CF₃,H][A2759;H,H,SC(═O)NPrOCH₂CH(CF₃)₂,H][A2760;H,H,SC(═O)NPrOCH₂CH═CH₂,H], [A2761;H,H,SC(═O)NPrOCH₂CH═CHF,H], [A2762;H,H,SC(═O)NPrOCH₂CH═CF₂,H], [A2763;H,H,SC(═O)NPrOCH₂CH═CHCl,H], [A2764;H,H,SC(═O)NPrOCH₂CH═CCl₂,H], [A2765;H,H,SC(═O)NPrOCH₂C═CH,H], [A2766;H,H,SC(═O)NPrOCH(Me)C═CH,H], [A2767;H,H,SC(═O)NPrOCH₂C═CMe,H], [A2768;H,H,SC(═O)N(i-Pr)Me,H], [A2769;H,H,SC(═O)N(i-Pr)Et,H], [A2770;H,H,SC(═O)N(i-Pr)(CH₂)₂Me,H], [A2771;H,H,SC(═O)N(i-Pr)(CH₂)₃Me,H], [A2772;H,H,SC(═O)N(i-Pr)(CH₂)₄Me,H], [A2773;H,H,SC(═O)N(i-Pr)CH(Me)₂,H], [A2774;H,H,SC(═O)N(i-Pr)CH₂CH(Me)₂,H], [A2775;H,H,SC(═O)N(i-Pr)(CH₂)₂CH(Me)₂,H], [A2776;H,H,SC(═O)N(i-Pr)C(Me)₃,H], [A2777;H,H,SC(═O)N(i-Pr) CH₂C(Me)₃,H], [A2778;H,H,SC(═O)N(i-Pr)(CH₂)₂C(Me)₃,H], [A2779;H,H,SC(═O)N(i-Pr)CH(Me)Et,H], [A2780;H,H,SC(═O)N(i-Pr)CH(Me)(CH₂)₂Me,H], [A2781;H,H,SC(═O)N(i-Pr)CH(Me)(CH₂)₃Me,H], [A2782;H,H,SC(═O)N(i-Pr)CH(Et)₂,H], [A2783;H,H,SC(═O)N(i-Pr)CH₂CH═CH₂,H], [A2784;H,H,SC(═O)N(i-Pr)(CH₂)₂CH═CH₂,H], [A2785;H,H,SC(═O)N(i-Pr)(CH₂)₃CH═CH₂,H], [A2786;H,H,SC(═O)N(i-Pr)CH₂CH═CHMe,H], [A2787;H,H,SC(═O)N(i-Pr)CH₂CH═CHEt,H], [A2788;H,H,SC(═O)N(i-Pr) CH₂CH═C(Me)₂,H], [A2789;H,H,SC(═O)N(i-Pr)CH₂CH═C(Me)Et,H], [A2790;H,H,SC(═O)N(i-Pr)OMe,H], [A2791;H,H,SC(═O)N(i-Pr)OEt,H], [A2792;H,H,SC(═O)N(i-Pr)O(CH₂)₂Me,H], [A2793;H,H,SC(═O)N(i-Pr)O(CH₂)₃Me,H], [A2794;H,H,SC(═O)N(i-Pr)O(CH₂)₄Me,H], [A2795;H,H,SC(═O)N(i-Pr) OCH(Me)₂,H], [A2796;H,H,SC(═O)N(i-Pr) OCH₂CH(Me)₂,H], [A2797;H,H,SC(═O)N(i-Pr)O(CH₂)₂CH(Me)₂,H], [A2798;H,H,SC(═O)N(i-Pr)OC(Me)₃,H], [A2799;H,H,SC(═O)N(i-Pr) OCH₂C(Me)₃,H], [A2800;H,H,SC(═O)N(i-Pr)O(CH₂)₂C(Me)₃,H],

[A2801;H,H,SC(═O)N(i-Pr) OCH(Me)Et,H], [A2802;H,H,SC(═O)N(i-Pr) OCH(Me)(CH₂)₂Me,H], [A2803;H,H,SC(═O)N(i-Pr) OCH(Me)(CH₂)₃Me,H], [A2804;H,H,SC(═O)N(i-Pr) OCH(Et)₂,H], [A2805;H,H,SC(═O)N(i-Pr) OCH₂CH═CH₂,H], [A2806;H,H,SC(═O)N(i-Pr)O(CH₂)₂CH═CH₂,H], [A2807;H,H,SC(═O)N(i-Pr)O(CH₂)₃CH═CH₂,H], [A2808;H,H,SC(═O)N(i-Pr) OCH₂CH═CHMe,H], [A2809;H,H,SC(═O)N(i-Pr)OCH₂CH═CHEt,H], [A2810;H,H,SC(═O)N(i-Pr)OCH₂CH═C(Me)₂,H], [A2811;H,H,SC(═O)N(i-Pr)OCH₂CH═C(Me)Et,H], [A2812;H,H,SC(═O)N(i-Pr)CH₂F,H], [A2813;H,H,SC(═O)N(i-Pr)CHF₂,H], [A2814;H,H,SC(═O)N(i-Pr)CF₃,H], [A2815;H,H,SC(═O)N(i-Pr)(CH₂)₂F,H], [A2816;H,H,SC(═O)N(i-Pr)CH₂CHF₂,H], [A2817;H,H,SC(═O)N(i-Pr)CH₂CF₃,H], [A2818;H,H,SC(═O)N(i-Pr)(CH₂)₃F,H], [A2819;H,H,SC(═O)N(i-Pr)(CH₂)₂CHF₂,H], [A2820;H,H,SC(═O)N(i-Pr)(CH₂)₂CF₃,H], [A2821;H,H,SC(═O)N(i-Pr)CH₂CH(CF₃)₂,H], [A2822;H,H,SC(═O)N(i-Pr) CH₂Cl,H], [A2823;H,H,SC(═O)N(i-Pr)(CH₂)₂Cl,H], [A2824;H,H,SC(═O)N(i-Pr)(CH₂)₃Cl,H], [A2825;H,H,SC(═O)N(i-Pr)(CH₂)₄Cl,H], [A2826;H,H,SC(═O)N(i-Pr)(CH₂)₅Cl,H], [A2827;H,H,SC(═O)N(i-Pr)CH₂Br,H], [A2828;H,H,SC(═O)N(i-Pr)(CH₂)₂Br,H], [A2829;H,H,SC(═O)N(i-Pr)(CH₂)₃Br,H], [A2830;H,H,SC(═O)N(i-Pr)(CH₂)₄Br,H], [A2831;H,H,SC(═O)N(i-Pr)(CH₂)₅Br,H], [A2832;H,H,SC(═O)N(i-Pr)CH₂OMe,H], [A2833;H,H,SC(═O)N(i-Pr)(CH₂)₂OMe,H], [A2834;H,H,SC(═O)N(i-Pr)(CH₂)₃OMe,H], [A2835;H,H,SC(═O)N(i-Pr)(CH₂)₄OMe,H], [A2836;H,H,SC(═O)N(i-Pr)CH₂OEt,H], [A2837;H,H,SC(═O)N(i-Pr)(CH₂)₂OEt,H], [A2838;H,H,SC(═O)N(i-Pr)(CH₂)₃OEt,H], [A2839;H,H,SC(═O)N(i-Pr)CH₂CN,H], [A2840;H,H,SC(═O)N(i-Pr)(CH₂)₂CN,H], [A2841;H,H,SC(═O)N(i-Pr)(CH₂)₃CN,H], [A2842;H,H,SC(═O)N(i-Pr)(CH₂)₄CN,H], [A2843;H,H,SC(═O)N(i-Pr)(CH₂)₅CN,H], [A2844;H,H,SC(═O)N(i-Pr)CH₂C═CHF,H], [A2845;H,H,SC(═O)N(i-Pr)CH₂C═CF₂,H], [A2846;H,H,SC(═O)N(i-Pr)(CH₂)₂C═CHF,H], [A2847;H,H,SC(═O)N(i-Pr) CHCH₂C═CF₂,H], [A2848;H,H,SC(═O)N(i-Pr)(CH₂)₃C═CHF,H], [A2849;H,H,SC(═O)N(i-Pr)CH₂CHCH₂C═CF₂,H], [A2850;H,H,SC(═O)N(i-Pr) CH₂C═CHCl,H], [A2851;H,H,SC(═O)N(i-Pr)CH₂C═CCl₂,H], [A2852;H,H,SC(═O)N(i-Pr)(CH₂₂C═CHCl,H], [A2853;H,H,SC(═O)N(i-Pr) CHCH₂C═CCl₂,H], [A2854;H,H,SC(═O)N(i-Pr)(CH₂)₃C═CHCl,H], [A2855;H,H,SC(═O)N(i-Pr)CH₂CHCH₂C═CCl₂,H], [A2856;H,H,SC(═O)N(i-Pr)CH₂C═CH,H], [A2857;H,H,SC(═O)N(i-Pr)CH(Me)C═CH,H], [A2858;H,H,SC(═O)N(i-Pr)CH₂C═CMe,H], [A2859;H,H,SC(═O)N(i-Pr)CH₂C=CEt,H], [A2860;H,H,SC(═O)N(i-Pr)CH₂C=CEt,H], [A2861;H,H,SC(═O)N(i-Pr)(CH₂)₂C═CH,H], [A2862;H,H,SC(═O)N(i-Pr)(CH₂)₂C═CMe,H], [A2863;H,H,SC(═O)N(i-Pr)(CH₂)₃C═CH,H], [A2864;H,H,SC(═O)N(i-Pr)(CH₂)₄C═CH,H], [A2865;H,H,SC(═O)N(i-Pr)c-Pr,H], [A2866;H,H,SC(═O)N(i-Pr)c-Bu,H], [A2867;H,H,SC(═O)N(i-Pr)c-Pen,H], [A2868;H,H,SC(═O)N(i-Pr)c-Hex,H], [A2869;H,H,SC(═O)N(i-Pr)c-Hep,H], [A2870;H,H,SC(═O)N(i-Pr) OCH₂F,H], [A2871;H,H,SC(═O)N(i-Pr)OCHF₂,H], [A2872;H,H,SC(═O)N(i-Pr)OCF₃,H], [A2873;H,H,SC(═O)N(i-Pr)O(CH₂)₂F,H], [A2874;H,H,SC(═O)N(i-Pr) OCH₂CHF₂,H], [A2875;H,H,SC(═O)N(i-Pr) OCH₂CF₃,H], [A2876;H,H,SC(═O)N(i-Pr)O(CH₂) sF,H], [A2877;H,H,SC(═O)N(i-Pr)O(CH₂)₂CHF₂,H], [A2878;H,H,SC(═O)N(i-Pr)O(CH₂)₂CF₃,H], [A2879;H,H,SC(═O)N(i-Pr) OCH₂CH(CF) 2,H], [A2880;H,H,SC(═O)N(i-Pr) OCH₂CH═CH₂,H], [A2881;H,H,SC(═O)N(i-Pr) OCH₂CH═CHF,H], [A2882;H,H,SC(═O)N (i-Pr) OCH₂CH═CF₂,H], [A2883;H,H,SC(═O)N(i-Pr) OCH₂CH═CHCl,H], [A2884;H,H,SC(═O)N(i-Pr) OCH₂CH═CCl₂,H], [A2885;H,H,SC(═O)N(i-Pr) OCH₂C═CH,H], [A2886;H,H,SC(═O)N(i-Pr) OCH(Me)C═CH,H], [A2887;H,H,SC(═O)N(i-Pr) OCH₂C═CMe,H], [A2888;H,H,SC(═S)NHMe,H], [A2889;H,H,SC(═S)NHEt,H], [A2890;H,H,SC(═S)NH(CH₂)₂Me,H], [A2891;H,H,SC(═S)NH(CH₂)₃Me,H], [A2892;H,H,SC(═S)NH(CH₂)₄Me,H], [A2893;H,H,SC(═S)NHCH(Me)₂,H], [A2894;H,H,SC(═S)NHCH₂CH(Me)₂,H], [A2895;H,H,SC(═S)NH(CH₂)₂CH(Me)₂,H], [A2896;H,H,SC(═S)NHC(Me)₃,H], [A2897;H,H,SC(═S)NHCH₂C(Me)₃,H], [A2898;H,H,SC(═S)NH(CH₂)₂C(Me)₃,H], [A2899;H,H,SC(═S)NHCH(Me)Et,H], [A2900;H,H,SC(═S)NHCH(Me)(CH₂)₂Me,H],

[A2901;H,H,SC(═S)NHCH(Me)(CH₂)₃Me,H], [A2902;H,H,SC(═S)NHCH(Et)₂,H], [A2903;H,H,SC(═S)NHCH₂CH═CH₂,H], [A2904;H,H,SC(═S)NH(CH₂)₂CH═CH₂,H], [A2905;H,H,SC(═S)NH(CH₂)CH═CH₂,H], [A2906;H,H,SC(═S)NHCH₂CH═CHMe,H], [A2907;H,H,SC(═S)NHCH₂CH═CHEt,H], [A2908;H,H,SC(═S)NHCH₂CH═C(Me)₂,H], [A2909;H,H,SC(═S)NHCH₂CH═C(Me)Et,H], [A2910;H,H,SC(═S)NHOMe,H], [A2911;H,H,SC(═S)NHOEt,H], [A2912;H,H,SC(═S)NHO(CH₂)₂Me,H], [A2913;H,H,SC(═S)NHO(CH₂)₃Me,H], [A2914;H,H,SC(═S)NHO(CH₂)₄Me,H], [A2915;H,H,SC(═S)NHOCH(Me)₂,H], [A2916;H,H,SC(═S)NHOCH₂CH(Me)₂,H], [A2917;H,H,SC(═S)NHO(CH₂)₂CH(Me)₂,H], [A2918;H,H,SC(═S)NHOC(Me)₃,H], [A2919;H,H,SC(═S)NHOCH₂C(Me)₃,H], [A2920;H,H,SC(═S)NHO(CH₂)₂C(Me)₃,H], [A2921;H,H,SC(═S)NHOCH(Me)Et,H], [A2922;H,H,SC(═S)NHOCH(Me)(CH₂)₂Me,H], [A2923;H,H,SC(═S)NHOCH(Me)(CH₂)₃Me,H], [A2924;H,H,SC(═S)NHOCH(Et)₂,H], [A2925;H,H,SC(═S)NHOCH₂CH═CH₂,H], [A2926;H,H,SC(═S)NHO(CH₂)₂CH═CH₂,H], [A2927;H,H,SC(═S)NHO(CH₂)₃CH═CH₂,H], [A2928;H,H,SC(═S)NHOCH₂CH═CHMe,H], [A2929;H,H,SC(═S)NHOCH₂CH═CHEt,H], [A2930;H,H,SC(═S)NHOCH₂CH═C(Me)₂,H], [A2931;H,H,SC(═S)NHOCH₂CH═C(Me)Et,H], [A2932;H,H,SC(═S)NHCH₂F,H], [A2933;H,H,SC(═S)NHCHF₂,H], [A2934;H,H,SC(═S)NHCF₃,H], [A2935;H,H,SC(═S)NH(CH₂)₂F,H], [A2936;H,H,SC(═S)NHCH₂CHF₂,H], [A2937;H,H,SC(═S)NHCH₂CF₃,H], [A2938;H,H,SC(═S)NH(CH₂)₃F,H], [A2939;H,H,SC(═S)NH(CH₂)₂CHF₂,H], [A2940;H,H,SC(═S)NH(CH₂)₂CF₃,H], [A2941;H,H,SC(═S)NHCH₂CH(CF₃)₂,H], [A2942;H,H,SC(═S)NHCH₂Cl,H], [A2943;H,H,SC(═S)NH(CH₂)₂Cl,H], [A2944;H,H,SC(═S)NH(CH₂)₃Cl,H], [A2945;H,H,SC(═S)NH(CH₂)₄Cl,H], [A2946;H,H,SC(═S)NH(CH₂)₅Cl,H], [A2947;H,H,SC(═S)NHCH₂Br,H], [A2948;H,H,SC(═S)NH(CH₂)₂Br,H], [A2949;H,H,SC(═S)NH(CH₂)₃Br,H], [A2950;H,H,SC(═S)NH(CH₂)₄Br,H], [A2951;H,H,SC(═S)NH(CH₂)₅Br,H], [A2952;H,H,SC(═S)NHCH₂OMe,H], [A2953;H,H,SC(═S)NH(CH₂)₂OMe,H], [A2954;H,H,SC(═S)NH(CH₂)₃OMe,H], [A2955;H,H,SC(═S)NH(CH₂)₄OMe,H], [A2956;H,H,SC(═S)NHCH₂OEt,H], [A2957;H,H,SC(═S)NH(CH₂)₂OEt,H], [A2958;H,H,SC(═S)NH(CH₂)₃OEt,H], [A2959;H,H,SC(═S)NHCH₂CN,H], [A2960;H,H,SC(═S)NH(CH₂)₂CN,H], [A2961;H,H,SC(═S)NH(CH₂)₃CN,H], [A2962;H,H,SC(═S)NH(CH₂)₄CN,H], [A2963;H,H,SC(═S)NH(CH₂)₅CN,H], [A2964;H,H,SC(═S)NHCH₂C═CHF,H], [A2965;H,H,SC(═S)NHCH₂C═CF₂,H], [A2966;H,H,SC(═S)NH(CH₂)₂C═CHF,H], [A2967;H,H,SC(═S)NHCHCH₂C═CF₂,H], [A2968;H,H,SC(═S)NH(CH₂)₃C═CHF,H], [A2969;H,H,SC(═S)NHCH₂CHCH₂C═CF₂,H], [A2970;H,H,SC(═S)NHCH₂C═CHCl,H], [A2971;H,H,SC(═S)NHCH₂C═CCl₂,H], [A2972;H,H,SC(═S)NH(CH₂)₂C═CHCl,H], [A2973;H,H,SC(═S)NHCHCH₂C═CCl₂,H], [A2974;H,H,SC(═S)NH(CH₂)₃C═CHCl,H], [A2975;H,H,SC(═S)NHCH₂CHCH₂C═CCl₂,H], [A2976;H,H,SC(═S)NHCH₂C═CH,H], [A2977;H,H,SC(═S)NHCH(Me)C═CH,H], [A2978;H,H,SC(═S)NHCH₂C═CMe,H], [A2979;H,H,SC(═S)NHCH₂C=CEt,H], [A2980;H,H,SC(═S)NHCH₂C=CEt,H], [A2981;H,H,SC(═S)NH(CH₂)₂C═CH,H], [A2982;H,H,SC(═S)NH(CH₂)₂C═CMe,H], [A2983;H,H,SC(═S)NH(CH₂)₃C═CH,H], [A2984;H,H,SC(═S)NH(CH₂)₄C═CH,H], [A2985;H,H,SC(═S)NHc-Pr,H], [A2986;H,H,SC(═S)NHc-Bu,H], [A2987;H,H,SC(═S)NHc-Pen,H], [A2988;H,H,SC(═S)NHc-Hex,H], [A2989;H,H,SC(═S)NHc-Hep,H], [A2990;H,H,SC(═S)NHOCH₂F,H], [A2991;H,H,SC(═S)NHOCHF₂,H], [A2992;H,H,SC(═S)NHOCF₃,H], [A2993;H,H,SC(═S)NHO(CH₂)₂F,H], [A2994;H,H,SC(═S)NHOCH₂CHF₂,H], [A2995;H,H,SC(═S)NHOCH₂CF₃,H], [A2996;H,H,SC(═S)NHO(CH₂)₃F,H], [A2997;H,H,SC(═S)NHO(CH₂)₂CHF₂,H][A2998;H,H,SC(═S)NHO(CH₂)₂CF₃,H], [A2999;H,H,SC(═S)NHOCH₂CH(CF₃)₂,H][A3000;H,H,SC(═S)NHOCH₂CH═CH₂,H]

[A3001;H,H,SC (═S)NHOCH₂CH═CHF,H][A3002;H,H,SC(═S)NHOCH₂CH═CF₂,H], [A3003;H,H,SC(═S)NHOCH₂CH═CHCl,H], [A3004;H,H,SC(═S)NHOCH₂CH═CCl₂,H], [A3005;H,H,SC(═S)NHOCH₂C═CH,H], [A3006;H,H,SC(═S)NHOCH(Me)C═CH,H], [A3007;H,H,SC(═S)NHOCH₂C═CMe,H], [A3008;H,H,SC(═S)NMeMe,H], [A3009;H,H,SC(═S)NMeEt,H], [A3010;H,H,SC(═S)NMe(CH₂)₂Me,H], [A3011;H,H,SC(═S)NMe(CH₂)₃Me,H], [A3012;H,H,SC(═S)NMe(CH₂)₄Me,H], [A3013;H,H,SC(═S)NMeCH(Me)₂,H], [A3014;H,H,SC(═S)NMeCH₂CH(Me)₂,H], [A3015;H,H,SC(═S)NMe(CH₂)₂CH(Me)₂,H], [A3016;H,H,SC(═S)NMeC(Me)₃,H], [A3017;H,H,SC(═S)NMeCH₂C(Me)₃,H], [A3018;H,H,SC(═S)NMe(CH₂)₂C(Me)₃,H], [A3019;H,H,SC(═S)NMeCH(Me)Et,H], [A3020;H,H,SC(═S)NMeCH(Me)(CH₂)₂Me,H], [A3021;H,H,SC(═S)NMeCH(Me)(CH₂)₃Me,H], [A3022;H,H,SC(═S)NMeCH(Et)₂,H], [A3023;H,H,SC(═S)NMeCH₂CH═CH₂,H], [A3024;H,H,SC(═S)NMe(CH₂)₂CH═CH₂,H], [A3025;H,H,SC(═S)NMe(CH₂)₃CH═CH₂,H], [A3026;H,H,SC(═S)NMeCH₂CH═CHMe,H], [A3027;H,H,SC(═S)NMeCH₂CH═CHEt,H], [A3028;H,H,SC(═S)NMeCH₂CH═C(Me)₂,H], [A3029;H,H,SC(═S)NMeCH₂CH═C(Me)Et,H], [A3030;H,H,SC(═S)NMeOMe,H], [A3031;H,H,SC(═S)NMeOEt,H], [A3032;H,H,SC(═S)NMeO(CH₂)₂Me,H], [A3033;H,H,SC(═S)NMeO(CH₂)₃Me,H], [A3034;H,H,SC(═S)NMeO(CH₂)₄Me,H], [A3035;H,H,SC(═S)NMeOCH(Me)₂,H], [A3036;H,H,SC(═S)NMeOCH₂CH(Me)₂,H], [A3037;H,H,SC(═S)NMeO(CH₂)₂CH(Me)₂,H][A3038;H,H,SC(═S)NMeOC(Me)₃,H], [A3039;H,H,SC(═S)NMeOCH₂C(Me)₃,H], [A3040;H,H,SC(═S)NMeO(CH₂)₂C(Me)₃,H][A3041;H,H,SC(═S)NMeOCH(Me)Et,H], [A3042;H,H,SC(═S)NMeOCH(Me)(CH₂)₂Me,H], [A3043;H,H,SC(═S)NMeOCH(Me)(CH₂)₃Me,H], [A3044;H,H,SC(═S)NMeOCH(Et)₂,H], [A3045;H,H,SC(═S)NMeOCH₂CH═CH₂,H], [A3046;H,H,SC(═S)NMeO(CH₂)₂CH═CH₂,H], [A3047;H,H,SC(═S)NMeO(CH₂)₃CH═CH₂,H], [A3048;H,H,SC(═S)NMeOCH₂CH═CHMe,H], [A3049;H,H,SC(═S)NMeOCH₂CH═CHEt,H], [A3050;H,H,SC (═S)NMeOCH₂CH═C(Me)₂,H], [A3051;H,H,SC (═S)NMeOCH₂CH═C(Me)Et,H][A3052;H,H,SC(═S)NMeCH₂F,H], [A3053;H,H,SC(═S)NMeCHF₂,H], [A3054;H,H,SC(═S)NMeCF₃,H], [A3055;H,H,SC(═S)NMe(CH₂₂F,H], [A3056;H,H,SC(═S)NMeCH₂CHF₂,H], [A3057;H,H,SC(═S)NMeCH₂CF₃,H], [A3058;H,H,SC(═S)NMe(CH₂)₃F,H][A3059;H,H,SC(═S)NMe(CH₂)₂CHF₂,H][A3060;H,H,SC(═S)NMe(CH₂)₂CF₃,H], [A3061;H,H,SC(═S)NMeCH₂CH(CF₃)₂,H], [A3062;H,H,SC(═S)NMeCH₂Cl,H], [A3063;H,H,SC(═S)NMe(CH₂)₂Cl,H], [A3064;H,H,SC(═S)NMe(CH₂)₃Cl,H][A3065;H,H,SC(═S)NMe(CH₂)C1,H][A3066;H,H,SC(═S)NMe(CH₂)₅Cl,H], [A3067;H,H,SC(═S)NMeCH₂Br,H], [A3068;H,H,SC(═S)NMe(CH₂)₂Br,H], [A3069;H,H,SC(═S)NMe(CH₂)₃Br,H], [A3070;H,H,SC(═S)NMe(CH₂)₄Br,H], [A3071;H,H,SC(═S)NMe(CH₂)₅Br,H], [A3072;H,H,SC(═S)NMeCH₂OMe,H], [A3073;H,H,SC(═S)NMe(CH₂)₂OMe,H], [A3074;H,H,SC(═S)NMe(CH₂)₃OMe,H], [A3075;H,H,SC(═S)NMe(CH₂)₄OMe,H], [A3076;H,H,SC(═S)NMeCH₂OEt,H][A3077;H,H,SC(═S)NMe(CH₂)₂OEt,H], [A3078;H,H,SC(═S)NMe(CH₂)₃OEt,H], [A3079;H,H,SC(═S)NMeCH₂CN,H], [A3080;H,H,SC(═S)NMe(CH)₂CN,H], [A3081;H,H,SC(═S)NMe(CH)₃CN,H], [A3082;H,H,SC(═S)NMe(CH₂)₄CN,H], [A3083;H,H,SC(═S)NMe(CH₂)₅CN,H], [A3084;H,H,SC(═S)NMeCH₂C═CHF,H], [A3085;H,H,SC(═S)NMeCH₂C═CF₂,H], [A3086;H,H,SC(═S)NMe(CH)₂C═CHF,H], [A3087;H,H,SC(═S)NMeCHCH₂C═CF₂,H], [A3088;H,H,SC(═S)NMe(CH₂)₃C═CHF,H], [A3089;H,H,SC(═S)NMeCH₂CHCH₂C═CF₂,H], [A3090;H,H,SC(═S)NMeCH₂C═CHCl,H], [A3091;H,H,SC(═S)NMeCH₂C═CCl₂,H], [A3092;H,H,SC(═S)NMe(CH₂)₂C═CHCl,H], [A3093;H,H,SC(═S)NMeCHCH₂C═CCl₂,H], [A3094;H,H,SC(═S)NMe(CH₂)₃C═CHCl,H], [A3095;H,H,SC(═S)NMeCH₂CHCH₂C═CCl₂,H], [A3096;H,H,SC(═S)NMeCH₂C═CH,H], [A3097;H,H,SC(═S)NMeCH(Me)C═CH,H], [A3098;H,H,SC(═S)NMeCH₂C═CMe,H], [A3099;H,H,SC(═S)NMeCH₂C=CEt,H], [A3100;H,H,SC(═S)NMeCH₂C=CEt,H],

[A3101;H,H,SC(═S)NMe(CH₂)₂C═CH,H][A3102;H,H,SC(═S)NMe(CH₂₂C═CMe,H], [A3103;H,H,SC(═S)NMe(CH)₃C═CH,H], [A3104;H,H,SC(═S)NMe(CH₂)₄C═CH,H], [A3105;H,H,SC(═S)NMec-Pr,H], [A3106;H,H,SC(═S)NMec-Bu,H], [A3107;H,H,SC(═S)NMec-Pen,H], [A3108;H,H,SC(═S)NMec-Hex,H], [A3109;H,H,SC(═S)NMec-Hep,H], [A3110;H,H,SC(═S)NMeOCH₂F,H], [A3111;H,H,SC(═S)NMeOCHF₂,H], [A3112;H,H,SC(═S)NMeOCF₃,H], [A3113;H,H,SC(═S)NMeO(CH₂)₂F,H][A3114;H,H,SC(═S)NMeOCH₂CHF₂,H], [A3115;H,H,SC(═S)NMeOCH₂CF₃,H], [A3116;H,H,SC(═S)NMeO(CH₂)₃F,H][A3117;H,H,SC(═S)NMeO(CH₂)₂CHF₂,H], [A3118;H,H,SC(═S)NMeO(CH₂)₂CF₃,H][A3119;H,H,SC(═S)NMeOCH₂CH(CF₃)₂,H], [A3120;H,H,SC (═S)NMeOCH₂CH═CH₂,H], [A3121;H,H,SC (═S)NMeOCH₂CH═CHF,H][A3122;H,H,SC(═S)NMeOCH₂CH═CF₂,H], [A3123;H,H,SC(═S)NMeOCH₂CH═CHCl,H], [A3124;H,H,SC(═S)NMeOCH₂CH═CCl₂,H], [A3125;H,H,SC(═S)NMeOCH₂C═CH,H], [A3126;H,H,SC (═S)NMeOCH(Me)C═CH,H][A3127;H,H,SC(═S)NMeOCH₂C═CMe,H], [A3128;H,H,SC(═S)NEtMe,H], [A3129;H,H,SC(═S)NEtEt,H], [A3130;H,H,SC(═S)NEt(CH₂)₂Me,H], [A3131;H,H,SC(═S)NEt(CH₂)₃Me,H][A3132;H,H,SC(═S)NEt(CH₂)₄Me,H][A3133;H,H,SC(═S)NEtCH(Me)₂,H], [A3134;H,H,SC(═S)NEtCH₂CH(Me)₂,H], [A3135;H,H,SC(═S)NEt(CH₂)₂CH(Me)₂,H], [A3136;H,H,SC(═S)NEtC(Me)₃,H][A3137;H,H,SC(═S)NEtCH₂C(Me)₃,H], [A3138;H,H,SC(═S)NEt(CH₂)₂C(Me)₃,H], [A3139;H,H,SC(═S)NEtCH(Me)Et,H], [A3140;H,H,SC(═S)NEtCH(Me)(CH₂)₂Me,H], [A3141;H,H,SC(═S)NEtCH(Me)(CH₂)₃Me,H], [A3142;H,H,SC(═S)NEtCH (Et)₂,H], [A3143;H,H,SC(═S)NEtCH₂CH═CH₂,H], [A3144;H,H,SC(═S)NEt(CH₂)₂CH═CH₂,H], [A3145;H,H,SC(═S)NEt(CH₂)₃CH═CH₂,H], [A3146;H,H,SC(═S)NEtCH₂CH═CHMe,H], [A3147;H,H,SC(═S)NEtCH₂CH═CHEt,H], [A3148;H,H,SC(═S)NEtCH₂CH═C(Me)₂,H], [A3149;H,H,SC(═S)NEtCH₂CH═C(Me)Et,H], [A3150;H,H,SC(═S)NEtOMe,H], [A3151;H,H,SC(═S)NEtOEt,H], [A3152;H,H,SC(═S)NEtO(CH₂)₂Me,H], [A3153;H,H,SC(═S)NEtO(CH₂)₃Me,H], [A3154;H,H,SC(═S)NEtO(CH₂)₄Me,H], [A3155;H,H,SC(═S)NEtOCH(Me)₂,H], [A3156;H,H,SC(═S)NEtOCH₂CH(Me)₂,H], [A3157;H,H,SC(═S)NEtO(CH₂)₂CH(Me)₂,H], [A3158;H,H,SC(═S)NEtOC(Me)₃,H], [A3159;H,H,SC(═S)NEtOCH₂C(Me)₃,H], [A3160;H,H,SC(═S)NEtO(CH₂)₂C(Me)₃,H], [A3161;H,H,SC(═S)NEtOCH(Me)Et,H], [A3162;H,H,SC(═S)NEtOCH(Me)(CH₂)₂Me,H], [A3163;H,H,SC(═S)NEtOCH(Me)(CH₂)₃Me,H], [A3164;H,H,SC(═S)NEtOCH(Et)₂,H], [A3165;H,H,SC(═S)NEtOCH₂CH═CH₂,H], [A3166;H,H,SC(═S)NEtO(CH₂)₂CH═CH₂,H], [A3167;H,H,SC(═S)NEtO(CH₂)₃CH═CH₂,H], [A3168;H,H,SC(═S)NEtOCH₂CH═CHMe,H], [A3169;H,H,SC(═S)NEtOCH₂CH═CH Et,H], [A3170;H,H,SC(═S)NEtOCH₂CH═C(Me)₂,H], [A3171;H,H,SC(═S)NEtOCH₂CH═C(Me)Et,H], [A3172;H,H,SC(═S)NEtCH₂F,H], [A3173;H,H,SC(═S)NEtCHF₂,H], [A3174;H,H,SC(═S)NEtCF₃,H], [A3175;H,H,SC(═S)NEt(CH₂)₂F,H], [A3176;H,H,SC(═S)NEtCH₂CHF₂,H], [A3177;H,H,SC(═S)NEtCH₂CF₃,H], [A3178;H,H,SC(═S)NEt(CH₂)₃F,H], [A3179;H,H,SC(═S)NEt(CH₂)₂CHF₂,H], [A3180;H,H,SC(═S)NEt(CH₂)₂CF₃,H], [A3181;H,H,SC(═S)NEtCH₂CH(CF₃)₂,H], [A3182;H,H,SC(═S)NEtCH₂Cl,H], [A3183;H,H,SC(═S)NEt(CH₂)₂Cl,H], [A3184;H,H,SC(═S)NEt(CH₂)₃Cl,H], [A3185;H,H,SC(═S)NEt(CH₂)₄Cl,H], [A3186;H,H,SC(═S)NEt(CH₂)₅Cl,H], [A3187;H,H,SC(═S)NEtCH₂Br,H], [A3188;H,H,SC(═S)NEt(CH₂)₂Br,H], [A3189;H,H,SC(═S)NEt(CH₂)₃Br,H], [A3190;H,H,SC(═S)NEt(CH₂)₄Br,H], [A3191;H,H,SC(═S)NEt(CH₂)₅Br,H], [A3192;H,H,SC(═S)NEtCH₂Me,H], [A3193;H,H,SC(═S)NEt(CH₂)₂OMe,H], [A3194;H,H,SC(═S)NEt(CH₂)₃OMe,H], [A3195;H,H,SC(═S)NEt(CH₂)₄Me,H], [A3196;H,H,SC(═S)NEtCH₂Et,H], [A3197;H,H,SC(═S)NEt(CH₂)₂OEt,H], [A3198;H,H,SC(═S)NEt(CH₂)₃OEt,H], [A3199;H,H,SC(═S)NEtCH₂CN,H], [A3200;H,H,SC(═S)NEt(CH₂)₂CN,H],

[A3201;H,H,SC(═S)NEt(CH₂)₃CN,H], [A3202;H,H,SC(═S)NEt(CH₂)₄CN,H], [A3203;H,H,SC(═S)NEt(CH₂)₅CN,H], [A3204;H,H,SC(═S)NEtCH₂C═CHF,H], [A3205;H,H,SC(═S)NEtCH₂C═CF₂,H], [A3206;H,H,SC(═S)NEt(CH₂)₂C═CHF,H], [A3207;H,H,SC(═S)NEtCHCH₂C═CF₂,H], [A3208;H,H,SC(═S)NEt(CH₂)₃C═CHF,H], [A3209;H,H,SC(═S)NEtCH₂CHCH₂C═CF₂,H], [A3210;H,H,SC(═S)NEtCH₂C═CHCl,H], [A3211;H,H,SC(═S)NEtCH₂C═CCl₂,H], [A3212;H,H,SC(═S)NEt(CH₂)₂C═CHCl,H], [A3213;H,H,SC(═S)NEtCHCH₂C═CCl₂,H], [A3214;H,H,SC(═S)NEt(CH₂)₃C═CHCl,H], [A3215;H,H,SC(═S)NEtCH₂CHCH₂C═CCl₂,H], [A3216;H,H,SC(═S)NEtCH₂C═CH,H], [A3217;H,H,SC(═S)NEtCH(Me)C═CH,H][A3218;H,H,SC(═S)NEtCH₂C═CMe,H], [A3219;H,H,SC(═S)NEtCH₂C=CEt,H], [A3220;H,H,SC(═S)NEtCH₂C=CEt,H], [A3221;H,H,SC(═S)NEt(CH₂)₂C═CH,H][A3222;H,H,SC(═S)NEt(CH₂₂C═CMe,H][A3223;H,H,SC(═S)NEt(CH₂)₃C═CH,H], [A3224;H,H,SC(═S)NEt(CH₂)₄C═CH,H], [A3225;H,H,SC(═S)NEtc-Pr,H], [A3226;H,H,SC(═S)NEtc-Bu,H], [A3227;H,H,SC(═S)NEtc-Pen,H], [A3228;H,H,SC(═S)NEtc-Hex,H], [A3229;H,H,SC(═S)NEtc-Hep,H], [A3230;H,H,SC(═S)NEtOCH₂F,H], [A3231;H,H,SC(═S)NEtOCHF₂,H], [A3232;H,H,SC(═S)NEtOCF₃,H], [A3233;H,H,SC(═S)NEtO(CH₂)₂F,H][A3234;H,H,SC(═S)NEtOCH₂CHF₂,H], [A3235;H,H,SC(═S)NEtOCH₂CF₃,H][A3236;H,H,SC(═S)NEtO(CH₂)₃F,H][A3237;H,H,SC(═S)NEtO(CH₂)₂CHF₂,H], [A3238;H,H,SC(═S)NEtO(CH₂)₂CF₃,H], [A3239;H,H,SC(═S)NEtOCH₂CH(CF₃)₂,H], [A3240;H,H,SC(═S)NEtOCH₂CH═CH₂,H], [A3241;H,H,SC (═S)NEtOCH₂CH═CHF,H][A3242;H,H,SC(═S)NEtOCH₂CH═CF₂,H][A3243;H,H,SC(═S)NEtOCH₂CH═CHCl,H], [A3244;H,H,SC(═S)NEtOCH₂CH═CCl₂,H], [A3245;H,H,SC(═S)NEtOCH₂C═CH,H], [A3246;H,H,SC(═S)NEtOCH(Me)C═CH,H][A3247;H,H,SC(═S)NEtOCH₂C═CMe,H], [A3248;H,H,SC(═S)NPrMe,H], [A3249;H,H,SC(═S)NPrEt,H], [A3250;H,H,SC(═S)NPr(CH₂)₂Me,H], [A3251;H,H,SC(═S)NPr(CH₂)₃Me,H], [A3252;H,H,SC(═S)NPr(CH₂)₄Me,H], [A3253;H,H,SC(═S)NPrCH(Me)₂,H], [A3254;H,H,SC(═S)NPrCH₂CH(Me)₂,H], [A3255;H,H,SC(═S)NPr(CH₂)₂CH(Me)₂,H], [A3256;H,H,SC(═S)NPrC(Me)₃,H], [A3257;H,H,SC(═S)NPrCH₂C(Me)₃,H], [A3258;H,H,SC(═S)NPr(CH₂)₂C(Me)₃,H], [A3259;H,H,SC(═S)NPrCH(Me)Et,H], [A3260;H,H,SC(═S)NPrCH(Me)(CH₂)₂Me,H], [A3261;H,H,SC(═S)NPrCH(Me)(CH₂)₃Me,H], [A3262;H,H,SC(═S)NPrCH(Et)₂,H], [A3263;H,H,SC(═S)NPrCH₂CH═CH₂,H], [A3264;H,H,SC(═S)NPr(CH₂)₂CH═CH₂,H], [A3265;H,H,SC(═S)NPr(CH₂)₃CH═CH₂,H], [A3266;H,H,SC(═S)NPrCH₂CH═CHMe,H], [A3267;H,H,SC(═S)NPrCH₂CH═CHEt,H], [A3268;H,H,SC(═S)NPrCH₂CH═C(Me)₂,H], [A3269;H,H,SC(═S)NPrCH₂CH═C(Me)Et,H], [A3270;H,H,SC(═S)NPrOMe,H], [A3271;H,H,SC(═S)NPrOEt,H], [A3272;H,H,SC(═S)NPrO(CH₂)₂Me,H], [A3273;H,H,SC(═S)NPrO(CH₂)₃Me,H], [A3274;H,H,SC(═S)NPrO(CH₂)₄Me,H], [A3275;H,H,SC(═S)NPrOCH(Me)₂,H], [A3276;H,H,SC(═S)NPrOCH₂CH(Me)₂,H], [A3277;H,H,SC(═S)NPrO(CH₂)₂CH(Me)₂,H], [A3278;H,H,SC(═S)NPrOC(Me)₃,H], [A3279;H,H,SC(═S)NPrOCH₂C(Me)₃,H], [A3280;H,H,SC(═S)NPrO(CH₂)₂C(Me)₃,H], [A3281;H,H,SC(═S)NPrOCH(Me)Et,H], [A3282;H,H,SC(═S)NPrOCH(Me)(CH₂)₂Me,H], [A3283;H,H,SC(═S)NPrOCH(Me)(CH₂)₃Me,H], [A3284;H,H,SC(═S)NPrOCH(Et)₂,H], [A3285;H,H,SC(═S)NPrOCH₂CH═CH₂,H], [A3286;H,H,SC(═S)NPrO(CH₂)₂CH═CH₂,H], [A3287;H,H,SC(═S)NPrO(CH₂)CH═CH₂,H], [A3288;H,H,SC(═S)NPrOCH₂CH═CHMe,H], [A3289;H,H,SC(═S)NPrOCH₂CH═CHEt,H], [A3290;H,H,SC(═S)NPrOCH₂CH═C(Me)₂,H], [A3291;H,H,SC(═S)NPrOCH₂CH═C(Me)Et,H], [A3292;H,H,SC(═S)NPrCH₂F,H], [A3293;H,H,SC(═S)NPrCHF₂,H], [A3294;H,H,SC(═S)NPrCF₃,H], [A3295;H,H,SC(═S)NPr(CH₂)₂F,H], [A3296;H,H,SC(═S)NPrCH₂CHF₂,H], [A3297;H,H,SC(═S)NPrCH₂CF₃,H], [A3298;H,H,SC(═S)NPr(CH₂)₃F,H], [A3299;H,H,SC(═S)NPr(CH₂)₂CHF₂,H], [A3300;H,H,SC(═S)NPr(CH₂)₂CF₃,H],

[A3301;H,H,SC(═S)NPrCH₂CH(CF₃)₂,H], [A3302;H,H,SC(═S)NPrCH₂Cl,H], [A3303;H,H,SC(═S)NPr(CH₂)₂Cl,H], [A3304;H,H,SC(═S)NPr(CH₂)₃C,H], [A3305;H,H,SC(═S)NPr(CH₂)₄Cl,H], [A3306;H,H,SC(═S)NPr(CH₂)₅Cl,H], [A3307;H,H,SC(═S)NPrCH₂Br,H], [A3308;H,H,SC(═S)NPr(CH₂)₂Br,H], [A3309;H,H,SC(═S)NPr(CH₂)₃Br,H], [A3310;H,H,SC(═S)NPr(CH₂)₄Br,H], [A3311;H,H,SC(═S)NPr(CH₂)₅Br,H], [A3312;H,H,SC(═S)NPrCH₂OMe,H], [A3313;H,H,SC(═S)NPr(CH₂)₂OMe,H], [A3314;H,H,SC(═S)NPr(CH₂)₃OMe,H], [A3315;H,H,SC(═S)NPr(CH₂)₄OMe,H], [A3316;H,H,SC(═S)NPrCH₂OEt,H], [A3317;H,H,SC(═S)NPr(CH₂)₂OEt,H][A3318;H,H,SC(═S)NPr(CH₂)₃OEt,H][A3319;H,H,SC(═S)NPrCH₂CN,H], [A3320;H,H,SC(═S)NPr(CH₂)₂CN,H], [A3321;H,H,SC(═S)NPr(CH₂)₃CN,H], [A3322;H,H,SC(═S)NPr(CH₂)₄CN,H], [A3323;H,H,SC(═S)NPr(CH₂)₅CN,H], [A3324;H,H,SC(═S)NPrCH₂)C═CH,H], [A3325;H,H,SC(═S)NPrCH₂C═CF₂,H][A3326;H,H,SC(═S)NPr(CH₂)₂,H][A3327;H,H,SC(═S)NPrC CH₂) C═CF₃,H], [A3328;H,H,SC(═S)NPrC(CH₂)₃C═CF₃,H], [A3329;H,H,SC(═S)NPrCH₂CHCH₂C═CF₃,H], [A3330;H,H,SC(═S)NPrCH₂C═CHCl,H], [A3331;H,H,SC(═S)NPrCH₂C═CCl₂,H][A3332;H,H,SC(═S)NPr(CH₂)C═CH₁,H], [A3333;H,H,SC(═S)NPrCHCH₂C═CCl₂,H], [A3334;H,H,SC(═S)NPr(CH₂)C═CHCl,H], [A3335;H,H,SC(═S)NPr(CH₂CHCH₂C═CCl₂,H], [A3336;H,H,SC(═S)NPrCH₂C≡CH,H], [A3337;H,H,SC(═S)NPrCH(Me)C═CH,H], [A3338;H,H,SC(═S)NPrCH₂C≡CMe,H], [A3339;H,H,SC(═S)NPrCH₂C=CEt,H], [A3340;H,H,SC(═S)NPrCH₂C=CEt,H], [A3341;H,H,SC(═S)NPr(CH₂)₂C═CH,H], [A3342;H,H,SC(═S)NPr(CH₂)₂C═CMe,H], [A3343;H,H,SC(═S)NPr(CH₂)₃C═CH,H], [A3344;H,H,SC(═S)NPr(CH₂)₄C═CH,H], [A3345;H,H,SC(═S)NPrc-Pr,H], [A3346;H,H,SC(═S)NPrc-Bu,H], [A3347;H,H,SC(═S)NPrc-Pen,H], [A3348;H,H,SC(═S)NPrc-Hex,H], [A3349;H,H,SC(═S)NPrc-Hep,H], [A3350;H,H,SC(═S)NPrOCH₂F,H], [A3351;H,H,SC(═S)NPrOCHF₂,H], [A3352;H,H,SC(═S)NPrOCF₃,H], [A3353;H,H,SC(═S)NPrO(CH₂)₂F,H][A3354;H,H,SC(═S)NPrOCH₂CHF₂,H], [A3355;H,H,SC(═S)NPrOCH₂CF₃,H], [A3356;H,H,SC(═S)NPrO(CH₂)₃F,H], [A3357;H,H,SC(═S)NPrO(CH₂)₂CHF₂,H], [A3358;H,H,SC(═S)NPrO(CH₂)₂CF₃,H][A3359;H,H,SC(═S)NPrOCH₂CH(CF₃)₂,H][A3360;H,H,SC(═S)NPrOCH₂CH═CH₂,H][A3361;H,H,SC(═S)NPrOCH₂CH═CHF,H], [A3362;H,H,SC(═S)NPrOCH₂CH═CF₂,H][A3363;H,H,SC(═S)NPrOCH₂CH═CHCl,H], [A3364;H,H,SC(═S)NPrOCH₂CH═CCl₂,H][A3365;H,H,SC(═S)NPrOCH₂C═CH,H], [A3366;H,H,SC(═S)NPrOCH(Me)C═CH,H], [A3367;H,H,SC(═S)NPrOCH₂C═CMe,H], [A3368;H,H,SC(═S)N(i-Pr)Me,H], [A3369;H,H,SC(═S)N(i-Pr)Et,H], [A3370;H,H,SC(═S)N(i-Pr)(CH₂)₂Me,H], [A3371;H,H,SC(═S)N(i-Pr)(CH₂)₃Me,H], [A3372;H,H,SC(═S)N(i-Pr)(CH₂)₄Me,H], [A3373;H,H,SC(═S)N(i-Pr)CH(Me)₂,H], [A3374;H,H,SC(═S)N(i-Pr)CH₂CH(Me)₂,H], [A3375;H,H,SC(═S)N(i-Pr)(CH₂)₂CH(Me)₂,H], [A3376;H,H,SC(═S)N(i-Pr)C(Me)₃,H], [A3377;H,H,SC(═S)N(i-Pr)CH₂C(Me)₃,H], [A3378;H,H,SC(═S)N(i-Pr)(CH₂)₂C(Me)₃,H], [A3379;H,H,SC(═S)N(i-Pr)CH(Me)Et,H], [A3380;H,H,SC(═S)N(i-Pr)CH(Me)(CH₂)₂Me,H], [A3381;H,H,SC(═S)N(i-Pr)CH(Me)(CH₂)₃Me,H], [A3382;H,H,SC(═S)N(i-Pr)CH(Et)₂,H], [A3383;H,H,SC(═S)N(i-Pr)CH₂CH═CH₂,H], [A3384;H,H,SC(═S)N(i-Pr)(CH₂)₂CH═CH₂,H], [A3385;H,H,SC(═S)N(i-Pr)(CH₂)₃CH═CH₂,H], [A3386;H,H,SC(═S)N(i-Pr)CH₂CH═CHMe,H], [A3387;H,H,SC(═S)N(i-Pr) CH₂CH═CHEt,H], [A3388;H,H,SC(═S)N(i-Pr) CH₂CH═C(Me)₂,H], [A3389;H,H,SC(═S)N(i-Pr) CH₂CH═C(Me)Et,H], [A3390;H,H,SC(═S)N(i-Pr)OMe,H], [A3391;H,H,SC(═S)N(i-Pr)OEt,H], [A3392;H,H,SC(═S)N(i-Pr)O(CH₂)₂Me,H], [A3393;H,H,SC(═S)N(i-Pr)O(CH₂)₃Me,H], [A3394;H,H,SC(═S)N(i-Pr)O(CH₂)₄Me,H], [A3395;H,H,SC(═S)N(i-Pr) OCH(Me)₂,H], [A3396;H,H,SC(═S)N(i-Pr) OCH₂CH(Me)₂,H], [A3397;H,H,SC(═S)N(i-Pr)O(CH₂)₂CH(Me)₂,H], [A3398;H,H,SC(═S)N(i-Pr)OC(Me)₃,H], [A3399;H,H,SC(═S)N(i-Pr) OCH₂C(Me)₃,H], [A3400;H,H,SC(═S)N(i-Pr)O(CH₂)₂C(Me)₃,H],

[A3401;H,H,SC(═S)N(i-Pr) OCH(Me)Et,H], [A3402;H,H,SC(═S)N(i-Pr) OCH(Me)(CH₂)₂Me,H], [A3403;H,H,SC(═S)N(i-Pr) OCH(Me)(CH₂)₃Me,H], [A3404;H,H,SC(═S)N(i-Pr) OCH(Et)₂,H], [A3405;H,H,SC(═S)N(i-Pr) OCH₂CH═CH₂,H], [A3406;H,H,SC(═S)N(i-Pr)O(CH₂)₂CH═CH₂,H], [A3407; H,H,SC(═S)N(i-Pr)O(CH₂)₃CH═CH₂,H], [A3408;H,H,SC(═S)N(i-Pr) OCH₂CH═CHMe,H], [A3409;H,H,SC(═S)N(i-Pr)OCH₂CH═CHEt,H], [A3410;H,H,SC(═S)N(i-Pr)OCH₂CH═C(Me)₂,H], [A3411;H,H,SC(═S)N(i-Pr)OCH₂CH═C(Me)Et,H], [A3412;H,H,SC(═S)N(i-Pr)CH₂F,H], [A3413;H,H,SC(═S)N(i-Pr)CHF₂,H], [A3414;H,H,SC(═S)N(i-Pr)CF₃,H], [A3415;H,H,SC(═S)N(i-Pr)(CH₂₂F,H], [A3416;H,H,SC(═S)N(i-Pr) CH₂CHF₂,H], [A3417;H,H,SC(═S)N(i-Pr)CH₂CF₃,H], [A3418;H,H,SC(═S)N(i-Pr)(CH₂)₃F,H], [A3419;H,H,SC(═S)N(i-Pr)(CH₂)₂CHF₂,H], [A3420;H,H,SC(═S)N(i-Pr)(CH₂)₂CF₃,H], [A3421;H,H,SC(═S)N(i-Pr)CH₂CH(CF₃)₂,H], [A3422;H,H,SC(═S)N(i-Pr) CH₂Cl,H], [A3423;H,H,SC(═S)N(i-Pr)(CH₂)₂Cl,H], [A3424;H,H,SC(═S)N(i-Pr)(CH₂)₃Cl,H], [A3425;H,H,SC(═S)N(i-Pr)(CH₂)₄Cl,H], [A3426;H,H,SC(═S)N(i-Pr)(CH₂)₅Cl,H], [A3427;H,H,SC(═S)N(i-Pr)CH₂Br,H], [A3428;H,H,SC(═S)N(i-Pr)(CH₂)₂Br,H], [A3429;H,H,SC(═S)N(i-Pr)(CH₂)₃Br,H], [A3430;H,H,SC(═S)N(i-Pr)(CH₂)₄Br,H], [A3431;H,H,SC(═S)N(i-Pr)(CH₂)₅Br,H], [A3432;H,H,SC(═S)N(i-Pr)CH₂OMe,H], [A3433;H,H,SC(═S)N(i-Pr)(CH₂)₂OMe,H], [A3434;H,H,SC(═S)N(i-Pr)(CH₂)₃OMe,H], [A3435;H,H,SC(═S)N(i-Pr)(CH₂)₄OMe,H], [A3436;H,H,SC(═S)N(i-Pr)CH₂OEt,H], [A3437;H,H,SC(═S)N(i-Pr)(CH₂)₂OEt,H], [A3438;H,H,SC(═S)N(i-Pr)(CH₂)₃OEt,H], [A3439;H,H,SC(═S)N(i-Pr)CH₂CN,H], [A3440;H,H,SC(═S)N(i-Pr)(CH₂)₂CN,H], [A3441;H,H,SC(═S)N(i-Pr)(CH₂)₃CN,H], [A3442;H,H,SC(═S)N(i-Pr)(CH₂)₄CN,H], [A3443;H,H,SC(═S)N(i-Pr)(CH₂)₅CN,H], [A3444;H,H,SC(═S)N(i-Pr)CH₂C═CHF,H], [A3445;H,H,SC(═S)N(i-Pr)CH₂C═CF₂,H], [A3446;H,H,SC(═S)N(i-Pr)(CH₂)₂C═CHF,H], [A3447;H,H,SC(═S)N(i-Pr) CHCH₂C═CF₂,H], [A3448;H,H,SC(═S)N(i-Pr)(CH₂)₃C═CHF,H], [A3449;H,H,SC(═S)N(i-Pr)CH₂CHCH₂C═CF₂,H], [A3450;H,H,SC(═S)N(i-Pr) CH₂C═CHCl,H], [A3451;H,H,SC(═S)N(i-Pr)CH₂C═CCl₂,H], [A3452;H,H,SC(═S)N(i-Pr)(CH₂₂C═CHCl,H], [A3453;H,H,SC(═S)N(i-Pr) CHCH₂C═CCl₂,H], [A3454;H,H,SC(═S)N(i-Pr)(CH₂)₃C═CHCl,H], [A3455;H,H,SC(═S)N(i-Pr) CH₂CHCH₂C═CCl₂,H], [A3456;H,H,SC(═S)N(i-Pr)CH₂C═CH,H], [A3457;H,H,SC(═S)N(i-Pr)CH(Me)C═CH,H], [A3458;H,H,SC(═S)N(i-Pr)CH₂C═CMe,H], [A3459;H,H,SC(═S)N(i-Pr) CH₂C=CEt,H], [A3460;H,H,SC(═S)N(i-Pr)CH₂C=CEt,H], [A3461;H,H,SC(═S)N(i-Pr)(CH₂)₂C═CH,H], [A3462;H,H,SC(═S)N(i-Pr)(CH₂)₂C═CMe,H], [A3463;H,H,SC(═S)N(i-Pr)(CH₂)₃C═CH,H], [A3464;H,H,SC(═S)N(i-Pr)(CH₂)₄C═CH,H], [A3465;H,H,SC(═S)N(i-Pr)c-Pr,H], [A3466;H,H,SC(═S)N(i-Pr)c-Bu,H], [A3467;H,H,SC(═S)N(i-Pr)c-Pen,H], [A3468;H,H,SC(═S)N(i-Pr)c-Hex,H], [A3469;H,H,SC(═S)N(i-Pr)c-Hep,H], [A3470;H,H,SC(═S)N(i-Pr) OCH₂F,H], [A3471;H,H,SC(═S)N(i-Pr)OCHF₂,H], [A3472;H,H,SC(═S)N(i-Pr)OCF₃,H], [A3473;H,H,SC(═S)N(i-Pr)O(CH₂)₂F,H], [A3474;H,H,SC(═S)N(i-Pr) OCH₂CHF₂,H], [A3475;H,H,SC(═S)N(i-Pr) OCH₂CF₃,H], [A3476;H,H,SC(═S)N(i-Pr)O(CH₂)₃F,H], [A3477;H,H,SC(═S)N(i-Pr)O(CH₂)₂CHF₂,H], [A3478;H,H,SC(═S)N(i-Pr)O(CH₂)₂CF₃,H], [A3479;H,H,SC(═S)N(i-Pr) OCH₂CH(CF₃)₂,H], [A3480;H,H,SC(═S)N(i-Pr) OCH₂CH═CH₂,H], [A3481;H,H,SC(═S)N(i-Pr) OCH₂CH═CHF,H], [A3482;H,H,SC(═S)N(i-Pr) OCH₂CH═CF₂,H], [A3483;H,H,SC(═S)N(i-Pr) OCH₂CH═CHCl,H], [A3484;H,H,SC(═S)N(i-Pr) OCH₂CH═CCl₂,H], [A3485;H,H,SC(═S)N(i-Pr)OCH₂C═CH,H], [A3486;H,H,SC(═S)N(i-Pr) OCH(Me)C═CH,H], [A3487;H,H,SC(═S)N(i-Pr) OCH₂C═CMe,H], [A3488;H,H,SC(═O)OMe,H], [A3489;H,H,SC(═O)OEt,H], [A3490;H,H,SC(═O)O(CH₂)₂Me,H], [A3491;H,H,SC(═O)O(CH₂)₃Me,H], [A3492;H,H,SC(═O)O(CH₂)₄Me,H], [A3493;H,H,SC(═O)OCH(Me)₂,H], [A3494;H,H,SC(═O)OCH₂CH(Me)₂,H], [A3495;H,H,SC(═O)O(CH₂)₂CH(Me)₂,H], [A3496;H,H,SC(═O)OC(Me)₃,H], [A3497;H,H,SC(═O)OCH₂C(Me)₃,H], [A3498;H,H,SC(═O)O(CH₂)₂C(Me)₃,H], [A3499;H,H,SC(═O)OCH(Me)Et,H][A3500;H,H,SC(═O)OCH(Me)(CH₂)₂Me,H],

[A3501;H,H,SC(═O)OCH(Me)(CH₂)₃Me,H], [A3502;H,H,SC(═O)OCH(Et)₂,H], [A3503;H,H,SC(═O)OCH₂CH═CH₂,H], [A3504;H,H,SC(═O)O(CH₂)₂CH═CH₂,H], [A3505;H,H,SC(═O)O(CH₂)₃CH═CH₂,H], [A3506;H,H,SC(═O)OCH₂CH═CHMe,H], [A3507;H,H,SC(═O)OCH₂CH═CHEt,H], [A3508;H,H,SC(═O)OCH₂CH═C(Me)₂,H][A3509;H,H,SC(═O)OCH₂CH═C(Me)Et,H], [A3510;H,H,SC(═O)OCH₂F,H], [A3511;H,H,SC(═O)OCHF₂,H], [A3512;H,H,SC(═O)OCF₃,H], [A3513;H,H,SC(═O)O(CH₂)₂F,H], [A3514;H,H,SC(═O)OCH₂CHF₂,H], [A3515;H,H,SC(═O)OCH₂CF₃,H], [A3516;H,H,SC(═O)O(CH₂)₃F,H], [A3517;H,H,SC(═O)O(CH₂)₂CHF₂,H][A3518;H,H,SC(═O)O(CH₂)₂CF₃,H], [A3519;H,H,SC(═O)OCH₂CH(CF₃)₂,H], [A3520;H,H,SC(═O)OCH₂Cl,H], [A3521;H,H,SC(═O)O(CH₂)₂Cl,H], [A3522;H,H,SC(═O)O(CH₂)₃Cl,H], [A3523;H,H,SC(═O)O(CH₂)₄Cl,H], [A3524;H,H,SC(═O)O(CH₂)₅Cl,H], [A3525;H,H,SC(═O)OCH₂Br,H], [A3526;H,H,SC(═O)O(CH₂)₂Br,H], [A3527;H,H,SC(═O)O(CH₂)₃Br,H], [A3528;H,H,SC(═O)O(CH₂)₄Br,H], [A3529;H,H,SC(═O)O(CH₂)₅Br,H], [A3530;H,H,SC(═O)OCH₂OMe,H], [A3531;H,H,SC(═O)O(CH₂)₂OMe,H], [A3532;H,H,SC(═O)O(CH₂)₃OMe,H], [A3533;H,H,SC(═O)O(CH₂)₄OMe,H], [A3534;H,H,SC(═O)OCH₂OEt,H], [A3535;H,H,SC(═O)O(CH₂)₂OEt,H], [A3536;H,H,SC(═O)O(CH₂)₃OEt,H], [A3537;H,H,SC(═O)OCH₂CN,H], [A3538;H,H,SC(═O)O(CH₂₂CN,H], [A3539;H,H,SC(═O)O(CH₂)₃CN,H], [A3540;H,H,SC(═O)O(CH₂)₄CN,H], [A3541;H,H,SC(═O)O(CH₂)₅CN,H], [A3542;H,H,SC(═O)OCH₂C═CHF,H], [A3543;H,H,SC(═O)OCH₂C═CF₂,H], [A3544;H,H,SC(═O)O(CH₂)₂C═CHF,H], [A3545;H,H,SC(═O)OCHCH₂C═CF₂,H], [A3546;H,H,SC(═O)O(CH₂)₃C═CHF,H], [A3547;H,H,SC(═O)OCH₂CHCH₂C═CF₂,H], [A3548;H,H,SC(═O)OCH₂C═CHCl,H], [A3549;H,H,SC(═O)OCH₂C═CCl₂,H], [A3550;H,H,SC(═O)O(CH₂)₂C═CHCl,H], [A3551;H,H,SC(═O)OCHCH₂C═CCl₂,H], [A3552;H,H,SC(═O)O(CH₂)₃C═CHCl,H], [A3553;H,H,SC(═O)OCH₂CHCH₂C═CCl₂,H], [A3554;H,H,SC(═O)OCH₂C═CH,H], [A3555;H,H,SC(═O)OCH(Me)C═CH,H], [A3556;H,H,SC(═O)OCH₂C═CMe,H], [A3557;H,H,SC(═O)OCH₂C=CEt,H], [A3558;H,H,SC(═O)OCH₂C=CEt,H][A3559;H,H,SC(═O)O(CH₂)₂C═CH,H], [A3560;H,H,SC(═O)O(CH₂)₂C═CMe,H], [A3561;H,H,SC(═O)O(CH₂)₃C═CH,H], [A3562;H,H,SC(═O)O(CH₂)₄C═CH,H], [A3563;H,H,SC(═O)Oc-Pr,H], [A3564;H,H,SC(═O)Oc-Bu,H], [A3565;H,H,SC(═O)Oc-Pen,H], [A3566;H,H,SC(═O)Oc-Hex,H], [A3567;H,H,SC(═O)Oc-Hep,H], [A3568;H,H,SC(═O)OMe,H], [A3569;H,H,SC(═O)OEt,H], [A3570;H,H,SC(═O)O(CH₂)₂Me,H], [A3571;H,H,SC(═O)O(CH₂)₃Me,H], [A3572;H,H,SC(═O)O(CH₂)₄Me,H], [A3573;H,H,SC(═O)OCH(Me)₂,H], [A3574;H,H,SC(═O)OCH₂CH(Me) 2,H][A3575;H,H,SC(═O)O(CH₂)₂CH(Me)₂,H], [A3576;H,H,SC(═O)OC(Me)₃,H], [A3577;H,H,SC(═O)OCH₂C(Me)₃,H], [A3578;H,H,SC(═O)O(CH₂)₂C(Me)₃,H], [A3579;H,H,SC(═O)OCH(Me)Et,H][A3580;H,H,SC(═O)OCH(Me)(CH₂)₂Me,H], [A3581;H,H,SC(═O)OCH(Me)(CH₂)₃Me,H], [A3582;H,H,SC(═O)OCH(Et)₂,H], [A3583;H,H,SC(═O)OCH₂CH═CH₂,H], [A3584;H,H,SC(═O)O(CH₂)₂CH═CH₂,H], [A3585;H,H,SC(═O)O(CH₂)₃CH═CH₂,H], [A3586;H,H,SC(═O)OCH₂CH═CHMe,H], [A3587;H,H,SC(═O)OCH₂CH═CHEt,H], [A3588;H,H,SC(═O)OCH₂CH═C(Me)₂,H], [A3589;H,H,SC(═O)OCH₂CH═C(Me)Et,H], [A3590;H,H,SC(═O)OCH₂F,H], [A3591;H,H,SC(═O)OCHF₂,H], [A3592;H,H,SC(═O)OCF₃,H], [A3593;H,H,SC(═O)O(CH₂₂F,H], [A3594;H,H,SC(═O)OCH₂CHF₂,H], [A3595;H,H,SC(═O)OCH₂CF₃,H], [A3596;H,H,SC(═O)O(CH₂)₃F,H], [A3597;H,H,SC(═O)O(CH₂)₂CHF₂,H], [A3598;H,H,SC(═O)O(CH₂)₂CF₃,H], [A3599;H,H,SC(═O)OCH₂CH(CF₃)₂,H], [A3600;H,H,SC(═O)OCH₂Cl,H],

[A3601;H,H,SC(═O)O(CH₂₂Cl,H], [A3602;H,H,SC(═O)O(CH₂)₃Cl,H], [A3603;H,H,SC(═O)O(CH₂)₄Cl,H], [A3604;H,H,SC(═O)O(CH₂)₅Cl,H], [A3605;H,H,SC(═O)OCH₂Br,H], [A3606;H,H,SC(═O)O(CH₂)₂Br,H], [A3607;H,H,SC(═O)O(CH₂)₃Br,H], [A3608;H,H,SC(═O)O(CH₂)₄Br,H], [A3609;H,H,SC(═O)O(CH₂)₅Br,H], [A3610;H,H,SC(═O)OCH₂OMe,H], [A3611;H,H,SC(═O)O(CH₂)₂OMe,H][A3612;H,H,SC(═O)O(CH₂)₃₀Me,H], [A3613;H,H,SC(═O)O(CH₂)₄OMe,H], [A3614;H,H,SC(═O)OCH₂OEt,H], [A3615;H,H,SC(═O)O(CH₂)₂OEt,H], [A3616;H,H,SC(═O)O(CH₂)₃OEt,H], [A3617;H,H,SC(═O)OCH₂CN,H], [A3618;H,H,SC(═O)O(CH₂)₂CN,H], [A3619;H,H,SC(═O)O(CH₂)₃CN,H], [A3620;H,H,SC(═O)O(CH₂)₄CN,H], [A3621;H,H,SC(═O)O(CH₂)₅CN,H], [A3622;H,H,SC(═O)OCH₂C═CHF,H], [A3623;H,H,SC(═O)OCH₂C═CF₂,H], [A3624;H,H,SC(═O)O(CH₂)₂C═CHF,H], [A3625;H,H,SC(═O)OCHCH₂C═CF₂,H], [A3626;H,H,SC(═O)O(CH₂)₃C═CHF,H], [A3627;H,H,SC(═O)OCH₂CHCH₂C═CF₂,H][A3628;H,H,SC(═O)OCH₂C═CHCl,H], [A3629;H,H,SC(═O)OCH₂C═CCl₂,H], [A3630;H,H,SC(═O)O(CH₂)₂C═CHCl,H], [A3631;H,H,SC(═O)OCHCH₂C═CCl₂,H][A3632;H,H,SC(═O)O(CH₂)₃C═CHCl,H], [A3633;H,H,SC(═O)OCH₂CHCH₂C═CCl₂,H], [A3634;H,H,SC(═O)OCH₂C═CH,H], [A3635;H,H,SC(═O)OCH(Me)C═CH,H], [A3636;H,H,SC(═O)OCH₂C═CMe,H], [A3637;H,H,SC(═O)OCH₂C=CEt,H], [A3638;H,H,SC(═O)OCH₂C=CEt,H][A3639;H,H,SC(═O)O(CH₂)₂C═CH,H], [A3640;H,H,SC(═O)O(CH₂)₂C═CMe,H], [A3641;H,H,SC(═O)O(CH₂)₃C═CH,H], [A3642;H,H,SC(═O)O(CH₂)₄C═CH,H], [A3643;H,H,SC(═O)Oc-Pr,H], [A3644;H,H,SC(═O)Oc-Bu,H], [A3645;H,H,SC(═O)Oc-Pen,H], [A3646;H,H,SC(═O)Oc-Hex,H], [A3647;H,H,SC(═O)Oc-Hep,H], [A3648;H,H,SC(═O)OMe,H], [A3649;H,H,SC(═O)OEt,H][A3650;H,H,SC(═O)O(CH₂)₂Me,H], [A3651;H,H,SC(═O)O(CH₂)₃Me,H], [A3652;H,H,SC(═O)O(CH₂)₄Me,H], [A3653;H,H,SC(═O)OCH(Me)₂,H], [A3654;H,H,SC(═O)OCH₂CH(Me)₂,H], [A3655;H,H,SC(═O)O(CH₂)₂CH(Me)₂,H], [A3656;H,H,SC(═O)OC(Me)₃,H], [A3657;H,H,SC(═O)OCH₂C(Me)₃,H], [A3658;H,H,SC(═O)O(CH₂)₂C(Me)₃,H], [A3659;H,H,SC(═O)OCH(Me)Et,H], [A3660;H,H,SC(═O)OCH(Me)(CH₂)₂Me,H], [A3661;H,H,SC(═O)OCH(Me)(CH₂)₃Me,H], [A3662;H,H,SC(═O)OCH(Et)₂,H], [A3663;H,H,SC(═O)OCH₂CH═CH₂,H], [A3664;H,H,SC(═O)O(CH₂)₂CH═CH₂,H], [A3665;H,H,SC(═O)O(CH₂)₃CH═CH₂,H], [A3666;H,H,SC (═O)OCH₂CH═CHMe,H], [A3667;H,H,SC(═O)OCH₂CH═CHEt,H], [A3668;H,H,SC(═O)OCH₂CH═C(Me)₂,H][A3669;H,H,SC(═O)OCH₂CH═C(Me)Et,H], [A3670;H,H,SC(═O)OCH₂F,H], [A3671;H,H,SC(═O)OCHF₂,H], [A3672;H,H,SC(═O)OCF₃,H], [A3673;H,H,SC(═O)O(CH₂)₂F,H], [A3674;H,H,SC(═O)OCH₂CHF₂,H], [A3675;H,H,SC(═O)OCH₂CF₃,H], [A3676;H,H,SC(═O)O(CH₂)₃F,H], [A3677;H,H,SC(═O)O(CH₂)₂CHF₂,H], [A3678;H,H,SC(═O)O(CH₂)₂CF₃,H], [A3679;H,H,SC(═O)OCH₂CH(CF₃)₂,H], [A3680;H,H,SC(═O)OCH₂Cl,H], [A3681;H,H,SC(═O)O(CH₂₂Cl,H], [A3682;H,H,SC(═O)O(CH₂)₃Cl,H], [A3683;H,H,SC(═O)O(CH₂)₄Cl,H], [A3684;H,H,SC(═O)O(CH₂)₅Cl,H], [A3685;H,H,SC(═O)OCH₂Br,H], [A3686;H,H,SC(═O)O(CH₂)₂Br,H], [A3687;H,H,SC(═O)O(CH₂)₃Br,H], [A3688;H,H,SC(═O)O(CH₂)₄Br,H], [A3689;H,H,SC(═O)O(CH₂)₅Br,H], [A3690;H,H,SC(═O)OCH₂OMe,H], [A3691;H,H,SC(═O)O(CH₂)₂OMe,H][A3692;H,H,SC(═O)O(CH₂)₃₀Me,H], [A3693;H,H,SC(═O)O(CH₂)₄OMe,H], [A3694;H,H,SC(═O)OCH₂OEt,H], [A3695;H,H,SC(═O)O(CH₂O Et,H], [A3696;H,H,SC(═O)O(CH₂)₃OEt,H], [A3697;H,H,SC(═O)OCH₂CN,H], [A3698;H,H,SC(═O)O(CH₂)₂CN,H], [A3699;H,H,SC(═O)O(CH₂)₃CN,H], [A3700;H,H,SC(═O)O(CH₂)₄CN,H],

[A3701;H,H,SC(═O)O(CH₂)₅CN,H], [A3702;H,H,SC(═O)OCH₂C═CHF,H], [A3703;H,H,SC(═O)OCH₂C═CF₂,H], [A3704;H,H,SC(═O)O(CH₂)₂C═CHF,H], [A3705;H,H,SC(═O)OCHCH₂C═CF₂,H], [A3706;H,H,SC(═O)O(CH₂)C═CHF,H], [A3707;H,H,SC(═O)OCH₂CHCH₂C═CF₂,H][A3708;H,H,SC(═O)OCH₂C═CHCl,H], [A3709;H,H,SC(═O)OCH₂C═CCl₂,H], [A3710;H,H,SC(═O)O(CH₂)₂C═CHCl,H], [A3711;H,H,SC(═O)OCHCH₂C═CCl₂,H], [A3712;H,H,SC(═O)O(CH₂)₃C═CHCl,H], [A3713;H,H,SC(═O)OCH₂CHCH₂C═CCl₂,H], [A3714;H,H,SC(═O)OCH₂C═CH,H], [A3715;H,H,SC(═O)OCH(Me)C═CH,H], [A3716;H,H,SC(═O)OCH₂C═CMe,H], [A3717;H,H,SC(═O)OCH₂C=CEt,H], [A3718;H,H,SC(═O)OCH₂C=CEt,H][A3719;H,H,SC(═O)O(CH₂)₂C═CH,H], [A3720;H,H,SC(═O)O(CH₂)₂C═CMe,H], [A3721;H,H,SC(═O)O(CH₂)₃C═CH,H], [A3722;H,H,SC(═O)O(CH₂)₄C═CH,H], [A3723;H,H,SC(═O)Oc-Pr,H], [A3724;H,H,SC(═O)Oc-Bu,H], [A3725;H,H,SC(═O)Oc-Pen,H], [A3726;H,H,SC(═O)Oc-Hex,H], [A3727;H,H,SC(═O)Oc-Hep,H], [A3728;H,H,SC(═O)OMe,H], [A3729;H,H,SC(═O)OEt,H], [A3730;H,H,SC(═O)O(CH₂)₂Me,H], [A3731;H,H,SC(═O)O(CH₂)₃Me,H], [A3732;H,H,SC(═O)O(CH₂)₄Me,H], [A3733;H,H,SC(═O)OCH(Me)₂,H], [A3734;H,H,SC(═OCH₂CH(Me)₂,H], [A3735;H,H,SC(═O)O(CH₂)₂CH(Me)₂,H], [A3736;H,H,SC(═O)OC(Me)₃,H], [A3737;H,H,SC(═O)OCH₂C(Me)₃,H], [A3738;H,H,SC(═)O(CH)₂C(Me)₃,H], [A3739;H,H,SC(═O)OCH(Me)Et,H][A3740;H,H,SC(═O)OCH(Me)(CH₂)₂Me,H], [A3741;H,H,SC(═O)OCH(Me)(CH₂)₃Me,H], [A3742;H,H,SC(═O)OCH(Et)₂,H], [A3743;H,H,SC(═O)OCH₂CH═CH₂,H], [A3744;H,H,SC(═O)O(CH₂)₂CH═CH₂,H], [A3745;H,H,SC(═O)O(CH₂)₃CH═CH₂,H], [A3746;H,H,SC(═O)OCH₂CH═CHMe,H], [A3747;H,H,SC(═O)OCH₂CH═CHEt,H][A3748;H,H,SC(═O)OCH₂CH═C(Me)₂,H], [A3749;H,H,SC(═O)OCH₂CH═C(Me)Et,H], [A3750;H,H,SC(═O)OCH₂F,H], [A3751;H,H,SC(═O)OCHF₂,H], [A3752;H,H,SC(═O)OCF₃,H], [A3753;H,H,SC(═)O(CH)₂F,H], [A3754;H,H,SC(═O)OCH₂CHF₂,H], [A3755;H,H,SC(═O)OCH₂CF₃,H], [A3756;H,H,SC(═O)O(CH)₃F,H], [A3757;H,H,SC(═O)O(CH₂)₂CHF₂,H], [A3758;H,H,SC(═O)O(CH₂)₂CF₃,H], [A3759;H,H,SC(═O)OCH₂CH(CF)₂,H], [A3760;H,H,SC(═O)OCH₂Cl,H], [A3761;H,H,SC(═O)O(CH₂)₂Cl,H], [A3762;H,H,SC(═O)O(CH₂)₃Cl,H], [A3763;H,H,SC(═O)O(CH₂)₄Cl,H], [A3764;H,H,SC(═O)O(CH₂)₅Cl,H], [A3765;H,H,SC(═O)OCH₂Br,H], [A3766;H,H,SC(═)O(CH)₂Br,H], [A3767;H,H,SC(═O)O(CH₂)₃Br,H], [A3768;H,H,SC(═)O(CH)₄Br,H], [A3769;H,H,SC(═O)O(CH₂)₅Br,H], [A3770;H,H,SC(═O)OCH₂OMe,H], [A3771;H,H,SC(═O)O(CH₂)₂OMe,H], [A3772;H,H,SC(═O)O(CH₂)₃OMe,H], [A3773;H,H,SC(═O)O(CH₂)₄OMe,H], [A3774;H,H,SC(═O)OCH₂OEt,H], [A3775;H,H,SC(═O)O(CH₂)₂OEt,H], [A3776;H,H,SC(═O)O(CH₂)₃OEt,H], [A3777;H,H,SC(═O)OCH₂CN,H], [A3778;H,H,SC(═O)O(CH₂)₂CN,H], [A3779;H,H,SC(═O)O(CH₂)₃CN,H], [A3780;H,H,SC(═O)O(CH₂)₄CN,H], [A3781;H,H,SC(═O)O(CH₂)₅CN,H], [A3782;H,H,SC(═O)OCH₂C═CHF,H], [A3783;H,H,SC(═O)OCH₂C═CF₂,H], [A3784;H,H,SC(═O)O(CH₂)₂C═CHF,H], [A3785;H,H,SC(═O) 0CHCH₂C═CF₂,H], [A3786;H,H,SC(═O)O(CH₂)₃C═CHF,H], [A3787;H,H,SC(═O)OCH₂CHCH₂C═CF₂,H][A3788;H,H,SC(═O)OCH₂C═CHCl,H], [A3789;H,H,SC(═O)OCH₂C═CCl₂,H][A3790;H,H,SC(═O)O(CH₂)₂C═CHCl,H], [A3791;H,H,SC(═O)OCHCH₂C═CCl₂,H], [A3792;H,H,SC(═O)O(CH₂)₃C═CHCl,H], [A3793;H,H,SC(═O)OCH₂CHCH₂C═CCl₂,H], [A3794;H,H,SC(═O)OCH₂C═CH,H], [A3795;H,H,SC(═O)OCH(Me)C═CH,H], [A3796;H,H,SC(═O)OCH₂C═CMe,H], [A3797;H,H,SC(═O)OCH₂C=CEt,H], [A3798;H,H,SC(═O)OCH₂C=CEt,H][A3799;H,H,SC(═O)O(CH₂)₂C═CH,H], [A3800;H,H,SC(═O)O(CH₂)₂C═CMe,H], [A3801;H,H,SC(═O)O(CH₂)₃C═CH,H], [A3802;H,H,SC(═O)O(CH₂)₄C═CH,H], [A3803;H,H,SC(═O)Oc-Pr,H], [A3804;H,H,SC(═O)Oc-Bu,H], [A3805;H,H,SC(═O)Oc-Pen,H], [A3806;H,H,SC(═O)Oc-Hex,H], [A3807;H,H,SC(═O)Oc-Hep,H], [A3808;H,H,SC(═O)OMe,H], [A3809;H,H,SC(═O)OEt,H], [A3810;H,H,SC(═O)O(CH₂)₂Me,H], [A3811;H,H,SC(═O)O(CH₂)₃Me,H], [A3812;H,H,SC(═O)O(CH₂)₄Me,H], [A3813;H,H,SC(═O)OCH(Me)₂,H], [A3814;H,H,SC(═O)OCH₂CH(Me) 2,H], [A3815;H,H,SC(═O)O(CH₂)₂CH(Me)₂,H], [A3816;H,H,SC(═O)OC(Me)₃,H], [A3817;H,H,SC(═O)OCH₂C(Me)₃,H], [A3818;H,H,SC(═O)O(CH₂)₂C(Me)₃,H], [A3819;H,H,SC(═O)OCH(Me)Et,H][A3820;H,H,SC(═O)OCH(Me)(CH₂)₂Me,H], [A3821;H,H,SC(═O)OCH(Me)(CH₂)₃Me,H], [A3822;H,H,SC(═O)OCH(Et)₂,H], [A3823;H,H,SC(═O)OCH₂CH═CH₂,H], [A3824;H,H,SC(═O)O(CH₂)₂CH═CH₂,H], [A3825;H,H,SC(═O)O(CH₂)₃CH═CH₂,H], [A3826;H,H,SC (═O)OCH₂CH═CHMe,H], [A3827;H,H,SC(═O)OCH₂CH═CHEt,H], [A3828;H,H,SC(═O)OCH₂CH═C(Me)₂,H][A3829;H,H,SC(═O)OCH₂CH═C(Me)Et,H], [A3830;H,H,SC(═O)OCH₂F,H], [A3831;H,H,SC(═O)OCHF₂,H], [A3832;H,H,SC(═O)OCF₃,H], [A3833;H,H,SC(═O)O(CH₂₂F,H], [A3834;H,H,SC(═O)OCH₂CHF₂,H], [A3835;H,H,SC(═O)OCH₂CF₃,H], [A3836;H,H,SC(═O)O(CH₂)₃F,H], [A3837;H,H,SC(═O)O(CH₂)₂CHF₂,H], [A3838;H,H,SC(═O)O(CH₂)₂CF₃,H], [A3839;H,H,SC(═O)OCH₂CH(CF₃)₂,H], [A3840;H,H,SC(═O)OCH₂Cl,H], [A3841;H,H,SC(═O)O(CH₂)₂Cl,H], [A3842;H,H,SC(═O)O(CH₂)₃Cl,H], [A3843;H,H,SC(═O)O(CH₂)₄Cl,H], [A3844;H,H,SC(═O)O(CH₂)₅Cl,H], [A3845;H,H,SC(═O)OCH₂Br,H], [A3846;H,H,SC(═O)O(CH₂)₂Br,H], [A3847;H,H,SC(═O)O(CH₂)₃Br,H], [A3848;H,H,SC(═O)O(CH₂)₄Br,H], [A3849;H,H,SC(═O)O(CH₂)₅Br,H], [A3850;H,H,SC(═O)OCH₂OMe,H], [A3851;H,H,SC(═O)O(CH₂)₂OMe,H], [A3852;H,H,SC(═O)O(CH₂)₃OMe,H], [A3853;H,H,SC(═O)O(CH₂)₄OMe,H], [A3854;H,H,SC(═O)OCH₂OEt,H], [A3855;H,H,SC(═O)O(CH₂)₂OEt,H], [A3856;H,H,SC(═O)O(CH₂)₃OEt,H], [A3857;H,H,SC(═O)OCH₂CN,H], [A3858;H,H,SC(═O)O(CH₂)₂CN,H], [A3859;H,H,SC(═O)O(CH₂)₃CN,H], [A3860;H,H,SC(═O)O(CH₂)₄CN,H], [A3861;H,H,SC(═O)O(CH₂)₅CN,H], [A3862;H,H,SC(═O)OCH₂C═CHF,H], [A3863;H,H,SC(═O)OCH₂C═CF₂,H], [A3864;H,H,SC(═O)O(CH₂)₂C═CHF,H][A3865;H,H,SC(═O)OCHCH₂C═CF₂,H], [A3866;H,H,SC(═O)O(CH₂)₃C═CHF,H], [A3867;H,H,SC(═O)OCH₂CHCH₂C═CF₂,H], [A3868;H,H,SC(═O)OCH₂C═CHCl,H], [A3869;H,H,SC(═O)OCH₂C═CCl₂,H], [A3870;H,H,SC(═O)O(CH₂)₂C═CHCl,H], [A3871;H,H,SC(═O)OCHCH₂C═CCl₂,H], [A3872;H,H,SC(═O)O(CH₂)₃C═CHCl,H], [A3873;H,H,SC(═O)OCH₂CHCH₂C═CCl₂,H], [A3874;H,H,SC(═O)OCH₂C═CH,H], [A3875;H,H,SC(═O)OCH(Me)C═CH,H], [A3876;H,H,SC(═O)OCH₂C═CMe,H], [A3877;H,H,SC(═O)OCH₂C=CEt,H], [A3878;H,H,SC(═O)OCH₂C=CEt,H], [A3879;H,H,SC(═O)O(CH₂)₂C═CH,H], [A3880;H,H,SC(═O)O(CH₂)₂C═CMe,H], [A3881;H,H,SC(═O)O(CH₂)₃C═CH,H], [A3882;H,H,SC(═O)O(CH₂)₄C═CH,H], [A3883;H,H,SC(═O)Oc-Pr,H], [A3884;H,H,SC(═O)Oc-Bu,H], [A3885;H,H,SC(═O)Oc-Pen,H], [A3886;H,H,SC(═O)Oc-Hex,H], [A3887;H,H,SC(═O)Oc-Hep,H], [A3888;H,H,SC(═S)OMe,H], [A3889;H,H,SC(═S)OEt,H], [A3890;H,H,SC(═S)O(CH₂)₂Me,H], [A3891;H,H,SC(═S)O(CH₂)₃Me,H], [A3892;H,H,SC(═S)O(CH₂)₄Me,H], [A3893;H,H,SC(═S)OCH(Me)₂,H], [A3894;H,H,SC(═S)OCH₂CH(Me) 2,H][A3895;H,H,SC(═S)O(CH₂)₂CH(Me)₂,H], [A3896;H,H,SC(═S)OC(Me)₃,H], [A3897;H,H,SC(═S)OCH₂C(Me)₃,H], [A3898;H,H,SC(═S)O(CH₂)₂C(Me)₃,H], [A3899;H,H,SC(═S)OCH(Me)Et,H][A3900;H,H,SC(═S)OCH(Me)(CH₂)₂Me,H],

[A3901;H,H,SC(═S)OCH(Me)(CH₂)₃Me,H], [A3902;H,H,SC(═S)OCH(Et)₂,H], [A3903;H,H,SC(═S)OCH₂CH═CH₂,H], [A3904;H,H,SC(═S)O(CH₂)₂CH═CH₂,H], [A3905;H,H,SC(═S)O(CH₂)₃CH═CH₂,H][A3906;H,H,SC(═S)OCH₂CH═CHMe,H], [A3907;H,H,SC(═S)OCH₂CH═CHEt,H], [A3908;H,H,SC(═S)OCH₂CH═C(Me)₂,H][A3909;H,H,SC(═S)OCH₂CH═C(Me)Et,H], [A3910;H,H,SC(═S)OCH₂F,H], [A3911;H,H,SC(═S)OCHF₂,H], [A3912;H,H,SC(═S)OCF₃,H], [A3913;H,H,SC(═S)O(CH₂)₂F,H], [A3914;H,H,SC(═S)OCH₂CHF₂,H], [A3915;H,H,SC(═S)OCH₂CF₃,H], [A3916;H,H,SC(═S)O(CH₂)₃F,H], [A3917;H,H,SC(═S)O(CH₂)₂CHF₂,H], [A3918;H,H,SC(═S)O(CH₂)₂CF₃,H], [A3919;H,H,SC(═S)OCH₂CH(CF₃)₂,H], [A3920;H,H,SC(═S)OCH₂Cl,H], [A3921;H,H,SC(═S)O(CH₂)₂Cl,H], [A3922;H,H,SC(═S)O(CH₂)₃Cl,H], [A3923;H,H,SC(═S)O(CH₂)₄Cl,H], [A3924;H,H,SC(═S)O(CH₂)₅Cl,H], [A3925;H,H,SC(═S)OCH₂Br,H], [A3926;H,H,SC(═S)O(CH₂)₂Br,H], [A3927;H,H,SC(═S)O(CH₂)₃Br,H], [A3928;H,H,SC(═S)O(CH₂)₄Br,H], [A3929;H,H,SC(═S)O(CH₂)₅Br,H], [A3930;H,H,SC(═S)OCH₂OMe,H], [A3931;H,H,SC(═S)O(CH₂)₂OMe,H], [A3932;H,H,SC(═S)O(CH₂)₃OMe,H][A3933;H,H,SC(═S)O(CH₂)₄OMe,H], [A3934;H,H,SC(═S)OCH₂OEt,H], [A3935;H,H,SC(═S)O(CH₂)₂OEt,H][A3936;H,H,SC(═S)O(CH₂)₃OEt,H], [A3937;H,H,SC(═S)OCH₂CN,H], [A3938;H,H,SC(═S)O(CH₂)₂CN,H], [A3939;H,H,SC(═S)O(CH₂)₃CN,H], [A3940;H,H,SC(═S)O(CH₂)₄CN,H], [A3941;H,H,SC(═S)O(CH₂)₅CN,H], [A3942;H,H,SC(═S)OCH₂C═CHF,H], [A3943;H,H,SC(═S)OCH₂C═CF₂,H], [A3944;H,H,SC(═S)O(CH₂)₂C═CHF,H][A3945;H,H,SC(═S)OCHCH₂C═CF₂,H], [A3946;H,H,SC(═S)O(CH₂)₃C═CHF,H], [A3947;H,H,SC(═S)OCH₂CHCH₂C═CF₂,H], [A3948;H,H,SC(═S)OCH₂C═CHCl,H], [A3949;H,H,SC(═S)OCH₂C═CCl₂,H], [A3950;H,H,SC(═S)O(CH₂)₂C═CHCl,H], [A3951;H,H,SC(═S)OCHCH₂C═CCl₂,H], [A3952;H,H,SC(═S)O(CH₂)₃C═CHCl,H], [A3953;H,H,SC(═S)OCH₂CHCH₂C═CCl₂,H], [A3954;H,H,SC(═S)OCH₂C═CH,H], [A3955;H,H,SC(═S)OCH(Me)C═CH,H], [A3956;H,H,SC(═S)OCH₂C═CMe,H], [A3957;H,H,SC(═S)OCH₂C=CEt,H], [A3958;H,H,SC(═S)OCH₂C=CEt,H], [A3959;H,H,SC(═S)O(CH₂)₂C═CH,H], [A3960;H,H,SC(═S)O(CH₂)₂C═CMe,H], [A3961;H,H,SC(═S)O(CH₂)₃C═CH,H], [A3962;H,H,SC(═S)O(CH₂)₄C═CH,H], [A3963;H,H,SC(═S)Oc-Pr,H], [A3964;H,H,SC(═S)Oc-Bu,H], [A3965;H,H,SC(═S)Oc-Pen,H], [A3966;H,H,SC(═S)Oc-Hex,H], [A3967;H,H,SC(═S)Oc-Hep,H], [A3968;H,H,SC(═S)OMe,H], [A3969;H,H,SC(═S)OEt,H], [A3970;H,H,SC(═S)O(CH₂)₂Me,H], [A3971;H,H,SC(═S)O(CH₂)₃Me,H], [A3972;H,H,SC(═S)O(CH₂)₄Me,H], [A3973;H,H,SC(═S)OCH(Me)₂,H], [A3974;H,H,SC(═S)OCH₂CH(Me)₂,H], [A3975;H,H,SC(═S)O(CH₂)₂CH(Me)₂,H], [A3976;H,H,SC(═S)OC(Me),H], [A3977;H,H,SC(═S)OCH₂C(Me),H], [A3978;H,H,SC(═S)O(CH₂)₂C(Me)₃,H], [A3979;H,H,SC(═S)OCH(Me)Et,H][A3980;H,H,SC(═S)OCH(Me)(CH₂)₂Me,H], [A3981;H,H,SC(═S)OCH(Me)(CH₂)₃Me,H], [A3982;H,H,SC(═S)OCH(Et)₂,H], [A3983;H,H,SC(═S)OCH₂CH═CH₂,H], [A3984;H,H,SC(═S)O(CH₂)₂CH═CH₂,H][A3985;H,H,SC(═S)O(CH₂)₃CH═CH₂,H][A3986;H,H,SC(═S)OCH₂CH═CHMe,H], [A3987;H,H,SC(═S)OCH₂CH═CHEt,H], [A3988;H,H, S C(═S)OCH₂CH═C(Me)₂,H][A3989;H,H,SC(═S)OCH₂CH═C(Me)Et,H], [A3990;H,H,SC(═S)OCH₂F,H], [A3991;H,H,SC(═S)OCHF₂,H], [A3992;H,H,SC(═S)OCF₃,H], [A3993;H,H,SC(═S)O(CH₂)₂F,H], [A3994;H,H,SC(═S)OCH₂CHF₂,H], [A3995;H,H,SC(═S)OCH₂CF₃,H], [A3996;H,H,SC(═S)O(CH₂)₃F,H], [A3997;H,H,SC(═S)O(CH₂)₂CHF₂,H], [A3998;H,H,SC(═S)O(CH₂)₂CF₃,H], [A3999;H,H,SC(═S)OCH₂CH(CF₃)₂,H], [A4000;H,H,SC(═S)OCH₂Cl,H],

[A4001;H,H,SC(═S)O(CH₂)₂Cl,H], [A4002;H,H,SC(═S)O(CH₂)₃Cl,H], [A4003;H,H,SC(═S)O(CH₂)₄Cl,H], [A4004;H,H,SC(═S)O(CH₂)₅Cl,H], [A4005;H,H,SC(═S)OCH₂Br,H], [A4006;H,H,SC(═S)O(CH₂)₂Br,H], [A4007;H,H,SC(═S)O(CH₂)₃Br,H], [A4008;H,H,SC(═S)O(CH₂)₄Br,H], [A4009;H,H,SC(═S)O(CH₂)₅Br,H], [A4010;H,H,SC(═S)OCH₂OMe,H], [A4011;H,H,SC(═S)O(CH₂)₂OMe,H][A4012;H,H,SC(═S)O(CH₂)₃OMe,H], [A4013;H,H,SC(═S)O(CH₂)₄OMe,H], [A4014;H,H,SC(═S)OCH₂OEt,H], [A4015;H,H,SC(═S)O(CH₂)₂OEt,H], [A4016;H,H,SC(═S)O(CH₂)₃OEt,H], [A4017;H,H,SC(═S)OCH₂CN,H], [A4018;H,H,SC(═S)O(CH₂)₂CN,H], [A4019;H,H,SC(═S)O(CH₂)₃CN,H], [A4020;H,H,SC(═S)O(CH₂)₄CN,H], [A4021;H,H,SC(═S)O(CH₂)₅CN,H], [A4022;H,H,SC(═S)OCH₂C═CHF,H], [A4023;H,H,SC(═S)OCH₂C═CF₂,H], [A4024;H,H,SC(═S)O(CH₂)₂C═CHF,H], [A4025;H,H,SC(═S)OCHCH₂C═CF₂,H], [A4026;H,H,SC(═S)O(CH₂)₃C═CHF,H], [A4027;H,H,SC(═S)OCH₂CHCH₂C═CF₂,H], [A4028;H,H,SC(═S)OCH₂C═CHCl,H], [A4029;H,H,SC(═S)OCH₂C═CCl₂,H], [A4030;H,H,SC(═S)O(CH₂)₂C═CHCl,H], [A4031;H,H,SC(═S)OCHCH₂C═CCl₂,H][A4032;H,H,SC(═S)O(CH₂)₃C═CHCl,H], [A4033;H,H,SC(═S)OCH₂CHCH₂C═CCl₂,H], [A4034;H,H,SC(═S)OCH₂C═CH,H], [A4035;H,H,SC(═S)OCH(Me)C═CH,H], [A4036;H,H,SC(═S)OCH₂C═CMe,H], [A4037;H,H,SC(═S)OCH₂C=CEt,H], [A4038;H,H,SC(═S)OCH₂C=CEt,H], [A4039;H,H,SC(═S)O(CH₂)₂C═CH,H], [A4040;H,H,SC(═S)O(CH₂)₂C═CMe,H], [A4041;H,H,SC(═S)O(CH₂)₃C═CH,H], [A4042;H,H,SC(═S)O(CH₂)₄C═CH,H], [A4043;H,H,SC(═S)Oc-Pr,H], [A4044;H,H,SC(═S)Oc-Bu,H], [A4045;H,H,SC(═S)Oc-Pen,H], [A4046;H,H,SC(═S)Oc-Hex,H], [A4047;H,H,SC(═S)Oc-Hep,H], [A4048;H,H,SC(═S)OMe,H], [A4049;H,H,SC(═S)OEt,H], [A4050;H,H,SC(═S)O(CH₂)₂Me,H], [A4051;H,H,SC(═S)O(CH₂)₃Me,H], [A4052;H,H,SC(═S)O(CH₂)₄Me,H], [A4053;H,H,SC(═S)OCH(Me)₂,H], [A4054;H,H,SC(═S)OCH₂CH(Me) 2,H][A4055;H,H,SC(═S)O(CH₂)₂CH(Me)₂,H], [A4056;H,H,SC(═S)OC(Me)₃,H], [A4057;H,H,SC(═S)OCH₂C(Me)₃,H], [A4058;H,H,SC(═S)O(CH₂)₂C(Me)₃,H], [A4059;H,H,SC(═S)OCH(Me)Et,H], [A4060;H,H,SC(═S)OCH(Me)(CH₂)₂Me,H], [A4061;H,H,SC(═S)OCH(Me)(CH₂)₃Me,H], [A4062;H,H,SC(═S)OCH(Et)₂,H], [A4063;H,H,SC(═S)OCH₂CH═CH₂,H], [A4064;H,H,SC(═S)O(CH₂)₂CH═CH₂,H], [A4065;H,H,SC(═S)O(CH₂)₃CH═CH₂,H], [A4066;H,H,SC(═S)OCH₂CH═CHMe,H], [A4067;H,H,SC(═S)OCH₂CH═CHEt,H], [A4068;H,H,SC(═S)OCH₂CH═C(Me)₂,H], [A4069;H,H,SC(═S)OCH₂CH═C(Me)Et,H], [A4070;H,H,SC(═S)OCH₂F,H], [A4071;H,H,SC(═S)OCHF₂,H], [A4072;H,H,SC(═S)OCF₃,H], [A4073;H,H,SC(═S)O(CH₂)₂F,H], [A4074;H,H,SC(═S)OCH₂CHF₂,H], [A4075;H,H,SC(═S)OCH₂CF₃,H], [A4076;H,H,SC(═S)O(CH₂)₃F,H], [A4077;H,H,SC(═S)O(CH₂)₂CHF₂,H], [A4078;H,H,SC(═S)O(CH₂)₂CF₃,H], [A4079;H,H,SC(═S)OCH₂CH(CF₃)₂,H], [A4080;H,H,SC(═S)OCH₂Cl,H], [A4081;H,H,SC(═S)O(CH₂)₂Cl,H], [A4082;H,H,SC(═S)O(CH₂)₃Cl,H], [A4083;H,H,SC(═S)O(CH₂)₄Cl,H], [A4084;H,H,SC(═S)O(CH₂)₅Cl,H], [A4085;H,H,SC(═S)OCH₂Br,H], [A4086;H,H,SC(═S)O(CH₂)₂Br,H], [A4087;H,H,SC(═S)O(CH₂)₃Br,H], [A4088;H,H,SC(═S)O(CH₂)₄Br,H], [A4089;H,H,SC(═S)O(CH₂)₅Br,H], [A4090;H,H,SC(═S)OCH₂OMe,H], [A4091;H,H,SC(═S)O(CH₂)₂OMe,H], [A4092;H,H,SC(═S)O(CH₂)₃OMe,H], [A4093;H,H,SC(═S)O(CH₂)₄OMe,H], [A4094;H,H,SC(═S)OCH₂OEt,H], [A4095;H,H,SC(═S)O(CH₂)₂OEt,H], [A4096;H,H,SC(═S)O(CH₂)₃OEt,H], [A4097;H,H,SC(═S)OCH₂CN,H], [A4098;H,H,SC(═S)O(CH₂)₂CN,H], [A4099;H,H,SC(═S)O(CH₂)₃CN,H], [A4100;H,H,SC(═S)O(CH₂)₄CN,H],

[A4101;H,H,SC(═S)O(CH₂)₅CN,H], [A4102;H,H,SC(═S)OCH₂C═CHF,H], [A4103;H,H,SC(═S)OCH₂C═CF₂,H], [A4104;H,H,SC(═S)O(CH₂)₂C═CHF,H], [A4105;H,H,SC(═S)OCHCH₂C═CF₂,H], [A4106;H,H,SC(═S)O(CH₂)₃C═CHF,H], [A4107;H,H,SC(═S)OCH₂CHCH₂C═CF₂,H], [A4108;H,H,SC(═S)OCH₂C═CHCl,H], [A4109;H,H,SC(═S)OCH₂C═CCl₂,H], [A4110;H,H,SC(═S)O(CH₂)₂C═CHCl,H], [A4111;H,H,SC(═S)OCHCH₂C═CCl₂,H], [A4112;H,H,SC(═S)O(CH₂)₃C═CHCl,H], [A4113;H,H,SC(═S)OCH₂CHCH₂C═CCl₂,H], [A4114;H,H,SC(═S)OCH₂C═CH,H], [A4115;H,H,SC(═S)OCH(Me)C═CH,H], [A4116;H,H,SC(═S)OCH₂C═CMe,H], [A4117;H,H,SC(═S)OCH₂C=CEt,H], [A4118;H,H,SC(═S)OCH₂C=CEt,H], [A4119;H,H,SC(═S)O(CH₂)₂C═CH,H], [A4120;H,H,SC(═S)O(CH₂)₂C═CMe,H], [A4121;H,H,SC(═S)O(CH₂)₃C═CH,H], [A4122;H,H,SC(═S)O(CH₂)₄C═CH,H], [A4123;H,H,SC(═S)Oc-Pr,H], [A4124;H,H,SC(═S)Oc-Bu,H], [A4125;H,H,SC(═S)Oc-Pen,H], [A4126;H,H,SC(═S)Oc-Hex,H], [A4127;H,H,SC(═S)Oc-Hep,H], [A4128;H,H,SC(═S)OMe,H], [A4129;H,H,SC(═S)OEt,H], [A4130;H,H,SC(═S)O(CH₂)₂Me,H], [A4131;H,H,SC(═S)O(CH₂)₃Me,H], [A4132;H,H,SC(═S)O(CH₂)₄Me,H], [A4133;H,H,SC(═S)OCH(Me)₂,H], [A4134;H,H,SC(═S)OCH₂CH(Me) 2,H][A4135;H,H,SC(═S)O(CH₂)₂CH(Me)₂,H], [A4136;H,H,SC(═S)OC(Me)₃,H], [A4137;H,H,SC(═S)OCH₂C(Me)₃,H], [A4138;H,H,SC(═S)O(CH₂)₂C(Me)₃,H], [A4139;H,H,SC(═S)OCH(Me)Et,H], [A4140;H,H,SC(═S)OCH(Me)(CH₂)₂Me,H], [A4141;H,H,SC(═S)OCH(Me)(CH₂)₃Me,H], [A4142;H,H,SC(═S)OCH(Et)₂,H], [A4143;H,H,SC(═S)OCH₂CH═CH₂,H], [A4144;H,H,SC(═S)O(CH₂)₂CH═CH₂,H], [A4145;H,H,SC(═S)O(CH₂)₃CH═CH₂,H], [A4146;H,H,SC(═S)OCH₂CH═CHMe,H], [A4147;H,H,SC(═S)OCH₂CH═CHEt,H], [A4148;H,H,SC(═S)OCH₂CH═C(Me)₂,H], [A4149;H,H,SC(═S)OCH₂CH═C(Me)Et,H], [A4150;H,H,SC(═S)OCH₂F,H], [A4151;H,H,SC(═S)OCHF₂,H], [A4152;H,H,SC(═S)OCF₃,H], [A4153;H,H,SC(═S)O(CH₂)₂F,H], [A4154;H,H,SC(═S)OCH₂CHF₂,H], [A4155;H,H,SC(═S)OCH₂CF₃,H], [A4156;H,H,SC(═S)O(CH₂)₃F,H], [A4157;H,H,SC(═S)O(CH₂)₂CHF₂,H], [A4158;H,H,SC(═S)O(CH₂)₂CF₃,H][A4159;H,H,SC(═S)OCH₂CH(CF₃)₂,H], [A4160;H,H,SC(═S)OCH₂Cl,H], [A4161;H,H,SC(═S)O(CH₂)₂Cl,H], [A4162;H,H,SC(═S)O(CH₂)₃Cl,H], [A4163;H,H,SC(═S)O(CH₂)₄Cl,H], [A4164;H,H,SC(═S)O(CH₂)₅Cl,H], [A4165;H,H,SC(═S)OCH₂Br,H], [A4166;H,H,SC(═S)O(CH₂)₂Br,H], [A4167;H,H,SC(═S)O(CH₂)₃Br,H], [A4168;H,H,SC(═S)O(CH₂)₄Br,H], [A4169;H,H,SC(═S)O(CH₂)₅Br,H], [A4170;H,H,SC(═S)OCH₂OMe,H], [A4171;H,H,SC(═S)O(CH₂)₂OMe,H], [A4172;H,H,SC(═S)O(CH₂)₃OMe,H], [A4173;H,H,SC(═S)O(CH₂)₄OMe,H], [A4174;H,H,SC(═S)OCH₂OEt,H], [A4175;H,H,SC(═S)O(CH₂)₂OEt,H][A4176;H,H,SC(═S)O(CH₂)₃OEt,H], [A4177;H,H,SC(═S)OCH₂CN,H], [A4178;H,H,SC(═S)O(CH₂)₂CN,H], [A4179;H,H,SC(═S)O(CH₂)₃CN,H], [A4180;H,H,SC(═S)O(CH₂)₄CN,H], [A4181;H,H,SC(═S)O(CH₂)₅CN,H], [A4182;H,H,SC(═S)OCH₂C═CHF,H], [A4183;H,H,SC(═S)OCH₂C═CF₂,H], [A4184;H,H,SC(═S)O(CH₂)₂C═CHF,H], [A4185;H,H,SC(═S)OCHCH₂C═CF₂,H][A4186;H,H,SC(═S)O(CH₂)₃C═CHF,H], [A4187;H,H,SC(═S)OCH₂CHCH₂C═CF₂,H], [A4188;H,H,SC(═S)OCH₂C═CHCl,H], [A4189;H,H,SC(═S)OCH₂C═CCl₂,H], [A4190;H,H,SC(═S)O(CH₂)₂C═CHCl,H], [A4191;H,H,SC(═S)OCHCH₂C═CCl₂,H][A4192;H,H,SC(═S)O(CH₂)₃C═CHCl,H], [A4193;H,H,SC(═S)OCH₂CHCH₂C═CCl₂,H], [A4194;H,H,SC(═S)OCH₂C═CH,H], [A4195;H,H,SC(═S)OCH(Me)C═CH,H], [A4196;H,H,SC(═S)OCH₂C═CMe,H], [A4197;H,H,SC(═S)OCH₂C=CEt,H], [A4198;H,H,SC(═S)OCH₂C=CEt,H], [A4199;H,H,SC(═S)O(CH₂)₂C═CH,H], [A4200;H,H,SC(═S)O(CH₂)₂C═CMe,H],

[A4201;H,H,SC(═S)O(CH₂)₃C═CH,H], [A4202;H,H,SC(═S)O(CH₂)₄C═CH,H], [A4203;H,H,SC(═S)Oc-Pr,H], [A4204;H,H,SC(═S)Oc-Bu,H], [A4205;H,H,SC(═S)Oc-Pen,H], [A4206;H,H,SC(═S)Oc-Hex,H], [A4207;H,H,SC(═S)Oc-Hep,H], [A4208;H,H,SC(═S)OMe,H], [A4209;H,H,SC(═S)OEt,H], [A4210;H,H,SC(═S)O(CH₂)₂Me,H], [A4211;H,H,SC(═S)O(CH₂)₃Me,H], [A4212;H,H,SC(═S)O(CH₂)₄Me,H], [A4213;H,H,SC(═S)OCH(Me)₂,H], [A4214;H,H,SC(═S)OCH₂CH(Me) 2,H][A4215;H,H,SC(═S)O(CH₂)₂CH(Me)₂,H], [A4216;H,H,SC(═S)OC(Me)₃,H], [A4217;H,H,SC(═S)OCH₂C(Me)₃,H], [A4218;H,H,SC(═S)O(CH₂)₂C(Me)₃,H], [A4219;H,H,SC(═S)OCH(Me)Et,H][A4220;H,H,SC(═S)OCH(Me)(CH₂)₂Me,H], [A4221;H,H,SC(═S)OCH(Me)(CH₂)₃Me,H], [A4222;H,H,SC(═S)OCH(Et)₂,H], [A4223;H,H,SC(═S)OCH₂CH═CH₂,H], [A4224;H,H,SC(═S)O(CH₂)₂CH═CH₂,H][A4225;H,H,SC(═S)O(CH₂)₃CH═CH₂,H][A4226;H,H,SC(═S)OCH₂CH═CHMe,H], [A4227;H,H,SC(═S)OCH₂CH═CHEt,H], [A4228;H,H, S C(═S)OCH₂CH═C(Me)₂,H], [A4229;H,H,SC(═S)OCH₂CH═C(Me)Et,H], [A4230;H,H,SC(═S)OCH₂F,H], [A4231;H,H,SC(═S)OCHF₂,H], [A4232;H,H,SC(═S)OCF₃,H], [A4233;H,H,SC(═S)O(CH₂₂F,H], [A4234;H,H,SC(═S)OCH₂CHF₂,H], [A4235;H,H,SC(═S)OCH₂CF₃,H], [A4236;H,H,SC(═S)O(CH₂)₃F,H], [A4237;H,H,SC(═S)O(CH₂)₂CHF₂,H], [A4238;H,H,SC(═S)O(CH₂)₂CF₃,H], [A4239;H,H,SC(═S)OCH₂CH(CF₃)₂,H], [A4240;H,H,SC(═S)OCH₂Cl,H], [A4241;H,H,SC(═S)O(CH₂)₂Cl,H], [A4242;H,H,SC(═S)O(CH₂)₃Cl,H], [A4243;H,H,SC(═S)O(CH₂)₄Cl,H], [A4244;H,H,SC(═S)O(CH₂)₅Cl,H], [A4245;H,H,SC(═S)OCH₂Br,H], [A4246;H,H,SC(═S)O(CH₂)₂Br,H], [A4247;H,H,SC(═S)O(CH₂)₃Br,H], [A4248;H,H,SC(═S)O(CH₂)₄Br,H], [A4249;H,H,SC(═S)O(CH₂)₅Br,H], [A4250;H,H,SC(═S)OCH₂OMe,H], [A4251;H,H,SC(═S)O(CH₂)₂OMe,H][A4252;H,H,SC(═S)O(CH₂)₃OMe,H], [A4253;H,H,SC(═S)O(CH₂)₄OMe,H], [A4254;H,H,SC(═S)OCH₂OEt,H], [A4255;H,H,SC(═S)O(CH₂)₂OEt,H][A4256;H,H,SC(═S)O(CH₂)₃OEt,H], [A4257;H,H,SC(═S)OCH₂CN,H], [A4258;H,H,SC(═S)O(CH₂)₂CN,H], [A4259;H,H,SC(═S)O(CH₂)₃CN,H], [A4260;H,H,SC(═S)O(CH₂)₄CN,H], [A4261;H,H,SC(═S)O(CH₂)₅CN,H], [A4262;H,H,SC(═S)OCH₂C═CHF,H], [A4263;H,H,SC(═S)OCH₂C═CF₂,H], [A4264;H,H,SC(═S)O(CH₂)₂C═CHF,H][A4265;H,H,SC(═S)OCHCH₂C═CF₂,H][A4266;H,H,SC(═S)O(CH₂)₃C═CHF,H], [A4267;H,H,SC(═S)OCH₂CHCH₂C═CF₂,H], [A4268;H,H,SC(═S)OCH₂C═CHCl,H], [A4269;H,H,SC(═S)OCH₂C═CCl₂,H][A4270;H,H,SC(═S)O(CH₂)₂C═CHCl,H], [A4271;H,H,SC(═S)OCHCH₂C═CCl₂,H][A4272;H,H,SC(═S)O(CH₂)₃C═CHCl,H], [A4273;H,H,SC(═S)OCH₂CHCH₂C═CCl₂,H][A4274;H,H,SC(═S)OCH₂C═CH,H], [A4275;H,H,SC(═S)OCH(Me)C═CH,H], [A4276;H,H,SC(═S)OCH₂C═CMe,H], [A4277;H,H,SC(═S)OCH₂C=CEt,H], [A4278;H,H,SC(═S)OCH₂C=CEt,H][A4279;H,H,SC(═S)O(CH₂)₂C═CH,H], [A4280;H,H,SC(═S)O(CH₂)₂C═CMe,H], [A4281;H,H,SC(═S)O(CH₂)₃C═CH,H], [A4282;H,H,SC(═S)O(CH₂)₄C═CH,H], [A4283;H,H,SC(═S)Oc-Pr,H], [A4284;H,H,SC(═S)Oc-Bu,H], [A4285;H,H,SC(═S)Oc-Pen,H], [A4286;H,H,SC(═S)Oc-Hex,H], [A4287;H,H,SC(═S)Oc-Hep,H], [A4288;H,H,SC(═O)SMe,H], [A4289;H,H,SC(═O)SEt,H], [A4290;H,H,SC(═O)S(CH₂)₂Me,H], [A4291;H,H,SC(═O)S(CH₂)₃Me,H], [A4292;H,H,SC(═O)S(CH₂)₄Me,H], [A4293;H,H,SC(═O)SCH(Me)₂,H], [A4294;H,H,SC(═O)SCH₂CH(Me)₂,H][A4295;H,H,SC(═O)S(CH₂)₂CH(Me)₂,H], [A4296;H,H,SC(═O)SC(Me)₃,H], [A4297;H,H,SC(═O)SCH₂C(Me)₃,H], [A4298;H,H,SC(═O)S(CH₂)₂C(Me)₃,H], [A4299;H,H,SC(═O)SCH(Me)Et,H][A4300;H,H,SC(═O)SCH(Me)(CH₂)₂Me,H],

[A4301;H,H,SC(═O)SCH(Me)(CH₂)₃Me,H], [A4302;H,H,SC(═O)SCH(Et)₂,H], [A4303;H,H,SC(═O)SCH₂CH═CH₂,H], [A4304;H,H,SC(═O) 5 (CH₂)₂CH═CH₂,H], [A4305;H,H,SC(═O)S(CH₂)₃CH═CH₂,H], [A4306;H,H,SC(═)SCH₂CH═CHMe,H], [A4307;H,H,SC(═O)SCH₂CH═CHEt,H], [A4308;H,H,SC(═O)SCH₂CH═C(Me)₂,H][A4309;H,H,SC(═O)SCH₂CH═C(Me)Et,H], [A4310;H,H,SC(═O)SCH₂F,H], [A4311;H,H,SC(═O)SCHF₂,H], [A4312;H,H,SC(═O)SCF₃,H], [A4313;H,H,SC(═O)S(CH₂)₂F,H], [A4314;H,H,SC(═O)SCH₂CHF₂,H], [A4315;H,H,SC(═O)SCH₂CF₃,H], [A4316;H,H,SC(═O)S(CH₂)₃F,H], [A4317;H,H,SC(═O)S(CH₂)₂CHF₂,H], [A4318;H,H,SC(═O)S(CH₂)₂CF₃,H], [A4319;H,H,SC(═O)SCH₂CH(CF)₂,H], [A4320;H,H,SC(═O)SCH₂Cl,H], [A4321;H,H,SC(═O)S(CH₂)₂Cl,H], [A4322;H,H,SC(═O)S(CH₂)₃Cl,H], [A4323;H,H,SC(═O)S(CH₂)₄Cl,H], [A4324;H,H,SC(═O)S(CH₂)₅Cl,H], [A4325;H,H,SC(═O)SCH₂Br,H], [A4326;H,H,SC(═O)S(CH₂)₂Br,H], [A4327;H,H,SC(═O)S(CH₂)₃Br,H], [A4328;H,H,SC(═O)S(CH₂)₄Br,H], [A4329;H,H,SC(═O)S(CH₂)₅Br,H], [A4330;H,H,SC(═O)SCH₂OMe,H], [A4331;H,H,SC(═O)S(CH₂)₂OMe,H], [A4332;H,H,SC(═O)S(CH₂)₃OMe,H], [A4333;H,H,SC(═O)S(CH₂)₄OMe,H], [A4334;H,H,SC(═O)SCH₂OEt,H], [A4335;H,H,SC(═O)S(CH₂)₂OEt,H], [A4336;H,H,SC(═O)S(CH₂)₃OEt,H], [A4337;H,H,SC(═O)SCH₂CN,H], [A4338;H,H,SC(═O)S(CH₂)₂CN,H], [A4339;H,H,SC(═O)S(CH₂)₃CN,H], [A4340;H,H,SC(═O)S(CH₂)₄CN,H], [A4341;H,H,SC(═O)S(CH₂)₅CN,H], [A4342;H,H,SC(═O)SCH₂C═CHF,H], [A4343;H,H,SC(═O)SCH₂C═CF₂,H], [A4344;H,H,SC(═O)S(CH₂)₂C═CHF,H], [A4345;H,H,SC(═O)SCHCH₂C═CF₂,H], [A4346;H,H,SC(═O)S(CH₂)₃C═CHF,H], [A4347;H,H,SC(═O)SCH₂CHCH₂C═CF₂,H], [A4348;H,H,SC(═O)SCH₂C═CHCl,H], [A4349;H,H,SC(═O)SCH₂C═CCl₂,H], [A4350;H,H,SC(═O)S(CH₂)₂C═CHCl,H], [A4351;H,H,SC(═O)SCHCH₂C═CCl₂,H], [A4352;H,H,SC(═O)S(CH₂)₃C═CHCl,H], [A4353;H,H,SC(═O)SCH₂CHCH₂C═CCl₂,H], [A4354;H,H,SC(═O)SCH₂C═CH,H], [A4355;H,H,SC(═O)SCH(Me)C═CH,H], [A4356;H,H,SC(═O)SCH₂C═CMe,H], [A4357;H,H,SC(═O)SCH₂C=CEt,H], [A4358;H,H,SC(═O)SCH₂C=CEt,H], [A4359;H,H,SC(═O)S(CH₂)₂C═CH,H], [A4360;H,H,SC(═O)S(CH₂)₂C═CMe,H], [A4361;H,H,SC(═O)S(CH₂)₃C═CH,H], [A4362;H,H,SC(═O)S(CH₂)₄C═CH,H], [A4363;H,H,SC(═O)Sc—Pr,H], [A4364;H,H,SC(═O)Sc—Bu,H], [A4365;H,H,SC(═O)Sc—Pen,H], [A4366;H,H,SC(═O)Sc—Hex,H], [A4367;H,H,SC(═O)Sc—Hep,H], [A4368;H,H,SC(═O)SMe,H], [A4369;H,H,SC(═O)SEt,H], [A4370;H,H,SC(═O)S(CH₂)₂Me,H], [A4371;H,H,SC(═O)S(CH₂)₃Me,H], [A4372;H,H,SC(═O)S(CH₂)₄Me,H], [A4373;H,H,SC(═O)SCH(Me)₂,H], [A4374;H,H,SC(═O)SCH₂CH(Me)₂,H], [A4375;H,H,SC(═O)S(CH₂)₂CH(Me)₂,H], [A4376;H,H,SC(═O)SC(Me)₃,H], [A4377;H,H,SC(═O)SCH₂C(Me)₃,H], [A4378;H,H,SC(═O)S(CH₂)₂C(Me)₃,H], [A4379;H,H,SC(═O)SCH(Me)Et,H][A4380;H,H,SC(═O)SCH(Me)(CH₂)₂Me,H], [A4381;H,H,SC(═O)SCH(Me)(CH₂)₃Me,H], [A4382;H,H,SC(═O)SCH(Et)₂,H], [A4383;H,H,SC(═O)SCH₂CH═CH₂,H], [A4384;H,H,SC(═O)S(CH₂)₂CH═CH₂,H], [A4385;H,H,SC(═O)S(CH₂)₃CH═CH₂,H], [A4386;H,H,SC(═O)SCH₂CH═CHMe,H], [A4387;H,H,SC(═O)SCH₂CH═CHEt,H], [A4388;H,H,SC(═O)SCH₂CH═C(Me)₂,H][A4389;H,H,SC(═O)SCH₂CH═C(Me)Et,H], [A4390;H,H,SC(═O)SCH₂F,H], [A4391;H,H,SC(═O)SCHF₂,H], [A4392;H,H,SC(═O)SCF₃,H], [A4393;H,H,SC(═O)S(CH₂)₂F,H], [A4394;H,H,SC(═O)SCH₂CHF₂,H], [A4395;H,H,SC(═O)SCH₂CF₃,H], [A4396;H,H,SC(═O)S(CH₂)₃F,H], [A4397;H,H,SC(═O)S(CH₂)₂CHF₂,H], [A4398;H,H,SC(═O)S(CH₂)₂CF₃,H], [A4399;H,H,SC(═O)SCH₂CH(CF₃)₂,H], [A4400;H,H,SC(═O)SCH₂Cl,H],

[A4401;H,H,SC(═O)S(CH₂)₂Cl,H], [A4402;H,H,SC(═O)S(CH₂)₃Cl,H], [A4403;H,H,SC(═O)S(CH₂)₄Cl,H], [A4404;H,H,SC(═O)S(CH₂)₅Cl,H], [A4405;H,H,SC(═O)SCH₂Br,H], [A4406;H,H,SC(═O)S(CH₂)₂Br,H], [A4407;H,H,SC(═O)S(CH₂)₃Br,H], [A4408;H,H,SC(═O)S(CH₂)₄Br,H], [A4409;H,H,SC(═O)S(CH₂)₅Br,H], [A4410;H,H,SC(═O)SCH₂OMe,H], [A4411;H,H,SC(═O)S(CH₂)₂OMe,H], [A4412;H,H,SC(═O)S(CH₂)₃OMe,H], [A4413;H,H,SC(═O)S(CH₂)₄OMe,H], [A4414;H,H,SC(═O)SCH₂OEt,H], [A4415;H,H,SC(═O)S(CH₂)₂OEt,H], [A4416;H,H,SC(═O)S(CH₂)₃OEt,H], [A4417;H,H,SC(═O)SCH₂CN,H], [A4418;H,H,SC(═O)S(CH₂)₂CN,H], [A4419;H,H,SC(═O)S(CH₂)₃CN,H], [A4420;H,H,SC(═O)S(CH₂)₄CN,H], [A4421;H,H,SC(═O)S(CH₂)₅CN,H], [A4422;H,H,SC(═O)SCH₂C═CHF,H], [A4423;H,H,SC(═O)SCH₂C═CF₂,H], [A4424;H,H, Sc (═O)S(CH₂)₂C═CHF,H], [A4425;H,H,SC(═O)SCHCH₂C═CF₂,H], [A4426;H,H,SC(═O)S(CH₂)₃C═CHF,H], [A4427;H,H,SC(═O)SCH₂CHCH₂C═CF₂,H], [A4428;H,H,SC(═O)SCH₂C═CHCl,H], [A4429;H,H,SC(═O)SCH₂C═CCl₂,H], [A4430;H,H,SC(═O)S(CH₂)₂C═CHCl,H], [A4431;H,H,SC(═O)SCHCH₂C═CCl₂,H], [A4432;H,H,SC(═O)S(CH₂)₃C═CHCl,H], [A4433;H,H,SC(═O)SCH₂CHCH₂C═CCl₂,H], [A4434;H,H,SC(═O)SCH₂C═CH,H], [A4435;H,H,SC(═O)SCH(Me)C═CH,H], [A4436;H,H,SC(═O)SCH₂C═CMe,H], [A4437;H,H,SC(═O)SCH₂C=CEt,H], [A4438;H,H,SC(═O)SCH₂C=CEt,H], [A4439;H,H,SC(═O)S(CH₂)₂C═CH,H], [A4440;H,H,SC(═O)S(CH₂)₂C═CMe,H], [A4441;H,H,SC(═O)S(CH₂)₃C═CH,H], [A4442;H,H,SC(═O)S(CH₂)₄C═CH,H], [A4443;H,H,SC(═O)Sc—Pr,H], [A4444;H,H,SC(═O)Sc—Bu,H], [A4445;H,H,SC(═O)Sc—Pen,H], [A4446;H,H,SC(═O)Sc—Hex,H], [A4447;H,H,SC(═O)Sc—Hep,H], [A4448;H,H,SC(═O)SMe,H], [A4449;H,H,SC(═O)SEt,H], [A4450;H,H,SC(═O)S(CH₂)₂Me,H], [A4451;H,H,SC(═O)S(CH₂)₃Me,H], [A4452;H,H,SC(═O)S(CH₂)₄Me,H], [A4453;H,H,SC(═O)SCH(Me)₂,H], [A4454;H,H,SC(═O)SCH₂CH(Me)₂,H][A4455;H,H,SC(═O)S(CH₂)₂CH(Me)₂,H], [A4456;H,H,SC(═O)SC(Me)₃,H], [A4457;H,H,SC(═O)SCH₂C(Me)₃,H], [A4458;H,H,SC(═O)S(CH₂)₂C(Me)₃,H], [A4459;H,H,SC(═O)SCH(Me)Et,H], [A4460;H,H,SC(═O)SCH(Me)(CH₂)₂Me,H], [A4461;H,H,SC(═O)SCH(Me)(CH₂)₃Me,H], [A4462;H,H,SC(═O)SCH(Et)₂,H], [A4463;H,H,SC(═O)SCH₂CH═CH₂,H], [A4464;H,H,SC(═O)S(CH₂)₂CH═CH₂,H], [A4465;H,H,SC(═O)S(CH₂)₃CH═CH₂,H], [A4466;H,H,SC(═O)SCH₂CH═CHMe,H], [A4467;H,H,SC(═O)SCH₂CH═CHEt,H], [A4468;H,H,SC(═O)SCH₂CH═C(Me)₂,H][A4469;H,H,SC(═O)SCH₂CH═C(Me)Et,H], [A4470;H,H,SC(═O)SCH₂F,H], [A4471;H,H,SC(═O)SCHF₂,H], [A4472;H,H,SC(═O)SCF₃,H], [A4473;H,H,SC(═O)S(CH₂)₂F,H], [A4474;H,H,SC(═O)SCH₂CHF₂,H], [A4475;H,H,SC(═O)SCH₂CF₃,H], [A4476;H,H,SC(═O)S(CH₂)₃F,H], [A4477;H,H,SC(═O) 5 (CH₂)₂CHF₂,H], [A4478;H,H,SC(═O)S(CH₂)₂CF₃,H], [A4479;H,H,SC(═O)SCH₂CH(CF₃)₂,H], [A4480;H,H,SC(═O)SCH₂Cl,H], [A4481;H,H,SC(═O)S(CH₂)₂Cl,H], [A4482;H,H,SC(═O)S(CH₂)₃Cl,H], [A4483;H,H,SC(═O)S(CH₂)₄Cl,H], [A4484;H,H,SC(═O)S(CH₂)₅Cl,H], [A4485;H,H,SC(═O)SCH₂Br,H], [A4486;H,H,SC(═O)S(CH₂)₂Br,H], [A4487;H,H,SC(═O)S(CH₂)₃Br,H], [A4488;H,H,SC(═O)S(CH₂)₄Br,H], [A4489;H,H,SC(═O)S(CH₂)₅Br,H], [A4490;H,H,SC(═O)SCH₂OMe,H], [A4491;H,H,SC(═O)S(CH₂)₂OMe,H], [A4492;H,H,SC(═O)S(CH₂)₃OMe,H], [A4493;H,H,SC(═O)S(CH₂)₄OMe,H], [A4494;H,H,SC(═O)SCH₂OEt,H], [A4495;H,H,SC(═O)S(CH₂)₂OEt,H], [A4496;H,H,SC(═O)S(CH₂)₃OEt,H], [A4497;H,H,SC(═O)SCH₂CN,H], [A4498;H,H,SC(═O)S(CH₂)₂CN,H], [A4499;H,H,SC(═O)S(CH₂)₃CN,H], [A4500;H,H,SC(═O)S(CH₂)₄CN,H],

[A4501;H,H,SC(═O)S(CH₂)₅CN,H], [A4502;H,H,SC(═O)SCH₂C═CHF,H], [A4503;H,H,SC(═O)SCH₂C═CF₂,H], [A4504;H,H,SC(═O)S(CH₂)₂C═CHF,H], [A4505;H,H,SC(═O)SCHCH₂C═CF₂,H], [A4506;H,H,SC(═O)S(CH₂)₃C═CHF,H], [A4507;H,H,SC(═O)SCH₂CHCH₂C═CF₂,H], [A4508;H,H,SC(═O)SCH₂C═CHCl,H], [A4509;H,H,SC(═O)SCH₂C═CCl₂,H], [A4510;H,H,SC(═O)S(CH₂)₂C═CHCl,H], [A4511;H,H,SC(═O)SCHCH₂C═CCl₂,H][A4512;H,H,SC(═O)S(CH₂)₃C═CHCl,H], [A4513;H,H,SC(═O)SCH₂CHCH₂C═CCl₂,H], [A4514;H,H,SC(═O)SCH₂C═CH,H], [A4515;H,H,SC(═O)SCH(Me)C═CH,H], [A4516;H,H,SC(═O)SCH₂C═CMe,H], [A4517;H,H,SC(═O)SCH₂C=CEt,H], [A4518;H,H,SC(═O)SCH₂C=CEt,H], [A4519;H,H,SC(═O)S(CH₂)₂C═CH,H], [A4520;H,H,SC(═O)S(CH₂)₂C═CMe,H], [A4521; H,H,SC(═O)S(CH₂)₃C═CH,H], [A4522;H,H,SC(═O)S(CH₂)₄C═CH,H], [A4523;H,H,SC(═O)Sc—Pr,H], [A4524;H,H,SC(═O)Sc—Bu,H], [A4525;H,H,SC(═O)Sc—Pen,H], [A4526;H,H,SC(═O)Sc—Hex,H], [A4527;H,H,SC(═O)Sc—Hep,H], [A4528;H,H,SC(═O)SMe,H], [A4529;H,H,SC(═O)SEt,H], [A4530;H,H,SC(═O)S(CH₂)₂Me,H], [A4531;H,H,SC(═O)S(CH₂)₃Me,H], [A4532;H,H,SC(═O)S(CH₂)₄Me,H], [A4533;H,H,SC(═O)SCH(Me)₂,H], [A4534;H,H,SC(═O)SCH₂CH(Me)₂,H][A4535;H,H,SC(═O)S(CH₂)₂CH(Me)₂,H], [A4536;H,H,SC(═O)SC(Me)₃,H], [A4537;H,H,SC(═O)SCH₂C(Me)₃,H], [A4538;H,H,SC(═O)S(CH₂)₂C(Me)₃,H], [A4539;H,H,SC(═O)SCH(Me)Et,H][A4540;H,H,SC(═O)SCH(Me)(CH₂)₂Me,H], [A4541;H,H,SC(═O)SCH(Me)(CH₂)₃Me,H], [A4542;H,H,SC(═O)SCH(Et)₂,H], [A4543;H,H,SC(═O)SCH₂CH═CH₂,H], [A4544;H,H,SC(═O) 5 (CH₂)₂CH═CH₂,H][A4545;H,H,SC(═O)S(CH₂)₃CH═CH₂,H], [A4546;H,H,SC(═O)SCH₂CH═CHMe,H], [A4547;H,H,SC(═O)SCH₂CH═CHEt,H], [A4548;H,H,SC(═O)SCH₂CH═C(Me)₂,H], [A4549;H,H,SC(═O)SCH₂CH═C(Me)Et,H], [A4550;H,H,SC(═O)SCH₂F,H], [A4551;H,H,SC(═O)SCHF₂,H], [A4552;H,H,SC(═O)SCF₃,H], [A4553;H,H,SC(═O)S(CH₂)₂F,H], [A4554;H,H,SC(═O)SCH₂CHF₂,H], [A4555;H,H,SC(═O)SCH₂CF₃,H], [A4556;H,H,SC(═O)S(CH₂)₃F,H], [A4557;H,H,SC(═O)S(CH₂)₂CHF₂,H], [A4558;H,H,SC(═O) 5 (CH₂)₂CF₃,H], [A4559;H,H,SC(═O)SCH₂CH(CF₃)₂,H], [A4560;H,H,SC(═O)SCH₂Cl,H], [A4561;H,H,SC(═O)S(CH₂)₂Cl,H], [A4562;H,H,SC(═O)S(CH₂)₃Cl,H], [A4563;H,H,SC(═O)S(CH₂)₄Cl,H], [A4564;H,H,SC(═O)S(CH₂)₅Cl,H], [A4565;H,H,SC(═O)SCH₂Br,H], [A4566;H,H,SC(═O)S(CH₂)₂Br,H], [A4567;H,H,SC(═O)S(CH₂)₃Br,H], [A4568;H,H,SC(═O)S(CH₂)₄Br,H], [A4569;H,H,SC(═O)S(CH₂)₅Br,H], [A4570;H,H,SC(═O)SCH₂OMe,H], [A4571;H,H,SC(═O)S(CH₂)₂OMe,H], [A4572;H,H,SC(═O)S(CH₂)₃OMe,H], [A4573;H,H,SC(═O)S(CH₂)₄OMe,H], [A4574;H,H,SC(═O)SCH₂OEt,H], [A4575;H,H,SC(═O)S(CH₂)₂OEt,H], [A4576;H,H,SC(═O)S(CH₂)₃OEt,H], [A4577;H,H,SC(═O)SCH₂CN,H], [A4578;H,H,SC(═O)S(CH₂)₂CN,H], [A4579;H,H,SC(═O)S(CH₂)₃CN,H], [A4580;H,H,SC(═O)S(CH₂)₄CN,H], [A4581;H,H,SC(═O)S(CH₂)₅CN,H], [A4582;H,H,SC(═O)SCH₂C═CHF,H], [A4583;H,H,SC(═O)SCH₂C═CF₂,H], [A4584;H,H,SC(═O) 5 (CH₂)₂C═CHF,H], [A4585;H,H,SC(═O)SCHCH₂C═CF₂,H], [A4586;H,H,SC(═O)S(CH₂)₃C═CHF,H], [A4587;H,H,SC(═O)SCH₂CHCH₂C═CF₂,H], [A4588;H,H,SC(═O)SCH₂C═CHCl,H], [A4589;H,H,SC(═O)SCH₂C═CCl₂,H], [A4590;H,H,SC(═O)S(CH₂)₂C═CHCl,H], [A4591;H,H,SC(═O)SCHCH₂C═CCl₂,H], [A4592;H,H,SC(═O)S(CH₂)₃C═CHCl,H], [A4593;H,H,SC(═O)SCH₂CHCH₂C═CCl₂,H], [A4594;H,H,SC(═O)SCH₂C═CH,H], [A4595;H,H,SC(═O)SCH(Me)C═CH,H], [A4596;H,H,SC(═O)SCH₂C═CMe,H], [A4597;H,H,SC(═O)SCH₂C=CEt,H], [A4598;H,H,SC(═O)SCH₂C=CEt,H], [A4599;H,H,SC(═O)S(CH₂)₂C═CH,H], [A4600;H,H,SC(═O)S(CH₂)₂C═CMe,H],

[A4601;H,H,SC(═O)S(CH₂)₃C═CH,H], [A4602;H,H,SC(═O)S(CH₂)₄C═CH,H], [A4603;H,H,SC(═O)Sc—Pr,H], [A4604;H,H,SC(═O)Sc—Bu,H], [A4605;H,H,SC(═O)Sc—Pen,H], [A4606;H,H,SC(═O)Sc—Hex,H], [A4607;H,H,SC(═O)Sc—Hep,H], [A4608;H,H,SC(═O)SMe,H], [A4609;H,H,SC(═O)SEt,H], [A4610;H,H,SC(═O)S(CH₂)₂Me,H], [A4611;H,H,SC(═O)S(CH₂)₃Me,H], [A4612;H,H,SC(═O)S(CH₂)₄Me,H], [A4613;H,H,SC(═O)SCH(Me)₂,H], [A4614;H,H,SC(═O)SCH₂CH(Me)₂,H], [A4615;H,H,SC(═O)S(CH₂)₂CH(Me)₂,H], [A4616;H,H,SC(═O)SC(Me)₃,H], [A4617;H,H,SC(═O)SCH₂C(Me)₃,H], [A4618;H,H,SC(═O)S(CH₂)₂C(Me)₃,H], [A4619;H,H,SC(═O)SCH(Me)Et,H][A4620;H,H,SC(═O)SCH(Me)(CH₂)₂Me,H], [A4621;H,H,SC(═O)SCH(Me)(CH₂)₃Me,H], [A4622;H,H,SC(═O)SCH(Et)₂,H], [A4623;H,H,SC(═O)SCH₂CH═CH₂,H], [A4624;H,H,SC(═O)S(CH₂)₂CH═CH₂,H], [A4625;H,H,SC(═O)S(CH₂)₃CH═CH₂,H], [A4626;H,H,SC(═O)SCH₂CH═CHMe,H], [A4627;H,H,SC(═O)SCH₂CH═CHEt,H], [A4628;H,H,SC(═O)SCH₂CH═C(Me)₂,H], [A4629;H,H,SC(═O)SCH₂CH═C(Me)Et,H], [A4630;H,H,SC(═O)SCH₂F,H], [A4631;H,H,SC(═O)SCHF₂,H], [A4632;H,H,SC(═O)SCF₃,H], [A4633;H,H,SC(═O)S(CH₂)₂F,H], [A4634;H,H,SC(═O)SCH₂CHF₂,H], [A4635;H,H,SC(═O)SCH₂CF₃,H], [A4636;H,H,SC(═O)S(CH₂)₃F,H], [A4637;H,H,SC(═O)S (CH₂)₂CHF₂,H], [A4638;H,H,SC(═O)S(CH₂)₂CF₃,H], [A4639;H,H,SC(═O)SCH₂CH(CF₃)₂,H], [A4640;H,H,SC(═O)SCH₂Cl,H], [A4641;H,H,SC(═O)S(CH₂)₂Cl,H], [A4642;H,H,SC(═O)S(CH₂)₃Cl,H], [A4643;H,H,SC(═O)S(CH₂)₄Cl,H], [A4644;H,H,SC(═O)S(CH₂)₅Cl,H], [A4645;H,H,SC(═O)SCH₂Br,H], [A4646;H,H,SC(═O)S(CH₂)₂Br,H], [A4647;H,H,SC(═O)S(CH₂)₃Br,H], [A4648;H,H,SC(═O)S(CH₂)₄Br,H], [A4649;H,H,SC(═O)S(CH₂)₅Br,H], [A4650;H,H,SC(═O)SCH₂OMe,H], [A4651;H,H,SC(═O) 5 (CH₂)₂OMe,H], [A4652;H,H,SC(═O)S(CH₂)₃OMe,H], [A4653;H,H,SC(═O)S(CH₂)₄OMe,H], [A4654;H,H,SC(═O)SCH₂OEt,H], [A4655;H,H,SC(═O)S(CH₂)₂OEt,H], [A4656;H,H,SC(═O)S(CH₂)₃OEt,H], [A4657;H,H,SC(═O)SCH₂CN,H], [A4658;H,H,SC(═O)S(CH₂)₂CN,H], [A4659;H,H,SC(═O)S(CH₂)₃CN,H], [A4660;H,H,SC(═O)S(CH₂)₄CN,H], [A4661;H,H,SC(═O)S(CH₂)₅CN,H], [A4662;H,H,SC(═O)SCH₂C═CHF,H], [A4663;H,H,SC(═O)SCH₂C═CF₂,H], [A4664;H,H,SC(═O)S(CH₂)₂C═CHF,H], [A4665;H,H,SC(═O)SCHCH₂C═CF₂,H], [A4666;H,H,SC(═O)S(CH₂)₃C═CHF,H], [A4667;H,H,SC(═O)SCH₂CHCH₂C═CF₂,H], [A4668;H,H,SC(═O)SCH₂C═CHCl,H], [A4669;H,H,SC(═O)SCH₂C═CCl₂,H], [A4670;H,H,SC(═O)S(CH₂)₂C═CHCl,H], [A4671;H,H,SC(═O)SCHCH₂C═CCl₂,H], [A4672;H,H,SC(═O)S(CH₂)₃C═CHCl,H], [A4673;H,H,SC(═O)SCH₂CHCH₂C═CCl₂,H], [A4674;H,H,SC(═O)SCH₂C═CH,H], [A4675;H,H,SC(═O)SCH(Me)C═CH,H], [A4676;H,H,SC(═O)SCH₂C═CMe,H], [A4677;H,H,SC(═O)SCH₂C=CEt,H], [A4678;H,H,SC(═O)SCH₂C=CEt,H], [A4679;H,H,SC(═O)S(CH₂)₂C═CH,H], [A4680;H,H,SC(═O)S(CH₂)₂C═CMe,H], [A4681;H,H,SC(═O)S(CH₂)₃C═CH,H], [A4682;H,H,SC(═O)S(CH₂)₄C═CH,H], [A4683;H,H,SC(═O)Sc—Pr,H], [A4684;H,H,SC(═O)Sc—Bu,H], [A4685;H,H,SC(═O)Sc—Pen,H], [A4686;H,H,SC(═O)Sc—Hex,H], [A4687;H,H,SC(═O)Sc—Hep,H], [A4688;H,H,SC(═O)Me,H], [A4689;H,H,SC(═O)Et,H], [A4690;H,H,SC(═O)(CH₂)₂Me,H], [A4691;H,H,SC(═O)(CH₂)₃Me,H], [A4692;H,H,SC(═O)(CH₂)₄Me,H], [A4693;H,H,SC(═O)CH(Me)₂,H], [A4694;H,H,SC(═O)CH₂CH(Me)₂,H], [A4695;H,H,SC(═O)(CH₂)₂CH(Me)₂,H], [A4696;H,H,SC(═O)C(Me)₃,H], [A4697;H,H,SC(═O) CH₂C(Me)₃,H], [A4698;H,H,SC(═O)(CH₂)₂C(Me)₃,H], [A4699;H,H,SC(═O)CH(Me)Et,H][A4700;H,H,SC(═O)CH(Me)(CH₂)₂Me,H],

[A4701;H,H,SC(═O)CH(Me)(CH₂)₃Me,H], [A4702;H,H,SC(═O)CH(Et)₂,H], [A4703;H,H,SC(═O)CH₂CH═CH₂,H], [A4704;H,H,SC(═O)(CH₂)₂CH═CH₂,H], [A4705;H,H,SC(═O)(CH₂)₃CH═CH₂,H], [A4706;H,H,SC(═O)CH₂CH═CHMe,H], [A4707;H,H,SC(═O)CH₂CH═CHEt,H], [A4708;H,H,SC(═O)CH₂CH═C(Me)₂,H][A4709;H,H,SC(═O)CH₂CH═C(Me)Et,H], [A4710;H,H,SC(═O)CH₂F,H], [A4711;H,H,SC(═O)CHF₂,H], [A4712;H,H,SC(═O) CF₃,H], [A4713;H,H,SC(═O)(CH₂₂F,H], [A4714;H,H,SC(═O)CH₂CHF₂,H], [A4715;H,H,SC(═O)CH₂CF₃,H], [A4716;H,H,SC(═O)(CH₂)₃F,H], [A4717;H,H,SC(═O)(CH₂)₂CHF₂,H], [A4718;H,H,SC(═O)(CH₂)₂CF₃,H], [A4719;H,H,SC(═O)CH₂CH(CF₃)₂,H], [A4720;H,H,SC(═O)CH₂Cl,H], [A4721;H,H,SC(═O)(CH₂)₂Cl,H], [A4722;H,H,SC(═O)(CH₂)₃Cl,H], [A4723;H,H,SC(═O)(CH₂)₄Cl,H], [A4724;H,H,SC(═O)(CH₂)₅Cl,H], [A4725;H,H,SC(═O)CH₂Br,H], [A4726;H,H,SC(═O)(CH₂)₂Br,H], [A4727;H,H,SC(═O)(CH₂)₃Br,H], [A4728;H,H,SC(═O)(CH₂)₄Br,H], [A4729;H,H,SC(═O)(CH₂)₅Br,H], [A4730;H,H,SC(═O)CH₂OMe,H], [A4731;H,H,SC(═O)(CH₂)₂OMe,H], [A4732;H,H,SC(═O)(CH₂)₃OMe,H], [A4733;H,H,SC(═O)(CH₂)₄OMe,H], [A4734;H,H,SC(═O)CH₂OEt,H], [A4735;H,H,SC(═O)(CH₂)₂OEt,H], [A4736;H,H,SC(═O)(CH₂)₃OEt,H], [A4737;H,H,SC(═O)CH₂CN,H], [A4738;H,H,SC(═O)(CH₂)₂CN,H], [A4739;H,H,SC(═O)(CH₂)₃CN,H], [A4740;H,H,SC(═O)(CH₂)₄CN,H], [A4741;H,H,SC(═O)(CH₂)₅CN,H], [A4742;H,H,SC(═O)CH₂C═CHF,H], [A4743;H,H,SC(═O)CH₂C═CF₂,H], [A4744;H,H,SC(═O)(CH₂)₂C═CHF,H], [A4745;H,H,SC(═O)CHCH₂C═CF₂,H], [A4746;H,H,SC(═O)(CH₂)₃C═CHF,H], [A4747;H,H,SC(═O)CH₂CHCH₂C═CF₂,H], [A4748;H,H,SC(═O)CH₂C═CHCl,H], [A4749;H,H,SC(═O)CH₂C═CCl₂,H], [A4750;H,H,SC(═O)(CH₂)₂C═CHCl,H], [A4751;H,H,SC(═O)CHCH₂C═CCl₂,H], [A4752;H,H,SC(═O)(CH₂)₃C═CHCl,H], [A4753;H,H,SC(═O)CH₂CHCH₂C═CCl₂,H], [A4754;H,H,SC(═O)CH₂C═CH,H], [A4755;H,H,SC(═O)CH(Me)C═CH,H], [A4756;H,H,SC(═O)CH₂C═CMe,H], [A4757;H,H,SC(═O)CH₂C=CEt,H], [A4758;H,H,SC(═O)CH₂C=CEt,H], [A4759;H,H,SC(═O)(CH₂)₂C═CH,H], [A4760;H,H,SC(═O)(CH₂)₂C═CMe,H], [A4761;H,H,SC(═O)(CH₂)₃C═CH,H], [A4762;H,H,SC(═O)(CH₂)₄C═CH,H], [A4763;H,H,SC(═O)c-Pr,H], [A4764;H,H,SC(═O)c-Bu,H], [A4765;H,H,SC(═O)c-Pen,H], [A4766;H,H,SC(═O)c-Hex,H], [A4767;H,H,SC(═O)c-Hep,H], [A4768;H,H,SC(═O)Me,H], [A4769;H,H,SC(═)Et,H], [A4770;H,H,SC(═O)(CH₂)₂Me,H], [A4771;H,H,SC(═O)(CH₂)₃Me,H], [A4772;H,H,SC(═O)(CH₂)₄Me,H], [A4773;H,H,SC(═O)CH(Me)₂,H], [A4774;H,H,SC(═O)CH₂CH(Me)₂,H], [A4775;H,H,SC(═O)(CH₂)₂CH(Me)₂,H], [A4776;H,H,SC(═O)₂C(Me)₃,H], [A4777;H,H,SC(═O)CH₂C(Me)₃,H], [A4778;H,H,SC(═O)(CH₂)₂C(Me)₃,H], [A4779;H,H,SC(═O)CH(Me)Et,H][A4780;H,H,SC(═O)CH(Me)(CH₂)₂Me,H], [A4781;H,H,SC(═O)CH(Me)(CH₂)₃Me,H], [A4782;H,H,SC(═O)CH(Et)₂,H], [A4783;H,H,SC(═O)CH₂CH═CH₂,H], [A4784;H,H,SC(═O)(CH₂)₂CH═CH₂,H], [A4785;H,H,SC(═O)(CH₂)₃CH═CH₂,H], [A4786;H,H,SC(═O)CH₂CH═CHMe,H], [A4787;H,H,SC(═O)CH₂CH═CHEt,H], [A4788;H,H,SC(═O)CH₂CH═C(Me)₂,H][A4789;H,H,SC(═O)CH₂CH═C(Me)Et,H], [A4790;H,H,SC(═O)CH₂F,H], [A4791;H,H,SC(═O)CHF₂,H], [A4792;H,H,SC(═O) CF₃,H], [A4793;H,H,SC(═O)(CH₂)₂F,H], [A4794;H,H,SC(═O)CH₂CHF₂,H], [A4795;H,H,SC(═O)CH₂CF₃,H], [A4796;H,H,SC(═O)(CH₂)₃F,H], [A4797;H,H,SC(═O)(CH₂)₂CHF₂,H], [A4798;H,H,SC(═O)(CH₂)₂CF₃,H], [A4799;H,H,SC(═O)CH₂CH(CF)₂,H], [A4800;H,H,SC(═O)CH₂Cl,H],

[A4801;H,H,SC(═O)(CH₂)₂Cl,H], [A4802;H,H,SC(═O)(CH₂)₃Cl,H], [A4803;H,H,SC(═O)(CH₂)₄Cl,H], [A4804;H,H,SC(═O)(CH₂)₅Cl,H], [A4805;H,H,SC(═O)CH₂Br,H], [A4806;H,H,SC(═O)(CH₂)₂Br,H], [A4807;H,H,SC(═O)(CH₂)₃Br,H], [A4808;H,H,SC(═O)(CH₂)₄Br,H], [A4809;H,H,SC(═O)(CH₂)₅Br,H], [A4810;H,H,SC(═O)CH₂OMe,H], [A4811;H,H,SC(═O)(CH₂)₂OMe,H], [A4812;H,H,SC(═O)(CH₂)₃OMe,H], [A4813;H,H,SC(═O)(CH₂)₄OMe,H], [A4814;H,H,SC(═O)CH₂OEt,H], [A4815;H,H,SC(═O)(CH₂)₂OEt,H], [A4816;H,H,SC(═O)(CH₂)₃OEt,H], [A4817;H,H,SC(═O)CH₂CN,H], [A4818;H,H,SC(═O)(CH₂)₂CN,H], [A4819;H,H,SC(═O)(CH₂)₃CN,H], [A4820;H,H,SC(═O)(CH₂)₄CN,H], [A4821;H,H,SC(═O)(CH₂)₅CN,H], [A4822;H,H,SC(═O)CH₂C═CHF,H], [A4823;H,H,SC(═O)CH₂C═CF₂,H], [A4824;H,H,SC(═O)(CH₂)₂C═CHF,H], [A4825;H,H,SC(═O)CHCH₂C═CF₂,H], [A4826;H,H,SC(═O)(CH₂)₃C═CHF,H], [A4827;H,H,SC(═O)CH₂CHCH₂C═CF₂,H][A4828;H,H,SC(═O)CH₂C═CHCl,H], [A4829;H,H,SC(═O)CH₂C═CCl₂,H], [A4830;H,H,SC(═O)(CH₂)₂C═CHCl,H], [A4831;H,H,SC(═O)CHCH₂C═CCl₂,H][A4832;H,H,SC(═O)(CH₂)₃C═CHCl,H], [A4833;H,H,SC(═O)CH₂CHCH₂C═CCl₂,H], [A4834;H,H,SC(═O)CH₂C═CH,H], [A4835;H,H,SC(═O)CH(Me)C═CH,H], [A4836;H,H,SC(═O)CH₂C═CMe,H], [A4837;H,H,SC(═O)CH₂C=CEt,H], [A4838;H,H,SC(═O)CH₂C=CEt,H], [A4839;H,H,SC(═O)(CH₂)₂C═CH,H], [A4840;H,H,SC(═O)(CH₂)₂C═CMe,H], [A4841;H,H,SC(═O)(CH₂)₃C═CH,H], [A4842;H,H,SC(═O)(CH₂)₄C═CH,H], [A4843;H,H,SC(═O) c-Pr,H], [A4844;H,H,SC(═O)c-Bu,H], [A4845;H,H,SC(═O)c-Pen,H], [A4846;H,H,SC(═O)c-Hex,H], [A4847;H,H,SC(═O)c-Hep,H], [A4848;H,H,SC(═O)Me,H], [A4849;H,H,SC(═O)Et,H], [A4850;H,H,SC(═O)(CH₂)₂Me,H], [A4851;H,H,SC(═O)(CH₂)₃Me,H], [A4852;H,H,SC(═O)(CH₂)₄Me,H], [A4853;H,H,SC(═O)CH(Me)₂,H], [A4854;H,H,SC(═O)CH₂CH(Me) 2,H][A4855;H,H,SC(═O)(CH₂₂CH(Me)₂,H], [A4856;H,H,SC(═O)₂C(Me)₃,H], [A4857;H,H,SC(═O)CH₂C(Me)₃,H], [A4858;H,H,SC(═O)(CH₂)₂C(Me)₃,H], [A4859;H,H,SC(═O)CH(Me)Et,H], [A4860;H,H,SC(═O)CH(Me)(CH₂)₂Me,H], [A4861;H,H,SC(═O)CH(Me)(CH₂)₃Me,H], [A4862;H,H,SC(═O)CH(Et)₂,H], [A4863;H,H,SC(═O)CH₂CH═CH₂,H], [A4864;H,H,SC(═O)(CH₂)₂CH═CH₂,H], [A4865;H,H,SC(═O)(CH₂)₃CH═CH₂,H], [A4866;H,H,SC(═O)CH₂CH═CHMe,H], [A4867;H,H,SC(═O)CH₂CH═CHEt,H], [A4868;H,H,SC(═O)CH₂CH═C(Me)₂,H], [A4869;H,H,SC(═O)CH₂CH═C(Me)Et,H], [A4870;H,H,SC(═O)CH₂F,H], [A4871;H,H,SC(═O)CHF₂,H], [A4872;H,H,SC(═O)CF₃,H], [A4873;H,H,SC(═O)(CH₂)₂F,H], [A4874;H,H,SC(═O)CH₂CHF₂,H], [A4875;H,H,SC(═O)CH₂CF₃,H], [A4876;H,H,SC(═O)(CH₂)₃F,H], [A4877;H,H,SC(═O)(CH₂)₂CHF₂,H], [A4878;H,H,SC(═O)(CH₂)₂CF₃,H], [A4879;H,H,SC(═O)CH₂CH(CF₃)₂,H], [A4880;H,H,SC(═O)CH₂Cl,H], [A4881;H,H,SC(═O)(CH₂)₂Cl,H], [A4882;H,H,SC(═O)(CH₂)₃Cl,H], [A4883;H,H,SC(═O)(CH₂)₄Cl,H], [A4884;H,H,SC(═O)(CH₂)₅Cl,H], [A4885;H,H,SC(═O)CH₂Br,H], [A4886;H,H,SC(═O)(CH₂)₂Br,H], [A4887;H,H,SC(═O)(CH₂)₃Br,H], [A4888;H,H,SC(═O)(CH₂)₄Br,H], [A4889;H,H,SC(═O)(CH₂)₅Br,H], [A4890;H,H,SC(═O)CH₂OMe,H], [A4891;H,H,SC(═O)(CH₂)₂OMe,H], [A4892;H,H,SC(═O)(CH₂)₃OMe,H], [A4893;H,H,SC(═O)(CH₂)₄OMe,H], [A4894;H,H,SC(═O)CH₂OEt,H], [A4895;H,H,SC(═O)(CH₂)₂OEt,H], [A4896;H,H,SC(═O)(CH₂)₃OEt,H], [A4897;H,H,SC(═O)CH₂CN,H], [A4898;H,H,SC(═O)(CH₂)₂CN,H], [A4899;H,H,SC(═O)(CH₂)₃CN,H], [A4900;H,H,SC(═O)(CH₂)₄CN,H],

[A4901;H,H,SC(═O)(CH₂)₅CN,H], [A4902;H,H,SC(═O)CH₂C═CHF,H], [A4903;H,H,SC(═O)CH₂C═CF₂,H], [A4904;H,H,SC(═O)(CH₂)₂C═CHF,H], [A4905;H,H,SC(═O)CHCH₂C═CF₂,H], [A4906;H,H,SC(═O)(CH₂)₃C═CHF,H], [A4907;H,H,SC(═O)CH₂CHCH₂C═CF₂,H], [A4908;H,H,SC(═O)CH₂C═CHCl,H], [A4909;H,H,SC(═O)CH₂C═CCl₂,H], [A4910;H,H,SC(═O)(CH₂)₂C═CHCl,H], [A4911;H,H,SC(═O)CHCH₂C═CCl₂,H], [A4912;H,H,SC(═O)(CH₂)₃C═CHCl,H], [A4913;H,H,SC(═O)CH₂CHCH₂C═CCl₂,H], [A4914;H,H,SC(═O)CH₂C═CH,H], [A4915;H,H,SC(═O)CH(Me)C═CH,H], [A4916;H,H,SC(═O)CH₂C═CMe,H], [A4917;H,H,SC(═O)CH₂C=CEt,H], [A4918;H,H,SC(═O)CH₂C=CEt,H], [A4919;H,H,SC(═O)(CH₂)₂C═CH,H], [A4920;H,H,SC(═O)(CH₂)₂C═CMe,H], [A4921;H,H,SC(═O)(CH₂)₃C═CH,H], [A4922;H,H,SC(═O)(CH₂)₄C═CH,H], [A4923;H,H,SC(═O) c-Pr,H], [A4924;H,H,SC(═O)c-Bu,H], [A4925;H,H,SC(═O)c-Pen,H], [A4926;H,H,SC(═O)c-Hex,H], [A4927;H,H,SC(═O)c-Hep,H], [A4928;H,H,SC(═O)Me,H], [A4929;H,H,SC(═O)Et,H], [A4930;H,H,SC(═O)(CH₂)₂Me,H], [A4931;H,H,SC(═O)(CH₂)₃Me,H], [A4932;H,H,SC(═O)(CH₂)₄Me,H], [A4933;H,H,SC(═O)CH(Me)₂,H], [A4934;H,H,SC(═O)CH₂CH(Me) 2,H][A4935;H,H,SC(═O)(CH₂)₂CH(Me)₂,H], [A4936;H,H,SC(═O)C(Me)₃,H], [A4937;H,H,SC(═O)CH₂C(Me)₃,H], [A4938;H,H,SC(═O)(CH₂)₂C(Me)₃,H], [A4939;H,H,SC(═O)CH(Me)Et,H][A4940;H,H,SC(═O)CH(Me)(CH₂)₂Me,H], [A4941;H,H,SC(═O)CH(Me)(CH₂)₃Me,H], [A4942;H,H,SC(═O)CH(Et)₂,H], [A4943;H,H,SC(═O)CH₂CH═CH₂,H], [A4944;H,H,SC(═O)(CH₂)₂CH═CH₂,H], [A4945;H,H,SC(═O)(CH₂)₃CH═CH₂,H], [A4946;H,H,SC (═O)CH₂CH═CHMe,H], [A4947;H,H,SC(═O)CH₂CH═CHEt,H][A4948;H,H,SC(═O)CH₂CH═C(Me)₂,H], [A4949;H,H,SC(═O)CH₂CH═C(Me)Et,H], [A4950;H,H,SC(═O)CH₂F,H], [A4951;H,H,SC(═O)CHF₂,H], [A4952;H,H,SC(═O)CF₃,H], [A4953;H,H,SC(═O)(CH₂)₂F,H], [A4954;H,H,SC(═O)CH₂CHF₂,H], [A4955;H,H,SC(═O)CH₂CF₃,H], [A4956;H,H,SC(═O)(CH₂)₃F,H], [A4957;H,H,SC(═O)(CH₂)₂CHF₂,H], [A4958;H,H,SC(═O)(CH₂)₂CF₃,H], [A4959;H,H,SC(═O)CH₂CH(CF₃)₂,H], [A4960;H,H,SC(═O)CH₂Cl,H], [A4961;H,H,SC(═O)(CH₂)₂Cl,H], [A4962;H,H,SC(═O)(CH₂)₃Cl,H], [A4963;H,H,SC(═O)(CH₂)₄Cl,H], [A4964;H,H,SC(═O)(CH₂)₅Cl,H], [A4965;H,H,SC(═O)CH₂Br,H], [A4966;H,H,SC(═O)(CH₂)₂Br,H], [A4967;H,H,SC(═O)(CH₂)₃Br,H], [A4968;H,H,SC(═O)(CH₂)₄Br,H], [A4969;H,H,SC(═O)(CH₂)₅Br,H], [A4970;H,H,SC(═O)CH₂OMe,H], [A4971;H,H,SC(═O)(CH₂)₂OMe,H], [A4972;H,H,SC(═O)(CH₂)₃OMe,H], [A4973;H,H,SC(═O)(CH₂)₄OMe,H], [A4974;H,H,SC(═O)CH₂OEt,H], [A4975;H,H,SC(═O)(CH₂)₂OEt,H], [A4976;H,H,SC(═O)(CH₂)₃OEt,H], [A4977;H,H,SC(═O)CH₂CN,H], [A4978;H,H,SC(═O)(CH₂)₂CN,H], [A4979;H,H,SC(═O)(CH₂)₃CN,H], [A4980;H,H,SC(═O)(CH₂)₄CN,H], [A4981;H,H,SC(═O)(CH₂)₅CN,H], [A4982;H,H,SC(═O)CH₂C═CHF,H], [A4983;H,H,SC(═O)CH₂C═CF₂,H], [A4984;H,H,SC(═O)(CH₂)₂C═CHF,H], [A4985;H,H,SC(═O)CHCH₂C═CF₂,H], [A4986;H,H,SC(═O)(CH₂)₃C═CHF,H], [A4987;H,H,SC(═O)CH₂CHCH₂C═CF₂,H], [A4988;H,H,SC(═O)CH₂C═CHCl,H], [A4989;H,H,SC(═O)CH₂C═CCl₂,H], [A4990;H,H,SC(═O)(CH₂)₂C═CHCl,H], [A4991;H,H,SC(═O)CHCH₂C═CCl₂,H], [A4992;H,H,SC(═O)(CH₂)₃C═CHCl,H], [A4993;H,H,SC(═O)CH₂CHCH₂C═CCl₂,H], [A4994;H,H,SC(═O)CH₂C═CH,H], [A4995;H,H,SC(═O)CH(Me)C═CH,H], [A4996;H,H,SC(═O)CH₂C═CMe,H], [A4997;H,H,SC(═O)CH₂C=CEt,H], [A4998;H,H,SC(═O)CH₂C=CEt,H], [A4999;H,H,SC(═O)(CH₂)₂C═CH,H], [A5000;H,H,SC(═O)(CH₂)₂C═CMe,H], [A5001;H,H,SC(═O)(CH₂)₃C═CH,H], [A5002;H,H,SC(═O)(CH₂)₄C═CH,H], [A5003;H,H,SC(═O) c-Pr,H], [A5004;H,H,SC(═O)c-Bu,H], [A5005;H,H,SC(═O)c-Pen,H], [A5006;H,H,SC(═O)c-Hex,H], [A5007;H,H,SC(═O)c-Hep,H], [A5008;H,H,SC(═O)Me,H], [A5009;H,H,SC(═O)Et,H], [A5010;H,H,SC(═O)(CH₂)₂Me,H], [A5011;H,H,SC(═O)(CH₂)₃Me,H], [A5012;H,H,SC(═O)(CH₂)₄Me,H], [A5013;H,H,SC(═O)CH(Me)₂,H], [A5014;H,H,SC(═O)CH₂CH(Me) 2,H][A5015;H,H,SC(═O)(CH₂)₂CH(Me)₂,H], [A5016;H,H,SC(═O)₂C(Me)₃,H], [A5017;H,H,SC(═O)CH₂C(Me)₃,H], [A5018;H,H,SC(═O)(CH₂)₂C(Me)₃,H], [A5019;H,H,SC(═O)CH(Me)Et,H][A5020;H,H,SC(═O)CH(Me)(CH₂)₂Me,H], [A5021;H,H,SC(═O)CH(Me)(CH₂)₃Me,H], [A5022;H,H,SC(═O)CH(Et)₂,H], [A5023;H,H,SC(═O)CH₂CH═CH₂,H], [A5024;H,H,SC(═O)(CH₂)₂CH═CH₂,H], [A5025;H,H,SC(═O)(CH₂)₃CH═CH₂,H], [A5026;H,H,SC (═O)CH₂CH═CHMe,H], [A5027;H,H,SC(═O)CH₂CH═CHEt,H][A5028;H,H,SC(═O)CH₂CH═C(Me)₂,H], [A5029;H,H,SC(═O)CH₂CH═C(Me)Et,H], [A5030;H,H,SC(═O)CH₂F,H], [A5031;H,H,SC(═O)CHF₂,H], [A5032;H,H,SC(═O) CF₃,H], [A5033;H,H,SC(═O)(CH₂)₂F,H], [A5034;H,H,SC(═O)CH₂CHF₂,H], [A5035;H,H,SC(═O)CH₂CF₃,H], [A5036;H,H,SC(═O)(CH₂)₃F,H], [A5037;H,H,SC(═O)(CH₂)₂CHF₂,H], [A5038;H,H,SC(═O)(CH₂)₂CF₃,H], [A5039;H,H,SC(═O)CH₂CH(CF₃)₂,H], [A5040;H,H,SC(═O)CH₂Cl,H], [A5041;H,H,SC(═O)(CH₂)₂Cl,H], [A5042;H,H,SC(═O)(CH₂)₃Cl,H], [A5043;H,H,SC(═O)(CH₂)₄Cl,H], [A5044;H,H,SC(═O)(CH₂)₅Cl,H], [A5045;H,H,SC(═O)CH₂Br,H], [A5046;H,H,SC(═O)(CH₂)₂Br,H], [A5047;H,H,SC(═O)(CH₂)₃Br,H], [A5048;H,H,SC(═O)(CH₂)₄Br,H], [A5049;H,H,SC(═O)(CH₂)₅Br,H], [A5050;H,H,SC(═O)CH₂OMe,H], [A5051;H,H,SC(═O)(CH₂)₂OMe,H], [A5052;H,H,SC(═O)(CH₂)₃OMe,H], [A5053;H,H,SC(═O)(CH₂)₄OMe,H], [A5054;H,H,SC(═O)CH₂OEt,H], [A5055;H,H,SC(═O)(CH₂)₂OEt,H], [A5056;H,H,SC(═O)(CH₂)₃OEt,H], [A5057;H,H,SC(═O)CH₂CN,H], [A5058;H,H,SC(═O)(CH₂)₂CN,H], [A5059;H,H,SC(═O)(CH₂)₃CN,H], [A5060;H,H,SC(═O)(CH₂))₄CN,H], [A5061;H,H,SC(═O)(CH₂)₅CN,H], [A5062;H,H,SC(═O)CH₂C═CHF,H], [A5063;H,H,SC(═O)CH₂C═CF₂,H], [A5064;H,H,SC(═O)(CH₂)₂C═CHF,H], [A5065;H,H,SC(═O)CHCH₂C═CF₂,H], [A5066;H,H,SC(═O)(CH₂)₃C═CHF,H], [A5067;H,H,SC(═O)CH₂CHCH₂C═CF₂,H], [A5068;H,H,SC(═O)CH₂C═CHCl,H], [A5069;H,H,SC(═O)CH₂C═CCl₂,H], [A5070;H,H,SC(═O)(CH₂)₂C═CHCl,H], [A5071;H,H,SC(═O)CHCH₂C═CCl₂,H], [A5072;H,H,SC(═O)(CH₂)₃C═CHCl,H], [A5073;H,H,SC(═O)CH₂CHCH₂C═CCl₂,H], [A5074;H,H,SC(═O)CH₂C═CH,H], [A5075;H,H,SC(═O)CH(Me)C═CH,H], [A5076;H,H,SC(═O)CH₂C═CMe,H], [A5077;H,H,SC(═O)CH₂C=CEt,H], [A5078;H,H,SC(═O)CH₂C=CEt,H], [A5079;H,H,SC(═O)(CH₂)₂C═CH,H], [A5080;H,H,SC(═O)(CH₂)₂C═CMe,H], [A5081;H,H,SC(═O)(CH₂)₃C═CH,H], [A5082;H,H,SC(═O)(CH₂)₄C═CH,H], [A5083;H,H,SC(═O)c-Pr,H], [A5084;H,H,SC(═O)c-Bu,H], [A5085;H,H,SC(═O)c-Pen,H], [A5086;H,H,SC(═O)c-Hex,H], [A5087;H,H,SC(═O)c-Hep,H].

It is possible to produce a compound in which a combination of R²⁰, R²¹, R²², and R²³ is a combination according to any one of substituent number A1 to substituent number A5087 in the compound represented by formula (2) (hereinafter, compounds of substituent number A1 to substituent number A5087 are referred to as the present compound A1 to the present compound A5087, and the present compound A1 to the present compound A5087 are collectively referred to as the compound group SX1 in the compound represented by formula (2)) in accordance with the production processes mentioned above.

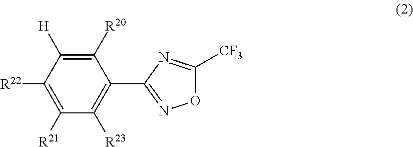

For example, the present compound A2 means a compound in which a substituent number is A2 in the compound represented by formula (2), and is a compound in which R²⁰, R²¹, and R²³ are hydrogen atoms and R²² is OC(═O)NHCHF₂ in the compound represented by formula (2), which is a compound shown below.

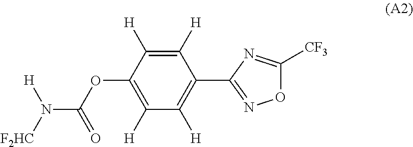 [SMILES: O=C(NC(F)F)Oc1ccc(-c2noc(C(F)(F)F)n2)cc1]

It is possible to produce a compound in which a combination of R²⁰, R²¹, R²², and R²³ is a combination of any one of substituent number A1 to substituent number A5087 in the compound represented by formula (3) (hereinafter, compounds of substituent number A1 to substituent number A5087 are referred to as the present compound B1 to the present compound B5087, and the present compound B1 to the present compound B5087 are collectively referred to as the compound group SX2 in the compound represented by formula (3)) in accordance with the production processes mentioned above.

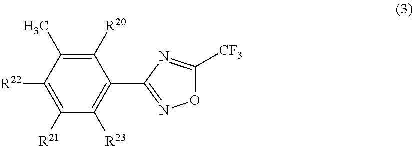

For example, the present compound B2 means a compound in which a substituent number is A2 in the compound represented by formula (3), and is a compound in which R²⁰, R²¹, and R²³ are hydrogen atoms and R²² is OC(═O)NHCHF₂ in the compound represented by formula (3), which is a compound shown below.

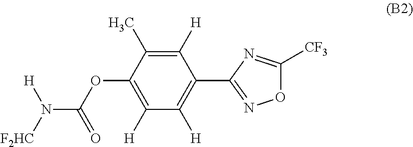 [SMILES: Cc1cc(-c2noc(C(F)(F)F)n2)ccc1OC(=O)NC(F)F]

It is possible to produce a compound in which a combination of R²⁰, R²¹, R²², and R²³ is a combination of any one of substituent number A1 to substituent number A5087 in the compound represented by formula (4) (hereinafter, compounds of substituent number A1 to substituent number A5087 are referred to as the present compound C1 to the present compound C5087, and the present compound C1 to the present compound C5087 are collectively referred to as the compound group SX3 in the compound represented by formula (4)) in accordance with the production processes mentioned above.

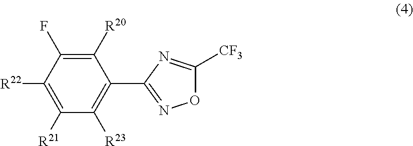

For example, the present compound C2 means a compound in which a substituent number is A2 in the compound represented by formula (4), and is a compound in which R²⁰, R²¹, and R²³ are hydrogen atoms and R²² is OC(═O)NHCHF₂ in the compound represented by formula (4), which is a compound shown below.

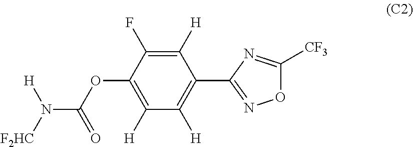 [SMILES: O=C(NC(F)F)Oc1ccc(-c2noc(C(F)(F)F)n2)cc1F]

It is possible to produce a compound in which a combination of R²⁰, R²¹, R²², and R²³ is a combination of any one of substituent number A1 to substituent number A5087 in the compound represented by formula (5) (hereinafter, compounds of substituent number A1 to substituent number A5087 are referred to as the present compound D1 to the present compound D5087, and the present compound D1 to the present compound D5087 are collectively referred to as the compound group SX4 in the compound represented by formula (5)) in accordance with the production processes mentioned above.

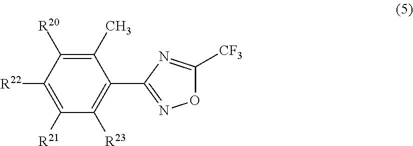

For example, the present compound D2 means a compound in which a substituent number is A2 in the compound represented by formula (5), and is a compound in which R²⁰, R²¹, and R²³ are hydrogen atoms and R²² is OC(═O)NHCHF₂ in the compound represented by formula (5), which is a compound shown below.

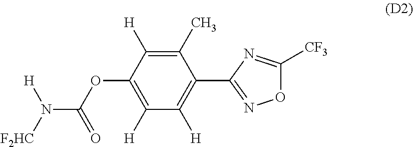 [SMILES: Cc1cc(OC(=O)NC(F)F)ccc1-c1noc(C(F)(F)F)n1]

It is possible to produce a compound in which a combination of R²⁰, R²¹, R²², and R²³ is a combination of any one of substituent number A1 to substituent number A5087 in the compound represented by formula (6) (hereinafter, compounds of substituent number A1 to substituent number A5087 are referred to as the present compound E1 to the present compound E5087, and the present compound E1 to the present compound E5087 are collectively referred to as the compound group SX5 in the compound represented by formula (6)) in accordance with the production processes mentioned above.

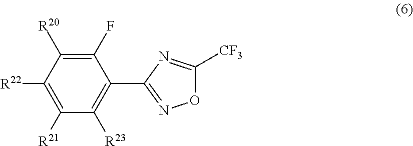

For example, the present compound E2 means a compound in which a substituent number is A2 in the compound represented by formula (6), and is a compound in which R²⁰, R²¹, and R²³ are hydrogen atoms and R²² is OC(═O)NHCHF₂ in the compound represented by formula (6), which is a compound shown below.

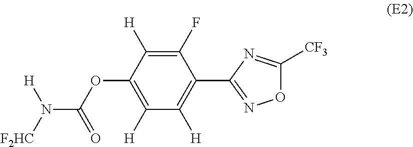 [SMILES: O=C(NC(F)F)Oc1ccc(-c2noc(C(F)(F)F)n2)c(F)c1]

It is possible to produce a compound in which a combination of R²⁰, R²¹, R²², and R²³ is a combination of any one of substituent number A1 to substituent number A5087 in the compound represented by formula (7) (hereinafter, compounds of substituent number A1 to substituent number A5087 are referred to as the present compound F1 to the present compound F5087, and the present compound F1 to the present compound F5087 are collectively referred to as the compound group SX6 in the compound represented by formula (7)) in accordance with the production processes mentioned above.

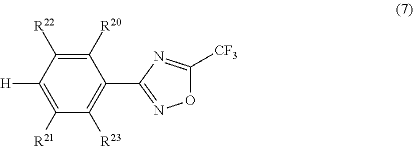

For example, the present compound F2 means a compound in which a substituent number is A2 in the compound represented by formula (7), and is a compound in which R²⁰, R²¹, and R²³ are hydrogen atoms and R²² is OC(═O)NHCHF₂ in the compound represented by formula (7), which is a compound shown below.

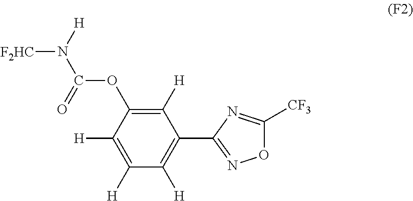 [SMILES: O=C(NC(F)F)Oc1cccc(-c2noc(C(F)(F)F)n2)c1]

It is possible to produce a compound in which a combination of R²⁰, R²¹, R²², and R²³ is a combination of any one of substituent number A1 to substituent number A5087 in the compound represented by formula (8) (hereinafter, compounds of substituent number A1 to substituent number A5087 are referred to as the present compound G1 to the present compound G5087, and the present compound G1 to the present compound G5087 are collectively referred to as the compound group SX7 in the compound represented by formula (8)) in accordance with the production processes mentioned above.

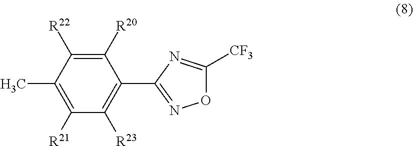

For example, the present compound G2 means a compound in which a substituent number is A2 in the compound represented by formula (8), and is a compound in which R²⁰, R²¹, and R²³ are hydrogen atoms and R²² is OC(═O)NHCHF₂ in the compound represented by formula (8), which is a compound shown below.

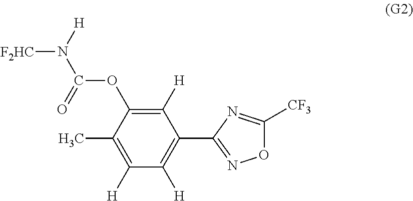 [SMILES: Cc1ccc(-c2noc(C(F)(F)F)n2)cc1OC(=O)NC(F)F]

It is possible to produce a compound in which a combination of R²⁰, R²¹, R²², and R²³ is a combination of any one of substituent number A1 to substituent number A5087 in the compound represented by formula (9) (hereinafter, compounds of substituent number A1 to substituent number A5087 are referred to as the present compound H1 to the present compound H5087, and the present compound H1 to the present compound H5087 are collectively referred to as the compound group SX8 in the compound represented by formula (9)) in accordance with the production processes mentioned above.

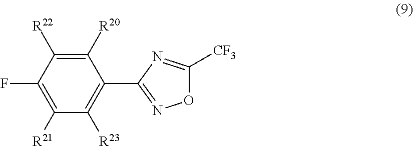

For example, the present compound H2 means a compound in which a substituent number is A2 in the compound represented by formula (9), and is a compound in which R²⁰, R²¹, and R²³ are hydrogen atoms and R²² is OC(═O)NHCHF₂ in the compound represented by formula (9), which is a compound shown below.

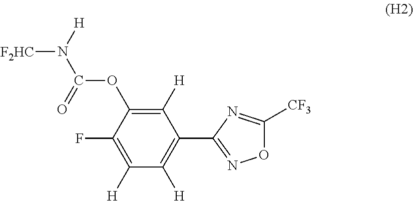 [SMILES: O=C(NC(F)F)Oc1cc(-c2noc(C(F)(F)F)n2)ccc1F]

The present compound can be used as a mixture with or together with plant disease control agents such as fungicides, insecticides, acaricides, nematicides, plant growth regulators, or synergists (hereinafter referred to as the present ingredient). Examples of the combination of the present compound and the present ingredient are shown below. For example, tebuconazole+SX means the combination of tebuconazole+SX. Abbreviation of SX means any one compound selected from the compound groups SX1 to SX8. Each numeral in parentheses means CAS Registry No.

tebuconazole+SX, prothioconazole+SX, metconazole+SX, ipconazole+SX, triticonazole+SX, difenoconazole+SX, imazalil+SX, triadimenol+SX, tetraconazole+SX, flutriafol+SX, bromuconazole+SX, propiconazole+SX, mefentrifluconazole+SX, ipfentrifluconazole+SX, epoxiconazole+SX, cyproconazole+SX, mandestrobin+SX, azoxystrobin+SX, pyraclostrobin+SX, trifloxystrobin+SX, fluoxastrobin+SX, picoxystrobin+SX, fenamidone+SX, dimoxystrobin+SX, metominostrobin+SX, pyribencarb+SX, sedaxane+SX, penflufen+SX, fluxapyroxad+SX, fluopyram+SX, benzovindiflupyr+SX, boscalid+SX, carboxin+SX, penthiopyrad+SX, flutolanil+SX, bixafen+SX, pydiflumetofen+SX, 3-difluoromethyl-N-(7-fluoro-1,1,3-trimethylindan-4-yl)-1-methylpyrazole-4-carboxamide (1383809-87-7)+SX, N-cyclopropyl-3-(difluoromethyl)-5-fluoro-N-(5-chloro-2-isopropylbenzyl)-1-methyl-1H-pyrazole-4-carboxamide (1255734-28-1)+SX, 3-difluoromethyl-1-methyl-N-(1,1,3-trimethylindan-4-yl)pyrazole-4-carboxamide (141573-94-6)+SX, 3-difluoromethyl-1-methyl-N-[(3R)-1,1,3-trimethylindan-4-yl]pyrazole-4-carboxamide (1352994-67-2)+SX, metalaxyl+SX, metalaxyl-M+SX, metrafenone+SX, cyflufenamid+SX, proquinazid+SX, 3-chloro-5-phenyl-6-methyl-4-(2,6-difluorophenyl)pyridazine (1358061-55-8)+SX, 1-(2-{[1-(4-chlorophenyl)-1H-pyrazol-3-yl]oxymethyl}-3-methylphenyl)-4-methyl-1,4-dihydrotetrazol-5-one (1472649-01-6)+SX, 4-(2-bromo-4-fluorophenyl)-N-(2-chloro-6-fluorophenyl)-1,3-dimethyl-1H-pyrazole-5-amine (1362477-26-6)+SX, fenpicoxamid+SX, N′-(2,5-dimethyl-4-phenoxyphenyl)-N-ethyl-N-methyl-methaneimidamide (1052688-31-9)+SX, isotianil+SX, oxolinic acid+SX, ferimzone+SX, phthalide+SX, kasugamycin+SX, tebufloquin+SX, quinofumelin+SX, fenpyrazamine+SX, procymidone+SX, fludioxonil+SX, tolclofos-methyl+SX, thiabendazole+SX, ethaboxam+SX, picarbutrazox+SX, oxathiapiprolin+SX, iminoctadine triacetate+SX, iminoctadine albesilate+SX, fenpropimorph+SX, fenpropidin+SX, spiroxamine+SX, chlorothalonil+SX, folpet+SX, captan+SX, thiram+SX, silthiofam+SX, mancozeb+SX, cartap+SX, clothianidin+SX, thiamethoxam+SX, imidacloprid+SX, thiacloprid+SX, flupyradifurone+SX, sulfoxaflor+SX, triflumezopyrim+SX, dicloromezotiaz+SX, beta-cyfluthrin+SX, tefluthrin+SX, fipronil+SX, chlorantraniliprole+SX, cyantraniliprole+SX, tetraniliprole+SX, thiodicarb+SX, carbofuran+SX, fluxametamide+SX, afoxolaner+SX, fluralaner+SX, broflanilide+SX, abamectin+SX, fluensulfone+SX, fluazaindolizine+SX, tioxazafen+SX, (E)-N-{1-[6-chloropyridin-3-yl]methyl}pyridine-2(H)-ylideen}-2,2,2-trifluoroacetamide (1689566-03-7)+SX, Mycorrhizal Fungi+SX, Bacillus firmus+SX, Bacillus amyloliquefaciens+SX, Pasteuria nishizawae+SX, Pasteuria penetrans+SX.

A ratio of the present compound to the present active ingredient is not particularly limited, and a weight ratio (the present compound:the present active ingredient) is, for example, within a range of 1000:1 to 1:1000, 500:1 to 1:500, 100:1 to 1:100, 50:1 to 1:50, 20:1 to 1:20, 10:1 to 1:10, 3:1 to 1:3, 1:1 to 1:500, 1:1 to 1:100, 1:1 to 1:50, 1:1 to 1:20, or 1:1 to 1:10.

A treatment of plants with the present compound exhibits the effect of promoting the growth of plants, such as an increase in the seedling establishment rate, an increase in the number of healthy leaves, an increase in the plant length, an increase in the plant body weight, an increase in the leaf area, an increase in the number or weight of seeds or fruits, an increase in the number of set flowers or fruits, and an increase in the growth of roots. A treatment of plants with the present compound leads to an improvement in resistance to temperature stress such as high-temperature stress or low-temperature stress, water stress such as drought stress or excessive moisture stress, or abiotic stress such as salt stress.

Formulation Examples will be shown below. In Formulation Examples, parts are by weight.

### Formulation Example 1

Fifty parts (50 parts) of SX, 3 parts of calcium ligninsulfonate, 2 parts of laurylmagnesium sulfate, and 45 parts of synthetic hydrated silicon oxide are thoroughly ground and mixed to obtain each formulation.

### Formulation Example 2

Twenty parts (20 parts) of SX and 1.5 parts of sorbitan trioleate are mixed with 28.5 parts of an aqueous solution containing 2 parts of polyvinyl alcohol, and the mixture was finely ground by a wet grinding method. Then, 40 parts of an aqueous solution containing 0.05 part of xanthan gum and 0.1 part of aluminum magnesium silicate is added thereto and 10 parts of propylene glycol is further added, followed by stirring and mixing to obtain each formulation.

### Formulation Example 3

Two parts (2 parts) of SX, 88 parts of kaolin clay, and 10 parts of talc are thoroughly ground and mixed to obtain each formulation.

### Formulation Example 4

Five parts (5 parts) of SX, 14 parts of polyoxyethylene styryl phenyl ether, 6 parts of calcium dodecylbenzenesulfonate, and 75 parts of xylene are thoroughly ground and mixed to obtain each formulation.

### Formulation Example 5

Two parts (2 parts) of SX, 1 part of synthetic hydrated silicon oxide, 2 parts of calcium ligninsulfonate, 30 parts of bentonite, and 65 parts of kaolin clay are thoroughly ground and mixed. After the addition of water, the mixture is thoroughly kneaded and further granulated and dried to obtain each formulation.

### Formulation Example 6

Twenty parts (20 parts) of SX, 35 parts of a mixture (weight ratio of 1:1) of white carbon and a polyoxyethylene alkyl ether sulfate ammonium salt, and water were mixed to make 100 parts, followed by a treatment using a mill to obtain each formulation.

The following Test Examples will show that the present compounds are useful for controlling plant diseases. The non-treated area in Test Examples 1 to 4 means the treated area in which the treatment was performed under the same conditions as those mentioned in each test example, except that dimethyl sulfoxide was suspended in place of the dimethyl sulfoxide dilution containing the present compound.

The non-treatment in Test Examples 5 to 20 means that foliage application of a mixture of a preparation containing the present compound and water was not performed.

### Test Example 1

Antifungal Test Against Wheat Leaf Blotch (Septoria tritici)

The present compound 2 was diluted with dimethyl sulfoxide so as to obtain a dilution having a concentration of 3,750 ppm and the dilution thus obtained was suspended into each well of a titer plate (with 96 wells) in the amount of 1 μL, and then 150 μL of a potato dextrose broth liquid medium (PDB medium) inoculated in advance with spores of wheat leaf blotch fungus was added. This plate was cultured at 18° C. for 5 days, thereby allowing wheat leaf blotch fungus to undergo proliferation, and then the absorbance at 550 nm of each well of the titer plate was measured, and the value thus obtained was regarded as the degree of growth of wheat leaf blotch fungus. As a result, the degree of growth in an area treated with the present compound 2 was 40% or less of that in a non-treated area.

### Test Example 2

### Antifungal Test Against Tomato Leaf Mold (Cladosporium Fulvum)

The present compound 2, 10, 12 to 15, 17, 18, 21, 29, 30, 33, 35, 36, 39, or 41 was diluted with dimethyl sulfoxide so as to obtain a dilution having a concentration of 1,500 ppm and the dilution thus obtained was suspended into each well of a titer plate (with 96 wells) in the amount of 1 μL, and then 150 μL of a potato dextrose broth liquid medium (PDB medium) inoculated in advance with spores of tomato leaf mold fungus (a QoI-resistant strain in which, among the genes encoding cytochrome b, the amino acid residue at position 129 of cytochrome b is mutated from phenylalanine to leucine) was added. This plate was cultured at 18° C. for 5 days, thereby allowing tomato leaf mold fungus to undergo proliferation, and then the absorbance at 550 nm of each well of the titer plate was measured, and the value thus obtained was regarded as the degree of growth of tomato leaf mold fungus. As a result, the degree of growth in an area treated with the present compound 2, 10, 12 to 15, 17, 18, 21, 29, 30, 33, 35, 36, 39, or 41 was 30% or less of that in a non-treated area.

### Test Example 3

Antifungal Test Against Wheat Leaf Blotch (Septoria tritici)

The present compound 2, 8, 10, 12, 13, 22, 24, 28, 30, 33, 35, 36, 39, 40, 41, or 44 was diluted with dimethyl sulfoxide so as to obtain a dilution having a concentration of 1,500 ppm and the dilution thus obtained was suspended into each well of a titer plate (with 96 wells) in the amount of 1 μL, and then 150 μL of a potato dextrose broth liquid medium (PDB medium) inoculated in advance with spores of wheat leaf blotch fungus was added. This plate was cultured at 18° C. for 5 days, thereby allowing wheat leaf blotch fungus to undergo proliferation, and then the absorbance at 550 nm of each well of the titer plate was measured, and the value thus obtained was regarded as the degree of growth of wheat leaf blotch fungus. As a result, the degree of growth in an area treated with present compound 2, 8, 10, 12, 13, 22, 24, 28, 30, 33, 35, 36, 39, 40, 41, or 44 was 40% or less of that in a non-treated area.

### Test Example 4

### Antifungal Test Against Soybean Anthracnose (Colletotrichum Truncatum)

The present compound 2, 3, 8, 15, 36, or 41 was diluted with dimethyl sulfoxide so as to obtain a dilution having a concentration of 1,500 ppm and the dilution thus obtained was suspended into each well of a titer plate (with 96 wells) in the amount of 1 μL, and then 150 μL of a potato dextrose broth liquid medium (PDB medium) inoculated in advance with spores of soybean anthracnose fungus was added. This plate was cultured at 18° C. for 4 days, thereby allowing soybean anthracnose fungus to undergo proliferation, and then the absorbance at 550 nm of each well of the titer plate was measured, and the value thus obtained was regarded as the degree of growth of soybean anthracnose fungus. As a result, the degree of growth in an area treated with the present compound 2, 3, 8, 15, 36, or 41 was 30% or less of that in a non-treated area.

### Test Example 5

Control Test on Rice Blast (Magnaporthe grisea)

Each of plastic pots was filled with soil and rice (cultivar: HINOHIKARI) was sowed and grown in a greenhouse for 20 days. Then, the present compound 3, 6, 8, 13, 36, or 44 formulated in accordance with the method mentioned in Formulation Example 6 was mixed with water so as to adjust the concentration to 200 ppm, and the mixture was sprayed over stems and leaves so that it sufficiently adhered to the surface of the leaves of the rice. After spraying, the rice was air-dried and subjected to a spraying treatment and the rice seedling (cultivar: HINOHIKARI) infected by rice blast fungus left to stand for 6 days at 24° C. in the daytime and 20° C. at night under high humidity condition, while being in contact with each other, and then the area of lesion spots was investigated. As a result, the lesion areas on the rice treated with the present compound 3, 6, 8, 13, 36, or 44 were 30% or less with respect to the lesion area on the non-treated rice.

### Test Example 6

Control Test on Wheat Leaf Blotch (Septoria tritici)

Each of plastic pots was filled with soil and wheat (cultivar: APOGEE) was sowed and grown in a greenhouse for 10 days. Then, the present compound 1, 3, 4, 6, 9 to 13, 20, 31, 33, 35, 36, 42, or 44 formulated in accordance with the method mentioned in Formulation Example 6 was mixed with water so as to adjust the concentration to 200 ppm, and the mixture was sprayed over stems and leaves so that it sufficiently adhered to the surface of the leaves of the wheat. After spraying, the wheat was air-dried. After 4 days, an aqueous suspension containing spores of wheat leaf blotch fungus was sprayed to inoculate the spores. After inoculation, the wheat was at first left to stand at 18° C. under high humidity condition for 3 days and then left to stand under illumination for 14 to 18 days, and the area of lesion spots was investigated. As a result, the area of lesion spots on the wheat treated with the present compound 1, 3, 4, 6, 9 to 13, 20, 31, 33, 35, 36, 42, or 44 was 30% or less of that on a non-treated wheat.

### Test Example 7

Control Test on Wheat Leaf Blotch (Septoria tritici)

Each of plastic pots was filled with soil and wheat (cultivar: APOGEE) was sowed and grown in a greenhouse for 10 days. Thereafter, an aqueous suspension containing spores of wheat leaf blotch fungus (Septoria tritici) was sprayed over the wheat to inoculate the spores. After inoculation, the wheat was left to stand at 18° C. under high humidity condition for 3 days. Then, the present compound 4, 5, or 10 formulated in accordance with the method mentioned in Formulation Example 6 was mixed with water so as to adjust the concentration to 200 ppm, and the mixture was sprayed over stems and leaves of the wheat so that it sufficiently adhered to the surface of the leaves of the wheat. After spraying, the wheat was air-dried. The plant was left to stand under illumination for 14 to 18 days, and then the area of lesion spots was investigated. As a result, the area of lesion spots on the wheat treated with the present compound 4, 5, or 10 was 30% or less of that on a non-treated wheat.

### Test Example 8

Control Test on Wheat Rust (Puccinia recondita)

Each of plastic pots was filled with soil and wheat (cultivar: SHIROGANE) was sowed and grown in a greenhouse for 9 days. Then, the present compound 4, 6, 16, 30, 31, 35, or 36 formulated in accordance with the method mentioned in Formulation Example 6 was mixed with water so as to adjust the concentration to 200 ppm, and the mixture was sprayed over stems and leaves of the wheat so that it sufficiently adhered to the surface of the leaves of the wheat. After spraying, the wheat was air-dried and cultivated at 20° C. for 5 days under illumination, and then inoculated by sprinkling with spores of wheat rust fungus. After inoculation, the wheat was left to stand at 23° C. for 1 day under dark and high humidity condition, and cultivated under illumination at 20° C. for 8 days, and then the area of lesion spots was investigated. As a result, the area of lesion spots on the wheat treated with the present compound 4, 6, 16, 30, 31, 35, or 36 was 30% or less of that on a non-treated wheat.

### Test Example 9

Control Test on Soybean Rust (Phakopsora pachyrhizi)

Each of plastic pots was filled with soil and soybean (cultivar: KUROSENGOKU) was sowed and grown in a greenhouse for 10 days. Thereafter, an aqueous suspension containing spores of soybean rust fungus was sprayed to inoculate the spores. After inoculation, the soybean was at first left to stand in a greenhouse at 23° C. in the daytime and 20° C. at night under high humidity condition for 1 day and cultivated in a greenhouse at room temperature for 2 days. Then, the present compound 1 to 9, 11 to 23, 26 to 33, 35 to 41, or 44 formulated in accordance with the method mentioned in Formulation Example 6 was mixed with water so as to adjust the concentration to 200 ppm, and the mixture was sprayed over stems and leaves of the soybean so that it sufficiently adhered to the surface of the leaves of the soybean. After spraying, the soybean was air-dried and cultivated in a greenhouse for 8 days, and then the area of lesion spots was investigated. As a result, it has been found that the area of lesion spots on the soybean treated with the present compound 1 to 9, 11 to 23, 26 to 33, 35 to 41, or 44 was 30% or less of that on a non-treated soybean.

### Test Example 10

Control Test on Soybean Rust (Phakopsora pachyrhizi)

Each of plastic pots was filled with soil and soybean (cultivar: KUROSENGOKU) was sowed and grown in a greenhouse for 13 days. Then, the present compound 1 to 9, 11 to 21, 29 to 37, or 44 formulated in accordance with the method mentioned in Formulation Example 6 was mixed with water so as to adjust the concentration to 200 ppm, and the mixture was sprayed over stems and leaves so that it sufficiently adhered to the surface of the leaves of the soybean. After spraying, the soybean was air-dried. After 4 days, an aqueous suspension containing spores of soybean rust fungus was sprayed to inoculate the spores. After inoculation, the soybean was at first left to stand in a greenhouse at 23° C. in the daytime and 20° C. at night under high humidity condition for 1 day, and then cultivated in a greenhouse for 10 days, and the area of lesion spots was investigated. As a result, the area of lesion spots on the soybean treated with the present compound 1 to 9, 11 to 21, 29 to 37, or 44 was 30% or less of that on a non-treated soybean.

### Test Example 11

Control Test on Barley Net Blotch (Pyrenophora teres)

Each of plastic pots was filled with soil and barley (cultivar: NISHINOHOSHI) was sowed and grown in a greenhouse for 7 days. Then, the present compound 2, 4, 7, or 36 formulated in accordance with the method mentioned in Formulation Example 6 was mixed with water so as to adjust the concentration to 500 ppm, and the mixture was sprayed over stems and leaves so that it sufficiently adhered to the surface of the leaves of the barley. After spraying, the barley was air-dried. After 2 days, an aqueous suspension containing spores of barley net blotch fungus was sprayed to inoculate the spores. After inoculation, the barley was at first left to stand in a greenhouse at 23° C. in the daytime and 20° C. at night under high humidity condition for 3 days, and then cultivated in a greenhouse for 7 days, and the area of lesion spots was investigated. As a result, the area of lesion spots on the barley treated with the present compound 2, 4, 7, or 36 was 30% or less of that on a non-treated barley.

### Test Example 12

Control Test on Barley Net Blotch (Pyrenophora teres)

Each of plastic pots was filled with soil and barley (cultivar: NISHINOHOSHI) was sowed and grown in a greenhouse for 7 days. Then, the present compound 10, 36, 42, or 44 formulated in accordance with the method mentioned in Formulation Example 6 was mixed with water so as to adjust the concentration to 200 ppm, and the mixture was sprayed over stems and leaves so that it sufficiently adhered to the surface of the leaves of the barley. After spraying, the barley was air-dried. After 2 days, an aqueous suspension containing spores of barley net blotch fungus was sprayed to inoculate the spores. After inoculation, the barley was at first left to stand in a greenhouse at 23° C. in the daytime and 20° C. at night under high humidity condition for 3 days, and then cultivated in a greenhouse for 7 days, and the area of lesion spots was investigated. As a result, the area of lesion spots on the barley treated with the present compound 10, 36, 42, or 44 was 30% or less of that on a non-treated barley.

### Test Example 13

Control Test on Soybean Powdery Mildew (Microsphaera diffusa)

Each of plastic pots was filled with soil and soybean (variety; KUROSENGOKU) was sowed and grown in a greenhouse for 10 days, and the spores of the soybean seedling (cultivar; KUROSENGOKU) infected by soybean powdery mildew fungus were sprinkling-inoculated. The soybean was cultivated in a greenhouse at 24° C. during the day and 20° C. at night. Then, the present compound 5, 6, 11, 16, or 35 formulated in accordance with the method mentioned in Formulation Example 6 was mixed with water so as to adjust the concentration to 200 ppm, and the mixture was sprayed over stems and leaves of the soybean so that it sufficiently adhered to the surface of the leaves of the soybean. After spraying, the soybean was air-dried and cultivated in a greenhouse for 9 days, and then the area of lesion spots was investigated. As a result, it has been found that the area of lesion spots on the soybean treated with the present compound 5, 6, 11, 16, or 35 was 30% or less of that on a non-treated soybean.

### Test Example 14

Control Test on Soybean Powdery Mildew (Microsphaera diffusa)

Each of plastic pots was filled with soil and soybean (variety; KUROSENGOKU) was sowed and grown in a greenhouse for 13 days. Then, the present compound 5, 6, 11, 12, 13, 16, 19, 20, 29, or 35 formulated in accordance with the method mentioned in Formulation Example 6 was mixed with water so as to adjust the concentration to 200 ppm, and the mixture was sprayed over stems and leaves of the soybean so that it sufficiently adhered to the surface of the leaves of the soybean. After spraying, the soybean was air-dried. After 1 day, the spores of the soybean seedling (cultivar; KUROSENGOKU) infected by soybean powdery mildew fungus were sprinkling-inoculated. After inoculation, the soybean was cultivated in a greenhouse at 24° C. during the day and 20° C. at night for 11 days, and then the area of lesion spots was investigated. As a result, it has been found that the area of lesion spots on the soybean treated with the present compound 5, 6, 11, 12, 13, 16, 19, 20, 29, or 35 was 30% or less of that on a non-treated soybean.

### Test Example 15

Control Test on Soybean Frogeye Leaf Spot (Cercospora sojina)

Each of plastic pots was filled with soil and soybean (cultivar: TACHINAGAHA) was sowed and grown in a greenhouse for 13 days. Thereafter, an aqueous suspension containing spores of soybean frogeye leaf spot (Cercospora sojina) fungus was sprayed to inoculate the spores. After inoculation, the soybean was at first left to stand in a greenhouse at 23° C. in the daytime under high humidity condition for 4 days, and cultivated in a greenhouse at 23° C. for 1 day. Then, the present compound 11 formulated in accordance with the method mentioned in Formulation Example 6 was mixed with water so as to adjust the concentration to 200 ppm, and the mixture was sprayed over stems and leaves of the soybean so that it sufficiently adhered to the surface of the leaves of the soybean. After spraying, the soybean was air-dried and cultivated in a greenhouse at 24° C. during the day and 20° C. at night for 21 days, and then the area of lesion spots was investigated. As a result, it has been found that the area of lesion spots on the soybean treated with the present compound 11 was 30% or less of that on a non-treated soybean.

### Test Example 16

Control Test on Tomato Late Blight (Phytophthora infestans)

Each of plastic pots was filled with soil and tomato (variety: PATIO) was sowed and grown in a greenhouse for 13 days. Then, the present compound 9 formulated in accordance with the method mentioned in Formulation Example 6 was mixed with water so as to adjust the concentration to 500 ppm, and the mixture was sprayed over stems and leaves so that it sufficiently adhered to the surface of the leaves of the tomato. After spraying, the tomato was air-dried. After 1 day, an aqueous suspension containing spores of tomato late blight fungus was sprayed to inoculate the spores. After inoculation, the tomato was at first left to stand in a greenhouse at 23° C. under high humidity condition for 1 day, and then cultivated in a greenhouse at 18° C. for 5 days, and the area of lesion spots was investigated. As a result, the area of lesion spots on the tomato treated with the present compound 9 was 30% or less of that on a non-treated tomato.

### Test Example 17

Control Test on Wheat Rust (Puccinia recondita)

Each of plastic pots was filled with soil and wheat (cultivar: SHIROGANE) was sowed and grown in a greenhouse for 9 days. Then, the present compound 2 to 13, 15, 18, 19, 30, 33, 35, 36, or 40 formulated in accordance with the method mentioned in Formulation Example 6 was mixed with water so as to adjust the concentration to 500 ppm, and the mixture was sprayed over stems and leaves of the wheat so that it sufficiently adhered to the surface of the leaves of the wheat. After spraying, the wheat was air-dried and cultivated at 20° C. for 5 days under illumination, and then inoculated by sprinkling with spores of wheat rust fungus.

After inoculation, the wheat was left to stand at 23° C. for 1 day under dark and high humidity condition, and cultivated under illumination at 20° C. for 8 days, and then the area of lesion spots was investigated. As a result, the area of lesion spots on the wheat treated with the present compound 2 to 13, 15, 18, 19, 30, 33, 35, 36, or 40 was 30% or less of that on a non-treated wheat.

### Test Example 18

Control Test on Wheat Leaf Blotch (Septoria tritici)

Each of plastic pots was filled with soil and wheat (cultivar: APOGEE) was sowed and grown in a greenhouse for 10 days. Then, the present compound 2 to 8, 10, 12, 30, 31, 34, 35, 36, 40, 42, 43, or 44 formulated in accordance with the method mentioned in Formulation Example 6 was mixed with water so as to adjust the concentration to 500 ppm, and the mixture was sprayed over stems and leaves so that it sufficiently adhered to the surface of the leaves of the wheat. After spraying, the wheat was air-dried. After 4 days, an aqueous suspension containing spores of wheat leaf blotch fungus was sprayed to inoculate the spores. After inoculation, the wheat was at first left to stand at 18° C. under high humidity condition for 3 days and then left to stand under illumination for 14 to 18 days, and the area of lesion spots was investigated. As a result, the area of lesion spots on the wheat treated with the present compound 2 to 8, 10, 12, 30, 31, 34, 35, 36, 40, 42, 43, or 44 was 30% or less of that on a non-treated wheat.

### Test Example 19

Control Test on Rice Blast (Magnaporthe grisea)

Each of plastic pots was filled with soil and rice (cultivar: HINOHIKARI) was sowed and grown in a greenhouse for 20 days. Then, the present compound 1 to 6, 9, 10, 20, 36, or 40 formulated in accordance with the method mentioned in Formulation Example 6 was mixed with water so as to adjust the concentration to 500 ppm, and the mixture was sprayed over stems and leaves so that it sufficiently adhered to the surface of the leaves of the rice. After spraying, the rice was air-dried and subjected to a spraying treatment and the rice seedling (cultivar: HINOHIKARI) infected by rice blast fungus left to stand for 6 days at 24° C. in the daytime and 20° C. at night under high humidity condition, while being in contact with each other, and then the area of lesion spots was investigated. As a result, the lesion areas on the rice treated with the present compound 1 to 6, 9, 10, 20, 36, or 40 were 30% or less with respect to the lesion area on the non-treated rice.

### Test Example 20

Control Test on Cucumber Powdery Mildew (Sphaerotheca fuliginea)

Each of plastic pots was filled with soil and cucumber (cultivar; SAGAMI HANJIRO) was sowed and grown in a greenhouse for 12 days. Then, the present compound 41 formulated in accordance with the method mentioned in Formulation Example 6 was mixed with water so as to adjust the concentration to 200 ppm, and the mixture was sprayed over stems and leaves of the cucumber so that it sufficiently adhered to the surface of the leaves of the cucumber. After spraying, the soybean was air-dried. After the cucumber was left to stand in a greenhouse at 24° C. during the day and 20° C. at night for 1 day, the spores of the cucumber seedling (cultivar; SAGAMI HANJIRO) infected by cucumber powdery mildew fungus were sprinkling-inoculated. After inoculation, the cucumber was cultivated in a greenhouse at 24° C. during the day and 20° C. at night for 8 days, and then the area of lesion spots was investigated. As a result, it has been found that the area of lesion spots on the cucumber treated with the present compound 41 was 30% or less of that on a non-treated cucumber.

### Reference Test Example 1

The present compound 11 or 31 formulated in accordance with the method mentioned in Formulation Example 6 was mixed with water so as to adjust the concentration to 500 ppm, and the mixture was sprayed over leaves of the cabbage so that it sufficiently adhered to the surface of the leaves of the cabbage seedling (second to three leaf development stage) planted in a container. After spraying, the cabbage was air-dried. The stems/leaves of this seedling were cut out and placed in a container in which filter paper was laid. Five second instar larvae of the diamondback moth were released in the container. After 5 days, the number of surviving insects was counted, and the mortality was determined by the following equation.

Mortality %=(1−number of surviving insects/5)×100

As a result, the mortality in the case of treating the present compound 11 or 31 was more than 80%.

### INDUSTRIAL APPLICABILITY

The present compound has control activity against plant diseases and is useful as an active ingredient of a plant disease control agent.

1. An oxadiazole compound represented by formula (I): 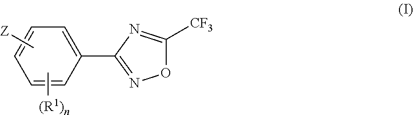
wherein
Z represents OC(═X)NR²R³, OC(═X)YR⁴, OC(═X)R⁵, NHC(═X)NR⁶R⁷, NHC(═X)YR⁸, NHC(═X)R⁹, SC(═X)NR¹⁰R¹¹, SC(═X)YR¹², or SC(═X)R¹³,
R¹ represents a halogen atom, a C1-C4 alkyl group optionally having one or more halogen atoms, a C1-C4 alkoxy group optionally having one or more halogen atoms, a cyano group, or a nitro group,
n represents an integer of any one of 0 to 4, wherein when n is 2 or more, plural R¹(s) may be the same or different to each other,
R² represents a C1-C10 alkyl group having one or more substituents selected from Group P¹, a C3-C4 alkenyl group having one or more substituents selected from Group P¹, a C5-C10 alkenyl group optionally having one or more substituents selected from Group P¹, a C3-C10 alkynyl group optionally having one or more substituents selected from Group P¹, a C3-C10 cycloalkyl group optionally having one or more substituents selected from Group P³, a C1-C4 alkoxy group having one or more substituents selected from Group P¹, a C5-C10 alkoxy group optionally having one or more substituents selected from Group P¹, a C3-C10 alkenyloxy group optionally having one or more substituents selected from Group P¹, or a C3-C10 alkynyloxy group optionally having one or more substituents selected from Group P¹,
R³, R⁷, and R¹¹ each independently represents a hydrogen atom, or a C1-C10 chain hydrocarbon group optionally having one or more substituents selected from Group P¹,
R⁴ and R⁵ each independently represents a C1-C10 alkyl group having one or more substituents selected from Group P¹, a C3-C4 alkenyl group having one or more substituents selected from Group P¹, a C5-C10 alkenyl group optionally having one or more substituents selected from Group P¹, a C3-C10 alkynyl group optionally having one or more substituents selected from Group P¹, a C3-C10 cycloalkyl group optionally having one or more substituents selected from Group P³, or a phenyl group having one or more substituents selected from Group P³,
R⁶ represents a C1-C4 alkyl group having one or more substituents selected from Group P², a C5-C10 alkyl group optionally having one or more substituents selected from Group P¹, a C3-C10 alkenyl group optionally having one or more substituents selected from Group P¹, a C3-C10 alkynyl group optionally having one or more substituents selected from Group P¹, a C3-C10 cycloalkyl group optionally having one or more substituents selected from Group P³, a C1-C4 alkoxy group having one or more substituents selected from Group P¹, a C5-C10 alkoxy group optionally having one or more substituents selected from Group P¹, or a phenyl group having one or more substituents selected from Group P³,
R⁸ and R⁹ each independently represents a C1-C10 alkyl group having one or more substituents selected from Group P², a C5-C10 haloalkyl group, a C3-C4 alkenyl group having one or more substituents selected from Group P¹, a C5-C10 alkenyl group optionally having one or more substituents selected from Group P¹, a C3-C10 alkynyl group optionally having one or more substituents selected from Group P¹, or a C3-C10 cycloalkyl group optionally having one or more substituents selected from Group P³,
R¹⁰, R¹², and R¹³ each independently represents a C1-C10 chain hydrocarbon group optionally having one or more substituents selected from Group P¹, a C3-C10 cycloalkyl group optionally having one or more substituents selected from Group P³, a C1-C10 alkoxy group optionally having one or more substituents selected from Group P¹, a C3-C10 alkenyloxy group optionally having one or more substituents selected from Group P¹, a C3-C10 alkynyloxy group optionally having one or more substituents selected from Group P¹, or a phenyl group having one or more substituents selected from Group P³, and
X and Y each independently represents an oxygen atom or a sulfur atom:
Group P¹: Group consisting of a halogen atom, a C1-C4 alkoxy group optionally having one or more halogen atoms, a cyano group, and a nitro group;
Group P²: Group consisting of a C1-C4 alkoxy group optionally having one or more halogen atoms, a cyano group, and a nitro group; and
Group P³: Group consisting of a halogen atom, a C1-C4 alkyl group optionally having one or more halogen atoms, a C1-C4 alkoxy group optionally having one or more halogen atoms, a cyano group, and a nitro group.

2. The compound according to claim 1, wherein Z is OC(═X)NR²R³.

3. The compound according to claim 2, wherein X is an oxygen atom and Z is bonded at the para-position with respect to the oxadiazole ring.

4. The compound according to claim 1, wherein n is 0.

5. The compound according to claim 1, wherein R³ is a methyl group.

6. The compound according to claim 1, wherein R² is a C1-C10 alkyl group which has a C1-C4 alkoxy group optionally having one or more halogen atoms.

7. The compound according to claim 1, wherein R² is a C1-C10 alkyl group having a cyano group.

8. The compound according to claim 1, wherein R² is a C3-C10 cycloalkyl group optionally having one or more substituents selected from Group P³.

9. The compound according to claim 1, wherein Z is OC(═X)NR²R³, OC(═X)R⁵, NHC(═X)NR⁶R⁷, or NHC(═X)R⁹, R¹ is a halogen atom or a C1-C4 alkyl group, n is 0 or 1, X is an oxygen atom, and Z is bonded at the meta- or para-position with respect to the oxadiazole ring.

10. The compound according to claim 9, wherein R¹ is a fluorine atom or a methyl group,
R² is a C1-C10 alkyl group having one or more substituents selected from Group P¹, a C3-C10 alkynyl group optionally having one or more substituents selected from Group P¹, or a C3-C10 cycloalkyl group optionally having one or more substituents selected from Group P³,
R³ is a hydrogen atom or a methyl group,
R⁵ is a C3-C10 cycloalkyl group optionally having one or more substituents selected from Group P³ or a phenyl group optionally having one or more substituents selected from Group P³,
R⁶ is a C1-C4 alkyl group having one or more substituents selected from Group P² or a C3-C10 cycloalkyl group optionally having one or more substituents selected from Group P³,
R⁷ is a methyl group,
R⁹ is a C1-C10 alkyl group having one or more substituents selected from Group P² or a C3-C10 cycloalkyl group optionally having one or more substituents selected from Group P³.

11. A plant disease control agent comprising the compound according to claim 1.

12. A method for controlling plant diseases, which comprises treating plants or soil with an effective amount of the compound according to claim 1.

13. Use of the compound according to claim 1 for controlling plant diseases.

14. A composition comprising the oxadiazole compound according to claim 1, and one or more selected from the group consisting of an insecticide, an acaricide, a nematicidal agent, a plant growth regulator, a synergist, and another plant disease control agent.
